SEQUENCE LISTING (505 sequences)

SEQ ID NO: 1 - RNA, 23 nt, Artificial Sequence
  Synthetic Sequence
ucgugcugaaaguauuuaauuga

SEQ ID NO: 2 - RNA, 23 nt, Artificial Sequence
  Synthetic Sequence
acuugcucgagucauaaauuaga

SEQ ID NO: 3 - RNA, 23 nt, Artificial Sequence
  Synthetic Sequence
acuaaccccuaauauuuauaaug

SEQ ID NO: 4 - RNA, 23 nt, Artificial Sequence
  Synthetic Sequence
cacuugccacucauauauuaacc

SEQ ID NO: 5 - RNA, 23 nt, Artificial Sequence
  Synthetic Sequence
aggcaagcggcgaagaauauucu

SEQ ID NO: 6 - RNA, 23 nt, Artificial Sequence
  Synthetic Sequence
caugucauuacacaaacuaaucu

SEQ ID NO: 7 - RNA, 23 nt, Artificial Sequence
  Synthetic Sequence
cuaggugaaacuuauaugaaagu

SEQ ID NO: 8 - RNA, 23 nt, Artificial Sequence
  Synthetic Sequence
gaaugcgcaacaccuaaacgaua

SEQ ID NO: 9 - RNA, 23 nt, Artificial Sequence
  Synthetic Sequence
cacugagcgguuucaaaagauca

SEQ ID NO: 10 - RNA, 23 nt, Artificial Sequence
  Synthetic Sequence
cuaugaccggcaccgauuugacg

SEQ ID NO: 11 - RNA, 23 nt, Artificial Sequence
  Synthetic Sequence
uaggaugccaucggucuuaaugu

SEQ ID NO: 12 - RNA, 23 nt, Artificial Sequence
  Synthetic Sequence
acguaugaacuggauccaauaug

SEQ ID NO: 13 - RNA, 23 nt, Artificial Sequence
  Synthetic Sequence
aagcggugccaaguacuucgaaa

SEQ ID NO: 14 - RNA, 23 nt, Artificial Sequence
  Synthetic Sequence
gaggcaaaugacgcgugaauacc

SEQ ID NO: 15 - RNA, 23 nt, Artificial Sequence
  Synthetic Sequence
cauccucugguuuucaucauaag

SEQ ID NO: 16 - RNA, 23 nt, Artificial Sequence
  Synthetic Sequence
ggucuucucucgaucaugauagc

SEQ ID NO: 17 - RNA, 23 nt, Artificial Sequence
  Synthetic Sequence
uaccuacacuaaucuagcauuga

SEQ ID NO: 18 - RNA, 23 nt, Artificial Sequence
  Synthetic Sequence
gcugaagcuaguaguucuguaac

SEQ ID NO: 19 - RNA, 23 nt, Artificial Sequence
  Synthetic Sequence
aaggaagggagaggaguucaaga

SEQ ID NO: 20 - RNA, 23 nt, Artificial Sequence
  Synthetic Sequence
cguuacucucggagcaaucuuuc

SEQ ID NO: 21 - RNA, 23 nt, Artificial Sequence
  Synthetic Sequence
caaugaugcaccaggaguucaag

SEQ ID NO: 22 - RNA, 23 nt, Artificial Sequence
  Synthetic Sequence
cuuuggaugaaaccuggaagaug

SEQ ID NO: 23 - RNA, 23 nt, Artificial Sequence
  Synthetic Sequence
gguuaucucuuccgagaugguca

SEQ ID NO: 24 - RNA, 23 nt, Artificial Sequence
  Synthetic Sequence
ccgagcaaguggugucgacuucc

SEQ ID NO: 25 - RNA, 23 nt, Artificial Sequence
  Synthetic Sequence
gccaauaucgauccuggaguaag

SEQ ID NO: 26 - RNA, 23 nt, Artificial Sequence
  Synthetic Sequence
ggugcccaaggaagucagcaaua

SEQ ID NO: 27 - RNA, 19 nt, Artificial Sequence
  Synthetic Sequence
uguaugcgaucgcagacua

SEQ ID NO: 28 - RNA, 21 nt, Artificial Sequence
  Synthetic Sequence
uagucugcgaucgcauacauu

SEQ ID NO: 29 - RNA, 19 nt, Artificial Sequence
  Synthetic Sequence
gugacacguucggagaauu

SEQ ID NO: 30 - RNA, 19 nt, Artificial Sequence
  Synthetic Sequence
aauucuccgaacgugucac

SEQ ID NO: 31 - RNA, 21 nt, Artificial Sequence
  Synthetic Sequence
gucaucacacugaauaccaau

SEQ ID NO: 32 - RNA, 21 nt, Artificial Sequence
  Synthetic Sequence
auugguauucagugugaugac

SEQ ID NO: 33 - RNA, 21 nt, Artificial Sequence
  Synthetic Sequence
uauuggaugauauccaauauu

SEQ ID NO: 34 - RNA, 21 nt, Artificial Sequence
  Synthetic Sequence
uauuggaugauaucaaauauu

SEQ ID NO: 35 - DNA, 21 bp, Artificial Sequence
  Synthetic Sequence
gucaucacacugagutccaau

SEQ ID NO: 36 - RNA, 21 nt, Artificial Sequence
  Synthetic Sequence
agagguagucagugugaugac

SEQ ID NO: 37 - RNA, 21 nt, Artificial Sequence
  Synthetic Sequence
gucaucacacugaguacgugu

SEQ ID NO: 38 - RNA, 21 nt, Artificial Sequence
  Synthetic Sequence
gucaucacacugacaagcuau

SEQ ID NO: 39 - RNA, 21 nt, Artificial Sequence
  Synthetic Sequence
gucaucacacugacccccugu

SEQ ID NO: 40 - RNA, 21 nt, Artificial Sequence
  Synthetic Sequence
gucaucacacugacuaccuau

SEQ ID NO: 41 - DNA, 22 bp, Artificial Sequence
  Synthetic Sequence
agagguagucaguagutccacu

SEQ ID NO: 42 - RNA, 21 nt, Artificial Sequence
  Synthetic Sequence
agagguagucaguguacgucu

SEQ ID NO: 43 - RNA, 21 nt, Artificial Sequence
  Synthetic Sequence
agagguagucagucaagcucu

SEQ ID NO: 44 - RNA, 21 nt, Artificial Sequence
  Synthetic Sequence
agagguagucagucccccucu

SEQ ID NO: 45 - RNA, 21 nt, Artificial Sequence
  Synthetic Sequence
agagguagucagucuaccucu

SEQ ID NO: 46 - RNA, 19 nt, Artificial Sequence
  Synthetic Sequence
ccaccacagcuagaacuua

SEQ ID NO: 47 - RNA, 19 nt, Artificial Sequence
  Synthetic Sequence
uaaguucuagcuguggugg

SEQ ID NO: 48 - RNA, 84 nt, Artificial Sequence
  Synthetic Sequence
cuggcugagguaguaguuugugcuguuggucggguugugacauugcccgcuguggagauaacugcgcaagcuacugccuugcua

SEQ ID NO: 49 - RNA, 22 nt, Artificial Sequence
  Synthetic Sequence
cugcgcaagcuacugccuugcu

SEQ ID NO: 50 - RNA, 22 nt, Artificial Sequence
  Synthetic Sequence
ugagguaguaguuugugcuguu

SEQ ID NO: 51 - RNA, 22 nt, Artificial Sequence
  Synthetic Sequence
cugcacaaacuacuaccucacu

SEQ ID NO: 52 - RNA, 22 nt, Artificial Sequence
  Synthetic Sequence
cagcacaaacuacuaccucacu

SEQ ID NO: 53 - RNA, 22 nt, Artificial Sequence
  Synthetic Sequence
ugagguaguaguuugugcuguu

SEQ ID NO: 54 - RNA, 19 nt, Artificial Sequence
  Synthetic Sequence
ccaccacagcuagaacuua

SEQ ID NO: 55 - RNA, 19 nt, Artificial Sequence
  Synthetic Sequence
uaaguucuagcuguggugg

SEQ ID NO: 56 - RNA, 22 nt, Artificial Sequence
  Synthetic Sequence
cugcgcaagcuacugccuugcu

SEQ ID NO: 57 - RNA, 22 nt, Artificial Sequence
  Synthetic Sequence
cagcacaaacuacuaccucacu

SEQ ID NO: 58 - RNA, 22 nt, Artificial Sequence
  Synthetic Sequence
ugagguaguaguuugugcuguu

SEQ ID NO: 59 - RNA, 10 nt, Artificial Sequence
  Synthetic Sequence
ccaccuauua

SEQ ID NO: 60 - RNA, 20 nt, Artificial Sequence
  Synthetic Sequence
cugcgcaagcuacugccuug

SEQ ID NO: 61 - RNA, 20 nt, Artificial Sequence
  Synthetic Sequence
cagcacaaacuacuaccuca

SEQ ID NO: 62 - RNA, 10 nt, Artificial Sequence
  Synthetic Sequence
cacauacuca

SEQ ID NO: 63 - RNA, 10 nt, Artificial Sequence
  Synthetic Sequence
cagaacuacc

SEQ ID NO: 64 - RNA, 20 nt, Artificial Sequence
  Synthetic Sequence
ugagguaguaguuugugcug

SEQ ID NO: 65 - RNA, 20 nt, Artificial Sequence
  Synthetic Sequence
cagcacaaacuacuaccuga

SEQ ID NO: 66 - RNA, 19 nt, Artificial Sequence
  Synthetic Sequence
ccaccacagcuagaacuua

SEQ ID NO: 67 - RNA, 19 nt, Artificial Sequence
  Synthetic Sequence
ccaccacagguagaacuua

SEQ ID NO: 68 - RNA, 19 nt, Artificial Sequence
  Synthetic Sequence
uaaguucuagcuguggugg

SEQ ID NO: 69 - RNA, 20 nt, Artificial Sequence
  Synthetic Sequence
cagcagaaacuacuaccuga

SEQ ID NO: 70 - RNA, 20 nt, Artificial Sequence
  Synthetic Sequence
ugagguaguaguuugugcug

SEQ ID NO: 71 - RNA, 20 nt, Artificial Sequence
  Synthetic Sequence
cagcacaaacuacuaccuga

SEQ ID NO: 72 - RNA, 20 nt, Artificial Sequence
  Synthetic Sequence
ugagguaguaguuugugcug

SEQ ID NO: 73 - RNA, 16 nt, Artificial Sequence
  Synthetic Sequence
ccaccacagcuagaac

SEQ ID NO: 74 - RNA, 16 nt, Artificial Sequence
  Synthetic Sequence
ccaccacagguagaac

SEQ ID NO: 75 - RNA, 16 nt, Artificial Sequence
  Synthetic Sequence
ccacgacagguagaac

SEQ ID NO: 76 - RNA, 16 nt, Artificial Sequence
  Synthetic Sequence
ccacgacagcuagaac

SEQ ID NO: 77 - RNA, 19 nt, Artificial Sequence
  Synthetic Sequence
uaaguucuagcuguggugg

SEQ ID NO: 78 - RNA, 19 nt, Artificial Sequence
  Synthetic Sequence
ccaccacagcuagaacuua

SEQ ID NO: 79 - RNA, 19 nt, Artificial Sequence
  Synthetic Sequence
ccaccacagguagaacuua

SEQ ID NO: 80 - RNA, 19 nt, Artificial Sequence
  Synthetic Sequence
ccacgacagguagaacuua

SEQ ID NO: 81 - RNA, 19 nt, Artificial Sequence
  Synthetic Sequence
ccacgacagcuagaacuua

SEQ ID NO: 82 - RNA, 17 nt, Artificial Sequence
  Synthetic Sequence
cagcagaaacuacuacc

SEQ ID NO: 83 - RNA, 20 nt, Artificial Sequence
  Synthetic Sequence
ugagguaguaguuugugcug

SEQ ID NO: 84 - RNA, 17 nt, Artificial Sequence
  Synthetic Sequence
cagcacaaacuacuacc

SEQ ID NO: 85 - RNA, 19 nt, Artificial Sequence
  Synthetic Sequence
ccaccacagcuagcaguua

SEQ ID NO: 86 - RNA, 19 nt, Artificial Sequence
  Synthetic Sequence
ccaccacagguagcaguua

SEQ ID NO: 87 - RNA, 19 nt, Artificial Sequence
  Synthetic Sequence
ccacgacagguagcaguua

SEQ ID NO: 88 - RNA, 19 nt, Artificial Sequence
  Synthetic Sequence
ccacgacagcuagcaguua

SEQ ID NO: 89 - RNA, 20 nt, Artificial Sequence
  Synthetic Sequence
cagcagaaacuacuacguga

SEQ ID NO: 90 - RNA, 20 nt, Artificial Sequence
  Synthetic Sequence
ugagguaguaguuugugcug

SEQ ID NO: 91 - RNA, 20 nt, Artificial Sequence
  Synthetic Sequence
cagcacaaacuacuacguga

SEQ ID NO: 92 - RNA, 19 nt, Artificial Sequence
  Synthetic Sequence
ccaccacagcuagaacuua

SEQ ID NO: 93 - RNA, 20 nt, Artificial Sequence
  Synthetic Sequence
cagcacaaacuacuaccuca

SEQ ID NO: 94 - RNA, 19 nt, Artificial Sequence
  Synthetic Sequence
caagugucaucacacugaa

SEQ ID NO: 95 - RNA, 19 nt, Artificial Sequence
  Synthetic Sequence
caagucucaucacacugaa

SEQ ID NO: 96 - RNA, 19 nt, Artificial Sequence
  Synthetic Sequence
uucagugugaugacacuug

SEQ ID NO: 97 - RNA, 19 nt, Artificial Sequence
  Synthetic Sequence
caagugucaucacacuuaa

SEQ ID NO: 98 - RNA, 19 nt, Artificial Sequence
  Synthetic Sequence
caagucucaucacacuuaa

SEQ ID NO: 99 - RNA, 19 nt, Artificial Sequence
  Synthetic Sequence
uucagugugaugacaguug

SEQ ID NO: 100 - RNA, 19 nt, Artificial Sequence
  Synthetic Sequence
gcggcacccucauaggccu

SEQ ID NO: 101 - RNA, 19 nt, Artificial Sequence
  Synthetic Sequence
gcggcacccucauaggcgu

SEQ ID NO: 102 - RNA, 19 nt, Artificial Sequence
  Synthetic Sequence
gcggcagccucauaggcgu

SEQ ID NO: 103 - RNA, 19 nt, Artificial Sequence
  Synthetic Sequence
aggccuaugagggugccgc

SEQ ID NO: 104 - RNA, 19 nt, Artificial Sequence
  Synthetic Sequence
gcggcacccucauagccgu

SEQ ID NO: 105 - RNA, 19 nt, Artificial Sequence
  Synthetic Sequence
gcggcagccucauagccgu

SEQ ID NO: 106 - RNA, 19 nt, Artificial Sequence
  Synthetic Sequence
gugcaagugcaaaccagac

SEQ ID NO: 107 - RNA, 19 nt, Artificial Sequence
  Synthetic Sequence
gugcaagugcaaaccagag

SEQ ID NO: 108 - RNA, 19 nt, Artificial Sequence
  Synthetic Sequence
gucugguuugcacuugcac

SEQ ID NO: 109 - RNA, 19 nt, Artificial Sequence
  Synthetic Sequence
gugcaagugcaaaccacag

SEQ ID NO: 110 - RNA, 19 nt, Artificial Sequence
  Synthetic Sequence
ccuccaggacgacuuugau

SEQ ID NO: 111 - RNA, 19 nt, Artificial Sequence
  Synthetic Sequence
ccucccggaggacuuugau

SEQ ID NO: 112 - RNA, 19 nt, Artificial Sequence
  Synthetic Sequence
ccucccggacgacuuugau

SEQ ID NO: 113 - RNA, 19 nt, Artificial Sequence
  Synthetic Sequence
aucaaagucguccuggagg

SEQ ID NO: 114 - RNA, 19 nt, Artificial Sequence
  Synthetic Sequence
ccuccaggacgacuaucau

SEQ ID NO: 115 - RNA, 19 nt, Artificial Sequence
  Synthetic Sequence
ccucccggaggacuaucau

SEQ ID NO: 116 - RNA, 19 nt, Artificial Sequence
  Synthetic Sequence
ccucccggacgacuaucau

SEQ ID NO: 117 - RNA, 20 nt, Artificial Sequence
  Synthetic Sequence
cagaccuauggaaacuacuu

SEQ ID NO: 118 - RNA, 20 nt, Artificial Sequence
  Synthetic Sequence
cagaccuaungaaacuacuu

SEQ ID NO: 119 - RNA, 15 nt, Artificial Sequence
  Synthetic Sequence
accuauggaaacuac

SEQ ID NO: 120 - RNA, 20 nt, Artificial Sequence
  Synthetic Sequence
aaguaguuuccauaggucug

SEQ ID NO: 121 - RNA, 20 nt, Artificial Sequence
  Synthetic Sequence
ccgucccaagcaauggacga

SEQ ID NO: 122 - RNA, 20 nt, Artificial Sequence
  Synthetic Sequence
ccguaccaaacaauggauga

SEQ ID NO: 123 - RNA, 20 nt, Artificial Sequence
  Synthetic Sequence
ccgucccaancaauggacga

SEQ ID NO: 124 - RNA, 15 nt, Artificial Sequence
  Synthetic Sequence
gucccaagcaaugga

SEQ ID NO: 125 - RNA, 20 nt, Artificial Sequence
  Synthetic Sequence
ucguccauugcuugggacgg

SEQ ID NO: 126 - RNA, 18 nt, Artificial Sequence
  Synthetic Sequence
ccgaguggaaggaaauuu

SEQ ID NO: 127 - RNA, 18 nt, Artificial Sequence
  Synthetic Sequence
aaauuuccuuccacucgg

SEQ ID NO: 128 - RNA, 18 nt, Artificial Sequence
  Synthetic Sequence
ggagaauauuucacccuu

SEQ ID NO: 129 - RNA, 18 nt, Artificial Sequence
  Synthetic Sequence
aagggugaaauauucucc

SEQ ID NO: 130 - RNA, 21 nt, Artificial Sequence
  Synthetic Sequence
ggagggagaauauuucacccu

SEQ ID NO: 131 - RNA, 22 nt, Artificial Sequence
  Synthetic Sequence
gagggugaaauauucucccucc

SEQ ID NO: 132 - RNA, 20 nt, Artificial Sequence
  Synthetic Sequence
ggacggaacagcuuugaggu

SEQ ID NO: 133 - RNA, 20 nt, Artificial Sequence
  Synthetic Sequence
accucaaagcuguuccgucc

SEQ ID NO: 134 - RNA, 21 nt, Artificial Sequence
  Synthetic Sequence
cauguucaagacagaagggcc

SEQ ID NO: 135 - RNA, 21 nt, Artificial Sequence
  Synthetic Sequence
ggcccuucugucuugaacaug

SEQ ID NO: 136 - DNA, 20 bp, Artificial Sequence
  Synthetic Sequence
ggaagacuguuacuacagtt

SEQ ID NO: 137 - RNA, 21 nt, Artificial Sequence
  Synthetic Sequence
agacuguaguaacagucuucc

SEQ ID NO: 138 - RNA, 20 nt, Artificial Sequence
  Synthetic Sequence
gugaugaaggacauggcuua

SEQ ID NO: 139 - RNA, 20 nt, Artificial Sequence
  Synthetic Sequence
uaagccauguccuucaucac

SEQ ID NO: 140 - RNA, 20 nt, Artificial Sequence
  Synthetic Sequence
gccaugaaggacauggcuua

SEQ ID NO: 141 - RNA, 20 nt, Artificial Sequence
  Synthetic Sequence
uaagccauguccuucauggc

SEQ ID NO: 142 - DNA, 20 bp, Artificial Sequence
  Synthetic Sequence
gaagcguaugacacauugat

SEQ ID NO: 143 - RNA, 20 nt, Artificial Sequence
  Synthetic Sequence
aucaaugugucauacgcuuc

SEQ ID NO: 144 - RNA, 19 nt, Artificial Sequence
  Synthetic Sequence
ggacauuacuagugacuca

SEQ ID NO: 145 - RNA, 19 nt, Artificial Sequence
  Synthetic Sequence
ugagucacuaguaaugucc

SEQ ID NO: 146 - RNA, 19 nt, Artificial Sequence
  Synthetic Sequence
uagacuuccugaauaacua

SEQ ID NO: 147 - RNA, 19 nt, Artificial Sequence
  Synthetic Sequence
uaguuauucaggaagucua

SEQ ID NO: 148 - RNA, 21 nt, Artificial Sequence
  Synthetic Sequence
aggguguauggcuucaacccu

SEQ ID NO: 149 - RNA, 21 nt, Artificial Sequence
  Synthetic Sequence
aggguugaagccauacacccu

SEQ ID NO: 150 - RNA, 23 nt, Artificial Sequence
  Synthetic Sequence
ggaguuugugacaaauaugggca

SEQ ID NO: 151 - RNA, 23 nt, Artificial Sequence
  Synthetic Sequence
ugcccauauuugucacaaacucc

SEQ ID NO: 152 - RNA, 20 nt, Artificial Sequence
  Synthetic Sequence
gauugauugaccuguccauu

SEQ ID NO: 153 - RNA, 20 nt, Artificial Sequence
  Synthetic Sequence
aauggacaggucaaucaauc

SEQ ID NO: 154 - RNA, 21 nt, Artificial Sequence
  Synthetic Sequence
guauucacacugaauaccaau

SEQ ID NO: 155 - RNA, 21 nt, Artificial Sequence
  Synthetic Sequence
auugguauucagugugaauac

SEQ ID NO: 156 - DNA, 22 bp, Artificial Sequence
  Synthetic Sequence
ggugcgaagcagacugaggcta

SEQ ID NO: 157 - DNA, 22 bp, Artificial Sequence
  Synthetic Sequence
tagccucagucugcuucgcacc

SEQ ID NO: 158 - RNA, 15 nt, Artificial Sequence
  Synthetic Sequence
ugcgaagcagacuga

SEQ ID NO: 159 - RNA, 21 nt, Artificial Sequence
  Synthetic Sequence
gccucagucugcuucgcaggc

SEQ ID NO: 160 - RNA, 19 nt, Artificial Sequence
  Synthetic Sequence
cggcauucggcuauguguu

SEQ ID NO: 161 - RNA, 19 nt, Artificial Sequence
  Synthetic Sequence
aacacauagccgaaugccg

SEQ ID NO: 162 - RNA, 16 nt, Artificial Sequence
  Synthetic Sequence
cggcauucggcuaugu

SEQ ID NO: 163 - RNA, 16 nt, Artificial Sequence
  Synthetic Sequence
acauagccgaaugccg

SEQ ID NO: 164 - RNA, 15 nt, Artificial Sequence
  Synthetic Sequence
cacagggcucacccu

SEQ ID NO: 165 - RNA, 20 nt, Artificial Sequence
  Synthetic Sequence
ucagggugagcccugugugu

SEQ ID NO: 166 - RNA, 18 nt, Artificial Sequence
  Synthetic Sequence
cuagaccuguuuugcuuu

SEQ ID NO: 167 - RNA, 18 nt, Artificial Sequence
  Synthetic Sequence
aaagcaaaacaggucuag

SEQ ID NO: 168 - RNA, 19 nt, Artificial Sequence
  Synthetic Sequence
gagguguaucuccuagaca

SEQ ID NO: 169 - RNA, 19 nt, Artificial Sequence
  Synthetic Sequence
ugucuaggagauacaccuc

SEQ ID NO: 170 - RNA, 19 nt, Artificial Sequence
  Synthetic Sequence
gagguguaucuccucgaca

SEQ ID NO: 171 - RNA, 19 nt, Artificial Sequence
  Synthetic Sequence
ugucgaggagauacaccuc

SEQ ID NO: 172 - RNA, 20 nt, Artificial Sequence
  Synthetic Sequence
ggguggucagcggccgggau

SEQ ID NO: 173 - RNA, 20 nt, Artificial Sequence
  Synthetic Sequence
aucccggccgcugaccaccc

SEQ ID NO: 174 - RNA, 20 nt, Artificial Sequence
  Synthetic Sequence
gcugcccacguggcuggcau

SEQ ID NO: 175 - RNA, 20 nt, Artificial Sequence
  Synthetic Sequence
augccagccacgugggcagc

SEQ ID NO: 176 - RNA, 15 nt, Artificial Sequence
  Synthetic Sequence
gcugcccacguggcu

SEQ ID NO: 177 - RNA, 20 nt, Artificial Sequence
  Synthetic Sequence
ccagccacgugggcagcagc

SEQ ID NO: 178 - RNA, 15 nt, Artificial Sequence
  Synthetic Sequence
ggugagggugucuac

SEQ ID NO: 179 - RNA, 21 nt, Artificial Sequence
  Synthetic Sequence
ggcguagacacccucaccgcc

SEQ ID NO: 180 - RNA, 20 nt, Artificial Sequence
  Synthetic Sequence
cguuagcagaaacaaaagga

SEQ ID NO: 181 - RNA, 20 nt, Artificial Sequence
  Synthetic Sequence
cguuagnagaaacaaaagga

SEQ ID NO: 182 - RNA, 20 nt, Artificial Sequence
  Synthetic Sequence
cguuagcagnaacaaaagga

SEQ ID NO: 183 - RNA, 20 nt, Artificial Sequence
  Synthetic Sequence
cguuaucagaaacaauagga

SEQ ID NO: 184 - RNA, 14 nt, Artificial Sequence
  Synthetic Sequence
uagcagaaacaaaa

SEQ ID NO: 185 - RNA, 20 nt, Artificial Sequence
  Synthetic Sequence
uccuuuuguuucugcuaacg

SEQ ID NO: 186 - RNA, 19 nt, Artificial Sequence
  Synthetic Sequence
aaguaaggaccagagacaa

SEQ ID NO: 187 - RNA, 15 nt, Artificial Sequence
  Synthetic Sequence
guaaggaccagagac

SEQ ID NO: 188 - RNA, 19 nt, Artificial Sequence
  Synthetic Sequence
uugucucugguccuuacuu

SEQ ID NO: 189 - RNA, 19 nt, Artificial Sequence
  Synthetic Sequence
uagguacagagacgucagu

SEQ ID NO: 190 - RNA, 19 nt, Artificial Sequence
  Synthetic Sequence
uagguacngagacgucagu

SEQ ID NO: 191 - RNA, 19 nt, Artificial Sequence
  Synthetic Sequence
acugacgucucuguaccua

SEQ ID NO: 192 - RNA, 19 nt, Artificial Sequence
  Synthetic Sequence
gggaaaauacauucuuucc

SEQ ID NO: 193 - RNA, 19 nt, Artificial Sequence
  Synthetic Sequence
gggaaaauacauucuuccc

SEQ ID NO: 194 - RNA, 19 nt, Artificial Sequence
  Synthetic Sequence
gggaaaauanauucuuccc

SEQ ID NO: 195 - RNA, 15 nt, Artificial Sequence
  Synthetic Sequence
gaaaauacauucuuc

SEQ ID NO: 196 - RNA, 19 nt, Artificial Sequence
  Synthetic Sequence
gggaagaauguauuuuccc

SEQ ID NO: 197 - RNA, 19 nt, Artificial Sequence
  Synthetic Sequence
ggguaaauacauucuucau

SEQ ID NO: 198 - RNA, 19 nt, Artificial Sequence
  Synthetic Sequence
ggguaaauanauucuucau

SEQ ID NO: 199 - RNA, 19 nt, Artificial Sequence
  Synthetic Sequence
gggcaaauacauucuccau

SEQ ID NO: 200 - RNA, 19 nt, Artificial Sequence
  Synthetic Sequence
gggcaaauacguucuccau

SEQ ID NO: 201 - RNA, 15 nt, Artificial Sequence
  Synthetic Sequence
guaaauacauucuuc

SEQ ID NO: 202 - RNA, 19 nt, Artificial Sequence
  Synthetic Sequence
augaagaauguauuuaccc

SEQ ID NO: 203 - RNA, 19 nt, Artificial Sequence
  Synthetic Sequence
ggguaaauacguucuucau

SEQ ID NO: 204 - RNA, 19 nt, Artificial Sequence
  Synthetic Sequence
ggguaaauanguucuucau

SEQ ID NO: 205 - RNA, 19 nt, Artificial Sequence
  Synthetic Sequence
ggguaaauacguucuccau

SEQ ID NO: 206 - RNA, 19 nt, Artificial Sequence
  Synthetic Sequence
gggcaaauacguucuccau

SEQ ID NO: 207 - RNA, 15 nt, Artificial Sequence
  Synthetic Sequence
guaaauacguucuuc

SEQ ID NO: 208 - RNA, 19 nt, Artificial Sequence
  Synthetic Sequence
augaagaacguauuuaccc

SEQ ID NO: 209 - RNA, 19 nt, Artificial Sequence
  Synthetic Sequence
gccuggaguuuauucggaa

SEQ ID NO: 210 - RNA, 19 nt, Artificial Sequence
  Synthetic Sequence
gccuggagunuauucggaa

SEQ ID NO: 211 - RNA, 19 nt, Artificial Sequence
  Synthetic Sequence
gccuguaguuuauucguaa

SEQ ID NO: 212 - RNA, 19 nt, Artificial Sequence
  Synthetic Sequence
gccuguagucuauucguaa

SEQ ID NO: 213 - RNA, 15 nt, Artificial Sequence
  Synthetic Sequence
cuggaguuuauucgg

SEQ ID NO: 214 - RNA, 19 nt, Artificial Sequence
  Synthetic Sequence
uuccgaauaaacuccaggc

SEQ ID NO: 215 - RNA, 19 nt, Artificial Sequence
  Synthetic Sequence
gaagcccagaggagcuaua

SEQ ID NO: 216 - RNA, 19 nt, Artificial Sequence
  Synthetic Sequence
gaagcucaguggaucuaua

SEQ ID NO: 217 - RNA, 19 nt, Artificial Sequence
  Synthetic Sequence
gaagcucagaggaucuaua

SEQ ID NO: 218 - RNA, 19 nt, Artificial Sequence
  Synthetic Sequence
gaagcccanaggagcuaua

SEQ ID NO: 219 - RNA, 14 nt, Artificial Sequence
  Synthetic Sequence
gcccagaggagcua

SEQ ID NO: 220 - RNA, 19 nt, Artificial Sequence
  Synthetic Sequence
uauagcuccucugggcuuc

SEQ ID NO: 221 - RNA, 19 nt, Artificial Sequence
  Synthetic Sequence
ucaaaguccgagagucagg

SEQ ID NO: 222 - RNA, 19 nt, Artificial Sequence
  Synthetic Sequence
ucaaaguccgagagucaga

SEQ ID NO: 223 - RNA, 19 nt, Artificial Sequence
  Synthetic Sequence
ucaaaguccgagagucaag

SEQ ID NO: 224 - RNA, 19 nt, Artificial Sequence
  Synthetic Sequence
ucaaaguccgagagucuag

SEQ ID NO: 225 - RNA, 19 nt, Artificial Sequence
  Synthetic Sequence
ucaaaguccnagagucuag

SEQ ID NO: 226 - RNA, 15 nt, Artificial Sequence
  Synthetic Sequence
aaaguccgagaguca

SEQ ID NO: 227 - RNA, 19 nt, Artificial Sequence
  Synthetic Sequence
ccugacucucggacuuuga

SEQ ID NO: 228 - RNA, 20 nt, Artificial Sequence
  Synthetic Sequence
uggaggagccgcagucagau

SEQ ID NO: 229 - RNA, 20 nt, Artificial Sequence
  Synthetic Sequence
uggaggagcngcagucagau

SEQ ID NO: 230 - RNA, 15 nt, Artificial Sequence
  Synthetic Sequence
gaggagccgcaguca

SEQ ID NO: 231 - RNA, 15 nt, Artificial Sequence
  Synthetic Sequence
gaggagcngcaguca

SEQ ID NO: 232 - RNA, 20 nt, Artificial Sequence
  Synthetic Sequence
aucugacugcggcuccucca

SEQ ID NO: 233 - RNA, 19 nt, Artificial Sequence
  Synthetic Sequence
auggaugauuugaugcugu

SEQ ID NO: 234 - RNA, 19 nt, Artificial Sequence
  Synthetic Sequence
auggaugauuugaugcagu

SEQ ID NO: 235 - RNA, 19 nt, Artificial Sequence
  Synthetic Sequence
auggaugaunugaugcugu

SEQ ID NO: 236 - RNA, 19 nt, Artificial Sequence
  Synthetic Sequence
auggaugaunugaugcagu

SEQ ID NO: 237 - RNA, 15 nt, Artificial Sequence
  Synthetic Sequence
ggaugauuugaugcu

SEQ ID NO: 238 - RNA, 19 nt, Artificial Sequence
  Synthetic Sequence
acagcaucaaaucauccau

SEQ ID NO: 239 - RNA, 21 nt, Artificial Sequence
  Synthetic Sequence
gucaucacacugaauaccaau

SEQ ID NO: 240 - RNA, 21 nt, Artificial Sequence
  Synthetic Sequence
gucaucacanugaauaccaau

SEQ ID NO: 241 - RNA, 15 nt, Artificial Sequence
  Synthetic Sequence
caucacacugaauac

SEQ ID NO: 242 - RNA, 15 nt, Artificial Sequence
  Synthetic Sequence
caucacanugaauac

SEQ ID NO: 243 - RNA, 21 nt, Artificial Sequence
  Synthetic Sequence
auugguauucagugugaugac

SEQ ID NO: 244 - RNA, 20 nt, Artificial Sequence
  Synthetic Sequence
gguguauggcuucaacccug

SEQ ID NO: 245 - RNA, 20 nt, Artificial Sequence
  Synthetic Sequence
gguguauggcuucaaccaug

SEQ ID NO: 246 - RNA, 20 nt, Artificial Sequence
  Synthetic Sequence
gguguauggnuucaacccug

SEQ ID NO: 247 - RNA, 15 nt, Artificial Sequence
  Synthetic Sequence
guauggcuucaaccc

SEQ ID NO: 248 - RNA, 20 nt, Artificial Sequence
  Synthetic Sequence
caggguugaagccauacacc

SEQ ID NO: 249 - RNA, 18 nt, Artificial Sequence
  Synthetic Sequence
ggaagaaaaaggaagccc

SEQ ID NO: 250 - RNA, 18 nt, Artificial Sequence
  Synthetic Sequence
ggaagaaaanggaagccc

SEQ ID NO: 251 - RNA, 18 nt, Artificial Sequence
  Synthetic Sequence
gggcuuccuuuuucuucc

SEQ ID NO: 252 - RNA, 22 nt, Artificial Sequence
  Synthetic Sequence
uggcucaguucagcaggaacag

SEQ ID NO: 253 - RNA, 22 nt, Artificial Sequence
  Synthetic Sequence
guuccugcugagcugagcuagu

SEQ ID NO: 254 - RNA, 22 nt, Artificial Sequence
  Synthetic Sequence
guuccugcugaacugagccagu

SEQ ID NO: 255 - RNA, 21 nt, Artificial Sequence
  Synthetic Sequence
cuggcucaguucagcaggaac

SEQ ID NO: 256 - RNA, 21 nt, Artificial Sequence
  Synthetic Sequence
cuggcucagnucagcaggaac

SEQ ID NO: 257 - RNA, 21 nt, Artificial Sequence
  Synthetic Sequence
cuggcgcagcucagcagguac

SEQ ID NO: 258 - RNA, 21 nt, Artificial Sequence
  Synthetic Sequence
cuggcacaguucagcagaaac

SEQ ID NO: 259 - RNA, 20 nt, Artificial Sequence
  Synthetic Sequence
cuggcucaguucagcaggaa

SEQ ID NO: 260 - RNA, 15 nt, Artificial Sequence
  Synthetic Sequence
gcucaguucagcagg

SEQ ID NO: 261 - RNA, 21 nt, Artificial Sequence
  Synthetic Sequence
guuccugcugaacugagccag

SEQ ID NO: 262 - RNA, 20 nt, Artificial Sequence
  Synthetic Sequence
uuccugcugaacugagccag

SEQ ID NO: 263 - RNA, 22 nt, Artificial Sequence
  Synthetic Sequence
uggcucaguucagcaggaacag

SEQ ID NO: 264 - RNA, 21 nt, Artificial Sequence
  Synthetic Sequence
uuccugcugagcugagcuagu

SEQ ID NO: 265 - RNA, 21 nt, Artificial Sequence
  Synthetic Sequence
uuccugcugaacugagccagu

SEQ ID NO: 266 - RNA, 22 nt, Artificial Sequence
  Synthetic Sequence
uagcaccaucugaaaucgguua

SEQ ID NO: 267 - RNA, 20 nt, Artificial Sequence
  Synthetic Sequence
acugauuucagaugguguua

SEQ ID NO: 268 - RNA, 20 nt, Artificial Sequence
  Synthetic Sequence
accgauuucagauggugcua

SEQ ID NO: 269 - RNA, 20 nt, Artificial Sequence
  Synthetic Sequence
gcaccaucugaaaucgguua

SEQ ID NO: 270 - RNA, 20 nt, Artificial Sequence
  Synthetic Sequence
gcaccaucunaaaucgguua

SEQ ID NO: 271 - RNA, 20 nt, Artificial Sequence
  Synthetic Sequence
gcaccagcuuaaaucguuua

SEQ ID NO: 272 - RNA, 20 nt, Artificial Sequence
  Synthetic Sequence
gcaccaucugaaaucgguua

SEQ ID NO: 273 - RNA, 20 nt, Artificial Sequence
  Synthetic Sequence
gcacccucugaaaucgauua

SEQ ID NO: 274 - RNA, 15 nt, Artificial Sequence
  Synthetic Sequence
accaucugaaaucgg

SEQ ID NO: 275 - RNA, 20 nt, Artificial Sequence
  Synthetic Sequence
uaaccgauuucagauggugc

SEQ ID NO: 276 - RNA, 23 nt, Artificial Sequence
  Synthetic Sequence
uagcaccauuugaaaucaguguu

SEQ ID NO: 277 - RNA, 20 nt, Artificial Sequence
  Synthetic Sequence
gcugguuucaaauggugcua

SEQ ID NO: 278 - RNA, 20 nt, Artificial Sequence
  Synthetic Sequence
acugauuucaaauggugcua

SEQ ID NO: 279 - RNA, 21 nt, Artificial Sequence
  Synthetic Sequence
gcaccauuugaaaucaguguu

SEQ ID NO: 280 - RNA, 21 nt, Artificial Sequence
  Synthetic Sequence
gcaccauuunaaaucaguguu

SEQ ID NO: 281 - RNA, 21 nt, Artificial Sequence
  Synthetic Sequence
gcacaauuunaaaucagcguu

SEQ ID NO: 282 - RNA, 21 nt, Artificial Sequence
  Synthetic Sequence
gcacaauuuaaaaucagcguu

SEQ ID NO: 283 - RNA, 21 nt, Artificial Sequence
  Synthetic Sequence
gcacaauuugaaaucaauguu

SEQ ID NO: 284 - RNA, 15 nt, Artificial Sequence
  Synthetic Sequence
ccauuugaaaucagu

SEQ ID NO: 285 - RNA, 21 nt, Artificial Sequence
  Synthetic Sequence
aacacugauuucaaauggugc

SEQ ID NO: 286 - RNA, 22 nt, Artificial Sequence
  Synthetic Sequence
uagcaccauuugaaaucgguua

SEQ ID NO: 287 - RNA, 22 nt, Artificial Sequence
  Synthetic Sequence
ugaccgauuucaaaugguguua

SEQ ID NO: 288 - RNA, 22 nt, Artificial Sequence
  Synthetic Sequence
uaaccgauuucaaauggugcua

SEQ ID NO: 289 - RNA, 20 nt, Artificial Sequence
  Synthetic Sequence
gcaccauuugaaaucgguua

SEQ ID NO: 290 - RNA, 20 nt, Artificial Sequence
  Synthetic Sequence
gcaccauuunaaaucgguua

SEQ ID NO: 291 - RNA, 20 nt, Artificial Sequence
  Synthetic Sequence
gcaccauuuaaaaucgguua

SEQ ID NO: 292 - RNA, 20 nt, Artificial Sequence
  Synthetic Sequence
gcaccacuuaaaaucaguua

SEQ ID NO: 293 - RNA, 20 nt, Artificial Sequence
  Synthetic Sequence
gcacccuuuaaaaucaguua

SEQ ID NO: 294 - RNA, 20 nt, Artificial Sequence
  Synthetic Sequence
gcacuauuugaaaucuguua

SEQ ID NO: 295 - RNA, 15 nt, Artificial Sequence
  Synthetic Sequence
accauuugaaaucgg

SEQ ID NO: 296 - RNA, 15 nt, Artificial Sequence
  Synthetic Sequence
accauuagaaaucgg

SEQ ID NO: 297 - RNA, 20 nt, Artificial Sequence
  Synthetic Sequence
uaaccgauuucaaauggugc

SEQ ID NO: 298 - RNA, 22 nt, Artificial Sequence
  Synthetic Sequence
uggagugugacaaugguguuug

SEQ ID NO: 299 - RNA, 20 nt, Artificial Sequence
  Synthetic Sequence
aaacgccauugucacacucc

SEQ ID NO: 300 - RNA, 20 nt, Artificial Sequence
  Synthetic Sequence
aaacaccauugucacacucc

SEQ ID NO: 301 - RNA, 20 nt, Artificial Sequence
  Synthetic Sequence
ggagugugacaaugguguuu

SEQ ID NO: 302 - RNA, 20 nt, Artificial Sequence
  Synthetic Sequence
ggagugugayaaugguguuu

SEQ ID NO: 303 - RNA, 20 nt, Artificial Sequence
  Synthetic Sequence
ggagugugagaauggucuuu

SEQ ID NO: 304 - RNA, 20 nt, Artificial Sequence
  Synthetic Sequence
ggagcgugagaauggucuuu

SEQ ID NO: 305 - RNA, 20 nt, Artificial Sequence
  Synthetic Sequence
ggagugcgagaauggucuuu

SEQ ID NO: 306 - RNA, 20 nt, Artificial Sequence
  Synthetic Sequence
ggagcgugacaauggucuuu

SEQ ID NO: 307 - RNA, 15 nt, Artificial Sequence
  Synthetic Sequence
agugugacaauggug

SEQ ID NO: 308 - RNA, 15 nt, Artificial Sequence
  Synthetic Sequence
agugugagaauggug

SEQ ID NO: 309 - RNA, 20 nt, Artificial Sequence
  Synthetic Sequence
aaacaccauugucacacucc

SEQ ID NO: 310 - RNA, 22 nt, Artificial Sequence
  Synthetic Sequence
uggagugugacaaugguguuug

SEQ ID NO: 311 - RNA, 68 nt, Artificial Sequence
  Synthetic Sequence
cuccggugccuacugagcugauaucaguucucauuucacacacuggcucaguucagcaggaacaggag

SEQ ID NO: 312 - RNA, 107 nt, Artificial Sequence
  Synthetic Sequence
gccucucuccgggcuccgccucccgugccuacugagcugaaacaguugauuccagugcacuggcucaguucagcaggaacaggaguccagcccccuaggagcuggca

SEQ ID NO: 313 - RNA, 23 nt, Artificial Sequence
  Synthetic Sequence
gugccuacugagcugauaucagu

SEQ ID NO: 314 - RNA, 22 nt, Artificial Sequence
  Synthetic Sequence
uggcucaguucagcaggaacag

SEQ ID NO: 315 - RNA, 22 nt, Artificial Sequence
  Synthetic Sequence
gugccuacugagcugaaacagu

SEQ ID NO: 316 - RNA, 22 nt, Artificial Sequence
  Synthetic Sequence
guuccugcugagcugagcuagu

SEQ ID NO: 317 - RNA, 22 nt, Artificial Sequence
  Synthetic Sequence
guuccugcugagcugagccagu

SEQ ID NO: 318 - RNA, 22 nt, Artificial Sequence
  Synthetic Sequence
guuccugcugaacugagccagu

SEQ ID NO: 319 - RNA, 22 nt, Artificial Sequence
  Synthetic Sequence
uggcucaguucagcaggaacag

SEQ ID NO: 320 - RNA, 23 nt, Artificial Sequence
  Synthetic Sequence
gugccuacunagcugauaucagu

SEQ ID NO: 321 - RNA, 23 nt, Artificial Sequence
  Synthetic Sequence
gugccuagugagcugauagcagu

SEQ ID NO: 322 - RNA, 16 nt, Artificial Sequence
  Synthetic Sequence
ccuacugagcugauau

SEQ ID NO: 323 - RNA, 22 nt, Artificial Sequence
  Synthetic Sequence
guuccugcunagcugagcuagu

SEQ ID NO: 324 - RNA, 22 nt, Artificial Sequence
  Synthetic Sequence
guuccugcugagcucagcuagu

SEQ ID NO: 325 - RNA, 15 nt, Artificial Sequence
  Synthetic Sequence
ccugcugagcugagc

SEQ ID NO: 326 - RNA, 22 nt, Artificial Sequence
  Synthetic Sequence
guuccugcuyaacugagccagu

SEQ ID NO: 327 - RNA, 22 nt, Artificial Sequence
  Synthetic Sequence
guucgugcugaacugaggcagu

SEQ ID NO: 328 - RNA, 15 nt, Artificial Sequence
  Synthetic Sequence
ccugcugaacugagc

SEQ ID NO: 329 - RNA, 22 nt, Artificial Sequence
  Synthetic Sequence
gugccuacunagcugaaacagu

SEQ ID NO: 330 - RNA, 16 nt, Artificial Sequence
  Synthetic Sequence
ccuacugagcugaaac

SEQ ID NO: 331 - RNA, 22 nt, Artificial Sequence
  Synthetic Sequence
guuccugcunagcugagccagu

SEQ ID NO: 332 - RNA, 22 nt, Artificial Sequence
  Synthetic Sequence
guucgugcugagcugaggcagu

SEQ ID NO: 333 - RNA, 15 nt, Artificial Sequence
  Synthetic Sequence
ccugcugagcugagc

SEQ ID NO: 334 - RNA, 22 nt, Artificial Sequence
  Synthetic Sequence
cggcuccggcugaacugagcca

SEQ ID NO: 335 - RNA, 22 nt, Artificial Sequence
  Synthetic Sequence
cggcuccggnugaacugagcca

SEQ ID NO: 336 - RNA, 22 nt, Artificial Sequence
  Synthetic Sequence
cggccccggnugaacugagaca

SEQ ID NO: 337 - RNA, 16 nt, Artificial Sequence
  Synthetic Sequence
cuccggcugaacugag

SEQ ID NO: 338 - RNA, 22 nt, Artificial Sequence
  Synthetic Sequence
ungcucaguucagcaggaacag

SEQ ID NO: 339 - RNA, 22 nt, Artificial Sequence
  Synthetic Sequence
uggcucaguucagccugagccg

SEQ ID NO: 340 - RNA, 22 nt, Artificial Sequence
  Synthetic Sequence
uggcucaguucagccggagccg

SEQ ID NO: 341 - RNA, 22 nt, Artificial Sequence
  Synthetic Sequence
uggcucaguucagccugaaccg

SEQ ID NO: 342 - RNA, 68 nt, Artificial Sequence
  Synthetic Sequence
cuccggugccuacugagcugauaucaguucucauuuuacacacuggcucaguucagcaggaacaggag

SEQ ID NO: 343 - RNA, 74 nt, Artificial Sequence
  Synthetic Sequence
cucugccucccgugccuacugagcugaaacacaguugguuuguguacacuuggcucaguucagcaggaacaggg

SEQ ID NO: 344 - RNA, 22 nt, Artificial Sequence
  Synthetic Sequence
ugccuacugagcugauaucagu

SEQ ID NO: 345 - RNA, 22 nt, Artificial Sequence
  Synthetic Sequence
ugccuacugagcugaaacacag

SEQ ID NO: 346 - RNA, 21 nt, Artificial Sequence
  Synthetic Sequence
uuccugcugagcugagcuagu

SEQ ID NO: 347 - RNA, 21 nt, Artificial Sequence
  Synthetic Sequence
uuccugcugaacugagccagu

SEQ ID NO: 348 - RNA, 22 nt, Artificial Sequence
  Synthetic Sequence
uggcucaguucagcaggaacag

SEQ ID NO: 349 - RNA, 22 nt, Artificial Sequence
  Synthetic Sequence
ugccuacunagcugauaucagu

SEQ ID NO: 350 - RNA, 22 nt, Artificial Sequence
  Synthetic Sequence
ugccuagugagcugauagcagu

SEQ ID NO: 351 - RNA, 15 nt, Artificial Sequence
  Synthetic Sequence
cuacugagcugauau

SEQ ID NO: 352 - RNA, 21 nt, Artificial Sequence
  Synthetic Sequence
uuccugcugngcugagcuagu

SEQ ID NO: 353 - RNA, 21 nt, Artificial Sequence
  Synthetic Sequence
uuccugcugagcucagcuagu

SEQ ID NO: 354 - RNA, 15 nt, Artificial Sequence
  Synthetic Sequence
ccugcugagcugagc

SEQ ID NO: 355 - RNA, 21 nt, Artificial Sequence
  Synthetic Sequence
uuccugcugnacugagccagu

SEQ ID NO: 356 - RNA, 21 nt, Artificial Sequence
  Synthetic Sequence
uucgugcugaacugaggcagu

SEQ ID NO: 357 - RNA, 15 nt, Artificial Sequence
  Synthetic Sequence
ccugcugaacugagc

SEQ ID NO: 358 - RNA, 21 nt, Artificial Sequence
  Synthetic Sequence
ugccuacugagcugaaacagu

SEQ ID NO: 359 - RNA, 21 nt, Artificial Sequence
  Synthetic Sequence
ugccuacugngcugaaacagu

SEQ ID NO: 360 - RNA, 16 nt, Artificial Sequence
  Synthetic Sequence
ccuacugagcugaaac

SEQ ID NO: 361 - RNA, 21 nt, Artificial Sequence
  Synthetic Sequence
uuccugcugagcugagccagu

SEQ ID NO: 362 - RNA, 21 nt, Artificial Sequence
  Synthetic Sequence
uuccugcugngcugagccagu

SEQ ID NO: 363 - RNA, 21 nt, Artificial Sequence
  Synthetic Sequence
uuccugcugagcugagccagu

SEQ ID NO: 364 - RNA, 15 nt, Artificial Sequence
  Synthetic Sequence
ccugcugngcugagc

SEQ ID NO: 365 - RNA, 89 nt, Artificial Sequence
  Synthetic Sequence
aaggccguggccucguucaaguaauccaggauaggcugugcaggucccaagggccuauucuugguuacuugcacggggacgcgggccug

SEQ ID NO: 366 - RNA, 84 nt, Artificial Sequence
  Synthetic Sequence
ggcugcggcuggauucaaguaauccaggauaggcuguguccguccaugaggccuguucuugauuacuuguuucuggaggcagcg

SEQ ID NO: 367 - RNA, 22 nt, Artificial Sequence
  Synthetic Sequence
ccuauucuugguuacuugcacg

SEQ ID NO: 368 - RNA, 22 nt, Artificial Sequence
  Synthetic Sequence
uucaaguaauccaggauaggcu

SEQ ID NO: 369 - RNA, 22 nt, Artificial Sequence
  Synthetic Sequence
ccuguucuugauuacuuguuuc

SEQ ID NO: 370 - RNA, 22 nt, Artificial Sequence
  Synthetic Sequence
ccuauucuggguuacuugaacg

SEQ ID NO: 371 - RNA, 22 nt, Artificial Sequence
  Synthetic Sequence
ccuguucuggauuacuugaauc

SEQ ID NO: 372 - RNA, 22 nt, Artificial Sequence
  Synthetic Sequence
ccuauccuggauuacuugaacg

SEQ ID NO: 373 - RNA, 22 nt, Artificial Sequence
  Synthetic Sequence
ccuauccuggauuacuugaauc

SEQ ID NO: 374 - RNA, 22 nt, Artificial Sequence
  Synthetic Sequence
uucaaguaauccaggauaggcu

SEQ ID NO: 375 - RNA, 22 nt, Artificial Sequence
  Synthetic Sequence
ccuauucuunguuacuugcacg

SEQ ID NO: 376 - RNA, 22 nt, Artificial Sequence
  Synthetic Sequence
ccuagucuugguuacuuacacg

SEQ ID NO: 377 - RNA, 15 nt, Artificial Sequence
  Synthetic Sequence
auucuugguuacuug

SEQ ID NO: 378 - RNA, 22 nt, Artificial Sequence
  Synthetic Sequence
ccuauucugnguuacuugaacg

SEQ ID NO: 379 - RNA, 22 nt, Artificial Sequence
  Synthetic Sequence
ccuagucuggguuacuuaaacg

SEQ ID NO: 380 - RNA, 15 nt, Artificial Sequence
  Synthetic Sequence
auucuggguuacuug

SEQ ID NO: 381 - RNA, 22 nt, Artificial Sequence
  Synthetic Sequence
ccuauccugnauuacuugaacg

SEQ ID NO: 382 - RNA, 22 nt, Artificial Sequence
  Synthetic Sequence
ccuagccuggauuacuuaaacg

SEQ ID NO: 383 - RNA, 15 nt, Artificial Sequence
  Synthetic Sequence
auccuggauuacuug

SEQ ID NO: 384 - RNA, 22 nt, Artificial Sequence
  Synthetic Sequence
ccuguucuunauuacuuguuuc

SEQ ID NO: 385 - RNA, 22 nt, Artificial Sequence
  Synthetic Sequence
ccuggucuugauuacuuauuuc

SEQ ID NO: 386 - RNA, 15 nt, Artificial Sequence
  Synthetic Sequence
guucuugauuacuug

SEQ ID NO: 387 - RNA, 22 nt, Artificial Sequence
  Synthetic Sequence
ccuguucugnauuacuugaauc

SEQ ID NO: 388 - RNA, 22 nt, Artificial Sequence
  Synthetic Sequence
ccuggucuggauuacuuaaauc

SEQ ID NO: 389 - RNA, 15 nt, Artificial Sequence
  Synthetic Sequence
guucuggauuacuug

SEQ ID NO: 390 - RNA, 22 nt, Artificial Sequence
  Synthetic Sequence
ccuauccugnauuacuugaauc

SEQ ID NO: 391 - RNA, 22 nt, Artificial Sequence
  Synthetic Sequence
ccuagccuggauuacuuaaauc

SEQ ID NO: 392 - RNA, 15 nt, Artificial Sequence
  Synthetic Sequence
auccuggauuacuug

SEQ ID NO: 393 - RNA, 22 nt, Artificial Sequence
  Synthetic Sequence
cucaaauaauggauuacuugaa

SEQ ID NO: 394 - RNA, 22 nt, Artificial Sequence
  Synthetic Sequence
cucaaauaanggauuacuugaa

SEQ ID NO: 395 - RNA, 22 nt, Artificial Sequence
  Synthetic Sequence
cucaaauaanggauuacuuuaa

SEQ ID NO: 396 - RNA, 16 nt, Artificial Sequence
  Synthetic Sequence
aaauaauggauuacuu

SEQ ID NO: 397 - RNA, 22 nt, Artificial Sequence
  Synthetic Sequence
uncaaguaauccaggauaggcu

SEQ ID NO: 398 - RNA, 22 nt, Artificial Sequence
  Synthetic Sequence
uucaaguaauccauuauuugag

SEQ ID NO: 399 - RNA, 22 nt, Artificial Sequence
  Synthetic Sequence
uucaaguaauccauuauaugag

SEQ ID NO: 400 - RNA, 76 nt, Artificial Sequence
  Synthetic Sequence
guggccucguucaaguaauccaggauaggcugugcaggucccaaugggccuauucuugguuacuugcacgggacgc

SEQ ID NO: 401 - RNA, 86 nt, Artificial Sequence
  Synthetic Sequence
ggcuguggcuggauucaaguaauccagggauaggcuguuuccaucugugaggccuauucuugauuacuuguuuccuggaggcagcu

SEQ ID NO: 402 - RNA, 22 nt, Artificial Sequence
  Synthetic Sequence
ccuauucuugguuacuugcacg

SEQ ID NO: 403 - RNA, 22 nt, Artificial Sequence
  Synthetic Sequence
uucaaguaauccaggauaggcu

SEQ ID NO: 404 - RNA, 22 nt, Artificial Sequence
  Synthetic Sequence
ccuauucuugauuacuuguuuc

SEQ ID NO: 405 - RNA, 22 nt, Artificial Sequence
  Synthetic Sequence
ccuauucuggguuacuugaacg

SEQ ID NO: 406 - RNA, 22 nt, Artificial Sequence
  Synthetic Sequence
ccuauucuggauuacuugaauc

SEQ ID NO: 407 - RNA, 22 nt, Artificial Sequence
  Synthetic Sequence
ccuauccuggauuacuugaacg

SEQ ID NO: 408 - RNA, 22 nt, Artificial Sequence
  Synthetic Sequence
ccuauccuggauuacuugaauc

SEQ ID NO: 409 - RNA, 22 nt, Artificial Sequence
  Synthetic Sequence
uucaaguaauccaggauaggcu

SEQ ID NO: 410 - RNA, 22 nt, Artificial Sequence
  Synthetic Sequence
ccuauucuunauuacuuguuuc

SEQ ID NO: 411 - RNA, 22 nt, Artificial Sequence
  Synthetic Sequence
ccuagucuugauuacuuauuuc

SEQ ID NO: 412 - RNA, 15 nt, Artificial Sequence
  Synthetic Sequence
auucuugauuacuug

SEQ ID NO: 413 - RNA, 22 nt, Artificial Sequence
  Synthetic Sequence
ccuauucugnauuacuugaauc

SEQ ID NO: 414 - RNA, 22 nt, Artificial Sequence
  Synthetic Sequence
ccuagucuggauuacuuaaauc

SEQ ID NO: 415 - RNA, 15 nt, Artificial Sequence
  Synthetic Sequence
auucuggauuacuug

SEQ ID NO: 416 - RNA, 88 nt, Artificial Sequence
  Synthetic Sequence
accccuuagaggaugacugauuucuuuugguguucagagucaauagaauuuucuagcaccaucugaaaucgguuauaaugauugggga

SEQ ID NO: 417 - RNA, 22 nt, Artificial Sequence
  Synthetic Sequence
acugauuucuuuugguguucag

SEQ ID NO: 418 - RNA, 22 nt, Artificial Sequence
  Synthetic Sequence
uagcaccaucugaaaucgguua

SEQ ID NO: 419 - RNA, 20 nt, Artificial Sequence
  Synthetic Sequence
acugauuucagaugguguua

SEQ ID NO: 420 - RNA, 20 nt, Artificial Sequence
  Synthetic Sequence
accgauuucagauggugcua

SEQ ID NO: 421 - RNA, 22 nt, Artificial Sequence
  Synthetic Sequence
uagcaccaucugaaaucgguua

SEQ ID NO: 422 - RNA, 71 nt, Artificial Sequence
  Synthetic Sequence
aggaagcugguuucauauggugguuuagauuuaaauagugauugucuagcaccauuugaaaucaguguucu

SEQ ID NO: 423 - RNA, 81 nt, Artificial Sequence
  Synthetic Sequence
cuucuggaagcugguuucacaugguggcuuagauuuuuccaucuuuguaucuagcaccauuugaaaucaguguuuuaggag

SEQ ID NO: 424 - RNA, 22 nt, Artificial Sequence
  Synthetic Sequence
gcugguuucauauggugguuua

SEQ ID NO: 425 - RNA, 23 nt, Artificial Sequence
  Synthetic Sequence
uagcaccauuugaaaucaguguu

SEQ ID NO: 426 - RNA, 25 nt, Artificial Sequence
  Synthetic Sequence
cugguuucacaugguggcuuagauu

SEQ ID NO: 427 - RNA, 20 nt, Artificial Sequence
  Synthetic Sequence
gcugguuucaaauggugcua

SEQ ID NO: 428 - RNA, 23 nt, Artificial Sequence
  Synthetic Sequence
cugguuucaaauggugcuagauu

SEQ ID NO: 429 - RNA, 20 nt, Artificial Sequence
  Synthetic Sequence
acugauuucaaauggugcua

SEQ ID NO: 430 - RNA, 23 nt, Artificial Sequence
  Synthetic Sequence
cugauuucaaauggugcuagauu

SEQ ID NO: 431 - RNA, 23 nt, Artificial Sequence
  Synthetic Sequence
uagcaccauuugaaaucaguguu

SEQ ID NO: 432 - RNA, 88 nt, Artificial Sequence
  Synthetic Sequence
aucucuuacacaggcugaccgauuucuccugguguucagagucuguuuuugucuagcaccauuugaaaucgguuaugauguaggggga

SEQ ID NO: 433 - RNA, 22 nt, Artificial Sequence
  Synthetic Sequence
ugaccgauuucuccugguguuc

SEQ ID NO: 434 - RNA, 22 nt, Artificial Sequence
  Synthetic Sequence
uagcaccauuugaaaucgguua

SEQ ID NO: 435 - RNA, 22 nt, Artificial Sequence
  Synthetic Sequence
ugaccgauuucaaaugguguua

SEQ ID NO: 436 - RNA, 22 nt, Artificial Sequence
  Synthetic Sequence
uaaccgauuucaaauggugcua

SEQ ID NO: 437 - RNA, 22 nt, Artificial Sequence
  Synthetic Sequence
uagcaccauuugaaaucgguua

SEQ ID NO: 438 - RNA, 64 nt, Artificial Sequence
  Synthetic Sequence
augacugauuucuuuugguguucagagucaauauaauuuucuagcaccaucugaaaucgguuau

SEQ ID NO: 439 - RNA, 82 nt, Artificial Sequence
  Synthetic Sequence
cuucaggaagcugguuucauauggugguuuagauuuaaauagugaauugucuagcaccauuugaaaucaguguucuuggggg

SEQ ID NO: 440 - RNA, 81 nt, Artificial Sequence; identical to SEQ ID NO: 423
  Synthetic Sequence

SEQ ID NO: 441 - RNA, 89 nt, Artificial Sequence
  Synthetic Sequence
aucucuuacacaggcugaccgauuucuccugguguucagagucuguuuuugucuagcaccauuugaaaaucgguuaugauguaggggga

SEQ ID NO: 442 - RNA, 66 nt, Artificial Sequence
  Synthetic Sequence
agcuguggagugugacaaugguguuuguguccaaaccaucaaacgccauuaucacacuaaauagcu

SEQ ID NO: 443 - RNA, 20 nt, Artificial Sequence
  Synthetic Sequence
aaacgccauuaucacacuaa

SEQ ID NO: 444 - RNA, 22 nt, Artificial Sequence
  Synthetic Sequence
uggagugugacaaugguguuug

SEQ ID NO: 445 - RNA, 20 nt, Artificial Sequence
  Synthetic Sequence
aaacgccauugucacacucc

SEQ ID NO: 446 - RNA, 20 nt, Artificial Sequence
  Synthetic Sequence
aaacaccauugucacacucc

SEQ ID NO: 447 - RNA, 22 nt, Artificial Sequence
  Synthetic Sequence
uggagugugacaaugguguuug

SEQ ID NO: 448 - RNA, 85 nt, Artificial Sequence
  Synthetic Sequence
ccuuagcagagcuguggagugugacaaugguguuugugucuaaacuaucaaacgccauuaucacacuaaauagcuacugcuaggc

SEQ ID NO: 449 - RNA, 22 nt, Artificial Sequence
  Synthetic Sequence
aacgccauuaucacacuaaaua

SEQ ID NO: 450 - RNA, 22 nt, Artificial Sequence
  Synthetic Sequence
uggagugugacaaugguguuug

SEQ ID NO: 451 - RNA, 22 nt, Artificial Sequence
  Synthetic Sequence
aacgccauugucacacuccaua

SEQ ID NO: 452 - RNA, 22 nt, Artificial Sequence
  Synthetic Sequence
aacaccauugucacacuccaua

SEQ ID NO: 453 - RNA, 22 nt, Artificial Sequence
  Synthetic Sequence
uggagugugacaaugguguuug

SEQ ID NO: 454 - RNA, 65 nt, Artificial Sequence
  Synthetic Sequence
agcucugagaacugaauuccauggguuauaucaaugucagaccugugaaauucaguucuucagcu

SEQ ID NO: 455 - RNA, 22 nt, Artificial Sequence
  Synthetic Sequence
ccugugaaauucaguucuucag

SEQ ID NO: 456 - RNA, 22 nt, Artificial Sequence
  Synthetic Sequence
ugagaacugaauuccauggguu

SEQ ID NO: 457 - RNA, 22 nt, Artificial Sequence
  Synthetic Sequence
ccuguggaauucaguucuuaag

SEQ ID NO: 458 - RNA, 22 nt, Artificial Sequence
  Synthetic Sequence
cccauggaauucaguucucaag

SEQ ID NO: 459 - RNA, 22 nt, Artificial Sequence
  Synthetic Sequence
ugagaacugaauuccauggguu

SEQ ID NO: 460 - RNA, 99 nt, Artificial Sequence
  Synthetic Sequence
ccgauguguauccucagcuuugagaacugaauuccauggguugugucagugucagaccucugaaauucaguucuucagcugggauaucucugucaucgu

SEQ ID NO: 461 - RNA, 22 nt, Artificial Sequence
  Synthetic Sequence
ccucugaaauucaguucuucag

SEQ ID NO: 462 - RNA, 22 nt, Artificial Sequence
  Synthetic Sequence
ugagaacugaauuccauggguu

SEQ ID NO: 463 - RNA, 22 nt, Artificial Sequence
  Synthetic Sequence
ccuauggaauucaguucuuaag

SEQ ID NO: 464 - RNA, 22 nt, Artificial Sequence
  Synthetic Sequence
cccauggaauucaguucucaag

SEQ ID NO: 465 - RNA, 22 nt, Artificial Sequence
  Synthetic Sequence
ugagaacugaauuccauggguu

SEQ ID NO: 466 - RNA, 76 nt, Artificial Sequence
  Synthetic Sequence
gccugguccagugguucuugacaguucaacaguucuguagcacaauugugaaauguuuaggaccacuagacccggc

SEQ ID NO: 467 - RNA, 22 nt, Artificial Sequence
  Synthetic Sequence
agugguucuugacaguucaaca

SEQ ID NO: 468 - RNA, 22 nt, Artificial Sequence
  Synthetic Sequence
gugaaauguuuaggaccacuag

SEQ ID NO: 469 - RNA, 21 nt, Artificial Sequence
  Synthetic Sequence
agugguucuagacauuucaca

SEQ ID NO: 470 - RNA, 21 nt, Artificial Sequence
  Synthetic Sequence
agugguccuaaacauuucaca

SEQ ID NO: 471 - RNA, 22 nt, Artificial Sequence
  Synthetic Sequence
gugaaauguuuaggaccacuag

SEQ ID NO: 472 - RNA, 110 nt, Artificial Sequence
  Synthetic Sequence
guguuggggacucgcgcgcuggguccagugguucuuaacaguucaacaguucuguagcgcaauugugaaauguuuaggaccacuagacccggcgggcgcggcgacagcga

SEQ ID NO: 473 - RNA, 22 nt, Artificial Sequence
  Synthetic Sequence
agugguucuuaacaguucaaca

SEQ ID NO: 474 - RNA, 22 nt, Artificial Sequence
  Synthetic Sequence
gugaaauguuuaggaccacuag

SEQ ID NO: 475 - RNA, 21 nt, Artificial Sequence
  Synthetic Sequence
agugguucuaaacauuucaca

SEQ ID NO: 476 - RNA, 22 nt, Artificial Sequence
  Synthetic Sequence
gugaaauguuuaggaccacuag

SEQ ID NO: 477 - RNA, 110 nt, Artificial Sequence
  Synthetic Sequence
ggccuggcuggacagaguugucaugugucugccugucuacacuugcugugcagaacauccgcucaccuguacagcaggcacagacaggcagucacaugacaacccagccu

SEQ ID NO: 478 - RNA, 22 nt, Artificial Sequence
  Synthetic Sequence
ugccugucuacacuugcugugc

SEQ ID NO: 479 - RNA, 22 nt, Artificial Sequence
  Synthetic Sequence
acagcaggcacagacaggcagu

SEQ ID NO: 480 - RNA, 22 nt, Artificial Sequence
  Synthetic Sequence
ugccugucugugcuugcugugc

SEQ ID NO: 481 - RNA, 22 nt, Artificial Sequence
  Synthetic Sequence
ugccugucugugccugcugugc

SEQ ID NO: 482 - RNA, 22 nt, Artificial Sequence
  Synthetic Sequence
acagcaggcacagacaggcagu

SEQ ID NO: 483 - RNA, 110 nt, Artificial Sequence; identical to SEQ ID NO: 477
  Synthetic Sequence

SEQ ID NO: 484 - RNA, 22 nt, Artificial Sequence
  Synthetic Sequence
ugccugucuacacuugcugugc

SEQ ID NO: 485 - RNA, 22 nt, Artificial Sequence
  Synthetic Sequence
acagcaggcacagacaggcagu

SEQ ID NO: 486 - RNA, 22 nt, Artificial Sequence
  Synthetic Sequence
ugccugucugugcuugcugugc

SEQ ID NO: 487 - RNA, 22 nt, Artificial Sequence
  Synthetic Sequence
ugccugucugugccugcugugc

SEQ ID NO: 488 - RNA, 22 nt, Artificial Sequence
  Synthetic Sequence
acagcaggcacagacaggcagu

SEQ ID NO: 489 - RNA, 79 nt, Artificial Sequence
  Synthetic Sequence
gggugggcagcuguuaagacuugcagugauguuuagcuccucugcaugugaacaucacagcaagucugugcugcugccu

SEQ ID NO: 490 - RNA, 23 nt, Artificial Sequence
  Synthetic Sequence
gaacaucacagcaagucugugcu

SEQ ID NO: 491 - RNA, 21 nt, Artificial Sequence
  Synthetic Sequence
uuaagacuugcagugauguuu

SEQ ID NO: 492 - RNA, 23 nt, Artificial Sequence
  Synthetic Sequence
gaacaucacugcaagucuuagcu

SEQ ID NO: 493 - RNA, 23 nt, Artificial Sequence
  Synthetic Sequence
aaacaucacugcaagucuuaacu

SEQ ID NO: 494 - RNA, 21 nt, Artificial Sequence
  Synthetic Sequence
uuaagacuugcagugauguuu

SEQ ID NO: 495 - RNA, 122 nt, Artificial Sequence
  Synthetic Sequence
gcccuguccccugugccuugggcgggcggcuguuaagacuugcagugauguuuaacuccucuccacgugaacaucacagcaagucugugcugcuucccgucccuacgcugccugggcagggu

SEQ ID NO: 496 - RNA, 22 nt, Artificial Sequence
  Synthetic Sequence
aacaucacagcaagucugugcu

SEQ ID NO: 497 - RNA, 21 nt, Artificial Sequence
  Synthetic Sequence
uuaagacuugcagugauguuu

SEQ ID NO: 498 - RNA, 21 nt, Artificial Sequence
  Synthetic Sequence
uuaagacuugcagugauguuu

SEQ ID NO: 499 - RNA, 22 nt, Artificial Sequence
  Synthetic Sequence
aacaucacagcaagucugugcu

SEQ ID NO: 500 - RNA, 22 nt, Artificial Sequence
  Synthetic Sequence
aacaucacugcaagucuuagcu

SEQ ID NO: 501 - RNA, 21 nt, Artificial Sequence
  Synthetic Sequence
uacagacuugcugugauguuu

SEQ ID NO: 502 - RNA, 22 nt, Artificial Sequence
  Synthetic Sequence
aacaucacugcaagucuuaacu

SEQ ID NO: 503 - RNA, 21 nt, Artificial Sequence
  Synthetic Sequence
cacagacuugcugugauguuu

SEQ ID NO: 504 - RNA, 21 nt, Artificial Sequence
  Synthetic Sequence
uuaagacuugcagugauguuu

SEQ ID NO: 505 - RNA, 22 nt, Artificial Sequence
  Synthetic Sequence
aacaucacagcaagucugugcu

(19) United States
(12) Patent Application Publication
(10) Pub. No.: US 2015/0038549 A1
(43) Pub. Date: Feb. 5, 2015

(54) Methods and Compositions for Modulating Gene Expression Using Components That Self Assemble in Cells and Produce RNAi Activity

(71) Applicant: Larry J. Smith, Omaha, NE (US)

(72) Inventor: Larry J. Smith, Omaha, NE (US)

(21) Appl. No.: 14/113,093

(22) PCT Filed: Apr. 20, 2012
(86) PCT No.: PCT/US12/34595

§ 371 Date: Jun. 9, 2014

Related U.S. Application Data
(60) Provisional application No. 61/477,283, filed on Apr. 20, 2011.
(60) Provisional application No. 61/477,291, filed on Apr. 20, 2011.
(60) Provisional application No. 61/477,875, filed on Apr. 21, 2011.

(51) Int. Cl. C12N 15/113

(52) U.S. Cl. CPC C12N 15/113
USPC 514/44 A

(57) ABSTRACT

Compositions and methods for downmodulating expression of target nucleic acids are disclosed.

This application claims priority to US Provisional Application Nos: 61/477,283, 61/477,291 each filed Apr. 20, 2011 and 61/477,875 filed Apr. 21, 2011 respectively, the disclosure of all of the foregoing applications being incorporated herein by reference as though set forth in full.

### FIELD OF THE INVENTION

This invention relates to the fields of medicine, drug development and modulation of gene expression. More specifically, the invention provides compositions and methods of use thereof that facilitate the modulation of gene expression using novel oligonucleotide based drugs that produce an inhibitory RNA (RNAi) mechanism of action.

### BACKGROUND OF THE INVENTION

Numerous publications and patent documents, including both published applications and issued patents, are cited throughout the specification in order to describe the state of the art to which this invention pertains. Each of these citations is incorporated herein by reference as though set forth in full.

RNA interference (RNAi) refers to molecules and mechanisms whereby certain double stranded RNA (dsRNA) structures (RNAi triggers) cause sequence specific gene inhibition. Two main categories of RNAi have been distinguished: small inhibitory RNA (siRNA) and microRNA (miRNA). In the case of naturally occurring siRNA the original source of the dsRNA is exogenous to the cell or it is derived from transposable elements within the cell. Cells may then process the dsRNA to produce siRNA that can specifically suppress the activity of the source of the dsRNA. The exogenous sources include certain viruses where the siRNA generated provides a defense mechanism against such invaders.

In contrast, naturally occurring miRNA is produced from precursor molecules that are generated from independent genes or from very short intron sequences found in some protein encoding genes. Unlike siRNA molecules, miRNA molecules broadly inhibit multiple different genes rather than being narrowly focused on a particular gene. Thus, naturally occurring siRNA characteristically performs more narrowly focused inhibitory actions than does miRNA.

These differences are reflected, in part, in the “targeting codes” that are associated with these two classes of RNAi. The targeting code can be briefly defined as the subset of the antisense strand sequence that is primarily or fully responsible for recognizing the target sequence by complementary base pairing. (Ambros et al., RNA, provide a more detailed description of how naturally occurring siRNA and miRNA can be experimentally distinguished and annotated 9: 277-279, 2003.)

The general mechanisms that underlie the implementation of siRNA and miRNA-dependent activity are substantially overlapping, but the particulars of how siRNA and miRNA function to suppress gene expression are substantially different. At the heart of the general mechanisms applicable to both of these types of RNAi is the RNA-induced silencing complex (RISC). The double stranded siRNA or miRNA is loaded into RISC. Next the sense strand is discarded and the antisense strand is used to direct RISC to its target(s).

In the case of siRNA typically and for a subset of miRNAs, the RISC complex includes an enzyme called argonaute-2 (AGO-2) that cleaves a specific mRNA target. Other enzymes recognize the bifurcated mRNA as abnormal and further degrade it. mRNA cleavage by AGO-2 requires a high degree of sequence complementarity between the guide strand and its target particularly with respect to the nucleosides adjacent to the AGO-2 cleavage side that are located a positions 10 and 11 counting from the 5′-end of the guide strand along with several of the nucleosides on either side of positions 10 and 11. The nucleoside sequence found at this location (central region) is the targeting code in this context. Typically a perfect complementarity between the targeting code nucleosides and the corresponding target nucleosides is required for AGO-2 based cleavage. Additional nucleosides out side of this targeting code can also affect the efficiency of the target recognition and functional inhibition by RISC but some mismatches can be tolerated in these flanking areas.

Genome wide identification of miRNA targets and computational predictions estimate that each mammalian miRNA on average inhibits the expression of hundreds of different mRNAs. Thus, miRNA can be involved in coordinating patterns of gene expression. The ability of particular miRNAs to produce a particular cellular phenotype, however, can be based on the modulation of the expression of as few genes as one. Most mammalian genes appear to be post-transcriptionally regulated by miRNAs. Abnormalities in the expression of particular miRNAs have pathogenic roles in a wide range of medical disorders.

The targeting code most commonly used by miRNA resides in a so called “seed sequence” that is made up of nucleosides 2-8(or 2-7) counting in from the 5′-end of the guide or antisense strand. This sequence is the major determinant of target recognition and is sufficient to trigger translational silencing. Target sequences are found in the 3′-untranslated region (3′UTR) of the mRNA targets. Infrequently, complementarity between nucleosides down-stream of the seed sequence and the target contribute to target recognition particularly when the seed sequence has a weak match with the target. These are called 3′-supplementary or 3′-compensatory sites.

Another category of miRNA utilizes a target code involving “centered sites” that consist of 11 or 12 consecutive nucleosides that begin at position 4 or 5 downstream from the 5′-end of the guide or antisense strand. To date no 3′-supplementary or 3′-compensatory sites have been uncovered that support target recognition by the targeting code.

MiRNA, other than the few with a siRNA-like inhibitory mechanism, can suppress the translation of specific sets of mRNA by interfering with the translation machinery without affecting mRNA levels and/or by causing the mRNA to be degraded by promoting the conditions necessary to activate the naturally occurring 5′-to-3′ mRNA decay pathway.

In addition to the common targeting of the 3′UTR of mRNA, some miRNAs have been found to target the 5′-UTR or to the coding region of some mRNAs. In some of these cases the miRNA/RISC complex inhibits the translation of the target mRNA and in others translation is promoted. Further, there are instances of certain miRNAs forming complexes with ribonucleoproteins and thus interfering with their RNA binding functions in a RISC-independent manner. Finally, there are also documented instances in which miRNAs can affect transcription of particular genes by binding to DNA.

Over the last dozen years, RNAi related mechanisms involving siRNA and miRNA have been substantially elucidated and found to occur widely in both plants and animals including in all human cell types. In turn, these advances have been applied to the design and use of RNAi based drugs for use as therapeutic candidates and as a tool for various research and drug development purposes. Tuschl's group first reported the administration of synthetic siRNA to cells more than 10 years ago (Elbashir et al., Nature 411: 494-498, 2001). Conventional siRNA therapeutics has very recently reached the stage where significant RNAi activity can be achieved in the livers of primates as well as man. The best of these results to date are based on the use of second-generation lipid nanoparticles (LNPs) that envelop the siRNA and promote its delivery to hepatic cells. These data come from interim results from a phase I trial of a siRNA directed to PCSK9.

MiRNA is comparatively a fundamentally more complex area of RNAi than siRNA and consequently attempts to acquire miRNA-based drug candidates for therapeutic as well as use as a tool for various research and drug development purposes have lagged behind siRNA. Potential miRNA therapeutics include miRNA inhibitors and miRNA mimics. Most advanced is the use of antisense oligonucleotides (oligos) with a steric hindrance mechanism to inhibit the function of certain miRNAs. One example is a mixed LNA/DNA nucleoside phosphorothioate oligo that inhibits miR-122 and which has completed phase II testing with promising results. Mir-122 is highly expressed by liver and is required for HCV production and increases the level of total cholesterol in plasma.

Least advanced is the delivery of miRNA mimics to tissues in vivo for therapeutic or research or drug development purposes. In part this is because the field is still in the early stages of elucidating the functions and identities of therapeutically relevant miRNAs. A relatively small number of miRNAs, however, have a substantial body of literature support for having key roles in certain medical conditions. A number of these miRNAs function as anti-oncogenes for particular types of cancer where they are pathologically under expressed. Importantly replacement of the deficient miRNA often has a substantial anti-cancer activity, for example, miR-34 and let-7 family members.

It is well recognized in the art that the single most important barrier to the development of siRNA and miRNA mimics as drugs is the very poor uptake of these compounds by tissues in the body (Aliabadi et al., Biomaterials 33: 2546, 2012; Kanasty et al., Mol Ther published online ahead of print Jan. 17, 2012). It is widely held that for general use complex carriers are needed that will envelop the siRNA or miRNA mimic and promote their delivery in to tissues in a bioavailable manner. To date the success of this approach is essentially limited to the delivery of such compounds to liver.

In contrast, steric hindrance antisense oligos being used to inhibit miRNAs are being successfully delivered tissues without the need for a carrier. Further, clinically important endpoints are being achieved. Such oligos, however, require high doses and perhaps most importantly very high affinity for their target miRNA (Elmen et al., Nature 452: 896, 2008; Lanford et al., Science 327: 198, 2010). Thus, miRNAs with relatively high G/C content should be most susceptible to this form of inhibition. It may not be possible to effectively target the majority or miRNAs using this approach and existing antisense oligo chemistries because of the high affinity requirement.

The miRNA sequences and nomenclature used herein are taken from the miRBase (www.mirbase.org) which has been described in Griffiths-Jones et al., Nucleic Acids Research 34: D140-D144, 2006. In brief, numbers that immediately follow the designation miR-, for example, miR-29, designate particular miRNAs. This designation is applied to the corresponding miRNAs across various species. Letters, for example in miR-34a and miR-34b, distinguish particular miRNAs differing in only one or two positions in the mature miRNA (antisense strand). Numbers following a second dash, for example in miR-24-1 and miR-24-2, distinguish distinct loci that give rise to identical mature miRNAs. These miRNAs can have different sense strands. Multiple miRNAs family members that differ in only one or two nucleoside positions from some other member(s) for the family in the mature miRNA and which also come from distinct hairpin loci have both letters and additional numbers following the letters, for example, miR-29b-1 and miR-29b-2 with the other family members being miR-29a and miR-29c. Finally, in some instances two different mature miRNA sequences are excised from the same hairpin precursor where one comes from the 5′ arm and the other from the 3′ arm. These are designated −5p and −3p respectively, for example, miR-17-5p and miR-17-3p.

### SUMMARY OF THE INVENTION

In accordance with the present invention, methods and compositions that provide RNAi activity in tissues in vivo are disclosed. The compositions of the present invention can be delivered to subjects as single strand oligos in a vehicle or physiological buffer, with out the requirement for a carrier or prodrug design while ultimately being capable of suppressing the intended target(s) in a wide variety of tissue types. Surprisingly, the present inventor has designed individual oligo strands with features that allow them survive administration, become bioavailable in a wide variety of tissues where they combine with a partner strand(s) to form duplexes that result in the efficient loading of the intended antisense oligo into RISC and produce robust intended silencing activity with minimized off-target effects.

The types of compositions of the present invention fall into three basic groups to include those that: (1) inhibit the expression of individual genes or small numbers of genes by an AGO-2 based cleavage mechanism; (2) inhibit the expression of particular miRNAs; and (3) provide miRNA-like functions through partially mimicking the actions of particular endogenous miRNAs of generating miRNA-like compounds with novel seed sequences. All three of these types of compounds are broadly defined as sequential RNAi (seqRNAi). They are individually distinguished by the terms seqsiRNA, seqIMiR and seqMiR respectively. Single stranded compounds with these three types of activity, ss-siRNA, ss-IMiR and ss-MiR respectively, are also provided.

Exemplary seqsiRNA, seqIMiR, seqMiR and ss-MiR compounds are based on the agents shown in FIGS. 8, 10, 12, 14, 16, 20-23 and 26-67; FIGS. 68-81; FIGS. 2, 9, 11, 13, 15, 17, 86-97; and FIGS. 2, 18 and 19 respectively. An exemplary method entails contacting a cell expressing the gene target, miRNA target or with a miRNA deficit with an effective amount of an appropriate seqRNAi compound, the seqRNAi being effective to inhibit expression of the target or to augment miRNA activity. SeqRNAi can include, without limitation, a single stranded or double stranded oligoribonucleotide or chimeric oligo with the properties provided for herein.

In a particularly preferred embodiment, a two-step administration method is disclosed. An exemplary method entails administration of a first oligo strand to a subject, waiting for a suitable time period, followed by administration of a second oligo strand to said subject, said first strand and said second strand forming an intracellular duplex in cells in vivo that is effective to achieve one of the following: (1) catalyze degradation of target gene mRNA or small number of mRNAs or inhibit translation of said mRNA(s); (2) catalyze degradation of a particular miRNA or small number of miRNAs; or (3) provide for miRNA activity. The oligo strands can be administered in a vehicle without a carrier or prodrug design, but a carrier may be used for special purposes such as the targeting of a particular tissue type to the exclusion of others.

### BRIEF DESCRIPTION OF THE DRAWINGS

FIG. 1: Key to Strand Modifications.

FIG. 2: Illustrations of Design of seqMiR Compounds with Novel Seed Sequences.

FIG. 3: Unmodified Strands Comprising a siRNA Compound Directed to Mouse PTEN.

FIG. 4: Unmodified Strands Comprising Human/Mouselet-7i.

FIG. 5: Strands Comprising Human/Mouselet-7i with Removal of Wobble Base Pairs and Mismatch.

FIG. 6: Application of Nuclease Resistance and Essential/preferred Architectural-Independent Rules to Strands for Design of seqsiRNA molecules Directed to Mouse PTEN.

FIG. 7: Application of Nuclease Resistance and Essential/preferred Architectural-Independent Rules to Strands for Design of seqMiR molecules Based on Human/Mouse Let-7i.

FIG. 8: Application of Thermodynamic Rules to Nuclease Resistant Strands Illustrating Preferred Steps in the Design of seqsiRNA molecules Directed to Mouse PTEN.

FIG. 9: Application of Thermodynamic Rules to Nuclease Resistant Strands Illustrating Preferred Steps in the Design of seqMiR molecules Based on Human/Mouse Let-7i.

FIG. 10: Application of Canonical Architecture-Dependent Algorithm to Strands Illustrating a Step in the Design of seqsiRNA molecules Directed to Mouse PTEN.

FIG. 11: Application of Canonical Architecture-Dependent Algorithm to Strands Illustrating a Step in the Design of seqMiR molecules Based on Human/Mouse Let-7i.

FIG. 12: Application of Asymmetric Architecture-Dependent Algorithm to Strands Illustrating a Step in the Design of seqsiRNA molecules Directed to Mouse PTEN.

FIG. 13: Application of Asymmetric Architecture-Dependent Algorithm to Strands Illustrating a Step in the Design of seqMiR molecules Based on Human/Mouse Let-7i.

FIG. 14: Application of Forked-variant Architecture-Dependent Algorithm to Canonical Architecture Strands Illustrating a Step in the Design of seqsiRNA molecules Directed to Mouse PTEN.

FIG. 15: Application of Forked-variant Architecture-Dependent Algorithm to Canonical Architecture Strands Illustrating a Step in the Design of seqMiR molecules Based on Human/Mouse Let-7i.

FIG. 16: Application of Small Internally Segmented Architecture-Dependent Algorithm Illustrating a Step in the Design of seqsiRNA molecules Directed to Mouse PTEN.

FIG. 17: Application of Small Internally Segmented Architecture-Dependent Algorithm Illustrating a Step in the Design of seqMiR molecules Based on Human/Mouse Let-7i.

FIG. 18: Application of ss-RNAi Architecture-Dependent Algorithm to an Antisense Strand Illustrating a Step in the Design of a ss-siRNA Directed to Mouse PTEN.

FIG. 19: Application of ss-RNAi Architecture-Dependent Algorithm to an Antisense Strand Illustrating a Step in the Design of a ss-MiR Based on Human/Mouse Let-7i.

FIG. 20: seqsiRNA Compounds Directed to Mouse Apo-B for sequential induction of RNAi Activity.

FIG. 21: seqsiRNA Compounds Directed to Human/Mouse PCSK9 for sequential induction of RNAi Activity.

FIG. 22: seqsiRNA Compounds Directed to Mouse Fas for sequential induction of RNAi Activity.

FIG. 23: seqsiRNA Compounds Directed to Mouse Stat3 for sequential induction of RNAi Activity.

FIG. 24: Boranophosphate Linkage.

FIG. 25: Boranophosphate Monomer with Native Ribose.

FIG. 26: seqsiRNA Compounds Directed to Human p53 for sequential induction of RNAi.

FIG. 27: seqsiRNA Compounds Directed to Human p53 for sequential induction of RNAi.

FIG. 28: seqsiRNA Compounds Directed to Human p53 for sequential induction of RNAi.

FIG. 29: seqsiRNA Compounds Directed to Human p53 for sequential induction of RNAi.

FIG. 30: seqsiRNA Compounds Directed to Human p53 for sequential induction of RNAi.

FIG. 31: seqsiRNA Compounds Directed to Human p53 for sequential induction of RNAi.

FIG. 32: seqsiRNA Compounds Directed to Human p53 for sequential induction of RNAi.

FIG. 33: seqsiRNA Compounds Directed to Human Fas for sequential induction of RNAi.

FIG. 34: seqsiRNA Compounds Directed to Human Fas for sequential induction of RNAi.

FIG. 35: seqsiRNA Compounds Directed to Human Fas for sequential induction of RNAi.

FIG. 36: seqsiRNA Compounds Directed to Human Fas for sequential induction of RNAi.

FIG. 37: seqsiRNA Compounds Directed to Human Fas for sequential induction of RNAi.

FIG. 38: seqsiRNA Compounds Directed to Murine ApoB for sequential induction of RNAi.

FIG. 39: seqsiRNA Compounds Directed to Human/Murine ApoB for sequential induction of RNAi.

FIG. 40: seqsiRNA Compounds Directed to Human/Murine ApoB for sequential induction of RNAi.

FIG. 41: seqsiRNA Compounds Directed to Human/Murine ApoB for sequential induction of RNAi.

FIG. 42: seqsiRNA Compounds Directed to Human/Murine ApoB for sequential induction of RNAi.

FIG. 43: seqsiRNA Compounds Directed to Human ApoB for sequential induction of RNAi.

FIG. 44: seqsiRNA Compounds Directed to Human ApoB for sequential induction of RNAi.

FIG. 45: seqsiRNA Compounds Directed to Human ApoB for sequential induction of RNAi.

FIG. 46: seqsiRNA Compounds Directed to Human ApoB for sequential induction of RNAi.

FIG. 47: seqsiRNA Compounds Directed to Human/Murine/Rat/Nonhuman Primate PCSK9 for sequential induction of RNAi.

FIG. 48: seqsiRNA Compounds Directed to Human/Murine/Rat/Nonhuman Primate PCSK9 for sequential induction of RNAi.

FIG. 49: seqsiRNA Compounds Directed to Human/Murine/Rat/Nonhuman Primate PCSK9 for sequential induction of RNAi.

FIG. 50: seqsiRNA Compounds Directed to Human PCSK9 for sequential induction of RNAi.

FIG. 51: seqsiRNA Compounds Directed to Human PCSK9 for sequential induction of RNAi.

FIG. 52: seqsiRNA Compounds Directed to Human PCSK9 for sequential induction of RNAi.

FIG. 53: seqsiRNA Compounds Directed to Human PCSK9 for sequential induction of RNAi.

FIG. 54: seqsiRNA Compounds Directed to Human PTEN for sequential induction of RNAi.

FIG. 55: seqsiRNA Compounds Directed to Human/Murine PTEN for sequential induction of RNAi.

FIG. 56: seqsiRNA Compounds Directed to Human PTP-1b for sequential induction of RNAi.

FIG. 57: seqsiRNA Compounds Directed to Human PTEN for sequential induction of RNAi.

FIG. 58: seqsiRNA Compounds Directed to Human/Non-Human Primate PTEN for sequential induction of RNAi.

FIG. 59: seqsiRNA Compounds Directed to Murine PTEN for sequential induction of RNAi.

FIG. 60: seqsiRNA Compounds Directed to Human/Murine PCSK9 for sequential induction of RNAi.

FIG. 61: seqsiRNA Compounds Directed to MurinePTP-1b for sequential induction of RNAi.

FIG. 62: seqsiRNA Compounds Directed to Human/MurinePTP-1b for sequential induction of RNAi.

FIG. 63: seqsiRNA Compounds Directed to Human p53 for sequential induction of RNAi.

FIG. 64: seqsiRNA Compounds Directed to Human p53 for sequential induction of RNAi.

FIG. 65: seqsiRNA Compounds Directed to Human/Mouse ApoB for sequential induction of RNAi.

FIG. 66: seqsiRNA Compounds Directed to Human/Mouse ApoB for sequential induction of RNAi.

FIG. 67: seqsiRNA Compounds Directed to HumanPTP-1b for sequential induction of RNAi.

FIG. 68: seqIMiR Compounds Based on Mouse miR-24 for sequential administration to inhibit the actions thereof.

FIG. 69: seqIMiR Compounds Based on Human miR-24 for sequential administration to inhibit the actions thereof.

FIG. 70: seqIMiR Compounds Based on Mouse miR-29a for sequential administration to inhibit the actions thereof.

FIG. 71: seqIMiR Compounds Based on Human miR-29a for sequential administration to inhibit the actions thereof

FIG. 72: seqIMiR Compounds Based on Mouse miR-29b for sequential administration to inhibit the actions thereof.

FIG. 73: seqIMiR Compounds Based on Human miR-29b for sequential administration to inhibit the actions thereof.

FIG. 74: seqIMiR Compounds Based on Mouse miR-29c for sequential administration to inhibit the actions thereof.

FIG. 75: seqIMiR Compounds Based on Human miR-29c for sequential administration to inhibit the actions thereof

FIG. 76: seqIMiR Compounds Based on Mouse miR-33 for sequential administration to inhibit the actions thereof.

FIG. 77: seqIMiR Compounds Based on Human miR-33 for sequential administration to inhibit the actions thereof.

FIG. 78: seqIMiR Compounds Based on Mouse miR-122 for sequential administration to inhibit the actions thereof.

FIG. 79: seqIMiR Compounds Based on Human miR-122 for sequential administration to inhibit the actions thereof.

FIG. 80: seqIMiR Compounds Based on Mouse miR-155 for sequential administration to inhibit the actions thereof.

FIG. 81: seqIMiR Compounds Based on Human miR-155 for sequential administration to inhibit the actions thereof.

FIG. 82: seqMiR Compounds Based on Mouse miR-24 for use in the sequential administration method described herein.

FIG. 83: seqMiR Compounds Based on Human miR-24 for use in the sequential administration method described herein.

FIG. 84: seqMiR Compounds Based on Mouse miR-26a for use in the sequential administration method described herein.

FIG. 85: seqMiR Compounds Based on Human miR-26a for use in the sequential administration method described herein.

FIG. 86: seqMiR Compounds Based on Mouse miR-29 for use in the sequential administration method described herein.

FIG. 87: seqMiR Compounds Based on Human miR-29 for use in the sequential administration method described herein.

FIG. 88: seqMiR Compounds Based on Mouse miR-122 for use in the sequential administration method described herein.

FIG. 89: seqMiR Compounds Based on Human miR-122 for use in the sequential administration method described herein.

FIG. 90: seqMiR Compounds Based on Mouse miR-146a for use in the sequential administration method described herein.

FIG. 91: seqMiR Compounds Based on Human miR-146a for use in the sequential administration method described herein.

FIG. 92: seqMiR Compounds Based on Mouse miR-203 for use in the sequential administration method described herein.

FIG. 93: seqMiR Compounds Based on Human miR-203 for use in the sequential administration method described herein.

FIG. 94: seqMiR Compounds Based on Mouse miR-214 for use in the sequential administration method described herein.

FIG. 95: seqMiR Compounds Based on Human miR-214 for use in the sequential administration method described herein.

FIG. 96: seqMiR Compounds Based on Mouse miR-499 for use in the sequential administration method described herein.

FIG. 97: seqMiR Compounds Based on Human miR-499 for use in the sequential administration method described herein.

### DETAILED DESCRIPTION OF THE INVENTION

### A. Overview of Prior Art

It is currently assumed in the art that the broad application of siRNA-based compounds and miRNA mimics as drugs will require the development of carriers that do not currently exist and that likely will involve different designs for different cell types. The existing carriers have primarily shown limited but meaningful success in obtaining siRNA activity at significant levels in the liver including in patients. It is generally believed that the carriers that will be needed to establish conventional siRNA and miRNA mimics as drug platforms will be of a complex structure and will envelop siRNA or miRNA duplexes. A possible tissue exception to the carrier requirement could be the proximal tubule cells of the kidney.

Carriers are believed to be needed for multiple reasons based on what happens when naked siRNA is injected into subjects including: (1) poor uptake by cells; (2) destruction by nucleases; and (3) rapid clearance of intact duplexes from the body. Further, the carriers being developed for general drug use have a variety of associated problems including, but not limited to, toxicity, difficulties in formulation, short shelf half-life and large size (siRNA/carrier or miRNA/carrier complexes are >100 nm in size while capillary pores are estimated to range from 5-60 nm). In addition, the published studies involving many carriers have common deficiencies making it difficult to draw firm conclusions; for example, it is uncommon to see proper dose response curves particularly ones that include comparing the test siRNA/carrier against an siRNA-control/carrier.

Hence, there is a pressing need for new approaches that will result in broad RNAi-dependent activity in tissues in vivo. The basic concept behind the present invention is that properly designed complementary sense and antisense strand drugs can be sequentially administered without a carrier or prodrug to a subject and will combine to form duplexes capable of producing RNAi activity in a wide range of cell types. Thus, in a preferred embodiment the compounds of the invention can be administered in the absence of a carrier (which facilitates cellular uptake) but are rather delivered in a vehicle, or physiological buffer such as saline Thus, this invention provides the means to generate sense and antisense strands with sufficient intrinsic nuclease stability such that they can be individually administered in vivo in a sequential manner and induce the production of RNAi activity in numerous tissues. This general approach has been termed seqRNAi.

In the field of miRNA mimics, there is also a pressing need for the rationale design of compounds which avoid suppressing desirable mRNA types while inhibiting the expression mRNA types where there is a commercial or medical interest in doing so. This is an intrinsic problem when the goal is to closely mimic particular endogenous miRNAs. Using miRNA-like compounds that are limited their range of mRNA target types (e.g., selected to better match particular commercial goals) can ameliorate this problem. The seqMiRs of the present invention can be designed to do this in particular through the use of novel seed sequences and by manipulating the affinity of the seed sequence for its mRNA targets.

Xu et al., (Biochem Biophys Res Comm 316: 680, 2004) studied the effects of the sequential administration of single strands by transfection of sense and antisense strands making up a chemically unmodified conventional siRNA duplex on cells grown in culture. They demonstrated the ability of such an approach to cause RNAi based silencing in cells under these conditions. The authors made the observation that single stranded siRNA (ss-siRNA) “has a remarkably lower efficacy of reconstituting RISC than duplex siRNA.” This led them to test the following notion: “cellular persistence (meaning short persistence) might not be the main reason of ss-siRNA having lower efficacy than duplex siRNA.” Instead the duplex structure itself might promote RISC loading. They tested this idea by sequentially administering the complementary strands of a conventional siRNA directed to Renilla luciferase or of one targeting human CD46 into a cell line expressing the target gene. These investigators did not disclose the sequential administration of individual strands in vivo nor the concept of using sequential strand administration to improve uptake compared to the administration of a duplex.

WO 2009/152500 primarily involves the use of short and/or non-canonical siRNA triggers and data is provided to show that ones shorter than the standard 21-mers have substantial activity. The filing also asserts that the two strands that make up conventional siRNA can be sequentially administered to cells and as a result the RNAi-based silencing effect of the parent siRNA duplex will be replicated in cells. The rationale that the text provides for doing this is the following: “Because the interferon pathway is triggered by cells exposed to double-stranded nucleic acids previous RNAi/gene silencing approaches using such agents could not rule out the concomitant activation of this pathway.” Accordingly, the inventors claim to provide compositions and methods for conducting gene silencing both in vitro and in vivo in the absence of an interferon response.” The idea that sequential administration of the strands could remedy the in vivo siRNA uptake problem was not considered, nor were specific compounds for use in this embodiment of the invention.

The sequential administration of complementary sense and antisense strands to achieve RNAi-dependent activity against a specific mRNA target in cells is clearly distinguishable from the practice of sequentially or co-administering conventional siRNA duplexes to cells in vitro or in vivo. As for drugs generally, there are multiple rationales for administering more than one conventional siRNA duplex to an animal or individual in either a sequential or in a simultaneous manner. These reasons include, for example, the desire to produce a more profound suppression of a given target at a given time, to extend the effect on a given target over time, to achieve a particular commercial purpose by inhibiting multiple targets in a sequential manner or simultaneously or to reduce the selection pressure for the production of mutations in the target gene that nullify the intended effect.

US 2009/0156529 discloses the sequential administration of established types of RNAi. In this application, “The term “co-administration” refers to administering to a subject two or more agents, and in particular two or more iRNA agents. The agents can be contained in a single pharmaceutical composition and be administered at the same time, or the agents can be contained in separate formulation and administered serially to a subject. So long as the two agents can be detected in the subject at the same time, the two agents are said to be co-administered.” Thus, the inventors have provided for the sequential administration of “iRNA agents” (abbreviation for “interfering RNA agent”) a term that is not established in the art but clearly means an agent that induces RNAi-dependent silencing activity. Indeed, the inventors defined iRNA agents as follows: “An iRNA agent as used herein, is an RNA agent, which can down-regulate the expression of a target gene, e.g. ENaC gene SCNN1A . . . an iRNA agent may act by one or more of a number of mechanisms, including post-transcriptional cleavage of a target mRNA sometimes referred to in the art as RNAi, or pre-transcriptional or pre-translational mechanisms.” Thus, the term iRNA agent must be an entity that can down-regulate the expression of a target gene and such agents may be co-administered in a sequential manner over time if the agents being so co-administered are present in the subject at the same time.

In connection with attempts to generate siRNA-based or miRNA mimic drug development platforms, investigators are focused on developing complex carriers that envelop the duplex to deliver conventional siRNA or miRNA mimics to subjects. The duplexed nature of these compounds provides a degree of nuclease stability that in turn affects the selection of specific chemical modifications to the strands, if any, in order to promote the various desirable drug attributes of the compound. The duplex structure also has an important bearing on the intracellular distribution of the compound with respect to parameters such as relative distribution between the cytoplasm and nucleus and general stickiness of proteins on a charge/charge basis. Further, the carrier itself introduces additional nuclease resistance and has a major influence on determining the details of the route followed by the duplex in becoming bioavailable in cells in vivo. Thus, the approaches that have been developed to promote the desirable drug attributes of conventional siRNA or miRNA mimics have been arrived at in the context of the these drugs being administered as a duplex by means of a complex carrier.

The problem of what chemical modifications to use and where to place them in strands is necessarily substantially greater for seqRNAi than for the strands that comprise conventional siRNA or miRNA mimics. Basic to the greater difficulty for seqRNAi are the following facts: (1) seqRNAi strands have a substantially greater need for nuclease resistance than the strands that make up conventional siRNA duplexes. As a result they are necessarily more heavily modified compared to conventional siRNA or miRNA mimics; and (2) essentially all the types of chemical modifications that are applicable for achieving single strand nuclease resistance are known to be capable of substantially inhibiting or eliminating the intended RNAi-dependent silencing activity. A number of these are compatible with conventional siRNA activity but they must be used sparingly and with suitable positioning in the strand. This has been possible because of the duplex structure and the nuclease protection provided by carriers. The lack of these factors in the use of seqRNAi, therefore, presents a novel challenge.

The present invention provides the means to achieve this by providing sufficient intrinsic nuclease resistance for each of the strands to survive long enough to become bioavailable duplexes in cells in vivo while not unduly adversely affecting the silencing activity against the intended target. This includes providing the means for the efficient removal of the sense strand form the seqRNAi-based duplex by RISC. Multiple seqRNAi-based duplex architectures are also enabled by the disclosure in the present application. The algorithms provided herein surprisingly allow these objectives to be achieved without undo experimentation and provide for the rationale design of compounds having seqRNAi activity against any mRNA or miRNA target as well as compounds with miRNA-like properties. The miRNA mimics of the present invention fall into two broad categories: (1) those that are based on the seed sequences of endogenous miRNA compounds; and (2) those that are based on novel seed sequences. So the term “miRNA mimics” in this context is used for compounds that provide miRNA-like activity rather than necessarily suggesting an attempt to exactly mimic the activity of any given endogenous miRNA. The miRNA mimics of the present invention are designed to serve as drugs that provide a wide range of miRNA activities that can be tailored to meet a variety of useful commercial or medical needs.

The seqRNAi designs of the present invention are configured for single strand in vivo administration in a vehicle without a carrier or prodrug design. This results in RNAi activity in many cell types. While this is frequently desirable, it is also important to have the ability to direct the seqRNAi strands to some cell or tissue types to the exclusion of others by using carriers with cell targeting characteristics. SeqRNAi strands are much better suited for use with carriers than is conventional siRNA or conventional miRNA mimics because of their smaller size and intrinsic nuclease resistance. Hence, the carrier can be simply conjugated to the seqRNAi strand and it can be relatively small and uncomplicated since it does not need to envelop the strand. Such relatively simple carriers capable of targeting oligos to particular tissues are well known in the art.

Select antisense seqRNAi strands can also be used as ss-siRNA or ss-miRNA. Certain modifications can promote this activity. Typically the activity will be less than that which can be achieved with the sequential administration of the complementary sense strand(s), but for some commercial applications the simplicity of a single administration out weighs the increased potency the sense strand can provide. This would include situations where a very rapid suppressive effect is desired.

It follows that the greater level of chemical modification that is required for seqRNAi strands compared to the strands in conventional siRNA and conventional miRNA must be more highly orchestrated such that potentially competing objectives are harmonized. The present invention surprisingly provides the means to broadly achieve substantial RNAi-dependent activity against targets of choice in multiple cell/tissue types in subjects without undo experimentation. The RNAi-dependent activity generated by seqRNAi sets or ss-RNAi based on seqRNAi antisense designs can occur in either a siRNA-like or miRNA-like format.

### B. Definitions

The following definitions and terms are provided to facilitate an understanding of the invention.

“2′-fluoro” refers to a nucleoside modification where the fluorine has the same stereochemical orientation as the hydroxyl in ribose. In instances where the fluorine has the opposite orientation, the associated nucleoside will be referred to as FANA or 2′-deoxy-2′fluoro-arabinonucleic acid.

“3′-supplementary or 3′-compensatory sites” refers to sites in some miRNA antisense strands down-stream of the seed sequence that are complementary to the target sequence and contribute to target selection particularly when the seed sequence has a weak match with the target.

3′UTR is an abbreviation for the 3′ untranslated region of an mRNA.

“5′-to-3′ mRNA decay pathway” refers to a naturally occurring pathway for degrading mRNA that is initiated by the removal of the poly(A) tail by deadenylases. This is followed by removal of the 5′-cap and subsequent 5′ to 3′ degradation of the rest of the mRNA.

“Antisense oligos or strands” are oligos that are complementary to sense oligos, pre-mRNA, mRNA or to mature miRNA and which bind to such nucleic acids by means of complementary base pairing. The antisense oligo need not base pair with every nucleoside in the target. All that is necessary is that there be sufficient binding to provide for a Tm of greater than or equal to 40° C. under physiologic salt conditions at submicromolar oligo concentrations unless otherwise stated herein.

“Algorithms” refers to sets of rules used to design oligo strands for use in the generation of seqRNAi sets or pairs.

“Antisense strand vehicle” is used to describe an antisense strand structure into which particular seed sequences can be inserted as a starting point for the design of ss-MiR compounds. These vehicles are designed and/or selected to minimize off target effects and to promote efficient RISC loading.

“Architecture” refers to one of the possible architectural configurations of the seqRNAi-based duplexes formed after a set of seqRNAi strands undergoes complementary base pairing or it refers to the group of such architectures.

“Asymmetry rule” refers to the naturally occurring mechanism whereby the likelihood of a particular strand in a siRNA, miRNA or seqRNAi-based duplex is selected by RISC as the antisense strand. It has been applied to the design of conventional siRNA compounds and it can apply to seqRNAi compounds. In brief, the relative Tm of the 4 terminal duplexed nucleosides at one end of the duplex compared to the corresponding nucleosides at the other terminus of the duplex plays a key role in determining the relative degree to which each strand will function as the antisense strand in RISC. The strand with its 5′-end involved in the duplexed terminus with the lower interstrand Tm more likely will be loaded into RISC as the antisense strand. The Tm effect, however, is not evenly distributed across the duplexed terminal nucleosides because the most terminal is the most important with the successive nucleosides being progressively less important with the terminal 4 duplexed nucleosides being the most significant.

“Backbone” refers to the alternating linker/sugar or sugar substitute structure of oligos while the normal bases or their substitutes occur as appendages to the backbone.

“Bulge structures or bulge” refers to regions in a miRNA duplex or seqMiR-based duplex where multiple interior contiguous nucleosides in one strand fail to base pair with the partner strand in a manner that results in the formation of a bulge in the duplex composed of these nucleosides. Bulge structures include bulge loops that occur when the nucleosides that fail to base pair with the partner strand are only in one strand and interior loops that occur when opposing nucleosides in both strands cannot base pair.

“Central region of the antisense stand” is defined as nucleosides 9 and 10 from the 5′end along with the adjacent three nucleosides on each side of these including all the intervening linkages.

“Chemically modified” is applied to oligos used as conventional antisense oligos, conventional siRNA, conventional miRNA or seqRNAi (seqsiRNA, seqMiRs, or seqIMiR) where the term refers to any chemical differences between what appears in such compounds and the corresponding standard natural components of native RNA and DNA (U, T, A, C and G bases, ribose or deoxyribose sugar and phosphodiester linkages). During manufacture chemical modifications of this type do not have to literally be made to native DNA or RNA components. Also included in this term are any nucleoside substitutes that can be used as units in overhang precursors.

“Chimeric oligonucleotides” are ones that contain ribonucleosides as well as 2′-deoxyribonucleosides.

“Compounds” refers to compositions of matter that include conventional siRNA, conventional miRNA, as well as the sense, antisense strands that make up particular seqRNAi sets in addition to the seqRNAi-based duplexes they can form by complementary base pairing with each other.

“Conventional antisense oligos” are single stranded oligos that inhibit the expression of the targeted gene by one of the following mechanisms: (1) Steric hindrance—e.g., the antisense oligo interferes with some step in the sequence of events involved in gene expression and/or production of the encoded protein by directly interfering with one of these steps. Such steps can include transcription of the gene, splicing of the pre-mRNA and translation of the mRNA; (2) Induction of enzymatic digestion of the RNA transcripts of the targeted gene by RNase H; (3) Induction of enzymatic digestion of the RNA transcripts of the targeted gene by RNase L; (4) Induction of enzymatic digestion of the RNA transcripts of the targeted gene by RNase P: (5) Induction of enzymatic digestion of the RNA transcripts of the targeted gene by double stranded RNase; and (6) Combined steric hindrance and induction of enzymatic digestion activity in the same antisense oligo.

“Conventional miRNA” are those compounds administered to cells in vitro or in vivo as an oligo duplex and the term excludes those unusual cases where it is delivered as single stranded miRNA (ss-miRNA)—i.e., where the antisense stand is administered without a sense strand and produces a substantial RNAi silencing effect. Administration of conventional miRNA nearly always requires the use of a carrier (in vitro or in vivo) or other means such as hydrodynamic delivery (in vivo) to get the compound into cells in an active form.

“Conventional siRNA” are those compounds administered to cells in vitro or in vivo as an oligo duplex and the term excludes those unusual cases where it is delivered as single stranded siRNA (ss-siRNA)—i.e., where the antisense stand is administered without a sense strand and produces a substantial RNAi silencing effect. Administration of conventional siRNA nearly always requires the use of a carrier (in vitro or in vivo) or other means such as hydrodynamic delivery (in vivo) to get the compound into cells in an active form.

“Duplex vehicle” is used to describe a duplex comprised of a sense and an antisense strand into which particular seed sequences and their sense strand complement can be inserted as a starting point for the design of seqMiR compounds. These vehicles are designed and/or selected to minimize off target effects and to promote efficient RISC loading and retention of the intended antisense strand.

“Exosomes” are endosome-derived vesicles that transport molecular species such as miRNA and siRNA from one cell to another. They have a particular composition that reflects the cells of origin and typically this directs the payload to particular cells. Once these secondary cells take up the siRNA or miRNA they exert their RNAi functions.

“FANA” refers to a nucleoside modification where the fluorine has the opposite stereochemical orientation as the hydroxyl in ribose. It can also be referred to as 2′-deoxy-2′fluoro-arabinonucleic acid.

“Gene target” or “target gene” refers to either the DNA sequence of a gene or its RNA transcript (processed or unprocessed) that is targeted by an RNAi trigger for suppression of its expression.

“Guide strand” is used interchangeably with antisense strand in the context of dsRNA, miRNA or siRNA compounds.

“Identity” as used herein and as known in the art, is the relationship between two or more oligo sequences, and is determined by comparing the sequences. Identity also means the degree of sequence relatedness between oligo sequences, as determined by the match between strings of such sequences. Identity can be readily calculated (see, e.g., Computation Molecular Biology, Lesk, A. M., eds., Oxford University Press, New York (1998), and Biocomputing: Informatics and Genome Projects, Smith, D. W., ed., Academic Press, New York (1993), both of which are incorporated by reference herein). While a number of methods to measure identity between two polynucleotide sequences are available, the term is well known to skilled artisans (see, e.g., Sequence Analysis in Molecular Biology, von Heinje, G., Academic Press (1987); and Sequence Analysis Primer, Gribskovm, M. and Devereux, J., eds., M. Stockton Press, New York (1991)). Methods commonly employed to determine identity between oligo sequences include, for example, those disclosed in Carillo, H., and Lipman, D., Siam J. Applied Math. (1988) 48:1073.

“Internal linkage sites” refers to linkage sites that are not at the 5′ or 3′-ends of an oligo strand. These sites are potentially subject to single strand endonuclease attack and to double strand endonuclease attack if they form a duplex with a partner strand. Such sites may also be simply referred to as linkage sites.

iPS cell or iPSC are abbreviations for induced pluripotent stem cells. They are created (induced) from somatic cells by experimental manipulation. Such manipulation has typically involved the use of expression vectors to cause altered (increased or decreased) expression of certain genes in the somatic cells. “Pluripotent” refers to the fact that such stem cells can produce daughter cells committed to one of several possible differentiation programs.

“Linkage site” refers to a particular linkage site or type of linkage site within an oligo that is defined by the nature of the linkage and the identities of the contiguous 5′ and 3′ nucleosides or nucleoside substitutes. Linkage sites are designated by “X-Y” where X and Y each represent nucleosides with one of the normal bases (A, C, G, T or U) or nucleoside substitutes and the dash indicates the linkage between them.

“Mismatch” refers to a nucleoside in an oligo that does not undergo complementary base pairing with a nucleoside in a second nucleic acid or with another nucleoside in the same oligo and where the effect is to antagonize interstrand or intrastrand duplex formation by setting up a repulsion of the opposing nucleoside base.

“MicroRNAs (miRNAs)” are a category of naturally occurring dsRNAs that typically trigger the post-transcriptional repression of protein encoding genes after one of the strands is loaded into RISC. This antisense strand can be referred to as mature miRNA. It directs RISC to specific mRNA targets as recognized by the seed region of the mature miRNA. Most commonly the seed sequence recognizes complete matched sequences in the 3′UTR of mRNAs transcribed from multiple genes.

“MicroRNA mimics or miRNA mimics” are a category of manufactured compounds that when administered to cells utilize the cellular mechanisms involved in implementing the activity of naturally occurring miRNA in order to produce a modulation in the expression of a particular set of genes. MicroRNA mimics of the present invention can be designed to modulate some or all of the same genes modulated by a particular naturally occurring miRNA or be designed to modulate the expression of a set of genes by using a novel seed sequence. The miRNA mimics of the present invention are referred to as seqMiRs or ss-MiRs depending on whether they involve one or two strands.

“Modulate”, “modulating” or “modulation” refer to changing the rate at which a particular process occurs, inhibiting a particular process, reversing a particular process, and/or preventing the initiation of a particular process. Accordingly, if the particular process is tumor growth or metastasis, the term “modulation” includes, without limitation, decreasing the rate at which tumor growth and/or metastasis occurs; inhibiting tumor growth and/or metastasis; reversing tumor growth and/or metastasis (including tumor shrinkage and/or eradication) and/or preventing tumor growth and/or metastasis.

“Native RNA” is naturally occurring RNA (i.e., RNA with normal C, G, U and A bases, ribose sugar and phosphodiester linkages).

“Nucleoside” is to be interpreted to include the nucleoside analogs provided for herein. Such analogs can be modified either in the sugar or the base or both. Further, in particular embodiments, the nucleotides or nucleosides within an oligo sequence may be abasic. In overhang precursors and overhangs in RNAi triggers, each nucleoside and its 5′ linkage can be referred to as a unit.

“Nucleoside substitute” refers to structures with radically different chemistries, such as the aromatic structures that may appear in the 3′-end overhang precursors or overhangs of seqRNAi-based siRNA duplexes, but which play at least one role typically undertaken by a nucleosides. It is to be understood that the scope of the rules that apply to 3′-end overhang precursors are broader than the rules that apply to structures that occur in the regions of the seqRNAi strand that would form a duplex with its partner strand(s). In overhang precursors and overhangs each nucleoside substitute and its 5′ linkage can be referred to as a unit.

“Oligo(s)” is an abbreviation for oligonucleotide(s).

“Overhang” in the context of conventional siRNA and conventional miRNA refers to any portion of the sense and/or antisense strand that extends beyond the duplex formed by these strands and that is comprised of nucleoside or nucleoside substitute units.

“Overhang precursor” refers to that portion, if any, of a seqRNAi strand that would form an overhang when combine with a partner seqRNAi strand to form a seqRNAi-based duplex. The term also applies to ss-RNAi based on seqRNAi antisense designs where there are one or more units at the 3′-end of the strand that do not undergo complementary base pairing with the intended target and which would form an overhang if the strand were duplexed with a seqRNAi sense strand.

“Passenger strand” is used interchangeably with “sense strand” in the context of dsRNA miRNA or siRNA compounds or their components. It forms a complex with its partner guide or antisense strand to form one of these compounds.

“Pharmaceutical composition” refers to an entity that comprises a pharmacologically effective amount of a single or double stranded oligo(s), optionally other drug(s), and a pharmaceutically acceptable carrier.

“Pharmacologically effective amount,” “therapeutically effective amount” or simply “effective amount” refers to that amount of an agent effective to produce a commercially viable pharmacological, therapeutic, preventive or other commercial result.

“Pharmaceutically acceptable carrier” refers to a carrier or diluent for administration of a therapeutic agent. Pharmaceutically acceptable carriers for therapeutic use are well known in the pharmaceutical art, and are described, for example, in Remington's Pharmaceutical Sciences, AR Gennaro (editor), 18^{th} edition, 1990, Mack Publishing or Remington: The Science and Practice of Pharmacy, University of the Sciences in Philadelphia (editor), 21^{st} edition, 2005, Lippincott Williams & Wilkins, which are hereby incorporated by reference herein.

“Prodrug” refers to a compound that is administered in a form that is inactive but becomes active in the body after undergoing chemical modifications typically through metabolic processes. In the context of RNAi-dependent compounds, prodrug designs have been proposed as a means of protecting such compounds from nucleases and/or promoting their uptake by cells. As for prodrugs generally any RNAi-dependent prodrugs have to undergo modification in the body to produce a compound capable of RISC loading and processing to induce silencing the intended target(s). The administration of RNAi-dependent compounds without 5′-end phosphorylation of the antisense strand is not considered to constitute the administration of a prodrug.

“RNAi” is an abbreviation for RNA-mediated interference or RNA interference. It refers to the system of cellular mechanisms that produces RNAi triggers and uses them to implement silencing activity. Multiple types of RNAi activities are recognized with the two most prominent being siRNA and miRNA. Nearly always the RNAi triggers associated with these activities are double stranded RNA oligos most commonly in the 20-23-mer-size range. A common feature of the RNAi mechanism is the loading of one of these double stranded molecules into RISC following by the sense or passenger strand being discarded and the antisense or guide strand being retained and used to direct RISC to the target(s) to be silenced.

“RNAi-dependent” refers to the use of an RNAi based mechanism to silence gene expression. Compounds using this mechanism include conventional siRNA, shRNA, dicer substrates, miRNA and the three types of seqRNAi (seqsiRNA, seqMiR and seqIMiR) as well as ss-siRNA, ss-IMiRs and ss-MiRs.

“RNAi trigger” refers to a double stranded RNA compound most commonly in the 20-23-mer size range that loads into RISC and provides the targeting entity (guide or antisense strand) used to direct RNAi activity.

“Seed sequence or seed region” comprises nucleosides 2-8(or 2-7) counting in from the 5′-end of the de facto antisense strand of conventional siRNA, miRNA or nonconventional seqRNAi or ss-RNAi.

“Seed duplex” refers to the duplex formed between the seed sequence in a de facto antisense stand and its complement in an mRNA 3′UTR.

“Sense oligos or strands” are oligos that are complementary to antisense oligos or antisense strands of particular genes and which bind to such nucleic acids by means of complementary base pairing. When binding to an antisense oligo, the sense oligo need not base pair with every nucleoside in the antisense oligo. All that is necessary is that there be sufficient binding to provide for a Tm of greater than or equal to 40° C. under physiologic salt conditions at submicromolar oligo concentrations unless otherwise provided for herein.

“Sequential” in the context of the administration of a seqRNAi compound refers to a “two-step administration or method” where cells are treated with one strand of a complementary sense and antisense oligo pair and after cellular uptake of this strand, the cells are treated with the other strand in a manner that also provides for its uptake into the cells. The two strands then form a functional RNAi trigger intracellularly to inhibit target gene expression in the cells containing the RNAi trigger.

“SeqIMiRs” are the subtype of seqRNAi compounds that are designed to inhibit the expression and/or function of particular endogenous miRNAs.

“SeqMiRs” are the subtype of seqRNAi compounds that are designed to mimic miRNA function. Such mimics may be based on a particular endogenous miRNA seed sequence. When based on a particular endogenous miRNA seqMiRs are typically designed to only inhibit a subset of the specific mRNAs inhibited by the endogenous miRNA in question. SeqMiRs can also be designed with a novel seed sequence and, therefore, not be based on any given endogenous miRNA.

“SeqRNAi” refers to a novel approach to siRNA and miRNA delivery where the individual sense and antisense strands making up the duplexes are sufficiently modified to have sufficient intrinsic nuclease resistance for in vivo sequential administration without a carrier or prodrug design and at the same time being able to produce an RNAi-dependent silencing effect on the intended target gene(s) in a wide range of cell/tissue types. There are three different types of seqRNAi (seqsiRNAs, seqMiRs, and seqIMiRs).

“SeqRNAi-based duplex” refers to the duplex formed when the strands in a seqRNAi set or pair combine with each other through complementary base pairing.

“SeqRNAi set” or “seqRNAi pair” refers to a group of two or three strands where the strands can combine to form a seqRNAi-based duplex on the basis of complementary base pairing.

“SeqsiRNA” is the subtype of seqRNAi that inhibits the expression of an individual gene or small number of genes by promoting direct cleavage of the transcripts of the genes by RISC. The targeting code is primarily composed of the central region of the antisense strand. Conventional siRNA compounds can be converted to seqsiRNA use or accessible sites in mRNA for oligo binding can be used as the starting point for designing seqsiRNA compounds.

“Silencing” refers to the inhibition of gene expression that occurs as a result of RNAi activity. It is commonly expressed as the concentration of the RNAi trigger that produces a 50% inhibition in the expression of the intended target at the optimum time point.

“Ss-IMiR” refers to an antisense strand that is designed according to the rules provided herein and is administered to a subject without a carrier or prodrug design and without the administration of a complementary sense strand. The compound is capable of being loaded into RISC in a subjects cells and subsequently directing RISC to a specific miRNA for silencing.

“Ss-MiR” refers to a single stranded miRNA mimic composed of an antisense strand designed according to the rules provided herein that is capable of being administered to a subject without a carrier or prodrug design and without a complementary sense strand. It can be loaded into RISC in subject cells and subsequently directed to a set of targets for silencing of target gene expression, e.g., inhibition of a particular set of mRNAs containing the complementary binding sequences in the 3′UTR. The targeting code is primarily or exclusively provided by the seed sequence.

“Ss-miRNA” refers to a single stranded miRNA mimic composed of an antisense or guide strand that is capable of being loaded into RISC and subsequently directed to a set of targets for silencing of target gene expression, e.g., inhibition of a particular set of mRNAs containing the complementary binding sequences in the 3′UTR. The targeting code is primarily if not exclusively provided by the seed sequence.

“Ss-RNAi” refers to ss-siRNA and/or to ss-miRNA and/or to ss-MiR and/or to ss-IMiR compounds.

“Ss-siRNA” refers to an antisense strand that is designed according to the rules provided herein and is administered to a subject without a carrier or prodrug design and without a complementary sense strand. Further, the compound is capable of being loaded into RISC in subjects cells and subsequently directing RISC to the transcript(s) of one or at most a small number of mRNA types for silencing of target gene expression. The targeting code is primarily or exclusively composed of the central region of the strand and it typically directs AGO-2 to an mRNA target(s) that is cleaved by this enzyme.

“Stem cell” refers to a rare cell type in the body that exhibits a capacity for self-renewal. Specifically when a stem cell divides the resulting daughter cells are either committed to undergoing a particular differentiation program or they undergo self-renewal in which case they produce a replica of the parent stem cell. By undergoing self-renewal, stem cells function as the source material for the maintenance and/or expansion of a particular tissue or cell type.

“Subject” refers to a mammal including man.

“Substantially identical,” as used herein, means there is a very high degree of homology preferably >90% sequence identity between two nucleic acid sequences.

“Synthetic” means chemically manufactured by man.

“Targeting code” refers to a contiguous nucleoside sequence that is a subset of the guide or antisense strand sequence of a siRNA, miRNA or seqRNAi compound that is primarily or exclusively responsible for directing RISC to a specific target(s). Targeting codes typically can be distinguished on the basis of their particular positions within the guide or antisense strand relative to its 5′-end.

“Tm” or melting temperature is the midpoint of the temperature range over which an oligo separates from a complementary nucleotide sequence. At this temperature, 50% helical (hybridized) and 50% coiled (unhybridized) forms are present. Tm is measured by using the UV spectrum to determine the formation and breakdown (melting) of hybridization using techniques that are well known in the art. There are also formulas available for estimating Tm on the basis of nearest neighbor considerations or in the case of very short duplexes in accordance with the relative G:C and U:A content. For the purposes of the present invention Tm measurements are based on physiological pH (about 7.4) and salt concentrations (about 150 mM).

“Treatment” refers to the application or administration of a single or double stranded oligo(s) or another drug to a subject or patient, or application or administration of an oligo or other drug to an isolated tissue or cell line from a subject or patient, who has a medical condition, e.g., a disease or disorder, a symptom of disease, or a predisposition toward a disease, with the purpose to inhibit the expression of one or more target genes for research and development purposes or to cure, heal, alleviate, relieve, alter, remedy, ameliorate, improve, or affect the disease, the symptoms of disease, or the predisposition toward disease. Tissues or cells or cell lines grown in vitro may also be “treated” by such compounds for these purposes.

“Unit” refers to the nucleoside or nucleoside substitutes that appear in overhang precursors and overhangs along with their 5′-end linkage. Nucleosides may appear in 5′-end or 3′-end overhangs but nucleoside substitutes can only appear in 3′-end overhang precursors and overhangs.

“Unlocked nucleic acids” (UNA) are a new class of oligos that contain nucleosides with a modification to the ribose sugar such that the ring becomes acyclic by virtue of lacking the bond between the 2′ and 3′ carbon atoms. The term can also be applied to individual nucleosides with this modification.

“Upstream” and “Downstream” respectively refer to moving along a nucleotide strand in a 3′ to 5′ direction or a 5′ to 3′ direction respectively.

“Vehicle” refers a substance of no therapeutic value that is used to convey an active medicine or compound for administration to a subject in need thereof.

### C. The Embodiments

In one embodiment, novel complementary sense and antisense oligo compounds are sequentially administered to a subject in a two-step sequential procedure whereby one strand is administered without a carrier or prodrug design and taken up by cells expressing the RNA target(s), followed by administration of the second complementary strand without a carrier or prodrug design which is taken up by the same cells resulting in the silencing of the function of specific RNA target(s) by an RNAi-dependent mechanism. Thus, the individual strands are taken up intact by a wide variety cell/tissue types in vivo in sufficient amounts in a bioavailable manner that allows them to generate commercially useful RNAi-dependent silencing activity against the intended RNA target(s). The types of RNA targets in question include, for example, pre-mRNA, mRNA and miRNA although in principle any RNA type could be targeted.

In a related embodiment, methods and algorithms are provided for modifying known conventional siRNA compounds to render them suitable for use in the sequential two-step sequential administration method described above. In particular these methods and algorithms provide for the creation of complementary sense and antisense strands that can be sequentially administered to subjects without a carrier or prodrug design and where they exhibit the following properties: (1) exhibit sufficient intrinsic nuclease resistance to survive long enough to carry out their intended drug function; (2) are widely taken up by many cell/tissue types in a manner that renders them bioavailable; and (3) produce the intended RNAi-dependent silencing activity in cells/tissues that express the relevant RNA target(s). This silencing activity is enhanced relative to the effects seen when the strands are administered without the partner strand or compared to the sequence identical conventional RNAi-dependent compound that has not been modified in accordance with the present invention and is delivered without a carrier.

In another embodiment, the methods and algorithms provided are applied to complementary sense and antisense strands that are not known conventional siRNA compounds. These same methods and design algorithms are also suitable for the generation of novel compounds that inhibit particular miRNAs. This approach can be applied to generating inhibitors of any RNA target(s) in subjects where RNAi is desirable. What is required is that the portion of the target be accessible to complementary base pairing by an antisense strand that along with a complementary sense strand, are suitable for being configured in accordance with guidance provided with the present invention. The means for determining those portions of the intended RNA target which are accessible to complementary base pairing are well known in the art. Conventional antisense oligo which have activity against RNA target(s) provide direct evidence of the particular binding site(s) on an RNA target accessible to such complementary base pairing. It also follows that conventional antisense oligos can be reconfigured as compounds of the present invention. In a preferred version of this embodiment an antisense strand compound of the present invention is directed to a hotspot in gene target mRNA transcripts where the hotspot is defined in U.S. Pat. No. 7,517,644.

In yet another embodiment, algorithms, methods and compositions of matter are provided for achieving miRNA mimic activity in cells/tissues in subjects using the sequential delivery method of suitably designed sense and antisense strands. In one version of this approach a particular endogenous miRNA is subjected to the methods and algorithms of the present invention. In a variant of this, the targeting code sequence of the endogenous miRNA is adjusted to improve the silencing profile of the compound for a particular commercial purpose.

In a related embodiment, algorithms, methods and compositions of matter are provided for achieving miRNA-like activity in cells/tissues in subjects using the sequential delivery method where the sense and antisense strands are not based on a particular endogenous miRNA. Nevertheless, these compounds are also referred to herein as miRNA mimics. The starting point for these compounds is a novel seed sequence selected to target the 3′-UTR of one or more mRNA types of commercial interest for silencing. This novel seed sequence along with its sense strand complement is inserted into the appropriate regions of a duplex that is capable of efficiently loading its antisense strand into RISC (duplex vehicle) and the resulting duplex is subjected to modification in accordance with the present invention.

In yet another embodiment an algorithm is used to further modify the antisense strands of the present invention so that they can induce the intended RNAi-dependent activity in subjects in the absence of a partner sense strand.

In a final embodiment, carriers are employed with individual strands in cases where it is desirable to restrict the cell and/or tissue types targeted in subjects in vivo as the same silencing effect in another cell/tissue type can produce an undesirable side effect. The rationale for and means to achieve this type of cell/tissue targeting is well understood in the art including its application to single strand oligo drugs (antisense or aptamers). In extensive review of carriers suitable for use with single strand oligos and for the targeting of particular cell/tissue types is provided in PCT/US2009/002365. Such relatively small and simple established carriers are to be contrasted with those in development for the delivery of conventional siRNA and conventional miRNA.

### D. Overview of Invention Details

### 1. Comments on Terminology

The term “nucleoside” is to be interpreted to cover normal ribonucleosides and deoxyribonucleosides as well as the nucleoside analogs provided. It is to be understood that the stereochemical orientations of the compound referred to are subject to the same assumptions as are found in the literature generally when short hand terminology is used, for example, when ribose is referred to it is to be understood as being D-ribose or when arabinonucleic acids (ANA) are referred to the are D-arabinonucleic acids.

“Nucleoside substitute” refers to structures with chemistries radically different from nucleosides, but which play at least one role undertaken by a nucleoside in other situations. In addition, it is to be understood that the scope of the modifications that apply to 3′-end overhangs are broader than those applying to structures that occur in the regions of the seqRNAi strand that will form a duplex with its partner strand(s).

Statements such as “unless otherwise specified” or “unless otherwise provided for” refer to other specified modifications described herein that provide for a different modification(s) under certain circumstances. In these and in other instances where two or more rules specifying different modifications for the same entity, the more narrowly applicable rule (applies to fewer seqRNAi strands) will dominate. For example, rules applicable to a particular architecture dominate architectural independent rules.

The terms “preferred” and “most preferred” are used to designate the optimal range of configurations for strands for the majority of possible seqRNAi sets. In some instances, due to factors such as those arising from sequence specific differences, the optimal variant for a particular specification will not be what is generally preferred or most preferred. In such instances the selected variant still will fall within the more general range of variants provided for herein. Any such decision related to the use of variants that are not otherwise preferred or most preferred will be primarily based on balancing the desired level of silencing potency for the intended target along with the desired duration of this silencing vs. reductions in off-target effects. Off-target effects include minimizing the suppression of the expression of unintended targets and minimizing unintended modulation of innate immunity. These undesired effects are commonly associated with conventional siRNA duplexes and/or their component strands. They can be measured using methods well known in the art.

“Silencing activity” refers to a level of silencing activity which is substantially specific to the intended target while minimizing off target effects. Preferably the target is silenced at greater than 50%, 60%, or 70% in cases where seqsiRNA, and seqImiRs used. In cases where seqMiRs are utilized, suppression of expression of at least 25%, 35%, 45% or >50% of 1, 2, 3, 4 or 5 of the targeted sequences is preferred. In the case of therapeutic seqRNAi compounds, for example, the commercial purpose is sufficiently suppressing the intended target to the point a therapeutic benefit is achieved. In the case of functional genomics, for example, this term refers to those levels of intended silencing activity required to suppress the target levels to the point that significant biologic changes can be measured that allow the biologic role(s) of the target to be better understood.

Rules that are explicitly stated to be applied to seqRNAi strands (sense, antisense or both) apply to the corresponding (sense, antisense or both) seqsiRNA, seqIMiRs and seqMiRs strands. Such rules are not to be assumed to apply to ss-RNAi strands unless otherwise stated. Some ss-RNAi strand modifications are differentiated on the basis of whether they are designed to produce siRNA-type or miRNA-type activity.

Unless otherwise specified it is to be understood that for simplicity certain linkage alternatives to the natural phosphodiester that are described herein (chirally specific phosphorothioate, boranophosphate) can substitute for one or more phosphorothioate linkages described in sections that refer to phosphorothioates generically. Unless otherwise specified, however, the linkages uniquely specified for use in seqRNAi strands that will become 3′-end overhangs in the seqRNAi-based siRNA duplex will only be applied in the context of 3′-end overhang protection (phosphonoacetate, thiophosphonoacetate, amide, carbamate and urea). When a linkage is not specified, it is assumed to be phosphodiester.

### 2. Basic Design Considerations

It is well established in the art that the types of chemical modifications used in seqRNAi strands to achieve nuclease resistance and to provide other essential features also have the potential to adversely affect function. For example, they can reduce and even to eliminate the silencing activity seen in a corresponding unmodified siRNA or miRNA duplex. Further, the proper use of modifications depends on factors such as the underlying sequence, which strand is being considered (sense or antisense) the frequency of use of a particular modification, the nature of the other chemical modifications being used, the overall placements of chemical modifications in the strand, the effects of such factors in one strand on the partner strand and the regional as well as overall interstrand thermodynamics generated when a duplex is formed. In addition to silencing activity these considerations also have a major impact on other functional features of seqRNAi strands and seqRNAi-based duplexes such as the extent to which potential off-target effects are engendered or suppressed. It follows that the greater level of chemical modification that is required for seqRNAi strands compared to the strands in conventional siRNA and conventional miRNA must be more highly orchestrated such that potentially competing objectives are harmonized. This harmonization can be achieved though the use of the algorithms provided herein.

seqRNAi sets are constructed by applying a series of algorithms in a logical order. Some algorithms, such as the one dealing with nuclease resistance are always applied while the application of others depends on particular preferences. A general principle for prioritizing the rules in particular combinations of algorithms applied to the design of a particular seqRNAi set is that more restrictive rule dominate less restrictive rules. Rules can be more restrictive in the sense of providing fewer options for the modification of a particular structure and/or they can be more restrictive in application. In practice once the sequences for a particular seqRNAi set is selected the appropriate series of algorithms directing design of the final seqRNAi strands are most efficiently applied in a logical order. For example the order for the application of particular algorithms could be the following: (1) providing for nuclease resistance other than resistance to double strand endoribonucleases; (2) providing for certain other essential/preferred architecture-independent rules; (3) providing for a selected stand alone architecture (canonical, blunt-ended, asymmetric or small internal segmented); (4) optional application of forked variant to any of these architectures except the small internally segmented; (5) provide for overall and regional interstrand thermodynamic optimization; (6) provide for double strand endoribonuclease protection if needed; and (7) possibly select other optional architectural independent rules.

Each of the possible seqRNAi-based duplex architectures has advantages and disadvantages over the others and a number of these attributes are presented in Table 1. For general purposes the asymmetric architectural design with only the 3′-end overhang is most preferred.

TABLE 1
SUMMARY OF seqRNAi-BASED DUPLEX ARCHITECTURES
THAT CAN BE FORMED IN CELLS FOLLOWING
SINGLE STRAND ADMINISTRATION TO SUBJECTS

| Duplex | | | |
|---|---|---|---|
| Architecture | | | |
| Generated in | General | | |
| Cells | Comments | Potential Advantages | Potential Disadvantages |
| Canonical | Based on the | Presence of overhangs adds | Sense strand overhang can help |
| | architecture of | versatility with respect to | promote its being loaded into |
| | naturally occurring | factors such as duration of | RISC as an antisense strand. |
| | siRNA that includes | silencing and/or increased | |
| | the presence of | 3'exonuclease protection as a | |
| | overhangs in both | function of various possible | |
| | strands. | chemical modifications. In | |
| | | addition, cytoplasmic | |
| | | duplexes with overhangs tend | |
| | | to be less immunostimulatory | |
| | | than those with blunt-ends. | |
| | | The presence of certain types | |
| | | of overhangs may promote the | |
| | | export of siRNA duplexes out | |
| | | of the nucleus. | |
| Blunt ended | Lacks overhangs in | Potential to shorten duration | Compared to the canonical |
| | both strands | of silencing affect other | architecture this architecture |
| | | factors being equal. This can | tends to be more |
| | | be an advantage in some | immunostimulatory when |
| | | commercial applications such | present in the cytoplasm. The |
| | | as when prolonged silencing | lack of overhangs may impede |
| | | may result in undesirable side | the transport of siRNA duplexes |
| | | effects. | out of the nucleus. |
| Asymmetric | Characterized by | When the desired antisense | |
| | antisense strand | strand has a 3′-end overhang | |
| | having 3′ and/or 5′- | and the sense stand does not | |
| | end overhangs while | the probability the desired | |
| | the sense strand does | antisense strand will load into | |
| | not have any | RISC for target recognition | |
| | overhang(s). | can be increased. The | |
| | | presence of a 3'-end overhang | |
| | | also adds versatility with | |
| | | respect to factors such as | |
| | | duration of silencing on the | |
| | | basis of various overhang | |
| | | chemistries. In some instances | |
| | | the selection preference for | |
| | | the desired antisense strand | |
| | | may be further increased | |
| | | through the use of a 5′-end | |
| | | overhang in the antisense | |
| | | strand. | |
| Forked Variant | A variant of | Increases the number of sites | |
| | Canonical, Blunt- | on the RNA target suitable for | |
| | ended and one type | siRNA attacks. This occurs | |
| | of Asymmetric | because the forked variant | |
| | architecture (one | provides the means to promote | |
| | without 5′-end | RISC loading of the desired | |
| | overhang) where the | antisense strand in situations | |
| | forked design | where the terminal sequences | |
| | involves the use of | are otherwise poorly | |
| | mismatches in the | compatible with the normal | |
| | 3′-end of the sense | loading preference mechanism | |
| | strand with the | that is the basis for the | |
| | complimentary | asymmetry rule. An advantage | |
| | antisense strand. It is | of this variant appears in | |
| | applied where the | situations where the choice of | |
| | conditions for the | a site(s) on the RNA target is | |
| | asymmetry rule are | restricted to those that cannot | |
| | particularly | meet the design preferences | |
| | unfavorable. | associated with the basic | |
| | | architectures. For example, it | |
| | | might be desirable to cleave | |
| | | an mRNA target between a | |
| | | primary and a secondary | |
| | | translational start site so that a | |
| | | truncated protein is produced. | |
| | | As another example, it might | |
| | | be desirable to cleave an | |
| | | abnormal mRNA while | |
| | | sparing the normal mRNA. In | |
| | | either of these situations the | |
| | | available target sites may not | |
| | | readily support the favorable | |
| | | thermodynamic asymmetry | |
| | | between the duplexed termini | |
| | | that is required for efficient | |
| | | silencing by the standard | |
| | | architectures unless the forked | |
| | | variant is used. | |
| Small Internally | Characterized by use | Two sense strands eliminates | By having two short strands the |
| Segmented | of two sense strands | possibility that the intended | binding affinity of each of the |
| | in combination with | sense strand will be loaded | two strands for the |
| | a single | into RISC as the antisense | complementary strand is |
| | complementary | strand and reduces or | substantially reduced and may |
| | antisense strand. In | eliminates the importance of | be inadequate for efficient |
| | the case of seqMiRs | the asymmetry rule. Two | duplex formation. This situation |
| | there can be two | antisense strands eliminate | tends to limit the sites on the |
| | antisense strands | any possible contribution of | RNA target for antisense/RISC |
| | and a single sense | the antisense strand forming a | binding to ones that are |
| | strand. | duplex with the 5′-end half of | relatively G/C rich. This can at |
| | | the sense strand from | least partially be compensated |
| | | contributing to target | for by using affinity increasing |
| | | recognition. This can simplify | modifications such as LNA. |
| | | the design of effective | |
| | | seqMiRs. | |

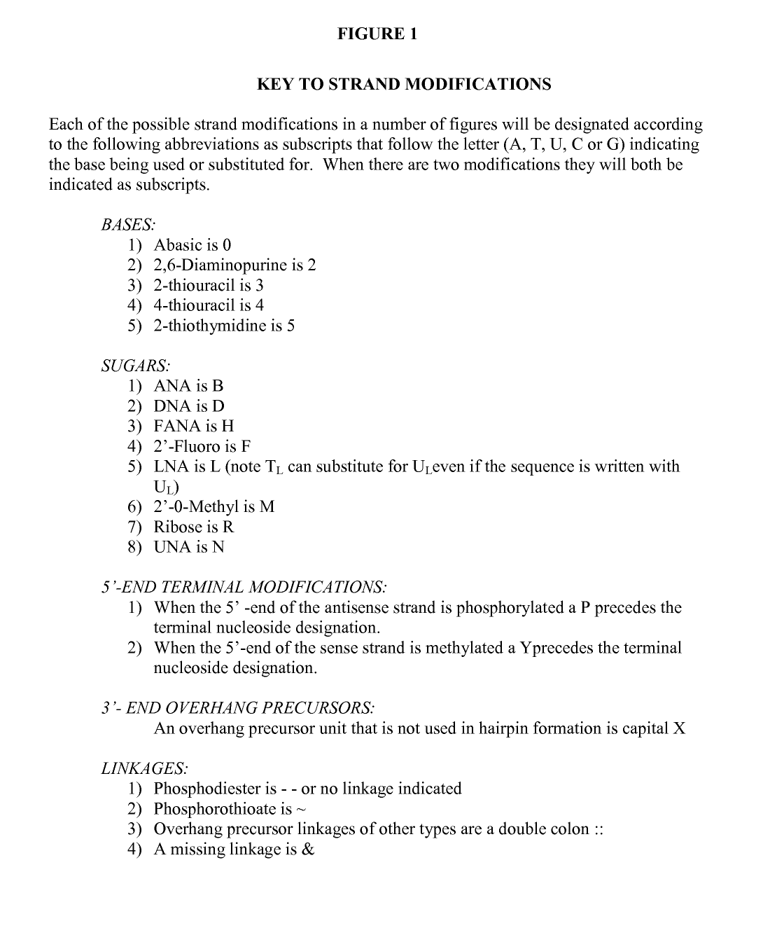

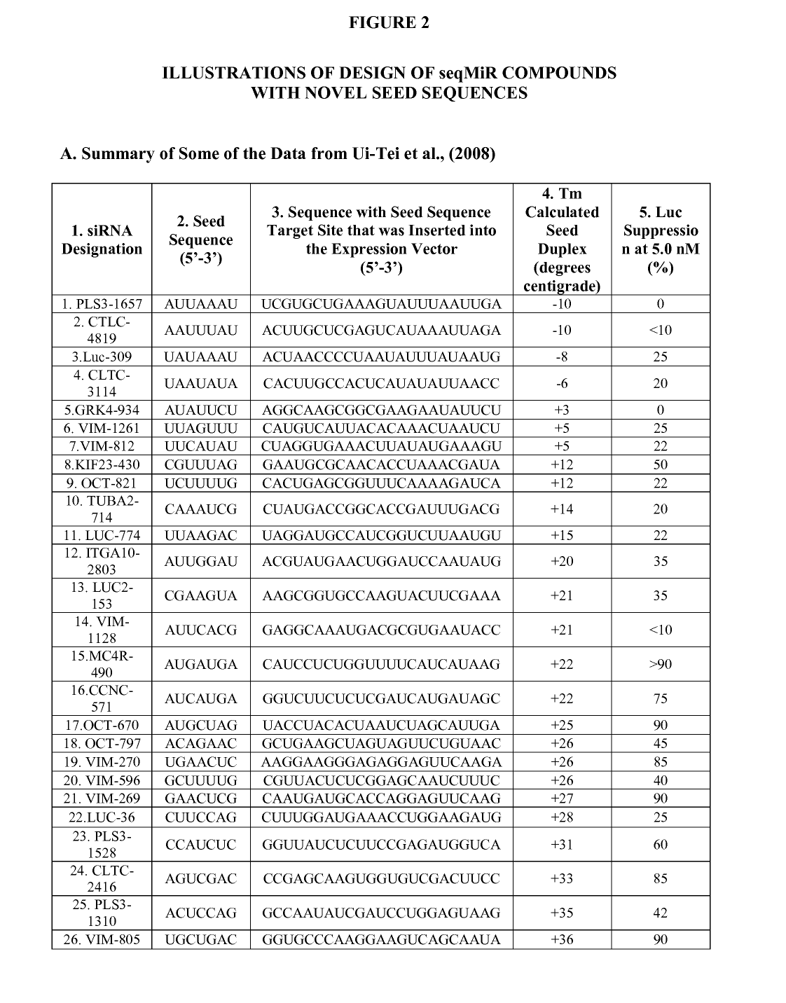

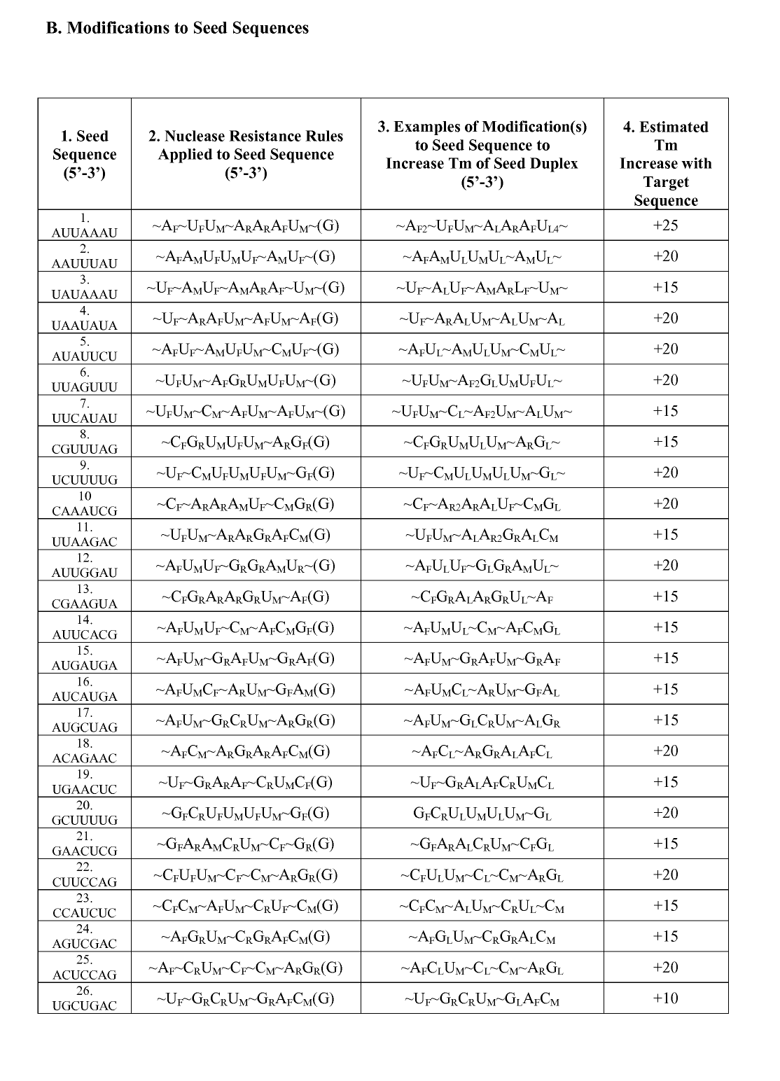

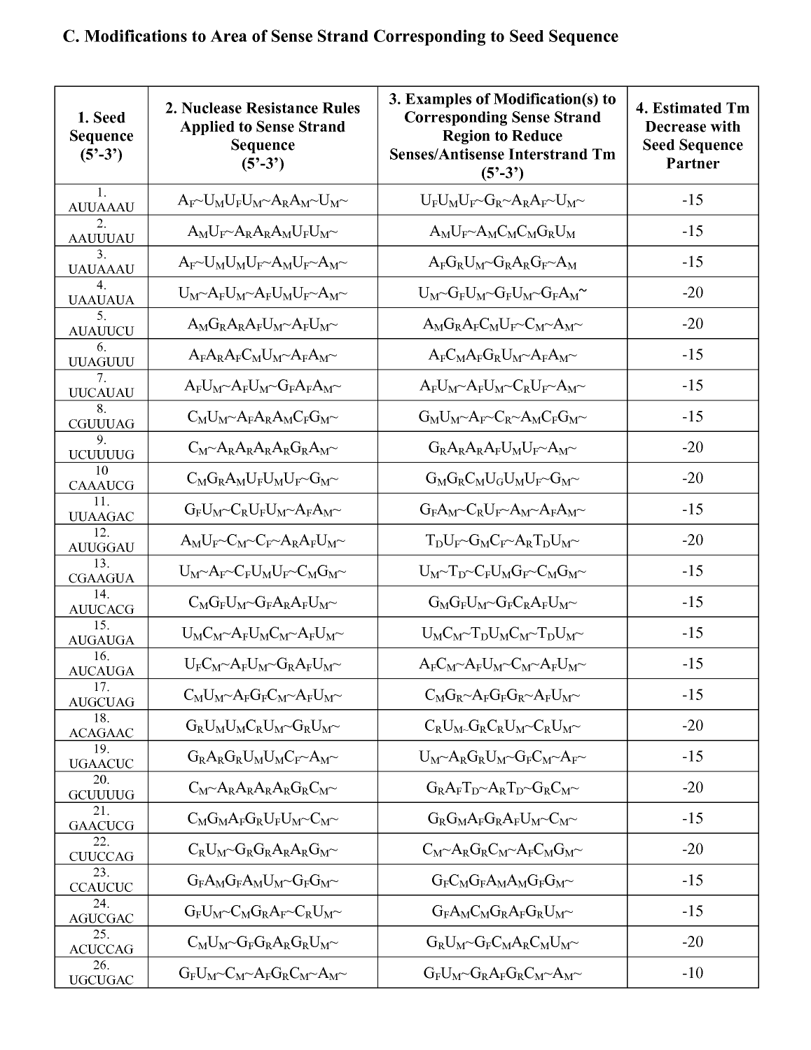

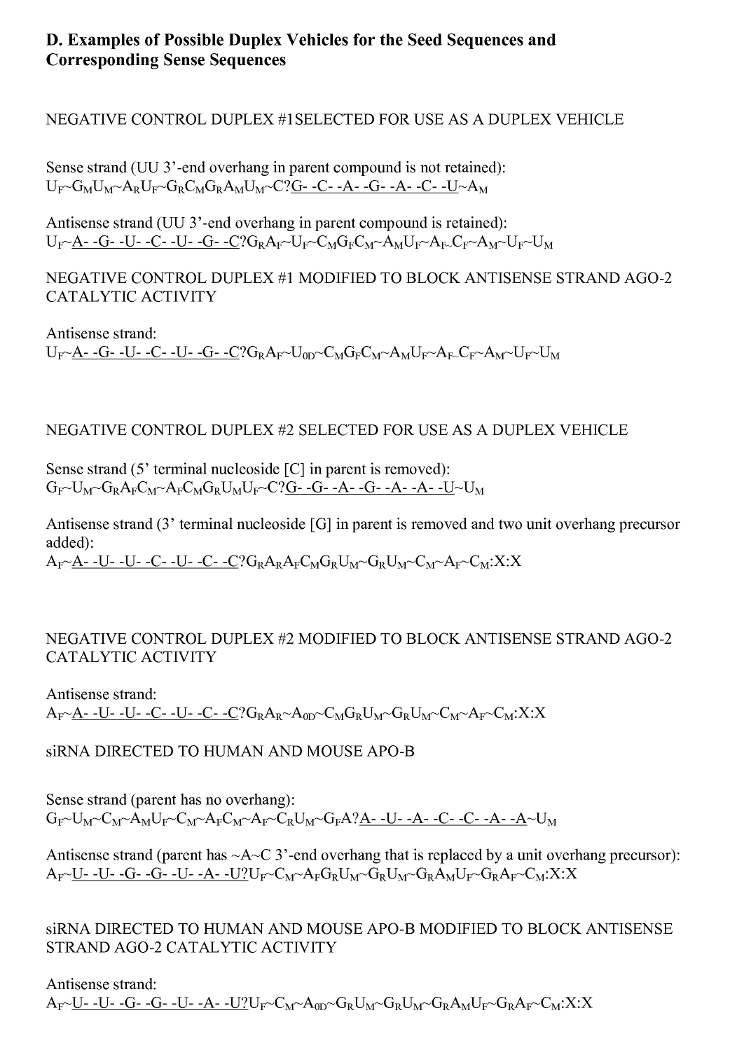

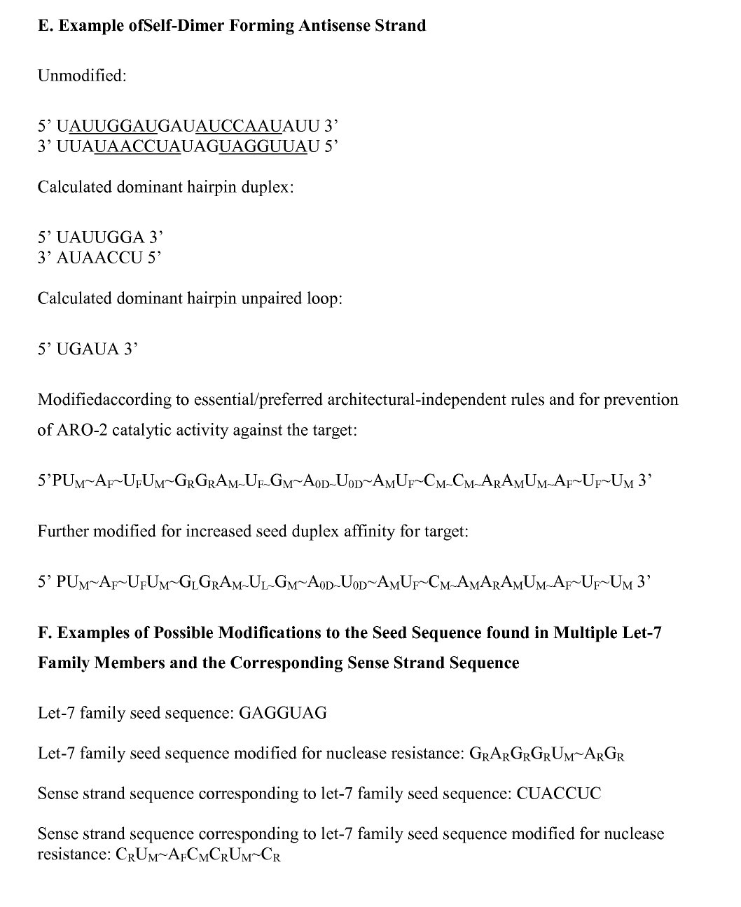

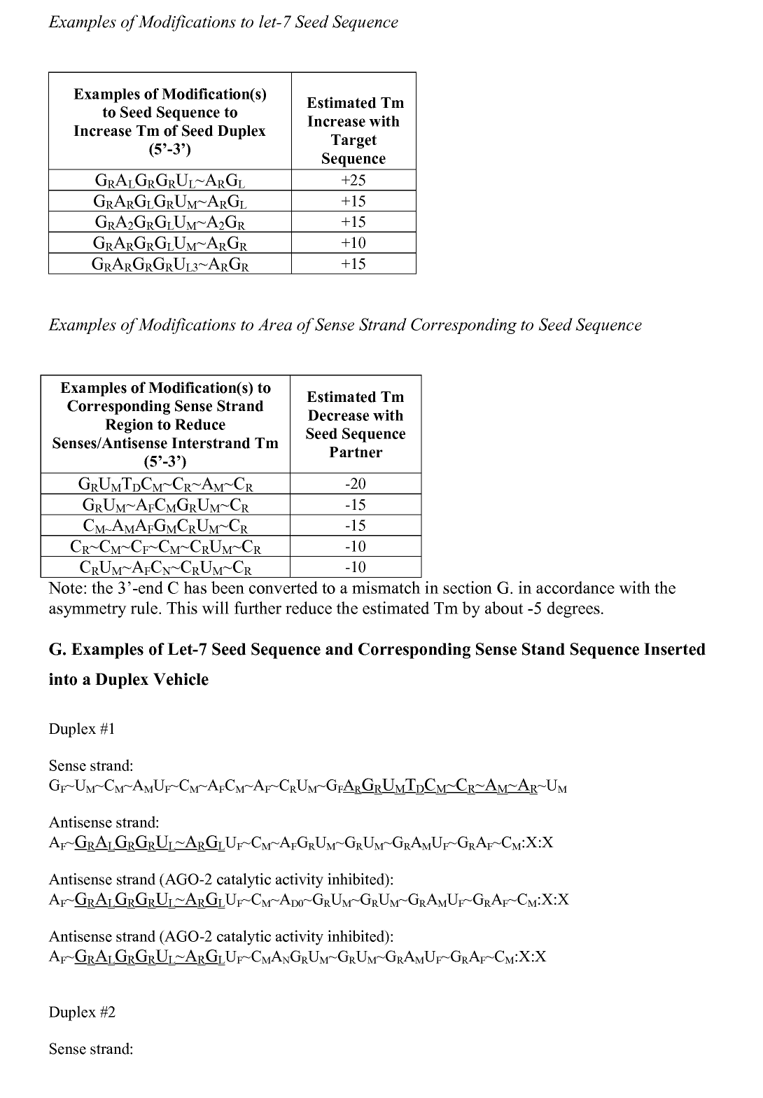

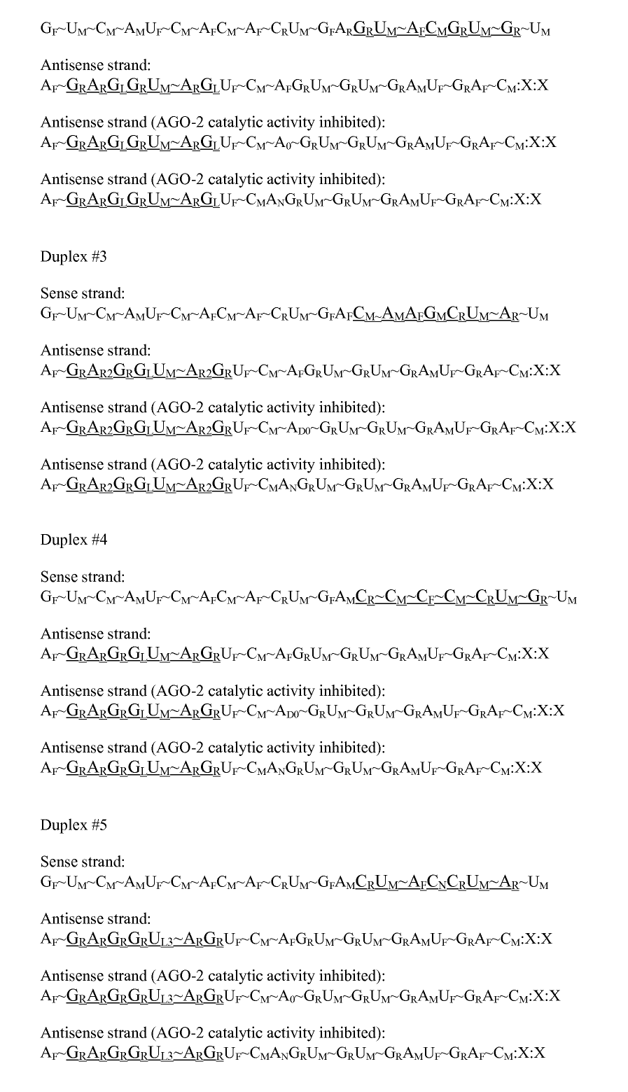

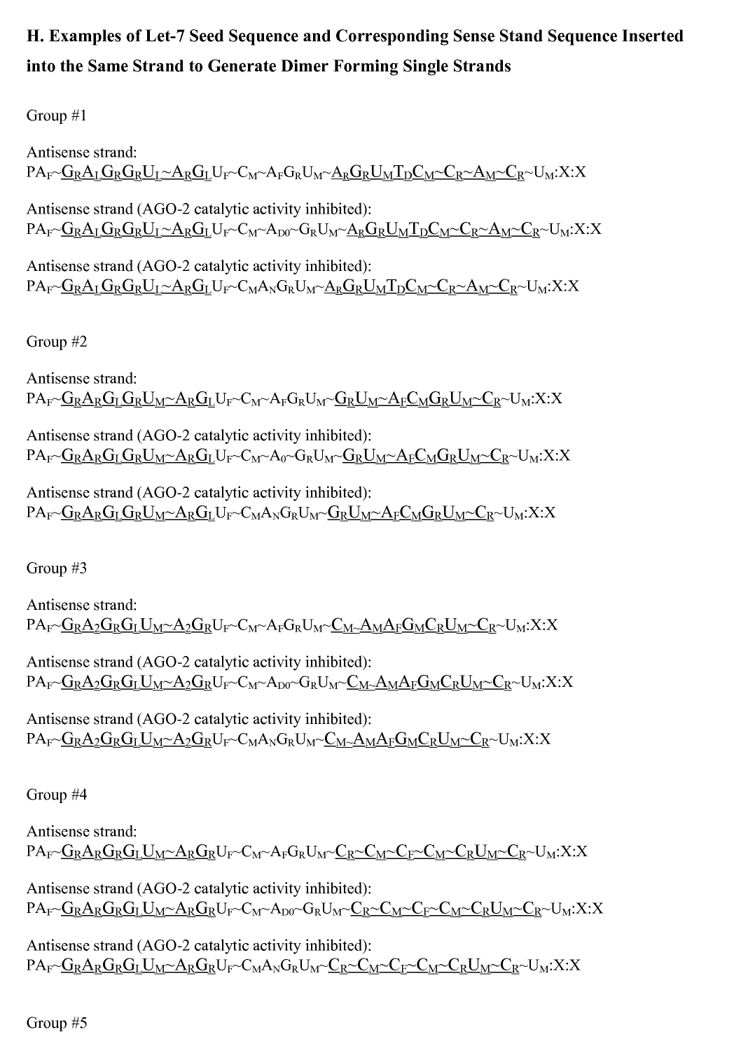

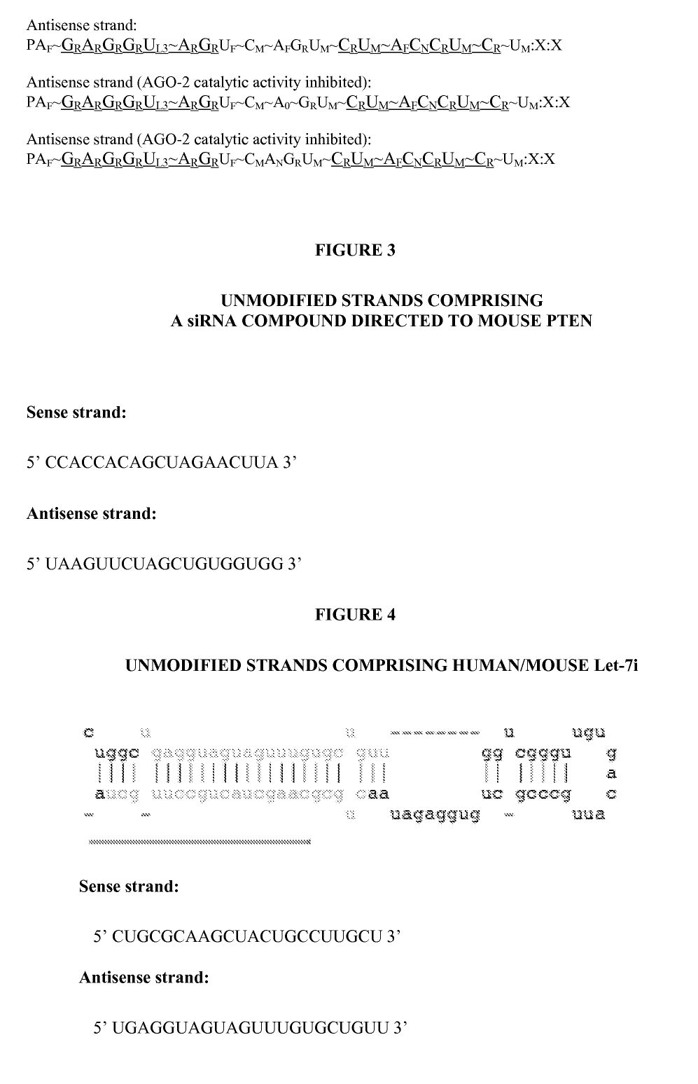

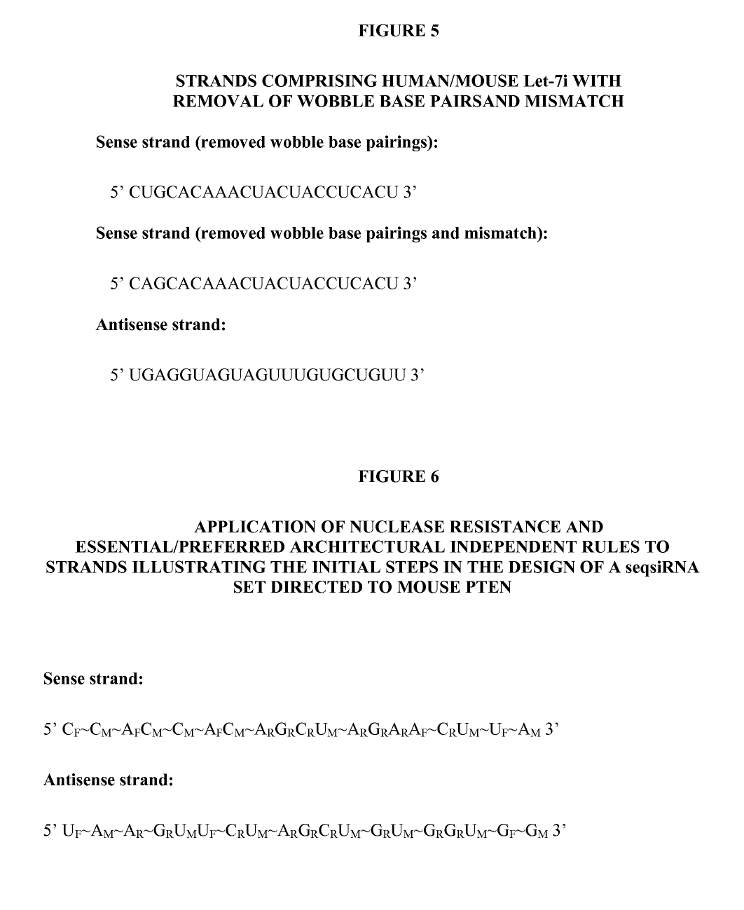

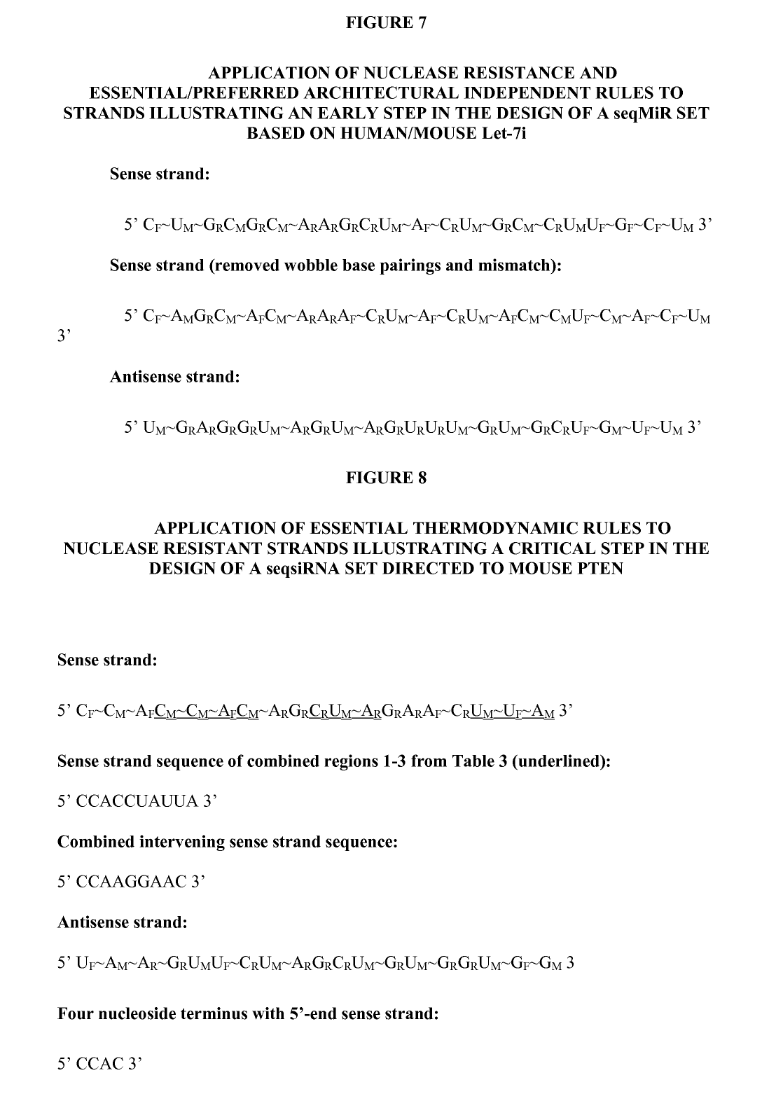

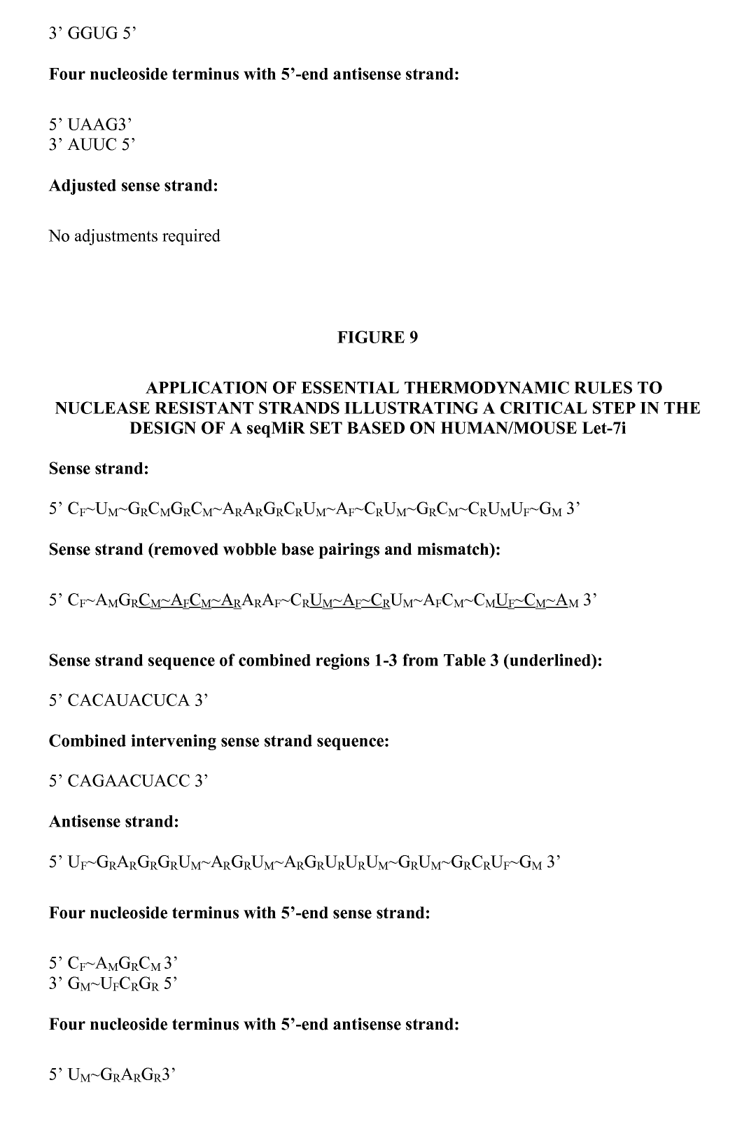

FIG. 1 provides a key for the modifications that can be made to the strands that applies to some but not all of the figures. FIG. 2 illustrates some of the more sophisticated approaches to the design of seqMiR sets that do not apply to other seqRNAi types. To illustrate the general design process a conventional siRNA directed to mouse PTEN has been selected along with the microRNA let-7i. The former compound is used to illustrate the design of a seqsiRNA or seqIMiR set of molecules and the latter compound is used to illustrate the design of a seqMiR set of molecules. The unmodified strands of the selected examples are shown in FIGS. 3 and 4. The optional removal of bulge structures, mismatches and/or wobble base pairs is the first design step in the construction of a seqMiR set. In the case of let-7i there are no bulge structures but there are five wobble base pairs in the duplex and one mismatch. The removal of these is illustrated in FIG. 5 and the effect on the specific nuclease resistance and certain other essential/preferred modifications is shown in FIG. 7. FIGS. 6 to 19 illustrate different aspects of the design process based on the chosen examples. FIGS. 8 and 9 are particularly noteworthy in that they provide examples of strands exhibiting the minimal requirements to qualify as a seqRNAi set of molecules.

### E. Algorithm: Generally Applicable Architectural Independent Rules—Achieving Nuclease Resistance

All the sense and antisense strands of the present invention (seqsiRNA, seqIMiR, seqMiR, ss-siRNA, ss-IMiR and ss-MiR) require certain chemical modifications that provide for nuclease protection while simultaneously being compatible with or supportive of other essential and optional modifications required for additional desirable properties. The required nuclease protections for certain linkage sites in a seqRNAi or ss-RNAi strand are the following:
1) Protection of certain internal linkage sites from single strand endonuclease attack;
2) Protection of linkages between the terminal two or more nucleosides or nucleoside substitutes at the 3′-end of the strand from 3′-end exonuclease attack depending on the size and nature of the 3′-end overhang precursor(s), if any;
3) Protection of the linkage site at the 5′-end of the strand from 5′-end exonuclease attack; and
4) Protection of certain linkage sites in seqRNAi strands that will form a seqRNAi-based duplex from double strand endoribonuclease attack where the needed modifications, if any, protect the strand(s) in the duplex.

The protection of particular internal linkage sites can be relaxed, if necessary, for the central region of seqsiRNA, seqIMiR, ss-siRNA and ss-IMiR as well as for the seed sequence of seqMiR and ss-MiR antisense strands compared to the rest of the antisense strand. These regions of the antisense strands primarily, if not exclusively, represent the targeting codes and they can be more sensitive to the chemical modifications used to generate nuclease resistance than the rest of the antisense or sense strand.

The internal linkage sites to be protected in order to establish nuclease resistance are defined by the ribonucleosides that bracket a given linkage. Thus, the frequency and positioning of the protective chemical modifications are affected by the underlying strand sequence. For general use the linkage sites (reading 5′ to 3′) to be protected from single strand endoribonucleases are those where:
1) A pyrimidine (U, C or T) containing ribonucleoside is followed by a purine (G or A) except C-G.
2) A linkage sites is defined by C-C and U-C.
3) A linkage sites is defined by C-G, A-C and A-U.

Hence, of the 16 possible linkage sites involving ribonucleosides with one or two of the 4 normally occurring bases in RNA (A, U, C and G) only half of them need to be protected to achieve the stipulated nuclease protection. In contrast the linkage sites that do not have to be protected from single strand endoribonucleases are A-A, U-U, G-G, G-C, G-U, G-A, A-G and C-U. T may replace U in a ribonucleoside in some applications described herein and when it does the nuclease protection rules treat it as a uridine.

Approaches for protecting particular linkage sites from single strand endoribonucleases include the following:
1) The 5′ nucleoside member of the linkage has a sugar that is selected from the group consisting of 2′-fluoro, 2′-0-methyl or 2′-deoxyribose unless otherwise specified.
2) When there are two or more contiguous nucleosides and one is preferably a 2′-O-methyl and the contiguous nucleosides include C then it is preferred that the C the be 2′-0-methyl unless otherwise specified.
3) When the 5′ nucleoside sugar is 2′-fluoro it is preferred that the intervening linkage with the 3′ nucleoside be phosphorothioate particularly when the 3′nucleoside is 2′-fluoro or ribose.
4) The intervening linkage can be phosphorothioate or phosphodiester when the 5′ nucleoside has a 2′-0-methyl or 2′-deoxyribose sugar with the phosphorothioate possibly providing added protection.
5) The phosphorothioate is preferred when the linkage site is defined in group 1 (U-G, U-A, C-A) or group 2 (C-C and U-C). In group 3 when the first nucleoside is 2′-fluoro or ribose (C-G, A-C and A-U) the phosphorothioate is preferred when the 3′-nucleoside in the linkage pair is ribose or 2′-fluroro.
6) Unless otherwise specified, the 3′ nucleoside member of the linkage site can have a sugar that is selected from the group consisting of ribose, 2′-fluoro, 2′-O-methyl or 2′-deoxyribose.

In the case of the central region of seqsiRNA, seqIMiR, ss-siRNA and ss-IMiR antisense strands all the indicated modifications can frequently be accommodated without an undo negative effect on the intended silencing activity. When an undo negative effect is seen then the order in which the linkage site protections are removed to achieve a higher silencing effect will be in the reverse of what is listed (i.e. group 3 then 2 then 1). So, for example, the protection of the C-G, A-C and A-U linkage sites (group 3) is the least important.

In the case of the seed sequence of seqMiR and ss-MiR antisense strands the chemical modifications involved in generating nuclease resistance can affect the range of mRNA types suppressed by a endogenously occurring or novel seed sequence and may affect the levels of silencing activity caused by particular miRNA types. When these affects are adverse to the intended commercial purpose, they can be avoided by reducing the level of nuclease protection. As for the central region of the other seqRNAi and ss-RNAi types, reducing the level of nuclease protection against endonucleases follows the reverse order in which they are presented with the third group being the least important.

The general means for protecting a strand of the present invention from single strand 3′-end exoribonucleases independently of any selected architecture requires that at a minimum the terminal 2 nucleosides or nucleoside substitutes (the maximum is 4) and at a minimum the terminal two linkages (the maximum is 4) to be ones that provide for nuclease resistance. Limiting the modifications to two nucleosides or nucleoside substitutes and two linkages is preferred.

The required 3′end exonuclease protection is provided by the following:
1) In the absence of a 3′-end overhang precursor the required 3′end protection can be provided by the use of two terminal nucleosides that are individually selected from the group 2′-fluoro, 2′-0-methyl or 2′-deoxyribose. Strands that have a 3′ terminal 2′-fluoro modification, however, can have a reduced yield with current manufacturing methods.
2) In the absence of a 3′-end overhang precursor the terminal two linkages will be phosphorothioate.
3) The 3′-end exonuclease protection can also be achieved in part or fully by the use of 3′-end overhang precursors as described in the section by that name. The overhang precursor can be 1-4 units long with 2 units being preferred. When there is only one unit the contiguous nucleoside is selected from the group 2′-fluoro, 2′-0-methyl or 2′-deoxyribose and the upstream linkage is phosphorothioate.

The terminal 5′end linkage site is protected entirely or in part as follows:
1) The 5′-end terminal nucleoside is selected from the group consisting of nucleosides with the following modifications: 2′-fluoro, 2′-0-methyl or 2′-deoxyribose unless otherwise specified.
2) When the 5′ nucleoside to be modified is cytidine, 2′-0-methyl is preferred unless otherwise stipulated.
3) The 3′ member of the linkage site can have a sugar that is selected from the group consisting of ribose, 2′-fluoro, 2′-0-methyl or 2′-deoxyribose.
4) The intervening linkage can be phosphodiester or phosphorothioate unless otherwise specified but when the 5′ nucleoside is 2′-fluoro it is preferred that the intervening linkage be phosphorothioate.

Protection from double strand endoribonucleases is important for the brief period between the formation of the seqRNAi-based duplex in cells and RISC association and processing. The relevant enzymes digest both strands of normal RNA duplexes. When a segment in a seqRNAi strand (single strand segment) possesses four sequential phosphodiester linkages contiguous to normal ribonucleosides forms a duplex with a complementary RNA strand and is base paired with such a segment of the same or longer size in the complementary strand, the resulting double strand segment can support low-level digestion by these enzymes. Shorter double strand segments than four do not support digestion. These enzymes, however, are significantly more active when such double strand segments have five to six or more phosphodiester linkages contiguous with normal ribonucleosides in opposition in each strand when the duplex is formed. These enzymes can also digest a single unprotected single strand segment in a duplex if phosphorothioate linkages protect the complementary RNA segment in the seqRNAi partner strand.

Modified nucleosides and 2′-deoxynucleotides of the types described herein, when employed in the single strand segment of at least one strand of a seqRNAi pair forming such a double stranded segment substantially inhibits digestion of the single strand segments of both strands by double strand endonucleases. Thus, seqRNAi strands are designed as follows:
1) When they form a seqRNAi-based duplex in cells with their partner strand there will be no complementary double stranded segments comprising 5 or more consecutive phosphodiester linkages contiguous with normal ribonucleosides in both strands and preferably no segment with 4 or more.
2) One or more modified nucleosides will be supplied to break up any single strand segment(s) in a seqRNAi strand(s) otherwise capable of forming any such double strand segment(s) with its partner strand such that the length of the double strand segment will be limited in length as described.
3) Such modifications can be limited to a single strand segment in one of the two strands in the resulting seqRNAi-based duplex or appear in both. Alternatively, or in addition, duplexed segments of these sizes can be broken up using phosphorothioate linkages, but if this is the only method of protection then it must be applied to the duplexed segments of both strands.

The rules for protecting seqRNAi-based duplexes from double strand endoribonuclease attack is different from the rules for protection from other nucleases in that they are applied after the modifications based on all the relevant rules are applied to a given seqRNAi set. This will help prevent the use of unnecessary modifications.

### F. Algorithms: Generally Applicable Architectural Independent Rules—Other

### 1. Essential/Preferred Modifications

a) Applicable to seqRNAi Sense and Antisense Strands as well as to ss-siRNA, ss-IMiR and ss-MiR:
   i) Unless otherwise specified, it is preferred that within the seqRNAi-based duplex that any 2′ ribose modified nucleoside in one strand is opposed to a nucleoside in the complementary strand that has a different ribose modification or is a normal ribonucleoside or 2′-deoxyribonucleoside.
   ii) It is preferred that uracil not be paired with ribose in the same nucleoside. When uracil is paired with 2′-deoxyribose then it is preferred that the any contiguous nucleoside not be a 2′-deoxyribonucleoside(s).
   iii) It is preferred that any guanine containing 2′-deoxyribonucleoside not be used on the 3′ side of a contiguous cytosine containing 2′-deoxyribonucleoside unless the cytosine is methylated.
   iv) When the use of phosphorothioates for nuclease protection results in less than half the linkages being of this type it is preferred that additional phosphorothioates be inserted to achieve this level.
b) Applicable to seqRNAi Sense Strands:
   i) It is preferred that there are no more than three guanine containing nucleosides in a row in any given sense strand but when four are required then one of the four preferably will be 7-deazaguanosine.
   ii) When a mismatch is indicated by a rule and multiple nucleosides with a standard base (A, T, U, C or G) can fill the role then the preferred nucleoside(s) is the one that produces the most stable linkage site against nuclease attack. For example G-G is more stable than C-G.
   iii) When the introduction of a mismatch is indicated by a rule and the nucleoside selected for a base change to generate a mismatch is an A then the nucleoside is preferred to be changed to one of the following:
      a T is preferred and it is further preferred that the sugar be 2′deoxyribonuclotide if it is in a position where that sugar is permitted by the applicable rules.
      a C is preferred and it is further preferred that the sugar be 2′-0-methyl if it is in a position where that sugar is permitted by the applicable rules
      a U is acceptable but not preferred and if used it is preferred that the sugar be 2′-0-methyl if it is in a position where that sugar is permitted by the applicable rules.
c) Applicable to seqRNAi Antisense Strands:
   i) The antisense strand is 16 to 23 nucleosides in length excluding any 3′-end overhang precursor unit(s) that may be employed.
   ii) More than four guanine-containing nucleosides in a row are not permitted. It is preferred that there are no more than three guanine-containing nucleosides in a row outside the central region but when four are required then one of the four preferably will be 7-deazaguanosine. Four guanine-containing nucleosides in a row or more are not permitted in the central region of the antisense strand.
d) Applicable to seqsiRNA and seqIMiR Antisense Strands:
   i) In the central region preferably none of the nucleoside positions are occupied by entities that would reduce the binding affinity with the intended target compared to a perfectly complementary central region comprised of the common normal nucleosides. Examples of excluded modifications are UNA and abasic entities as well as nucleosides with mismatched bases with the target RNA.
   ii) Preferably no more than two contiguous nucleosides in the central region have the 2′-0-methyl modification.
   iii) There are no restrictions on the number or positions of nucleosides in a seed sequence that can be a 2′-deoxyribonucleoside, however, there is a limit of no more than 40% of an antisense strand, exclusive of any overhang precursors, can be 2′-deoxyribonucleoside. In addition it is preferred that there is a limit of two such nucleosides in the central region and when there are two they are not contiguous. In the rest of the strand, exclusive of any overhang precursor(s), there is a limit of one such nucleoside.
e) Applicable to seqMiR and ss-MiR Antisense Strands:
   i) LNA(s) can be used in the seed sequence, as needed, to increase the binding affinity with the mRNA 3′-UTR target sequence(s) with a maximum of three per seed sequence. It is preferred that when there are multiple LNAs in a given seed sequence that they be separated by at least one nucleoside that does not have the LNA modification. Note T can substitute for U in LNA.
   ii) A 2-thiouracil containing LNA can be used in place of uridine LNA to further boost seed sequence binding affinity with its mRNA target when the corresponding base in the target is adenine.
   iii) LNA or other ribose modified ribose nucleosides of the type provided for herein normal ribose nucleosides can be used in the seed sequence and paired with the following modified bases when the base is complementary to the corresponding base in the target: 2,6-diaminopurine (pairs with adenine), 2-thiouracil, 4-thiouracil, 2-thiothymine.
   iv) It is preferred there not be any G:U base pairs between the seed sequence and the intended target sequence(s).
   v) It is preferred that there are no 2′-deoxyribonucleosides in the seed sequence particularly if there are no LNA modifications in the seed sequence. There is a limit of four 2′-deoxyribonucleosides in the central region and when there are more than two they are not contiguous. In the rest of the strand, exclusive of any overhang precursor(s), there is a limit of one 2′-deoxyribonucleoside.
   vi) It is preferred that the second nucleoside from the 5‘end not be 2’-0-methyl or LNA and that it be 2′-fluoro or ribose.

The application of the nuclease resistance and the essential/preferred architectural independent rules to illustrative seqsiRNA and seqMiR examples is provided in FIGS. 6 and 7 respectively.

### 2. Nonessential/Optional Modifications

The level of nuclease resistance for seqRNAi strands and seqRNAi-based duplexes can be adjusted through the selective use of chirally specific phosphorothioate linkages. The Sp diastereoisomer phosphorothioate linkage is much more nuclease resistant than the Rp diastereoisomer. The mixed chirality of the standard phosphorothioate linkages results in sites where the Rp linkages are cleaved first in susceptible linkage sites. Given that there are often multiple susceptible linkage sites the overall stability of a strand or duplex is thus substantially reduced compared to an Sp chirally pure strand. Hence, when higher levels of nuclease resistance are desired for a particular commercial purpose, compared to what is provided by the standard chirally mixed phosphorothioate linkages, Sp linkages are preferably used to protect those linkage sites susceptible to cleavage.

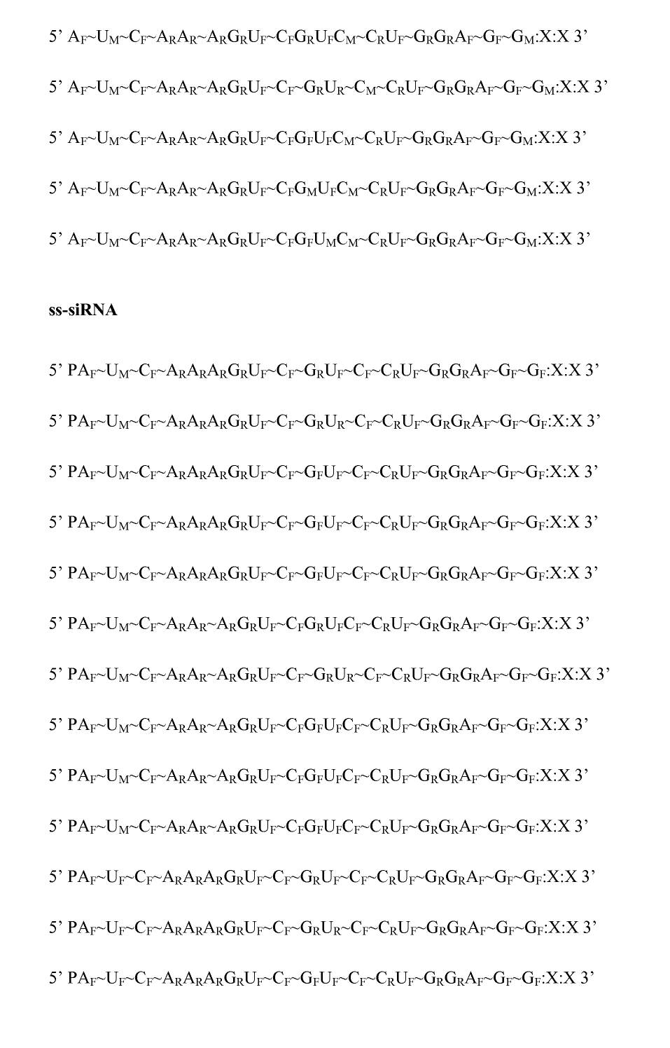

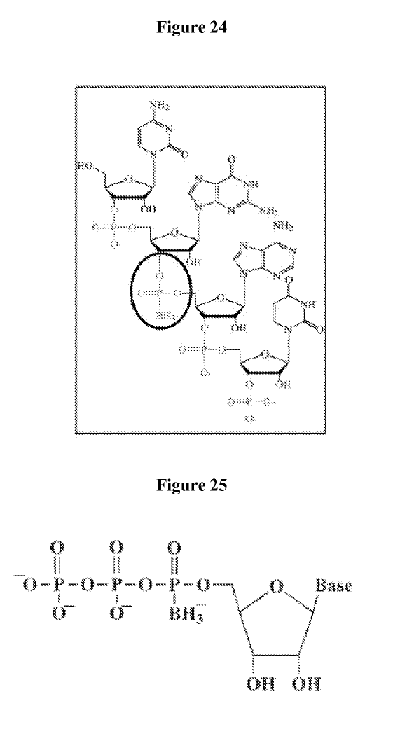

Another possible alternative to the standard phosphorothioate linkage is the boranophoshate linkage with the Sp stereoisomer configuration being preferred. Boranophosphate linkages, (FIG. 24) differ from native DNA and RNA in that a borane (BH₃⁻) group replaces one of the non-bridging oxygen atoms in the native phosphodiester linkage. Such linkages can be inserted in oligos via two general methods: (1) template directed enzymatic polymerization; and (2) chemical synthesis using solid supports. A boranophosphate nucleoside monomer is illustrated in FIG. 25.

Boranophosphate oligo production can be achieved by a variety of solid phase chemical synthetic schemes including methods that involve modifications to the very commonly used approaches employing phosphoramidites or H-phosphonates in the production of phosphodiesters, phosphorothioates and phosphorodithioates among other chemistries (Li et al., Chem Rev 107: 4746, 2007). Other solid phase synthesis techniques more precisely directed to boranophosphates have also been developed over the last few years. Wada and his colleagues, for example, have developed what they call the boranophosphotriester method that can make use of H-phosphonate intermediates (Shimizu et al., J Org Chem 71: 4262, 2006; Kawanaka et al., Bioorg Med Chem Lett 18: 3783, 2008). This method can also be applied to the synthesis of diastereometically pure boranophosphates (Enya et al., Bioorg Med Chem 16: 9154, 2008).

The generation of oligos with mixed linkages such as boranophosphate and phosphate linkages has been accomplished by several solid phase methods including one involving the use of bis(trimethylsiloxy)cyclododecyloxysilyl as the 5′-0-protecting group (Brummel and Caruthers, Tetrahedron Lett 43: 749, 2002). In another example the 5′-hydroxyl is initially protected with a benzhydroxybis(trimethylsilyloxy)silyl group and then deblocked by Et₃N:HF before the next cycle (McCuen et al., J Am Chem Soc 128: 8138, 2006). This method can result in a 99% coupling yield and can be applied to the synthesis of oligos with pure boranophosphate linkages or boranophosphate mixed with phosphodiester, phosphorothioate, phosphorodithioate or methyl phosphonate linkages.

The boranophosphorylating reagent 2-(4-nitrophenyl)ethyl ester of boranophosphoramidate can be used to produce boranophosphate linked oligoribonucleosides (Lin, Synthesis and properties of new classes of boron-containing nucleic acids, PhD Dissertation, Duke University, Durham N.C., 2001). This reagent readily reacts with a hydroxyl group on the nucleosides in the presence of 1H-tetrazole as a catalyst. The 2-(4-nitrophenyl)ethyl group can be removed by 1,4-diazabicyclo[5.4.0]undec-7-ene (DBU) through beta-elimination, producing the corresponding nucleoside boranomonophosphates (NMPB) in good yield.

The stereo-controlled synthesis of oligonucleotide boranophosphates can be achieved using an adaptation of the oxathiaphospholane approach originally developed for the stereo-controlled synthesis of phosphorothioates (Li et al., Chem Rev 107: 4746, 2007). This method involves a tricoordinate phosphorus intermediate that allows for the introduction of a borane group. Other approaches include stereo-controlled synthesis by means of chiral indole-oxazaphosphorine or chiral oxazaphospholidine. Both of these approaches initially used for the stereocontrolled synthesis of phosphorothioates have been successfully adapted to boranophosphates (Li et al., Chem Rev 107: 4746, 2007). In yet another approach to the production of the stereocontrolled synthesis of oligos linked by boranophosphates involves the use of H-phosphonate intermediates (Iwamato et al., Nucleic Acids Sym Ser 53: 9, 2009).

Modifications Applicable to seqRNAi Sense and Antisense Strands as Well as to ss-siRNA, ss-IMiR and ss-MiR:
a) Unless otherwise provided for the terminal 3′-end nucleoside modification in a seqRNAi strand is preferably not 2′-fluoro. This is a manufacturing and not a functional issue. Using existing standard synthesis methods strands having a 2′-fluoro at the 3′-end terminus typically results in a reduced yield.
b) Phosphorothioate linkages can be used to replace phosphodiesters in positions where they are not required to increase nuclease resistance. This can be done, for example, to increase the stickiness of an oligo for certain proteins such as albumin.
c) The Sp diastereoisomer phosphorothioate linkage can be used in linkage sites selected for protection from nuclease cleavage in accordance with the present invention rather than the standard chirally mixed phosphorothioate linkages when a higher level of nuclease resistance is desired.
d) Boranophosphate linkages may replace some or all phosphorothioate linkages.
   Applicable to seqRNAi Antisense Strands:

The 5′-end nucleoside can be phosphorylated at the 5′ ribose position.

### G. Thermodynamic Considerations

### 1. Overview

Thermodynamic considerations related to complementary base pairing are of importance in the design of seqRNAi strands. Most importantly, efficient silencing activity for all the classes of seqRNAi compounds is dependent on optimizing thermodynamic parameters. Such parameters also play a key role in the optimization of the design of seqMiR seed sequences for particular commercial purposes. Thermodynamic stability is reflected in the melting temperature (Tm) or the standard free energy change (AG) for duplex formation. These parameters are highly correlated with each other and can be calculated using well established nearest neighbor calculations or be experimentally determined.

The starting point for constructing strands with the desired thermodynamic properties for use in the present invention is the basic RNA sequence of the strand where it is comprised of the normal ribonucleosides with the most common bases (U, C, G and A) and phosphodiester linkages. Nearest-neighbor calculations can be used to calculate the overall Tm(s) of the strand with its partner strand under physiologic conditions as well as its ability to interact with itself through hairpin and dimer formation (Panjkovich and Melo Bioinformatics 21: 711, 2005; Freier et al., Proc Natl Acad Sci USA 83: 9373, 1986; Davis & Znosko, Biochemistry 46: 13425, 2007; Christiansen & Znosko, Nuc Acids Res published online Jun. 9, 2009). Nearest-neighbor calculations can be undertaken through the use a number of readily available computer programs for oligo analysis.

Regional interstrand Tms play an important role in the design of seqRNAi compounds. Individually these regions can be too short for the nearest-neighbor calculation to be reliably applied. When this is the case a basic Tm calculation based on A:U and G:C content can be applied using the following formula where w, x, y and z are the number of bases of the indicated type: Tm=2(wA+xU)+4(yG+zC)

Tm calculations are adjusted using the approximations shown in Table 2 which accounts for the effects of particular chemical modifications. The table can then be used as guide for making design adjustments to the strands that will result in the desired overall and regional Tms when they combine to form a duplex with the selected architecture.

TABLE 2
Approximation of Effects of Particular Chemical Modification to an RNA Oligo
on Tm Measured when Duplexed with a Complementary Native RNA Oligo

| | Degrees | |
|---|---|---|
| | Change in | |
| | Tm per | |
| Modification | Modification | Comments |
| 2′ fluoro | plus 1.0-plus | |
| | 2.0 | |
| 2′-0-methyl | plus 0.5-plus | |
| | 1.0 | |
| Deoxyribose | | |
| LNA | plus 4.0-plus | Any terminal 5′-end duplexed LNA is poorly |
| | 8.0 | stabilizing as are terminal 3′-end duplexed uracil |
| | | LNAs. These are excluded from the indicated Tm |
| | | range and are not preferred. LNA with adenine has |
| | | about one-half of the stabilizing effect of LNAs |
| | | with other standard bases. Using 2,6- |
| | | diaminopurine or replacing a complementary uracil |
| | | containing nucleoside with an LNA with a thymine |
| | | base can reverse this. Using a 2-thiothymine |
| | | replacement for a thymine can increase the affinity |
| | | of a LNA brining it to the upper end of the |
| | | indicated Tm range. |
| 2,6-diaminopurine | plus 1.0 -plus | Replacement of adenine with 2,6-diaminopurine |
| | 3.5 | increases the Tm. It can be paired with any of the |
| | | ribose modifications provided for herein to form a |
| | | nucleoside. The complementary partner nucleoside |
| | | can have uracil or thymine. |
| 2-thiouracil | plus 2.0-plus | 2-thiouracil can be paired with any of the ribose |
| | 6.0 | modifications provided for herein to form a |
| | | nucleoside. The complimentary nucleoside in the |
| | | partner strand should contain adenine rather than |
| | | guanine when the goal is to optimize stability. The |
| | | most stabilizing nucleosides have 2-thiouridine |
| | | paired with LNA where the use of this base further |
| | | increases the stabilizing effect of LNA. Internal 2- |
| | | thiouridine containing nucleosides are more than |
| | | two fold more stable than are ones found at the |
| | | most 5′-terminal position of an oligo duplex. 2- |
| | | thiouridine containing nucleosides at the most 3′- |
| | | terminal position in an oligo duplex have little or |
| | | no stabilizing effect. |
| 4-thiouracil | plus 3.0-plus | 4-thiouracil can be paired with any of the ribose |
| | 8.0 | modifications provided for herein to form a |
| | | nucleoside. The complimentary nucleoside in the |
| | | partner strand should contain guanine rather than |
| | | adenine when the goal is to increase stability. The |
| | | most stabilizing nucleosides have 4-thiouracil |
| | | paired with LNA where the use of this base further |
| | | increases the stabilizing effect of the LNA |
| | | modification. |
| 2-thiothymine | plus 2.0-plus | 2-thiothymine can be paired with any of the ribose |
| | 6.0 | modifications provided for herein to form a |
| | | nucleoside. The most stabilizing nucleosides have |
| | | 2-thiothymine paired with LNA where the use of |
| | | this base further increases the stabilizing effect of |
| | | LNA. |
| UNA | minus 2.0- | A single UNA nucleoside will reduce the Tm for |
| | minus 10.0 | the seqRNAi duplex with lower Tm reductions for |
| | | UNA placed near the termini and higher Tm |
| | | reductions for UNA placed near the center of the |
| | | duplex |
| Arabinonucleoside (ANA) | minus 1.5-2.0 | |
| Mismatch | minus 2.0- | The effect of the same mismatch depends on the |
| (involving nucleosides with | minus 12.0 | nature of the mismatch and on where it falls in the |
| standard A, C, G, U or T bases) | | duplex with internal mismatches being two fold or |
| | | more destabilizing than mismatches at the |
| | | duplexed termini Substitution of a nucleoside with |
| | | an adenine for one with a guanine will at most |
| | | reduce the Tm about 1.0 degree given that a |
| | | partner nucleoside with a uracil has a wobble base. |
| | | This will have little if any effect. Individual LNA |
| | | mismatches are about one-third less destabilizing |
| | | than mismatches involving nucleosides with 2′- |
| | | modifications to the ribose or with ribose itself. |
| Phosphorothioate | minus 0.4- | |
| | minus 1.2 | |

* Tm is measured in degrees centigrade under physiologic conditions. The numbers provided are approximations and the actual affects on Tm are influenced by a number of parameters including but not limited to the length of the strand, the position of the modification in the duplex and the presences of other modifications in the strand. It is to be assumed that the affinity effects of the indicated nucleoside modifications are with respect to a complementary nucleoside in an oligonucleotide strand unless the modification is specifically stated to be a mismatch.

### 2. Overall and Regional Interstrand Binding Affinities

The overall Tm for the seqRNAi-based duplex formed by a particular seqRNAi set is important. As the Tm increases above about 55 degrees centigrade, for example, the likelihood that AGO-2 will be preferentially loaded into RISC relative to the other argonautes increases. AGO-2 is the only argonaute with the catalytic activity that is important for seqsiRNA and seqIMiR activity. In contrast, the large majority of seqMiRs are relatively indifferent to which argonaute is incorporated into RISC, however, loading of AGO-2 has the potential to generate off target effects by these compounds if it's catalytic activity is not blocked using the appropriate design considerations. Accordingly, overall Tms of about 65 degrees centigrade and above are preferred for seqsiRNA and seqIMiR sets to optimize AGO-2 loading. Lower Tms are preferred for seqMiRs unless the direct catalytic activity of AGO-2 is inhibited. The latter can be achieved by preventing the nucleosides in positions 10 and/or 11 from the 5′-end of the antisense strand from effectively base paring with an unintended target.

Certain relative differences in interstrand affinities in particular regions of the seqRNAi-based duplex (regions explicitly defined in Table 3) are also important for all the seqRNAi-based duplex architectural variants other than small internally segmented. The three regions explicitly defined by Table 3 with respect to the sense strand are the areas in the duplex where collectively a relatively lower binding affinity compared to the overall interstrand affinity can promote efficient RISC loading and retention of the antisense strand with the removal of the sense strand. Lower Tms in regions 1 and 3 appear to promote unwinding of the duplex and a substantially lower Tm in region 2, such as can be produced by a mismatch, UNA or abasic nucleoside can help promote removal of the passenger strand. When AGO-2 is loaded into RISC and there is an appropriate cleavage site in the sense strand (opposite the linkage between nucleosides 10 and 11 of the antisense strand), however, it can promote the efficient removal of the sense strand when there is no mismatch, UNA or an abasic nucleoside to region 2.

These are also the primary regions to look to for affinity lowering modifications particularly in the sense strand when it is important to reduce the overall Tm of a seqRNAi duplex if it is above the preferred range. The overall Tm of a seqRNAi-based duplex can be too high to be directly measured (over about 95 degrees centigrade under physiologic conditions), however, and the compound can still produce the desired silencing effect if these regional interstrand affinities are properly managed.

The general rule is that it is preferable for the combined three regions explicitly defined by Table 3 to have a lower Tm than the combined intervening regions when both are considered as a continuous sequence and are corrected for any size difference. These combined sequences are large enough to be evaluated using the more accurate nearest neighbor calculation. It is also preferred that all three of the explicitly defined regions will have a relatively lower Tm corrected for size than the Tm of the combined intervening sequences. The small size of the individual regions explicitly defined by Table 3, however, requires the use of a basic Tm calculation that does not take the nearest-neighbor effects into account.

When the overall Tm for the compete duplex is above the preferred upper level modifications to reduce it should be preferentially made in the regions explicitly defined by the Table. Even when the overall duplex Tm is in the preferred range mismatch(s) with the antisense strand, a single UNA or a single abasic nucleoside in one or more of these regions can promote the intended silencing activity. In seqsiRNA/seqIMiR sets, however, a low relative Tm in region 2 can be less important when the positions in the sense strand opposite positions 10 and 11 counting from the 5′-end of the antisense strand have a phosphodiester linkage and the nucleoside on the 5′side of this linkage site in the sense strand is not 2′-0-methyl and is preferably ribose or 2′-fluoro. This configuration facilitates the cleavage of the sense strand by AGO-2 and in turn this facilitates the removal of the sense strand from RISC.

TABLE 3
Regions Suitable for Modifications that Provide
for Regional Reductions in Interstrand Affinity (Tm) in
seqRNAi-based Duplexes

| Length of | | | |
|---|---|---|---|
| Sense Strand | | | |
| (exclusive of | Nucleoside Position Counting | | |
| any overhang | from 5′-end of Sense Strand | | |
| precursor) | Region 1 | Region 2 | Region 3 |
| 23 | 4-7 | 14-16 | 21-23 |
| 22 | 4-7 | 13-15 | 20-22 |
| 21 | 4-7 | 12-14 | 19-21 |
| 20 | 4-7 | 11-13 | 18-20 |
| 19 | 4-7 | 10-12 | 17-19 |
| 18 | 4-6 | 9-11 | 16-18 |
| 17 | 4-5 | 8-10 | 15-17 |
| 16 | 4 | 7-9 | 14-16 |

* It is to be understood that the regions being identified include the corresponding duplexed portion of the antisense strand. The sense strand is used as reference because the widest range of possible chemical modifications and other manipulations, such as mismatches, that can be used to reduce the interstrand affinity in these regions can be applied to the sense strand without reducing silencing activity. The indicated length of the sense strand is exclusive of any 3′-end nucleosides or nucleoside substitutes that will form an overhang precursor. The range of indicated nucleoside positions includes all of those indicated in the given region. So, for example, 4-7 is to be read to include both the 4th and 7th nucleosides.

Designs for the strands that make up a seqRNAi set of molecules must include means to promote the selection of the desired antisense strand by RISC from the seqRNAi-based duplex. One of the means used to promote the intended antisense strand being loaded into RISC as the de facto antisense strand is based on the primary mechanism for antisense strand selection from endogenous siRNA and miRNA duplexes. The principle behind this mechanism is sometimes referred to as the asymmetry rule. According to this rule the relative Tm of the 4 terminal duplexed nucleosides at one end of the duplex compared to the corresponding nucleosides at the other end of the duplex plays a key role in determining the relative degree to which each strand will function as the antisense strand in RISC. The strand with its 5′-end involved in the duplexed terminus with the lower Tm more likely will be loaded into RISC as the antisense strand. The Tm effect, however, is not evenly distributed across the duplexed terminal nucleosides because the most terminal nucleoside is the most important with the successive nucleosides being progressively less important. Violations to this rule do not necessarily render a particular siRNA or miRNA duplexe nonfunctional but they likely will exhibit suboptimum activity because there will be more loading of the intended sense strand into RISC as the de facto antisense strand and loading of the intended sense strand can increase the likelihood of off target effects.

The asymmetry rule is important for the majority of seqRNAi architectural types. The simplest way of establishing it for a seqRNAi set against a particular target is simply to select sequences that will result in compliance with the asymmetry rule following the application of the necessary rules for chemical modifications to the strand. When necessary the information in Table 2 can be used to bring a strand set into compliance with the asymmetry rule. In situations where the strands are exceptionally out of compliance with the asymmetry rule the forked-variant can be employed with most of the architectures.

The two 4 nucleoside duplexes involved in determining compliance with the asymmetry rule are too short to apply the nearest neighbor calculation with a reasonable degree of confidence in determining the Tm values. Instead the more basic calculation can be used to approximate the Tms for the unmodified duplexes. Once the Tm for the unmodified duplexes is determined then it can be adjusted based on the Tm affects of the modifications provided in Table 2. This determination, however, does not take into account the decreasing importance of the nucleosides as one moves away from the terminus. To account for this in a simple way it is preferred that the overall Tm for the 4 nucleoside duplex be lower for the one containing the 5′ end of the antisense strand and that the most terminal two nucleoside pairs of this duplex have a lower affinity for their partner nucleoside than the corresponding pairs at the other terminus.

RISC requires that the selected antisense strand be phosphorylated at the 5′ CH₂OH position of the 5′-end ribose or ribose substitute in order for the strand to be active in silencing.

Thus the simplest method to inhibit the loading of the desired sense strand into RISC as the antisense strand is to 5′-methylate the sense strand at this position. The desired antisense strand can be manufactured to be 5′-phosphorylated or an intracellular enzyme can be relied on to provide the phosphorylation after the strand has entered the cell. This is to be used as a supplement to the implementation of the asymmetry rule in strands designed with particular architectures in mind.

Methods for measuring the relative contributions of each of the strands in a siRNA or miRNA duplex to silencing are well established in the art. These techniques can also be applied to seqRNAi-based duplexes to ensure that the intended antisense strand is being efficiently used by RISC. For example, expression vectors with a read out protein such as luciferase or enhanced green fluorescent protein can be constructed with target sequences capable of being recognized by the targeting code for any strand that directs RISC silencing. Two such vectors with read out proteins that can be discriminated in the same cells can be constructed where each one responds to a different strands in a seqRNAi pair. Next these expression vectors can be transfected into a cell line along with or just prior to the administration of the seqRNAi-based duplex that is comprised of the test strands. By measuring the relative level of silencing of each of the read out proteins it is possible to determine the relative efficiencies with which each of the strands silences their respective targets. Such an assay provides the means to evaluate the extent to which an intended sense strand is being loaded into RISC as an antisense strand.

### 3. Targeting Codes and Targets

The central region and the seed sequence are the principal if not exclusive targeting codes for conventional siRNA and miRNA respectively. Modifications to these regions of the antisense strand, therefore, are particularly momentous in terms of their ability to affect the silencing of the intended target(s). These basic concepts also apply to seqRNAi, ss-siRNA, ss-IMiR and ss-MiR antisense strands.

siRNA, seqsiRNA, seqIMiR, ss-siRNA and ss-IMiR antisense strands are most effective when they are loaded into RISC with AGO-2 because this argonaute is unique in having catalytic activity against the RISC target. AGO-2 specifically cleaves the target mRNA at the linkage opposite the one joining nucleoside positions 10 and 11 counting from the 5′-end of the antisense strand. To be effective the nucleosides in positions 10 and 11 along with several of the contiguous nucleosides must be fully complementary with the mRNA target. Thus, mismatches in the central region of the antisense strand in particular will undermine the intended silencing activity. The binding affinity of the central region for the mRNA target, however, appears to be comparatively unimportant for silencing activity within the range of affinities generated by the types of chemistries allowed by the present invention. Outside the central region of the antisense strand and exclusive of any overhangs it is preferable that the sequence have a high degree of complementarity with the mRNA target. A small number of mismatches, however, typically can be tolerated.

The typical normal endogenous mechanisms that support miRNA, seqMiR and ss-MiR activity involve the induction of mRNA degradation processes where the antisense strand loaded RISC acts simply to recognize particular mRNA types as targets. Once this occurs other cellular elements form complexes with RISC that result in mRNA degradation that often starts with the poly-A tail. In this context, the details of the thermodynamic interactions involved in the complementary base pairing between the seed sequence and the complementary sequence in the mRNA 3′-UTR require more attention than the complementary base pairing between the central region of other RNAi types and their mRNA target.

One way to construct seqMiRs is simply to apply the key architectural independent algorithms and a selected architectural dependent algorithm to a particular endogenous miRNA or a version of it that has been stripped of bulge structures, other mismatches between the otherwise complementary strands and/or wobble base pairings. Alternatively, the seed sequence from a particular endogenous miRNA or a novel seed sequence can be placed in a duplex vehicle along with a complementary sequence into the corresponding area of the sense strand. Any AGO-2 based catalytic activity exhibited by the duplex vehicle can be inhibited, for example, by replacing nucleosides 10 and/or 11 counting from the 5′-end of the antisense strand with ones that are abasic, UNA and/or FANA. The abasic nucleosides can have any of the sugar modifications provided for herein including the unlocked variant (the sugar in UNA), 2-deoxyribose and FANA. Abasic nucleosides preferably are joined to adjacent nucleosides by phosphorothioate linkages.

Novel seed sequences can be constructed for particular purposes using a combination of recently developed computer and molecular biologic techniques that have been used to study the details of the interactions of the seed sequence of endogenous miRNAs and the complementary sequences in mRNA species that are subject to silencing (Chi et al., Nature Structural & Mol Biol 19: 321, 2012 provides some specific examples). Potential novel seed sequences initially can be identified by examining the 3′-UTRs for complementary sequences in the collection of mRNAs that are of interest for silencing. These complementary sequences will have to meet certain thermodynamic criteria as described below. Next a prototype of the novel miRNA can be constructed, for example, by placing the seed sequence in a selected antisense strand that meet the design criteria for seqMiR and ss-MiR compounds. The ability of the prototype seqMiR to physically recognize the collection of mRNAs of interest is then analyzed. Prototype compounds capable of binding to a desired collection of mRNAs can be then tested in silencing studies. Finally, adjustments in the binding affinity of the seed sequence for its mRNA target sequences can then be made as needed.

There is a substantial literature that describes the interactions of the seed sequence of endogenous miRNA with its mRNA targets. It has also been discovered that the seed sequence in conventional siRNA is a common major contributor to the off-target effects seen with this class of RNAi. Thus, it is clear siRNA can also function as a novel type of miRNA albeit one where the resulting silencing activity is usually not desired. It follows from this that the sequences of particular miRNA antisense strands that lie outside the seed sequence are not required for achieving miRNA-type silencing.

Ui-Tei et al., (Nucleic Acids Research 36: 7100, 2008) have illuminated some key thermodynamic considerations that affect the efficacy of particular seed sequences in siRNA with respect to engendering miRNA-type activity against mRNA targets. They demonstrated that the thermodynamic stability of the duplex formed between the seed sequence and the mRNA target sequence has a strong positive correlation with the degree of seed sequence dependent silencing. The range of calculated seed region/mRNA target duplexes tested ranged from −10° C. to 36° C. while the corresponding ΔG values ranged from −16 to −7 kcal/mol. This demonstrates there is roughly a 5° C. increase in Tm per −1 kcal/mol change in AG. The ΔG value can be converted to the dissociation constant for the seed duplex using the established formula ΔG=−RT ln(1/K_{d}) where T is 298.15 K. The results showed that there is a 10⁶ fold difference in the dissociation constant between the seed/target duplex with the highest ΔG value and the lowest.

All of the 26 siRNA compounds tested had seed region dependent off target effects when used at high concentration (50 nM) but only 5 of 26 (35%) had off-target effects when used at a low dose (0.5 nM). These siRNA compounds were divided into two groups based on whether they resulted in greater than or less than 50% seen region based target suppression. It was found that a calculated Tm of 21.5 degrees centigrade for the seed duplex distinguished the two groups with the higher Tm positively correlating with the higher silencing activity (r=−0.72 in linear regression analysis of silencing activity vs. Tm). This high level of correlation is surprising given the fact each of the siRNAs tested were distinctly different compounds that could be expected to vary in terms of factors such as the efficiency of antisense strand loading into RISC.

The seed duplex Tm calculated in exactly the same way for 733 human miRNAs in the miRBase database showed that 75% of them had values above 21.5 degrees centigrade. Twenty percent were above 40 degrees centigrade and 5% were below 10 degrees. Indeed, 13 of the 733 (2%) had calculated seed duplex Tms above 50 degrees centigrade.

These thermodynamic parameters assist in the optimization of seqMiRs and ss-MiRs that are based on a particular endogenous miRNA seed sequences or to generate miRNA activity based on a novel seed sequence. When they are based on a seed sequence from endogenous mRNA the overall level of silencing activity can be increased or decreased by increasing or decreasing respectively the overall seed duplex Tm with respect to the mRNA types to be silenced. When the complementary sequence to the seed sequence in the mRNA 3′UTRs varies the relatively affinity of the seed sequence for such target sequences can be adjusted to have a higher affinity for some and a lower affinity for others based on the desired pattern of silencing activity. Based on the Ui-Tei et al. (2008) data it is clear that seed duplex affinity between a seqMiR or ss-MiR seed sequence and its mRNA target sequence is preferably above 21.5 degrees centigrade for Tm and/or below a ΔG of −12 for those mRNAs that are to be silenced and preferably below 15 degrees Tm and above −11 ΔG for those that are not to be silenced.

The basis for adjusting the binding affinity for a particular seed sequence and its mRNA target sequence(s) are the chemical and other modifications provided herein that affect complementary base pairing affinity. Approaches for a number of these modifications are provided in Table 2. The use of these modifications must also take into consideration all the other design rules that apply to seqMiRs and ss-MiRs including other thermodynamic considerations. The seed region of an antisense strand involves nucleoside positions 2-8 counting from the 5′-end and the asymmetry rule, where it applies, involves nucleosides 1-4 from the 5′end and in the case of the forked variant nucleosides 1-6 from the 5′end. Any modifications to the overlapping nucleoside positions must be made compatible. Another example is the preference for a comparatively low interstrand affinity in region 3 defined by Table 3. This also puts an affinity preference on seqRNAi-based duplexes that involves the seed sequence of the antisense strand that potentially conflicts with any desire to boost the affinity of the seed sequence with its mRNA 3′UTR target.

The solution to these potential conflicts is to design seqMiR strands so that any modifications to the seed sequence that increase binding affinity for the mRNA 3′UTR target sequence do not proportionally increase the overall or regional interstrand affinity with the seqMiR partner sense strand. For example, one or more LNA modifications can be used in the antisense strand seed sequence where they are compensated for by mismatches, UNA, abasic nucleosides or other permissible affinity lowering modifications in the corresponding area of the partner sense strand. With this type of compensation it is preferred that the affinity reducing modification involves either the binding partner or a nucleoside contiguous with the binding partner that has the affinity increasing modification.

The seed sequences, mRNA 3′-UTR sequences, calculations and experimental design used by Ui-Tei et al., (2008) can be used to help illustrate aspects of the design and testing of seqMiR compounds including those based on novel seed sequences (i.e., ones not found in endogenous miRNA). The particular methods used in the example are not meant to be limiting but rather to show one approach to reducing some of the design concepts for seqMiRs reduced to practice. The seed sequences taken from Ui-Tei et al., have little commercial value but are valuable as an example given that they have been used to generate real data that ground the example in actual facts. The same basic approach can be used with novel or endogenous miRNA seed sequences that are directed to the 3′UTRs of actual mRNA types that are to be silenced by a seqMiR compound.

FIG. 2A summarizes some of the data from Ui-Tei et al., (2008). The first column lists the names of 26 different siRNA compounds. The next two columns list the seed sequences from each of these compounds and the sequence containing the complement to the seed sequence that was constructed for insertion into an expression vector. The fourth column provides the calculated Tm for the seed duplex and the final column provides the percent suppression of the expression vector product produced by the siRNA when transfected into cells that express it. As previously stated there is a strong positive correlation between a higher Tm for the seed duplex and a higher level of target suppression.

Key observations based on these data include the following: (1) the fact the binding affinity of the seed duplex appears to be a much more important determinant of the level of target suppression than is the nature of the rest of the siRNA compound; and (2) a specific seed sequence from an endogenous miRNA antisense strand is not necessary in order to obtain miRNA-like silencing activity. So what appears to be important with respect to the duplex is that it simply has the necessary properties to result in the loading of the desired antisense strand into RISC. Indeed, it is not even necessary for the duplex structure to mimic any particular features of endogenous miRNA duplexes that are different from siRNA compounds in order to get miRNA-like silencing activity.

The experimental design upon which the suppression data shown in FIG. 2A were generated involves the use of expression vectors for a gene with an easily quantifiable product. Ui-Tei et al., (2008) used the Renilla luc gene inserted into the commercially available psiCHECK-1 plasmid (Promega). Twenty-one nucleoside sequences, shown in column 3 of FIG. 2A, that include an 8 nucleoside stretch complementary to the 5′-end nucleoside and the contiguous seed sequence were inserted in the plasmid in the 3′UTR of the luc gene in the plasmid as three tandem repeats. The remaining 13 nucleosides in the inserted target sequence had no homology to the rest of the siRNA antisense strand. This was repeated for each of the 26 siRNA compounds involved in the evaluation and listed in column 1 of the figure. These plasmids were then transfected into HeLa cells that were subsequently treated with the siRNA compound with the seed sequence matching the target sequence in the transfected plasmid. The ability of the siRNA to suppress the luc gene product was determined for various doses and the 5.0 nM dose result is shown in the fifth column of Table A in FIG. 2.

FIG. 2B provides a table that illustrates two possible steps in the modification of seed sequences for use in seqMiR or ss-MiR compounds. In the actual practice of producing commercially useful compounds the seed sequences can come from endogenous miRNA antisense strands or they can be novel seed sequences designed to target a particular group of endogenous mRNA types. The basic rules provided for achieving nuclease resistance and the other essential/preferred architectural-independent rules are applied to the seed sequences shown in the first column and the results are shown in the second column. The sugar in the most 5′ nucleoside in the seed sequence can be ribose or 2′-fluoro depending on the intrinsic nuclease stability of the first two linkage sites in the strand. Since this cannot be fully determined without knowing the 5-end nucleoside in the strand the examples all have the 2′-fluoro modification in the first seed position. The modifications, if any, to the most 3′ nucleoside in the seed sequence and the nature of its linkage to the contiguous nucleoside that is not part of the seed sequence clearly depends on the nature of the contiguous non-seed nucleoside. For the sake of this illustration it is assume the contiguous nucleoside is a G because this matches the situation if either of the negative control duplexes shown in FIG. 2D are used as the duplex vehicle. To indicate this situation a G is shown in parenthesis in column 2 of FIG. 2B. If another duplex vehicle were used the contiguous nucleoside with the 3-end of the seed sequence could be U, C or A. In the case of the siRNA based duplex vehicle shown in FIG. 2D the contiguous nucleoside would be U. In column 3 examples of possible modifications selected from Table 2 that can be added to substantially increase the Tm of the seed sequence with the target. The estimated increase in Tm compared to the unmodified sequence is shown in column 4. In the actual practice of producing commercially useful seqMiR compounds the particulars of such modifications, if any, would be tailored to optimize the silencing of the intended group of mRNA types.

FIG. 2C provides a table that illustrates two possible steps in the modification of the portion of the sense strand for use in seqMiR compounds that corresponds to the seed sequence of the complementary strand. The principal goal here is to reduce the effect of the affinity enhancing modifications made to the seed sequence in FIG. 2B on the regional and overall affinity of the sense and antisense components of the seqMiR compound. The preferred level of Tm reduction in practice will depend on the exact structure of the seqMiR-based duplex. Examples of possible modifications are shown in column 3 and the estimated reduction in Tm between the modified sense and antisense strands is shown in column 4. Since the preferred way to reduce affinity in this situation is to introduce mismatches the nuclease resistance modifications may have to change accordingly. Further, the modifications, if any, to the most 3′ nucleoside in the sense strand sequence in column 2 and the nature of its linkage to the contiguous 3′ nucleoside (not shown) that is not part of this section of the sense strand sequence can depend on the nature of the contiguous 3′ nucleoside. This occurs if there is an overhang precursor in the sense strand. If so then the rules required to provide endonuclease protection apply to the 3′end of the sense sequence shown in column 2. In the absence of an overhang precursor then the rules for protecting the 3′end of the strand in the absence of an overhang precursor apply. This is the case with the example in FIGS. 2B, 2C and 2D because the strand lacks an overhang precursor. As a consequence the sense strand sequences shown in 2C must end with a modified nucleoside and be connected to the contiguous 3′ nucleoside (not shown) by a phosphorothioate linkage. Finally, in the actual practice of producing commercially useful seqMiR compounds such modifications to this portion of the sense strand would be tailored to a particular duplex vehicle and the full complement of design requirements provided herein as applied to the entire duplex vehicle with the desired seed sequence inserted.

FIG. 2D provides three examples of duplex vehicles that are used to illustrate features of the design of seqMiR compounds that make use of such structures. These examples are based on two established negative controls for multiple species including mouse and human and a siRNA targeting human and mouse Apo-B. As shown these parent compounds have been modified in accordance with the essential/preferred architectural independent rules (from sections E and F). The question marks indicate places where the preceding nucleoside and/or its 3′ linkage modification cannot be determined in the absence of a specific insert sequence. Each of the duplex vehicles is shown with and without a modification in the antisense strand intended to inhibit catalytic AGO-2 based silencing activity. In these instances this is specifically illustrated by the placement of an abasic 2′deoxyribonucleoside in position 11 counting from the 5′-end of the antisense strand. In practice the means to inhibit AGO-2 catalytic activity can be prophylactically made to the strand or only be made if the need arises.

The portions of the strands to be replaced by the selected seed sequence and the corresponding sense strand sequence are underlined. As shown the rules for generating nuclease resistance along with the essential/preferred architectural-independent rules have been applied to the strands of the duplex vehicles with the exception of the underlined portion. After the selected seed sequence and the corresponding sequence in the sense strand have been inserted and an architecture selected then the design of a particular seqMiR compound can be finalized. Examples of seed sequences and the corresponding sense strand sequences for the purposes of this illustration are provided by FIGS. 2B and 2C respectively. The insertion of a new seed sequence into a negative control has the potential to generate a compound with off target AGO-2 catalytic activity. If this occurs it can be inhibited by the methods provide herein.

In the actual practice of producing commercially useful seqMiR compounds the pool of potential duplex vehicles can be comprised of any duplex capable of meeting the design criteria provide herein and where the duplex results in the efficient loading of the duplex and retention of the desired antisense strand by RISC. Sources of duplex vehicles include endogenous miRNA duplexes, conventional siRNA compounds and duplexes that are established to be miRNA/siRNA negative controls for the subject species of interest for treatment with seqMiR compounds. Negative controls will need to be rechecked for a lack of induction of unintended siRNA-based silencing activity once the selected seed sequence and corresponding sense strand sequence are inserted. Any AGO-2 based catalytic silencing activity generated by a duplex vehicle can be inhibited by replacing the nucleosides in positions 10 and/or 11 counting from the 5′-end of the antisense strand with modified nucleosides that will inhibit this catalytic activity without preventing duplex formation by the strands. Suitable modifications for this purpose include abasic, UNA and FANA. The abasic nucleosides can have any of the sugar modifications provided for herein including the unlocked variant (i.e., the sugar in UNA), 2-deoxyribose and FANA. Abasic nucleosides preferably are joined to adjacent nucleosides by phosphorothioate linkages.

FIG. 2E provides another seqMiR design variant that is based on the use of a dimer forming antisense strand. One of the ways this variant is unusual is that it functions as a seqMiR but only requires a single strand. This design involves placing both the seed sequence and the complementary sequence in the same strand rather than separating them between a sense and an antisense strand.

In the illustrative example shown in FIG. 2E seed sequence number 12 (from siRNA ITGA10-2803) in the Table in 2B and the corresponding portion of the sense sequence shown in FIG. 2C are placed in the antisense strand in the first duplex vehicle shown in FIG. 2D. The placement of the sequence previously associated with the sense strand is placed in same position in the antisense strand that it would be in a sense strand. These seed and corresponding sense strand sequences are underlined in the first illustration in FIG. 2E.

Two things are immediately clear from FIG. 2E: (1) The antisense strand forms a dimer or more specifically a duplex with itself: and (2) The antisense stand will also form a hairpin with itself. These factors will also be true of any other seqMiR designed in this manner. The calculated overall Tm for the unmodified two-stranded duplex is 58 degrees centigrade under physiological salt conditions and 50 nM compound concentration using the nearest-neighbor calculation. The portion of the strand supporting the hairpin is represented along with the intervening unpaired loop.

Two potential advantages to this design are the following: (1) Only one strand has to be used in treatment; and (2) The hairpin can provide nuclease protection to the seed sequence. As a result the seed sequence does not have to be chemically modified to protect it from nuclease attack. This would allow, for example, seed sequences from endogenous miRNA to be used without chemical modification. A disadvantage of this design is that it is cannot be efficiently administered to the circulation because the kidneys will rapidly clear the double strand duplexed portion of the interchanging double and single strand forms. This approach is more likely to be most useful in situations were the compound is inserted into comparatively static environments such as the cerebral spinal fluid, joint fluids, ascites and bladder rather than into the circulation. Here the double strand species in effect serves as a reservoir for the single strand species that can be more efficiently taken up by cells.

This approach can be used with architectures other than small internally segmented and the asymmetric variant where there is a 5′-end overhang. The asymmetry rule that applies to these architectures, however, has to be modified because it does matter which strand is loaded since they are the same. Thus, the requirement for a differential Tm between the duplex termini is lowered in this situation. The concept that the 4 most terminal nucleosides have a graded affinity with the lowest affinity being relegated to the 2 most terminal nucleosides, however, is retained. Since the 5′end terminal nucleoside is not part of the seed region it can be configured as a mismatch with the 3′end terminal nucleoside with no effect on the seed duplex Tm. This is preferred when the one or both of the nucleosides in the 2 terminal positions are G and/or C. The most 5′ of the seed sequence nucleosides can also be mismatched with the corresponding nucleoside on the 3′-end but not with the target sequence. This is preferred if the seed sequence starts with Gs and Cs in the initial 2 positions from the 5′end.

It is also preferred that the strands be inhibited from supporting AGO-2 catalytic activity that could generate off target effects. This can be achieved by replacing the nucleosides in positions 10 and/or 11 from the 5′-end with modified nucleosides that will inhibit this catalytic activity without preventing duplex formation by the strands. Suitable modifications for this purpose include abasic, UNA and FANA. The abasic nucleosides can have any of the sugar modifications provided for herein including the unlocked variant (i.e., the sugar in UNA), 2-deoxyribose and FANA. Abasic nucleosides preferably are joined to adjacent nucleosides by phosphorothioate linkages. The design rules affecting regional interstrand affinities just discussed in this and the preceding paragraph also fulfill the preference for lower regional affinities in regions 1, 2 and 3 defined by Table 3.

In the illustration in FIG. 2E, the A in position 10 and the U in position 11 are rendered abasic 2′-deoxyribonucleotides as indicated by the OD subscript (FIG. 1). Such modifications involving two positions can result in overall Tm drops of 10-20 degrees centigrade. When required such a drop can be compensated for by using a slightly longer strand and/or by adding one or more modifications that increase Tm. This is not necessary in the present example given the starting Tm of 58 and the increases to Tm provided by the other modified nucleotides. It is also not necessary if the compound does not produce unacceptable off-target effects due to AGO-2 catalytic activity.

As for seqMiR sets generally, modifications can be made to the seed sequence that will increase the Tm of the seed duplex without undermining important thermodynamic considerations with respect to overall and regional interstrand affinities. This is achieved by making compensatory changes in the sequence complementary with the seed sequence in the seqMiR strand set that in this case is in the same strand. Various means to enhance or reduce interstrand or antisense strand/target affinities are listed in Table 2.

One specific example or increasing the Tm of the seed duplex out of the numerous possibilities is shown in the last illustration in FIG. 2E. Here the LNA modification is used in positions 5 and 8 counting from the 5′-end. These are compensated for by the U_{OD} in position 11 and by the use of a mismatch in position 15. The U_{OD} in position 11 is contiguous with the binding partner for the LNA in position 8 and the mismatch in position 15 is the binding partner for the LNA in position 5. This illustrates that this type of compensation can involve either the binding partner or a nucleoside contiguous with the binding partner.

FIG. 2F provides examples of the application of these design principles to a seed sequence taken from an endogenous miRNA that has potential relevance for drug development. Let-7 family members can act as anti-oncogenes and the levels of one or more family members is suppressed in a number of cancer types. Experimentally increased levels of the suppressed family member(s) has been shown to produce a variety of anticancer effects.

The seed sequence illustrated in FIG. 2F is common to multiple members of the let-7 miRNA family and to multiple species such as human and mouse. By inserting this sequence and the corresponding sense strand sequence into a duplex vehicle a seqMiR can be constructed that can mimic features of multiple let-7 family members. Further, the affinity of this seed sequence for the target mRNAs can be increased with a resulting increase in silencing activity. Five examples of this are shown along with 5 examples of compensatory reductions in binding affinity capacity in the corresponding area of the sense strand.

In FIG. 2G these sequences are inserted into the appropriate places in the duplex vehicle shown in 2D that is based on a siRNA to Apo-B. The antisense strands are shown with and without examples of blocking AGO-2 catalytic activity against any unintended mRNA target. Further, the antisense strands are shown with 2 overhang unit precursors. These can be selected from those provided in the overhang precursor section, for example, ˜U˜U or ˜dT˜dT.

Examples of dimer forming single strands based on the antisense strands shown in 2G are illustrated in 2H. As described in the description associated with 2E such dimer forming single strands are most suitable for used in compartments, such as the CNS, in subjects other than the circulation where the dimer form can be cleared in a matter of minutes by the kidneys.

### 4. Summary of Minimal Essential Rules for seqRNAi Compounds

In addition to the essential/preferred architectural-independent rules provided in sections E and F there are minimal thermodynamic requirements for the most basic seqRNAi compound suitable for use in accordance with the present invention. The stand-alone architectures provided differ in the following: (1) whether or not they provide for an overhang precursor(s) in strands and if so how many units are there and where are they; and (2) whether or not they provide for one sense strand and one antisense strand or for two sense strands and one antisense strand or for two antisense strands and one sense strand as members of the same seqRNAi set. It is obviously necessary for a seqRNAi-based duplex to have an architecture. From a thermodynamic point of view the blunt-ended architecture is the simplest in terms of describing the minimal set or rules for a seqRNAi set. This is because dual sense or antisense strands in the same seqRNAi set require additional thermodynamic considerations and an overhang longer than one unit has the potential to affect interstrand binding affinity of the seqRNAi-based duplex. This can occur when the overhang is long enough to double back on the duplex and interact with it. The overhang effect, however, is typically not a major concern and can be ignored in general design considerations. Thus, nearly all situations the canonical and asymmetric architecture (with only a 3′-end overhang precursor) are no more thermodynamically complex in terms of the rules presented than the blunt-end architecture.

Given these stipulations the minimal requirements for seqRNAi compounds requires the essential/preferred architectural-independent rules provided in sections E and F along with the essential/preferred rules for the blunt-end architecture. In this simplest case the length of the strands will be assumed to be 19-mers since this length corresponds to that of the largest proportion of conventional siRNA and miRNA compounds exclusive of any overhangs. The architectural-dependent algorithms include rules with additional thermodynamic considerations that are not considered here as the simplest case. The thermodynamic rules for the simplest case seqRNAi set can be summarized as follows:
1) Table 3 explicitly defines three regions in a seqRNAi-based duplex based on the sense strand where it is preferred that the combined contribution of the three regions have a Tm that is lower than the Tm for the overall duplex when corrected for the smaller number of contributing nucleosides. It is preferred that all three regions have relatively lower Tms but they are individually too short to allow for reasonably reliable Tm determinations. Adjustments in affinity can be achieved by using affinity-lowering modifications in the sense strand portion of one of these explicitly defined regions and/or by increasing the affinity in the intervening areas in the sense strand. When the overall Tm of a seqRNAi duplex is above the preferable range then the use of affinity lowering modifications to reduce the overall Tm preferably are made to one or more of the regions explicitly defined in Table 3. The general steps involved in achieving these goals are the following:
   a. The collective interstrand duplex Tm for the 3 regions explicitly defined by Table 3 and the overall duplex Tm are determined for the unmodified strands using the nearest-neighbor calculation.
   b. Next the effects of the chemical modifications on the regional and overall Tms are adjusted for the modifications made following the applications of the nuclease resistance and essential/preferred architectural-independent rules using the information in Table 2.
   c. Finally, the information in Table 2 is used to reduce the combined regional Tm and/or to increase the intervening Tms as needed. These modifications should be evenly distributed as much as possible. The modifications are made to the sense strand.
2) The asymmetry rule is applied next.
   a. The Tm between the duplexed 4 nucleosides at each terminus based on the unmodified RNA sequence is estimated using the following equation: Tm=2(wA+xU)+4(yG+zC), where w, x, y and z are the numbers of the indicated nucleosides in the 4 nucleoside duplex.
   b. Table 2 is used to make adjustments in the overall 4 nucleoside duplex Tm based on the modifications applied to these nucleosides and to the intervening linkages following the applications of the nuclease resistance, essential/preferred architectural-independent and the thermodynamic rules just provided in (1).
   c. The determinations in (a) and (b), however, do not take into account the decreasing importance of the nucleosides as one moves away from the terminus. To account for this in a simple way it is preferred that the overall Tm for the 4 nucleoside duplex be lower for the one containing the 5′end of the antisense strand and that the most terminal two nucleoside pairs of this duplex have a lower affinity for their partner nucleoside than the corresponding pairs at the other terminus. If it is necessary to make an adjustment either in one or both of the terminal nucleoside pairs or in the overall Tm for the terminal 4 nucleosides the needed modification information can be obtained from Table 2. In general, the magnitude of the modification should be in alignment with the magnitude of the needed adjustment.
   d. When major affinity adjustments of this type are in order mismatches are preferred over UNA and abasic and they are made to the sense strand.

Not all the permitted strand modifications provided for herein have been well characterized with respect to their impact on interstrand or antisense strand/target affinity and they do not appear in Table 2. Those that have been characterized typically vary in their effect with their position within the strand (terminal positions, for example, typically result in a reduced effect) and by other details of the adjacent strand/duplex context.

The next most basic considerations to seqRNAi set design involve adding the essential/preferred rules for the targeting codes. These are the central region of the antisense strand of seqsiRNA and seqIMiRs and the seed sequence of seqMiRs. These rules also apply to the corresponding ss-siRNA, ss-IMiR and ss-MiR antisense strands respectively.

The application of these essential rules to an example of an seqsiRNA (seqIMiRs follow the same design process only the type of target RNA is different) in FIG. 8 where the target is mouse PTEN and in FIG. 9 where the seqMiR example is based on let-7i. These figures illustrate the essential basic design of seqsiRNA/seqIMiR and seqMiR compounds. In standard practice the some or all of the thermodynamic rules can be left to later in the design process.

FIG. 8 carries over the three strands from FIG. 6 as a starting point. The three regions defined by Table 3 are underlined in the sense strand. The sequence of the combined three regions are shown next followed by the combined intervening regions. Table 4 provides the Tm calculation results for the overall duplex and for the combined regional and combined intervening sequences with and with out adjustments for the modifications made to the strands. Table 2 is used to provide the estimated effects of the various modifications on the Tm. The combined regions 1-3 sequence is 10 nucleosides in length while the combined intervening sequence is 9 nucleosides in length so the Tm for the former has been proportionally decreased.

TABLE 4

| | Tm | |
|---|---|---|
| | (degrees centigrade) | |
| Duplex | Unmodified | Modified |
| Overall | 70 | 79 |
| Combined Regions 1-3 | 34* | 38* |
| Combined Intervening | 37 | 41 |
| Regions | | |

*Reduced 10% to compensate for longer length compared to combined intervening regions

It can be seen in Table 4 that both the differential Tms for the two combined regions and the overall Tm are within the preferred parameters without further modification. Further, the Tm calculations for the two termini of the duplex meet the requirements of the asymmetry rule. The terminus with the 5′-end of the sense strand has a calculated Tm of 28 degrees that increases to 32 with the modifications while the other terminus has a calculated Tm of 20 degrees that increases to 22 degrees with the modifications. In general practice, the asymmetry rule would not be applied at this point if the small internally segmented or asymmetric architecture with a 5′-end overhang had been selected as part of the design.

FIG. 9 carries over the three strands from FIG. 7 as a starting point except the overhang precursors have been removed because the simplest case is being considered in the example. The three regions defined by Table 3 are underlined in the version of the sense strand that has the wobble base pairs and mismatch with the antisense strand removed. The sequences of the combined three regions are shown next followed by the combined intervening regions. Table 5 provides the Tm calculation results for the overall duplex and for the combined regional and combined intervening sequences with and with out adjustments for the modifications made to the strands. Table 2 is used to provide the estimated effects of the various modifications on the Tm.

The data shows that the estimated overall duplex Tm is high (82 degrees) so there will be an expected preference for loading AGO-2. This can increase the likelihood that this seqMiR compound without further modification could have off-target siRNA like activity. If this is a problem for the intended commercial purpose the overall Tm of the duplex can be reduced to the preferred range or the nucleosides in positions 10 and/or 11 from the 5′-end of the antisense strand can be modified to inhibit AGO-2 from carrying out a direct cleavage of an unintended mRNA target(s).

The data also shows that combined Tm of the three regions defined by Table 3 is lower than the Tm of the combined intervening region. Thus, this duplex meets this thermodynamic preference without further modification.

TABLE 5

| | Tm | |
|---|---|---|
| | (degrees centigrade) | |
| Duplex | Unmodified | Modified |
| Overall | 70 | 82 |
| Combined Regions 1-3 | 30 | 36 |
| Combined Intervening | 41 | 47 |
| Regions | | |

FIG. 9 also provides the 4-nucleoside duplexes from each terminus for consideration of their compatibility with the asymmetry rule. The terminus with the 5′-end of the sense strand has a calculated Tm of 14 degrees centigrade unmodified and 16 degrees with the modifications shown while the other terminus has Tms of 12 degrees and 14 degrees respectively. Thus, termini are in general compliance with the broader requirement of the asymmetry rule, but the second pair of nucleosides from the termini are suboptimum in that the pair in the terminus with the 5′end of the antisense strand has a comparatively higher affinity than the corresponding pair in the other terminus. Given that the terminus with the 5′end of the sense strand already has a high Tm with 3 of the 4 nucleoside pairs being G:C the second pair in the other terminus can equally well have an A or a G to replace the C but G is selected for this example. The indifference to the A or G replacement is that neither provides an advantage over the other with respect to introducing a more nuclease resistant linkage pair.

### H. Algorithms: Architectural Dependent—Canonical

### I. Description

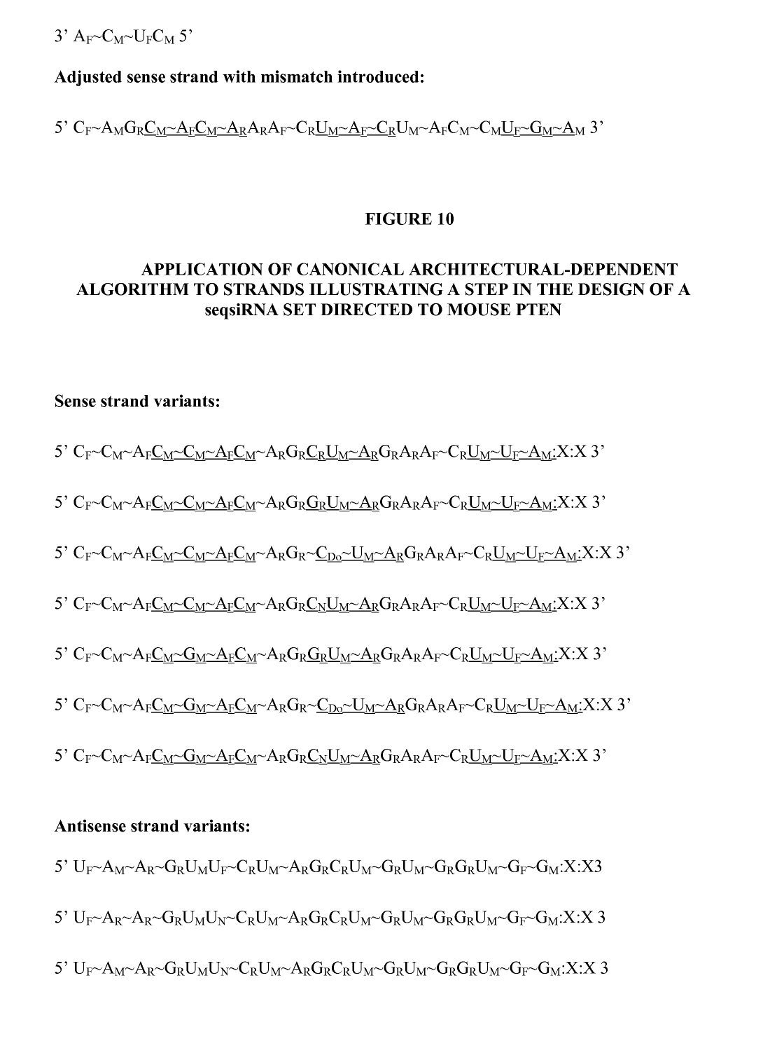

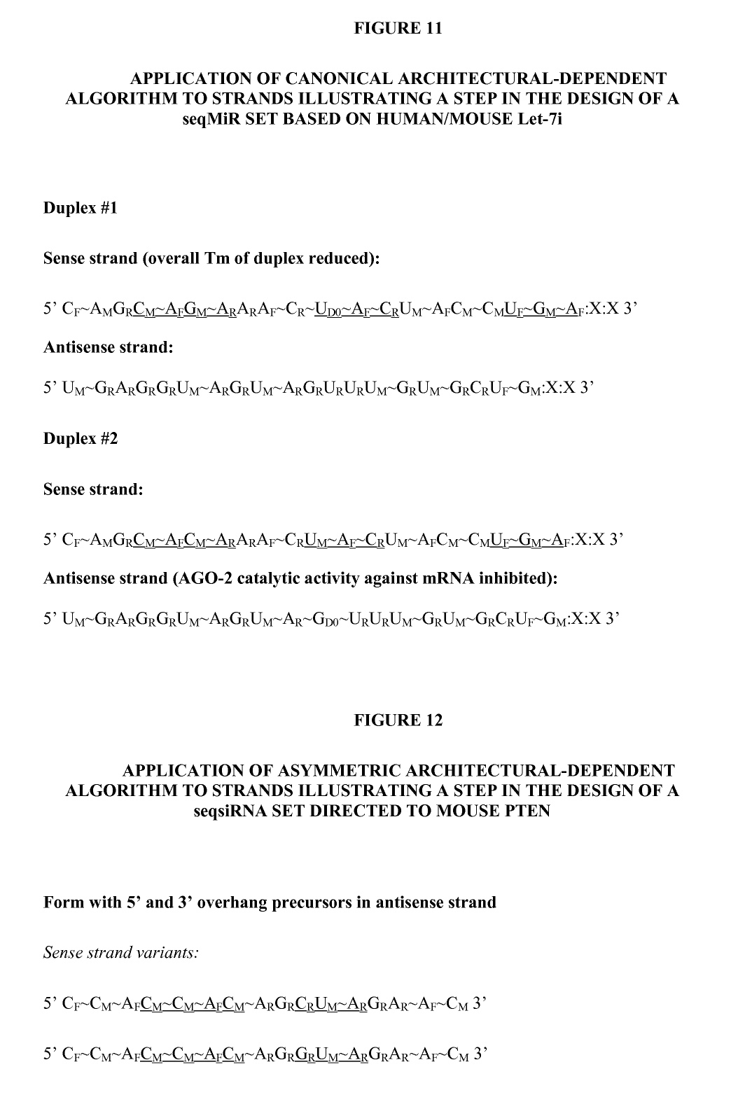

Canonical is the naturally occurring siRNA architecture. It is also the commonly used architecture for manufactured conventional siRNA. This architecture is defined by the presence of 1 to 4 nucleosides or nucleoside substitutes called overhangs on the 3′-ends of both strands that extend beyond the duplexed portion of the compound. It is generally preferred that overhangs be 2-3 nucleosides or nucleoside substitutes in number.
   With the seqRNAi approach the compounds delivered to subjects are single strands rather than duplexes so it is meaningless to talk about such strands having overhangs. Instead they have overhang precursors and in the case of the canonical architecture format both seqRNAi strands have overhang precursors. Exclusive of the overhang precursors the two strand of a given seqRNAi set have the same length. Overhang precursors are discussed in more detail in the section by that name.
   The asymmetry rule is important for the canonical architecture. This and other thermodynamic considerations relevant to the canonical architecture are considered in more detail in the thermodynamics section.
   The application of the canonical architecture dependent algorithm to the illustrative seqsiRNA and seqMiR examples is provided in FIGS. 10 and 11 respectively.
   The sense and antisense strands from FIG. 8 are carried over as the starting point for the modifications introduced in FIG. 10. The latter figure illustrates 7 of the sense strand variants and 3 of the antisense strand variants that are consistent with the canonical architecture. Any of these sense strands can be used with any antisense strand. As required by the canonical architecture both strand types are shown with overhang precursors. These can be any of those described in the section by that name. For the sake of illustration those in the example can be said to be ˜U_{F}˜U_{M}. The same strands can be used according to the blunt-end architecture simply by dropping the overhang precursors.
   FIG. 11 carries over the adjusted sense strand and the antisense strand from FIG. 9. The sense strand with the wobble bases and mismatch retained could be used but it is not continued to simply the illustration. The canonical architecture requires 3′-end overhang precursors on both the sense and antisense strands. In the illustration 2 overhang units are shown since this is the preferred number. The units and the intervening linkages can be any of those provided for in the overhang precursor section. For the sake of illustration those in the example can be said to be ˜U_{F}˜Um.
   Two duplexes are shown to illustrate the two principal ways that unintended off target effects due to a siRNA-like activity can be reduced in a seqMiR set. In duplex one the overall Tm is reduced to below 60 degrees centigrade. One additional mismatch and one abasic nucleoside are added to the mismatch inserted in the sense strand in accordance with the asymmetry rule. The new modifications are within the regions 1 and 2 that are explicitly defined by Table 3. These will have the effect of reducing the 82 degree Tm to a Tm under 60 degrees. In the second duplex position 11 from the 5′end of the antisense strand is converted to an abasic nucleoside.

### 2. Applicable to seqRNAi Sense Strands

a) The strand is required to have at least one overhang precursor unit at the 3′-end.
b) Unless otherwise provided for the strand can have one modification per region in one or more of the three regions explicitly defined by Table 3 where the modifications are selected from the group consisting of a nucleoside mismatched with its partner (opposite) nucleoside in the antisense strand, an abasic nucleoside, UNA and ANA. When a UNA is used in region 1 it is preferred that it be in the most downstream position from the 5′-end that is allowed by the Table. Abasic nucleosides preferably are joined to adjacent nucleosides by phosphorothioate linkages.
c) Except when one of the modifications just described in (b) is used in region 2, the following is preferred: The two nucleoside positions opposite positions 10 and 11 from the 5′-end of the antisense strand when the strands are duplexed is joined by a phosphodiester linkage and the nucleoside in the sense strand opposite position 11 in the antisense strand is selected from the group consisting of ribose and 2′-fluoro and the nucleoside in position 11 is selected from the group consisting of ribose, 2′-fluoro or 2-0-methyl. So, for example, if the sense strand is a 19-mer exclusive of any overhang precursors then position 9 from the 5′-end of the sense strand would be opposite position 11 in the antisense strand. Further, when the linkage site opposite positions 10 and 11 of the antisense strand is so configured, it is preferred that the four sense strand nucleoside positions opposite nucleoside positions 9-12 from the 5′-end of the antisense strand when the strands are duplexed not have any mismatches with the antisense strand.
d) When the strand has only one 3′-end overhang precursor unit then the 3′-end terminal nucleoside or nucleoside substitute and the terminal two linkages are provided by the 3′-end overhang section herein and the nucleoside next to the overhang precursor will be selected from the group 2′-fluoro, 2′-0-methyl or 2′-deoxyribose.
e) When the strand has 3′end overhang precursors that are at least two nucleoside or nucleoside substitute units in length the required 3′-end exonuclease protection is provided by the 3′-end overhang designs described herein.
f) The terminal 5′-end nucleoside preferably is chemically modified, for example, by methylation to prevent its 5′ ribose position from being phosphorylated by endogenous enzymes.
g) Should they occur, undesirable off target silencing due to the seed sequence promoting miRNA-like activity can be inhibited using one or more of three alternatives that can inhibit the interaction with the unintended mRNA target(s): (i) one or both of the following stipulations are met: the second nucleoside from the 5′-end is not ribose or 2-fluoro and preferably is 2′-0-methyl and/or one of the nucleosides in positions 3-7 from the 5′-end is UNA or abasic. Destabilizing modifications, however, should not fall in the central region of the antisense strand; (ii) If the target sequence in the unintended mRNA target site(s) complementary to the seed sequence has one or more U and/or G containing nucleosides then the seed sequence can be adjusted to generate at least one G:U wobble base pair between it and the target sequence; or (3) a multiplicity of the nucleosides in the seed sequence can be 2′deoxyribonucleosides. The presence of 5 or more consecutive 2′-deoxyribonucleosides is discouraged, however, since it has the potential to promote RNaseH based degradation of endogenous RNA complementary to the strand. Abasic nucleosides preferably are joined to adjacent nucleosides by phosphorothioate linkages.

### 3. Applicable to seqRNAi Antisense Strands

a) The strand is required to have at least one but not more than four overhang precursor units at the 3′-end with two units being preferred.
b) When the strand has only one 3′-end overhang precursor unit then the 3′-end terminal nucleoside or nucleoside substitute and the terminal two linkages are provided by the 3′-end overhang section herein and the nucleoside next to the overhang precursor will be selected from the group 2′-fluoro, 2′-0-methyl or 2′-deoxyribose.
c) When the strand has 3 ‘end overhang precursors that are at least two nucleoside or nucleoside substitute units in length the required 3’-end exonuclease protection is provided by the 3′-end overhang designs described herein.

### 4. Applicable to seqsiRNA and seqIMiR Antisense Strands

Should they occur, undesirable off target silencing due to the seed sequence promoting miRNA-like activity can be inhibited using one of two alternatives that can inhibit the interaction with the unintended mRNA target(s): (i) one or both of the following stipulations are met: the second nucleoside from the 5′-end is not ribose or 2-fluoro and preferably is 2′-0-methyl and/or one of the nucleosides in positions 3-7 from the 5′-end is UNA or abasic; or (ii) If the target sequence in the unintended mRNA target site(s) complementary to the seed sequence has one or more U and/or G containing nucleosides then the seed sequence can be adjusted to generate at least one G:U wobble base pair between it and the target sequence. Abasic nucleosides preferably are joined to adjacent nucleosides by phosphorothioate linkages.

### 5. Applicable to seqMiR Antisense Strands

Particularly, for strands that will generate an overall Tm of greater than 60 degrees centigrade with their partner strand it is preferred that any catalytic activity of AGO-2 directed against an endogenous RNA target by the antisense strand is inhibited. This can be achieved through making certain modifications to the nucleosides in positions 10 and/or 11 from the 5′-end of the antisense strand. When off target activity against a known target is to be avoided this can be achieved by making one or both of the indicated nucleosides be mismatches with the target. It is preferred in this situation that there not be a single A:C mismatch. Any AGO-2 based catalytic silencing activity can be inhibited by replacing the nucleosides in positions 10 and/or 11 with modified nucleosides that will inhibit this catalytic activity without preventing duplex formation by the strands. Suitable modifications for this purpose include abasic, UNA and FANA. The abasic nucleosides can have any of the sugar modifications provided for herein including the unlocked variant (i.e., the sugar in UNA), 2-deoxyribose and FANA. Abasic nucleosides preferably are joined to adjacent nucleosides by phosphorothioate linkages.

### 6. Applicable to seqsiRNA-Based and seqIMiR-Based Duplexes

The overall Tm, under physiological conditions, will be at least 55 and preferably at least 65 degrees but preferably under about 95 degrees centigrade. The means to adjust overall Tm is presented in the thermodynamics section.

### 7. Applicable to seqMiR-Based Duplexes

The overall Tm under physiological conditions will be at least 45 and preferably under 60 degrees centigrade unless the antisense strand is modified to prevent AGO-2 from having a direct catalytic action on mRNA when it is loaded as such into RISC. In the latter case the preference for an overall Tm limit of 60 degrees is removed.

### I. Algorithms: Architectural Dependent—Blunt-End

### 1. Description

Sense and antisense strands for a given seqRNAi set have the same length and do not have 3′-end overhang precursors. The asymmetry rule is important for the blunt-end architecture. This and other thermodynamic considerations relevant to the blunt architecture are considered in more detail in the thermodynamics section. The application of the blunt-end architecture dependent algorithm to the illustrative seqsiRNA and seqMiR is the same as the canonical illustrated in FIGS. 10 and 11 respectively except there are no overhang precursors.

### 2. Applicable to seqRNAi Sense Strands

a) the required 3′end protection from exonuclease attack can be provided by the use of two terminal nucleosides that are individually selected from the group 2′-fluoro, 2′-0-methyl or 2′-deoxyribose and where the terminal two linkages will be phosphorothioate. Strands that have a 3′ terminal 2′-fluoro modification, however, often have a reduced yield with current manufacturing methods so this modification is not preferred in this position.
b) In other respects the rules for the canonical architecture apply here except the strand lacks an overhang precursor.

### 3. Applicable to seqsiRNA and seqIMiR Antisense Strands

a) the required 3′end protection from exonuclease attack can be provided by the use of two terminal nucleosides that are individually selected from the group 2′-fluoro, 2′-0-methyl or 2′-deoxyribose and where the terminal two linkages will be phosphorothioate. Strands that have a 3′ terminal 2′-fluoro modification, however, often have a reduced yield with current manufacturing methods so this modification is not preferred in this position.
b) In other respects the rules for the canonical architecture apply here except the strand lacks an overhang precursor.

### 4. Applicable to seqMiR Antisense Strands

a) the required 3′end protection from exonuclease attack can be provided by the use of two terminal nucleosides that are individually selected from the group 2′-fluoro, 2′-0-methyl or 2′-deoxyribose and where the terminal two linkages will be phosphorothioate. Strands that have a 3′ terminal 2′-fluoro modification, however, often have a reduced yield with current manufacturing methods so this modification is not preferred in this position.
b) In other respects the rules for the canonical architecture apply here except the strand lacks an overhang precursor.

### J. Algorithms: Architectural Dependent—Asymmetric

### 1. Description

seqRNAi antisense strands have 1-4 unit overhang precursors at the 5′ or 3′ ends or both while the sense strands in the same set do not have overhang precursors. With respect to antisense strands with 3′-end overhang precursors the terminal sense strand 5-end nucleoside preferably is paired with the 3′-end nucleoside in the antisense strand that is contiguous with the overhang precursor. It is preferred for most strand sequences that the overhang precursor only occurs at the 3′-end of the antisense strand. When there is only a 3′-end overhang precursor, it is preferred that it be 2-3 nucleosides and/or nucleoside substitutes in number. 5′-end overhang precursors follow the same rules that apply to the rest of the strand save the 3′-end overhang precursor that can follow other rules. Overhang precursors are discussed in more detail in the section by that name.

The asymmetry rule applies to seqRNAi strand sets designed according to the asymmetric architecture when the antisense strand lacks a 5′-end overhang precursor. When the asymmetric architecture provides for a 5′-end overhang precursor with or without a 3′-end overhang precursor the importance of the asymmetry rule basis for antisense strand selection is nullified. As a consequence, the importance of other factors that affect the level of efficiency in the removal of the intended sense strand and the retention of the intended antisense strand by RISC is increased, for example, by introducing reductions in interstrand affinities in particular regions explicitly defined by the Table 3 relative to other interstrand areas particularly in region 2. These and other thermodynamic considerations relevant to the asymmetric architecture are considered in more detail in the thermodynamics section.

Hence, all three forms of the asymmetric architecture have essentially the same antisense strands differing only in having a 3′-end overhang precursor or not. The permitted canonical or blunt-end antisense strands can simply be transposed to the asymmetric architecture. The sense strands used in the asymmetric architecture are either simply transposed from the blunt-end architecture or they are shorted at the 3′end to generate a 5′-end overhang precursor in the partner antisense strand when the duplex forms. When the sense strand is truncated in this way, it is particularly preferred that regions 1 and 2, defined in Table 3, have relatively low Tms compared to the rest of the strand unless the result is to reduce the overall interstrand Tm below the preferred range. The positioning of regions 1 and 2 in this case are based on the length of the blunt-ended sense strand even though this sense strand is truncated at the 3′-end.

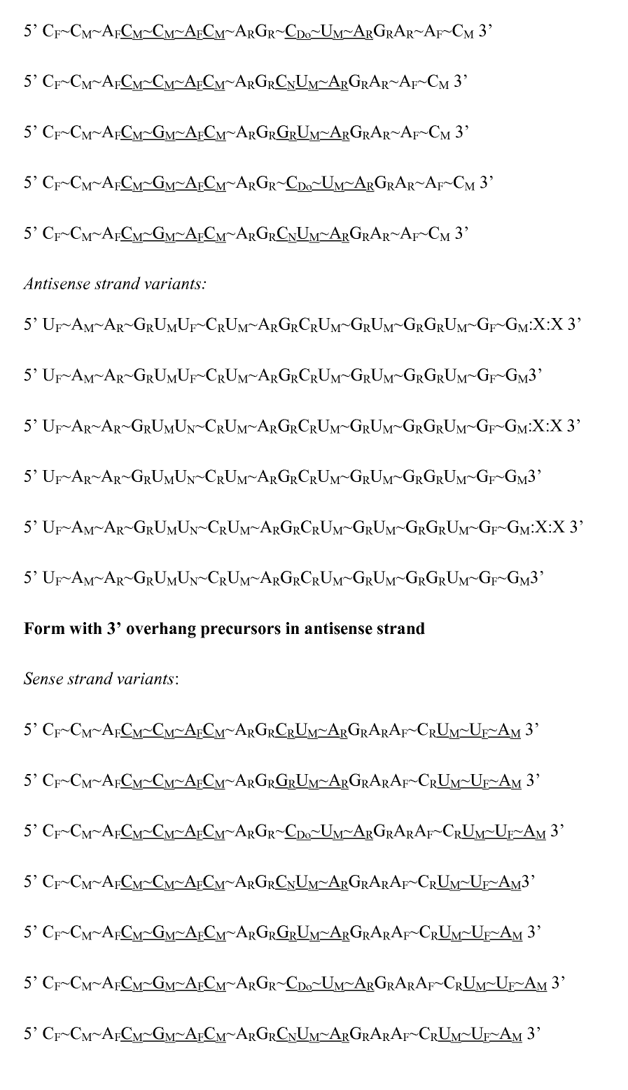

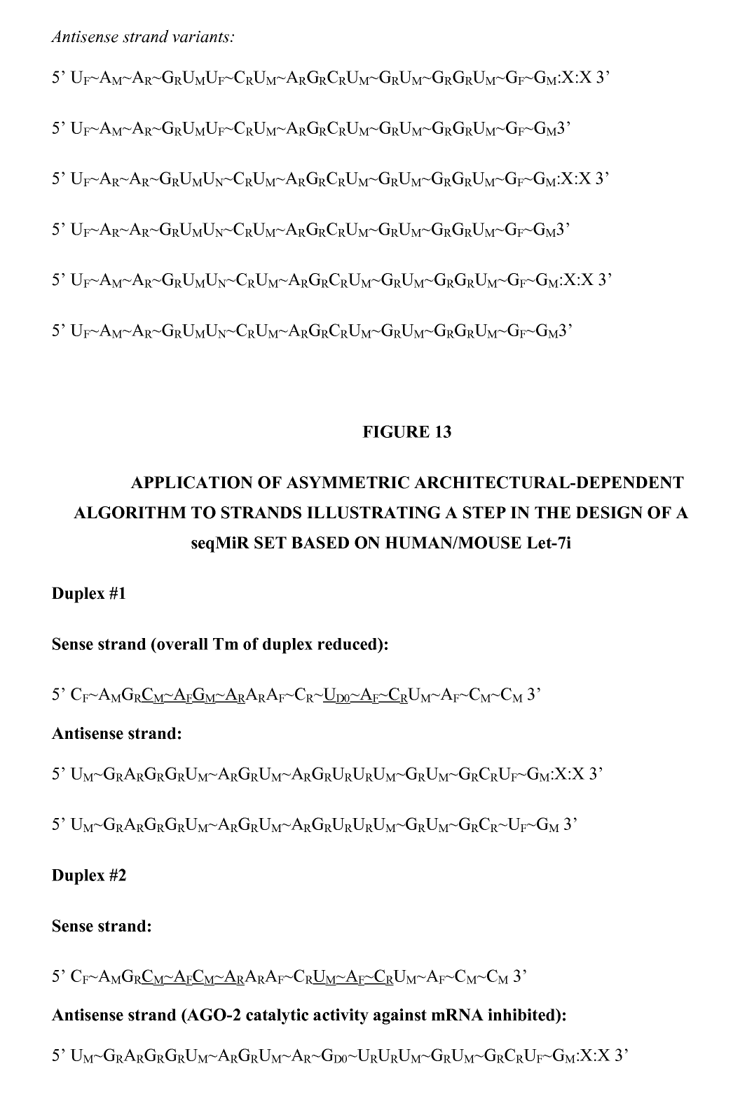

Thus it is only necessary to show the seqsiRNA and seqMiR sets in FIGS. 12 and 13 that only illustrate the case where the sense strand is shortened at the 3′-end. In the specific examples used it is shortened by 3 nucleosides. The antisense strand partner is illustrated as having either a 3 ‘-end overhang or being blunt-ended with the 5’-end of the sense strand. Where the 3′-end has been modified the appropriate changes have been made to comply with the nuclease resistance and essential/preferred architectural independent rules.

### 2. Applicable to seqRNAi Sense Strands that are Paired with Antisense Strands without a 5′-End Overhang Precursor

Same rules apply as for blunt-end architecture.

### 3. Applicable to seqRNAi Sense Strands that are Paired with Antisense Strands with a 5‘-Endoverhang Precursor with or without a 3’-End Overhang Precursor

a) Is at least 13 nucleosides long and is no more than 6 nucleosides shorter than the antisense strand in the set. It is preferred that the 3 ‘end be shorted by no more than 3 nucleosides and that the 5’-end not be shortened.
b) Unless otherwise provided for the strand can have one modification per region in one or more of the three regions explicitly described by Table 3 where the modifications are selected from the group consisting of a nucleoside mismatched with its partner (opposite) nucleoside in the antisense strand, an abasic nucleoside, UNA and an ANA. When a UNA is used in region 1 it is preferred that it be in the most downstream position from the 5′-end that is allowed by the Table. Abasic nucleosides preferably are joined to adjacent nucleosides by phosphorothioate linkages.

### 4. Applicable to seqsiRNA and seqIMiR Antisense Strands

Same rules apply as for canonical or blunt-end architecture depending on whether or not there is a 3′-end overhang precursor.

### 5. Applicable to seqMiR Antisense Strands

Same rules apply as for canonical or blunt-end architecture depending on whether or not there is a 3′-end overhang precursor.

### K. Algorithms: Architectural Dependent—Forked-variant

### 1. Description

The forked-variant algorithm is the most radical solution to fulfilling the asymmetry rule for those seqRNAi architectures where it is important. Thus, its use is limited to being a supplemental variant of these architectures. It is applied to strands that will form seqRNAi-based duplexes where the asymmetry between the duplexed termini is so severely the opposite of what is desired that it cannot be corrected by using the types of chemical modifications used to achieve nuclease resistance in accordance with the present invention. Instead, it involves interrupting the complementary base pairing between some or all of the terminal 6 nucleosides at the 3′-end of the sense strand with the 5′-end of the otherwise complimentary antisense strand by introducing between 2 and 6 mismatches in the sense strand. Thus, the forked variant is an exception to the general rule that destabilizing modifications are not preferred between regions 2 and 3 as defined by Table 3. The specific thermodynamic considerations are discussed in more detail in the section by that name.

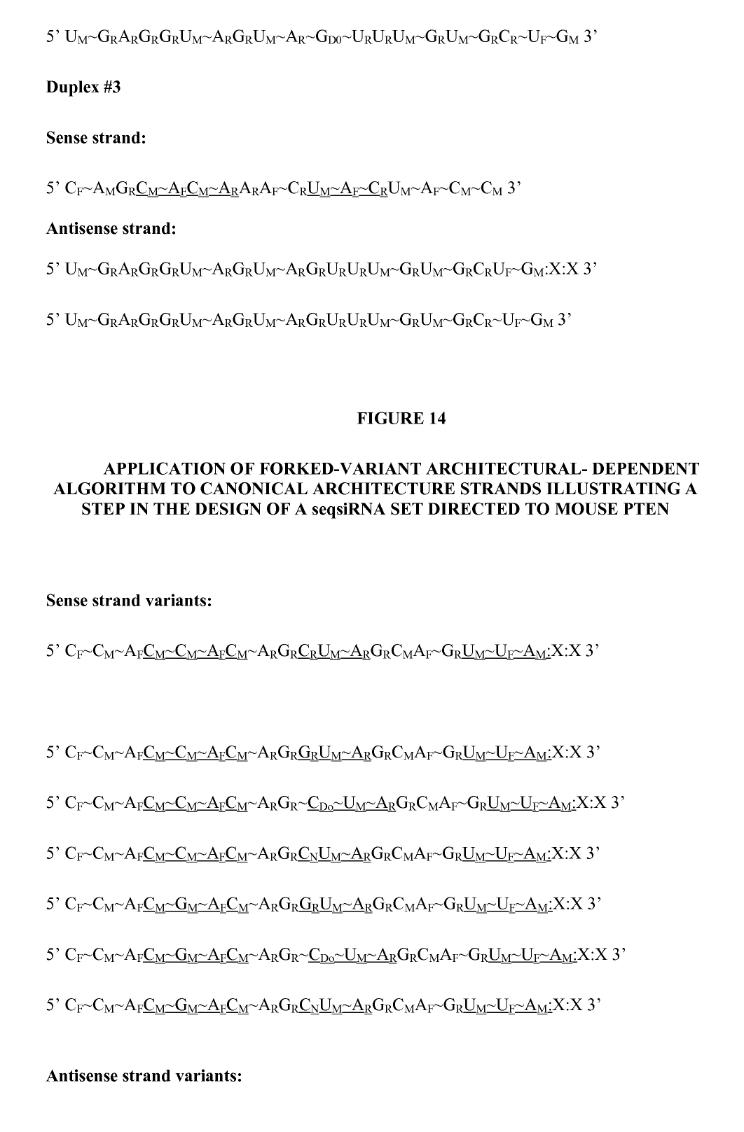

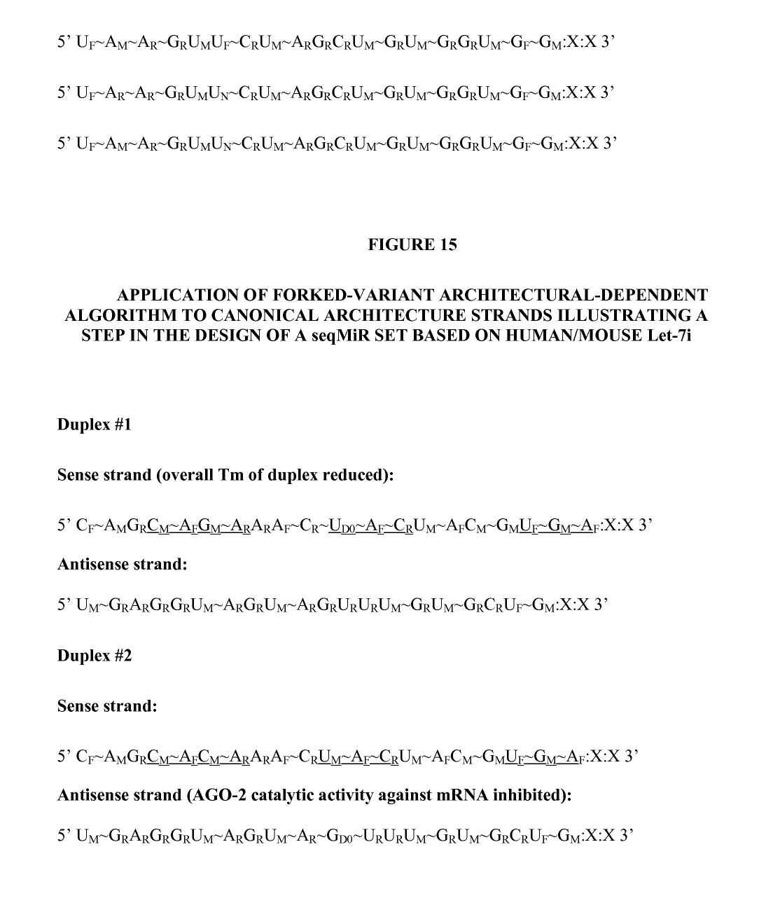

The application of the forked-variant architecture dependent algorithm to the illustrative seqsiRNA and seqMiR examples is provided in FIGS. 14 and 15 respectively.

FIG. 14 carries over the canonical architecture sense and antisense strands from FIG. 10. In the discussion of FIG. 8 it was pointed out the terminal duplex differential Tm for the seqsiRNA Mouse PTEN compounds serves the asymmetry rule well without any added modification. Nevertheless it is conceivable that a modest application of the forked variant could further boost the activity of this these highly related compounds. Accordingly, the A_{R} in position 14 and the C_{R} in position 16 of the sense strand are changed to C_{M} and G_{R} respectively.

FIG. 15 carries over the two duplexes from FIG. 11 using the canonical architecture as the example of an architecture where the asymmetry rule is applicable. These duplexes have already been adjusted for the asymmetry rule in FIG. 9 but conceivably could benefit further from having a greater differential between the two termini. Accordingly a second mismatch is introduced into position 17 of the sense strands counting from the 5′-end.

### 2. Applicable to seqRNAi Sense Strands that Form a seqRNAi-Based Duplex with their Partner Strand that has an Architecture where the Asymmetry Rule is Important Particularly when the Given Duplex is Too Out of Alignment with the Rule to be Corrected by Less Radical Means

The complementary base pairing between some or all of the terminal 6 nucleosides at the 3′-end of the sense strand (exclusive of any overhang precursor) with the corresponding nucleosides in the 5′-end of the antisense partner strand is interrupted by introducing between 2 and 6 mismatches in the sense strand.

### L. Algorithms: Architectural Dependent—Small Internally Segmented

### 1. Description

The more general form of this architecture is characterized by the use of two short sense strands that are complementary to a single antisense strand. In the case of seqMiRs this arrangement can be reversed, i.e., there can be two short antisense strands that are complementary to a single sense strand. In either case these short strands are separated by no more than two nucleoside positions when they form a seqRNAi-based duplex with their partner strand. It is preferred that the short strands be immediately contiguous when duplexed with the partner strand. This can be achieved by simply omitting one linkage in what would otherwise be a single seqRNAi sense strand.

Further, the opposing termini of short strands as they appear in the duplex with the partner strand can be modified to prevent the possibility that they will be ligated in vivo. The likelihood of this occurring, however, has not been established. One method to prevent the possibility of RNA ligation is to use an inverted abasic residue (such as 3′-2′ or 3′-3′) at one of the opposing termini or to have a one or two nucleoside separation between the short strands when they form a duplex with the partner strand.

For more general use in seqsiRNA and seqIMiRs this architecture has the effect of essentially eliminating the possibility that the desired sense strand is loaded into RISC as the antisense strand. In the case of seqMiRs the use of two short antisense strands can eliminate any contribution of 3′-supplementary sites to mRNA target recognition. In instances where a 3′-supplementary site would otherwise be used, for example, this approach can be employed to restrict the range of targets being recognized particularly in cases when the restriction reduces the number of undesired targets.

The short size of the two sense or antisense strands can reduce their affinity with the partner strand to the point that the resulting duplex is not efficiently stable. Often this can be compensated for by judiciously using modifications to the sense strand(s) that are particularly efficacious in increasing the affinity between them and the full-length partner strand. Thermodynamic considerations are discussed in more detail in the section by that name.

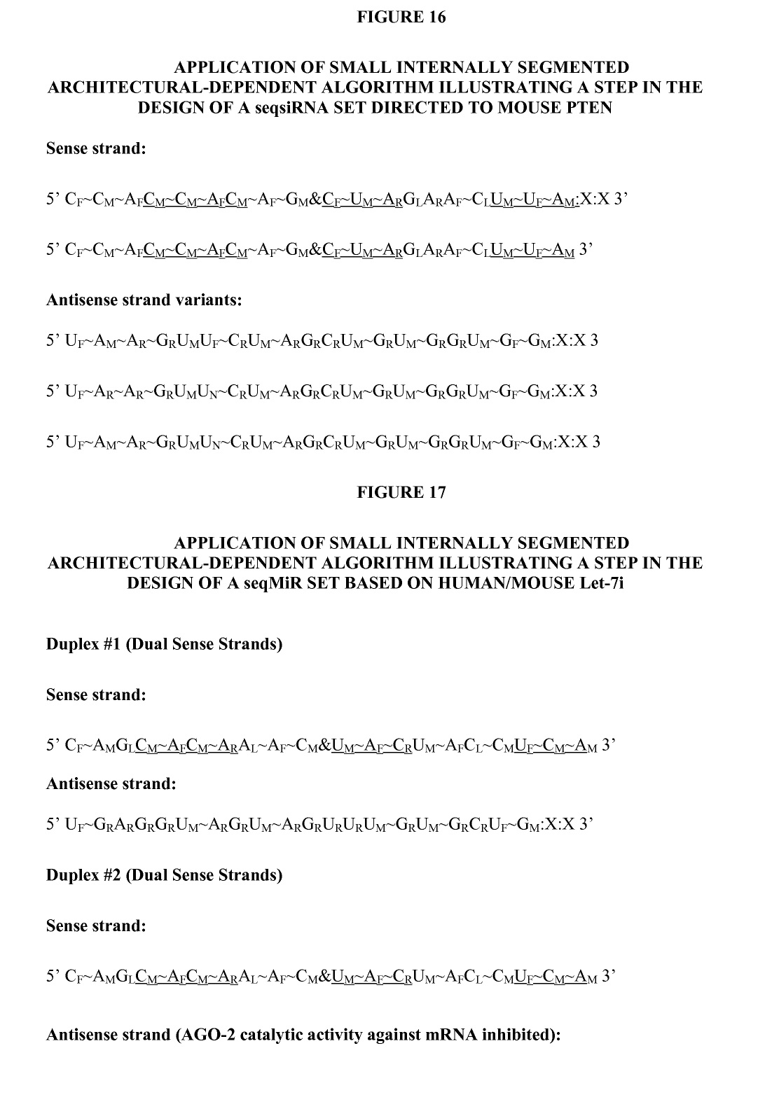

The application of the small internally segmented architecture dependent algorithm to the illustrative seqsiRNA and seqMiR examples is provided in FIGS. 16 and 17 respectively.

The starting two sense and three antisense strands for the application of the small internally segmented architecture as shown in FIG. 16 come from FIG. 10. The sense strand only differed with respect to the presence or absence of overhang precursors were divided into two strands by removing the linkage between nucleoside positions 9 and 10. Next the nuclease resistance rules were applied to the two new termini. The Tms for each of these dual sense strands was determined using the nearest neighbor calculation followed by an adjustment for the chemical modifications. The final Tms were 51 degrees and 34 degrees for the sense strand forming a duplex with the 3′end of the antisense strand or the 5′-end of the antisense strand respectively. To bring the second sense strand above the 40 degree lower limit and to make the Tms similar, the LNA modification was used in positions 4 and 7 counting from the 5′-end of the second sense strand.

In FIG. 17 the starting sense strands for the application of this architecture are the sense strand with the wobble base pairings and mismatches removed in FIG. 9. If the design began with an endogenous miRNA with a bulge structure(s) this structure would also have been removed at the start of the application of the small internally segmented architecture. The two antisense strands come from FIG. 11. One of these strands has a modification that inhibits AGO-2 catalytic activity while the other does not.

The sense strand is split between positions 10 and 11 as indicated by &. The calculated Tm for the unmodified dual sense strands is 32 or 39 and 42 degrees respectively for the strands with the single strand 5′-end and 3′-end. The basis for the alternative Tms for the sense strand with the former single sense strand 5′-end is the presence (Duplex #1) or absence (Duplex #2) of the abasic nucleoside in the antisense strand. The 3′-end nucleosides in each of the sense strands are modified and phosphorothioate linkages are added between nucleoside positions 8-9 and 9-10. These modifications are in keeping with the nuclease resistance rules and the preference for 2′-0-methyl modifications in the terminal nucleoside where there is no overhang precursor with based on a chemistry not permitted in the duplex. The 5-end nucleoside in the antisense strand is change to a 2′-fluoro to meet the preference for 2′-0-methyls to not be in both members of a complementary nucleoside pair. The chemical modifications add about 5 degrees in Tm to each of the sense strands with their partner antisense strand. To increase the Tms for the two sense strands two LNA modifications are added to the first strand and one to the second.

Splitting the antisense strand in FIG. 17 into two strands at the 10-11 linkage does not alter the basic Tm calculations made for the dual sense strands since the deleted linkage in each case opposes the other. The single sense strand in Duplex 3 has the same LNA modifications and the switch of the A_{F} for an A_{M} at the terminal 3′-position and a switch in the U_{M} in position 11 for U_{F} to accommodate the change in the complementary nucleoside in the antisense strand. In keeping with the nuclease resistance rules the A in position 10 becomes 2′-0-methyl, the G in position 11 becomes 2′-fluoro and phosphorothioate linkages are inserted between positions 8-9 and 11-12 basing the count on a single antisense strand.

### 2. Applicable to seqRNAi Sense Strands when Two Sense Strands are Used

a) It is required that there be two sense strands that are separated by no more than two nucleoside positions when they form a seqRNAi-based duplex with their partner antisense strand but it is preferred that they be contiguous. An inverted abasic residue (such as 3′-2′ or 3′-3′) can be used to replace a nucleoside at one of the two termini that will be in opposition when the seqRNAi-based duplex is formed.
b) The sequence of the strands and their chemical modifications determine the Tm of each of the strands with the partner antisense strand. These factors must result in a minimum Tm of 40 degrees centigrade for each sense strand with the antisense strand under physiologic conditions with 50-65 degrees being preferred. It is also preferred the Tms for each of the sense strands with the partner antisense strand be at most only a few degrees apart.
c) LNA(s) can be used in one or both sense strands, as needed, to stabilize the seqRNAi-based duplex under physiologic conditions with a maximum of three per strand. It is preferred that: (1) when there are two or three LNAs in a given strand that they be separated by at least one nucleoside that does not have the LNA modification; (2) LNAs not be in the first position at the 5′-end of the strand; (3) they not be in the terminal 3′-end position if the base is a uracil; and (4) considering the two sense strands as a single unit LNAs preferably are placed between the three regions explicitly defined by Table 3.
d) A 2-thiouridine containing nucleoside can be used in place of LNA to boost interstrand binding affinity when the nucleoside in question has a uracil base and it forms a complementary base pair with an adenine containing nucleoside in the antisense strand. In such an instance the nature of any modifications to the sugar in this nucleoside will follow the relevant architectural independent rules provided herein.
e) The sense strand undergoing complementary base pairing with the 5′-end of the antisense strand can have an overhang precursor.

### 3. Applicable to seqRNAi Antisense Strand when Two Sense Strands are Used

Can follow the rules relevant for the canonical, blunt-end or asymmetric architectures depending on the presence or absence of 5′ and/or 3′-end overhang precursors. A 2-3-unit 3′-end overhang precursor is preferred.

### 4. Applicable to seqsiRNA and seqIMiR Antisense Strands when Two Sense Strands are Used

Can follow the rules relevant for the canonical, blunt-end or asymmetric architectures depending on the presence or absence of 5′ and/or 3′-end overhang precursors.

### 5. Applicable to seqMiR Antisense Strand when Two Sense Strands are Used

Can follow the rules relevant for the canonical, blunt-end or asymmetric architectures depending on the presence or absence of 5′ and/or 3′-end overhang precursors.

### 6. Applicable to seqMiR Sense Strand when Two Antisense Strands are Used

a) The sequence of the strands and their chemical modifications determine the Tm of the strand with the two partner antisense strands. These factors must result in a minimum Tm of 40 degrees centigrade for each antisense strand with the sense strand under physiologic conditions with 50-65 degrees being preferred. It is also preferred the Tms for each of the antisense strands with the partner sense strand be at most only a few degrees apart.
b) LNA(s) can be used in one or both sense strands, as needed, to stabilize the seqRNAi-based duplex under physiologic conditions with a maximum of three per strand. It is preferred that: (1) when there are two or three LNAs in a given strand that they be separated by at least one nucleoside that does not have the LNA modification; (2) LNAs not be in the first position at the 5′-end of the strand; (3) they not be in the terminal 3′-end position if the base is a uracil; and (4) considering the two sense strands as a single unit LNAs are placed between the three regions explicitly defined by Table 3 if possible.
c) A 2-thiouridine containing nucleoside can be used in place of LNA to boost interstrand binding affinity when the nucleoside in question has a uracil base and it forms a complementary base pair with an adenine containing nucleoside in the antisense strand. In such an instance the nature of any modifications to the sugar in this nucleoside will follow the relevant architectural independent rules provided herein.
d) The terminal 5′-end nucleoside preferably is chemically modified, for example, by methylation to prevent its 5′ ribose position from being phosphorylated by endogenous enzymes.
e) In other respects the sense strand will follow the design of the canonical or blunt-end architectures depending on whether it has an overhang precursor or not.

### 7. Applicable to seqMiR Antisense Strands when Two are Used

a) It is required that there be two antisense strands that are separated by no more than two nucleoside positions when they form a seqRNAi-based duplex with their partner sense strand but it is preferred that they be contiguous. An inverted abasic residue (such as 3′-2′ or 3′-3′) can be used to replace a nucleoside at one of the two termini that will be in opposition when the seqRNAi-based duplex is formed.
b) It can otherwise follow the rules relevant for the canonical or blunt-end architecture depending on the presence or absence of a 3′-end overhang precursor.

### M. Algorithms: Architectural Dependent—seqRNAi Antisense Strand Based ss-RNAi

### 1. Description

A seqRNAi antisense strand based ss-RNAi has three general features: (1) it can be administered to a subject with out a carrier or prodrug design; (2) a complementary partner sense strand is not administered to the same subject over a timeframe where both strands can combine in the subject's cells; and (3) it produces the intended silencing effect in cells in a subject. Such antisense strands occur in three specific versions: ss-MiR, ss-IMiR and ss-siRNA depending on whether the antisense strand functions as a miRNA mimic, miRNA inhibitor or a siRNA when loaded into RISC.

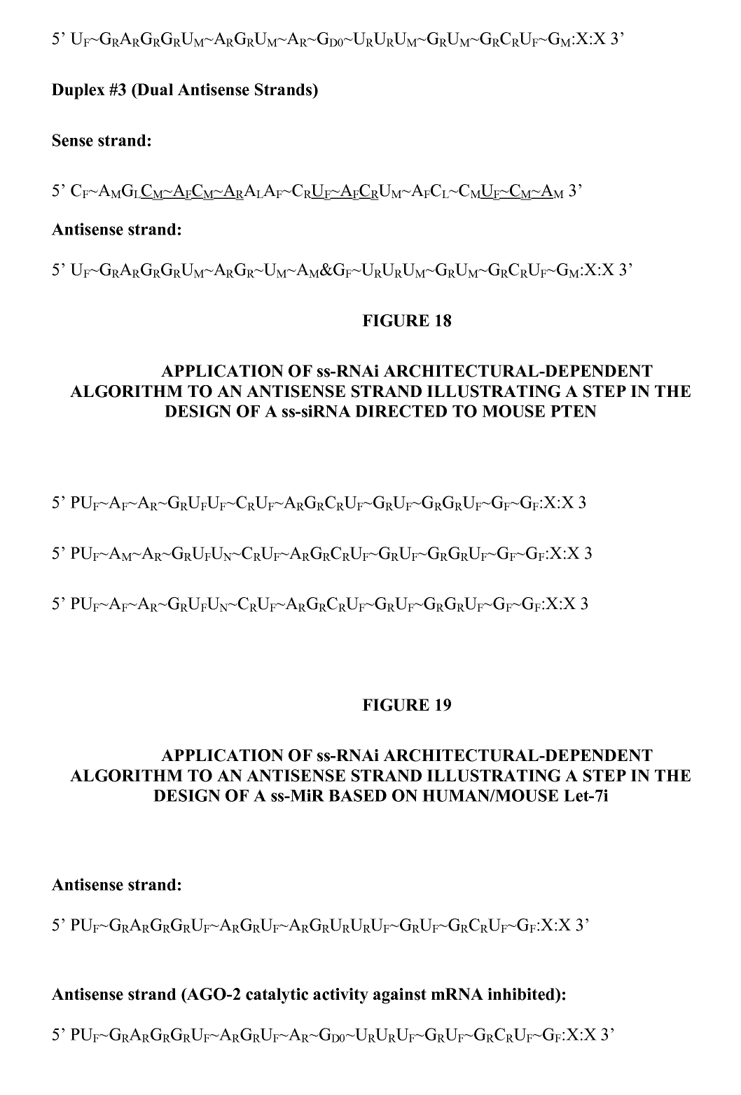

The application of the ss-RNAi architecture dependent algorithm to the illustrative ss-siRNA and ss-MiR examples is provided in FIGS. 18 and 19 respectively.

FIG. 18 shows how the antisense strands shown in FIG. 10 can be adjusted for ss-siRNA use.

FIG. 19 shows examples of several variants of a ss-MiR based on let-7i with and without potential AGO-2 catalytic activity prevented prophylactically and with and without modifications that increase the binding affinity of the seed sequence for its targets. The starting strands came from the antisense strands in FIG. 11 that illustrate the application of the canonical architecture.

### 3. Applicable to ss-RNAi

a) The 5′-end nucleoside is phosphorylated at the 5′ ribose position.
b) Preferably the strand is 16-20 nucleosides in length with a 2-3 unit overhang precursor for a total length of 18-23. Most preferred are overhang precursors that have a relatively high affinity for the PAZ domain of RISC. These can be distinguished by their ability to extend the duration of the intended silencing activity.

### 4. Applicable to ss-siRNA and ss-IMiRs

The nuclease resistance rules, the essential/preferred architecturally independent rules and the canonical or blunt ended rules appropriate to a seqsiRNA/seqIMiR antisense strand are applied. However, 2′-fluoro modifications are preferred over other modifications save ribose and save the overhang precursors if any. There are two exceptions as follows: (1) the use of a minimal number of 2′-0-methyl modifications, if needed, to reduce activation of any unacceptable innate immune response; and (2) the use of an UNA in the seed region and/or a 2′-0-methyl in position 2 from the 5′-end to inhibit miRNA-like off target effects.

### 5. Applicable to ss-MiRs

The nuclease resistance rules, the essential/preferred architecturally independent rules and the canonical or blunt ended rules appropriate to a seqMiR antisense strand are applied. However, 2′-fluoro modifications are preferred over other modifications save ribose and save the overhang precursors if any. There are three exceptions as follows: (1) the use of a minimal number of 2′-0-methyl modifications, if needed, to reduce activation of any unacceptable innate immune response; (2) the use of modifications such as LNA in the seed sequence to increase the seed duplex Tm; and (3) the use of the modifications supplied herein to inhibit the catalytic activity of AGO-2 against unintended RNA targets.

### N. Overhang Precursors

Overhangs in naturally occurring siRNA are typically complementary to their target RNA. Overhangs, however, appear to play little, if any, role in target recognition. The oldest and most used conventional siRNA architecture (canonical) for synthetic compounds is comprised of a 19-mer duplex with two deoxythymidine 3′-end overhangs (dTdT) on both strands. These overhangs were selected because of their convenience and low cost. Nuclease resistant linkages to protect against the 3′-end exonucleases in biologic fluids commonly join the nucleosides in overhangs.

It was originally thought that overhangs were required for siRNA activity in all cell types and that they could be comprised of any native ribonucleoside or deoxyribonucleoside without affecting activity. Subsequently, it was discovered that 3′-end overhangs were not required for siRNA activity in mammalian cells when it was shown siRNA with a blunt-end architecture is capable of producing substantial silencing activity against the intended target.

Endogenous miRNAs have 3′-end overhangs that are generated during the processing of miRNA precursors to become duplexed miRNA that is ready for RISC loading. As for siRNA the overhangs in miRNA are not involved in recognizing the target. Instead the 3′-end antisense strand overhang in siRNA or miRNA has been shown to interact with the PAZ domain in the RNA binding pocket of RISC in a manner that prevents interaction with the target transcript. As a result of this interaction this 3′end overhang can affect RISC loading and antisense strand retention.

Variations in overhang design and chemistry, as well as the option of not using overhangs, can be used to modulate the activity of seqRNAi compounds in commercially useful ways. For example, seqRNAi treatments that sensitize cancers to other therapeutics (typically targeting molecules that inhibit apoptosis) would only be required to be active during the comparatively short period of time required for producing such sensitization. By limiting the duration of such an effect some possible side effects might be reduced or eliminated. In contrast, it would generally be advantageous to structure seqRNAi strands to produce a comparatively long silencing effect when treating chronic diseases such as diabetes or cardiovascular diseases such as atherosclerosis. In addition, particular overhang precursors and designs can be used to promote the selection of the desired antisense stand by RISC and/or to boost the peak silencing activity of the antisense strand/RISC complex as well as its duration.

Overhang precursors in seqRNAi can be of 1 to 4 nucleosides in length, can involve neither, either or both of the 3′-ends of a strand pair as well as the 5′-end of the antisense strand. 3′-end overhangs can have substantially different chemical modifications compared to the rest of the strand while 5′-end overhangs are based on the same nucleoside and linkage chemistries as the portion of the strand that forms a duplex with its partner strand.

The 3′-end overhang precursors in seqRNAi can be comprised of any of the naturally occurring deoxyribonucleosides. In addition, several groups have described variations in overhang design/chemistry that can affect the duration of the silencing effect of conventional siRNA. These same structures can be used as overhang precursors in seqRNAi strands. Zhang et al., (Bioorganic & Medicinal Chemistry 17: 2441, 2009), for example, showed that two nucleoside 3′-end overhangs with morpholine rings replacing the ribose in both the sense and antisense strands or just the antisense strands of conventional siRNA can result in a longer lasting silencing effect than the same siRNA with the standard dTdT overhangs. Strapps et al., (Nucl Acids Res 38: 4788, 2010), in another example, found that the dTdT overhangs were associated with a significantly reduced silencing period both in vitro and in vivo compared to the other overhang types tested. The latter consisted of the following: two 2′-0-methyl uridines; two 2′-0-methyl modified nucleosides complementary to the RNA target; or unmodified ribonucleosides complementary to the RNA target. Differences in duration of effect were found to not be due to either a difference in IC₅₀ values or to variable degrees of maximal target silencing. These data suggest that ribonucleosides may have a stronger binding to the PAZ domain than deoxyribonucleosides.

Numerous other 3′-end overhang precursor chemistries can promote seqRNAi activity and nuclease resistance. These include but are not limited to the following where the indicated nucleoside analog chemistries can be used with any of the normal bases: (1) 2′-0-Methyl; (2) 2′-fluoro; (3) FANA; (4) 2′-0-methyoxyethyl (5) LNA; (6) morpholino; (7) tricyclo-DNA (Ittig et al., Artif DNA, PNA & XNA 1: 9, 2010); (8) ribo-difluorotoluyl (Xia et al., ACS Chem Biol 1: 176, 2006); (9) 4′-thioribonucleotides (Hoshika et al., Chem Bio Chem 8: 2133, 2007); (10) 2′-0-methyl-4′-thioribonucleotide (Takahashi et al., Nucleic Acids Res 37: 1353, 2009; Matsuda, Yakugaku Zasshi 131: 285, 2011); (11) altritol-nucleoside (ANA) (Fisher et al., Nucleic Acids Res 35: 1064, 2007); (12) cyclohexenyl-nucleoside (CeNA) (Nauwelaerts et al., J Am Chem Soc 129; 9340, 2007; (13) piperazine (U.S. Pat. No. 6,841,675); and (14) 5-bis(aminoethyl) aminoethylcarbamoylmethyl-2′-deoxyuridine or 5-bis(aminoethyl) aminoethylcarbamoylmethyl-thymidine (Masud et al., Bioorg Med Chem Lett 21: 715, 2010).

The nucleosides used in overhang precursors in seqRNAi strands can be used in various combinations in 3′-end overhangs and are preferably joined together and to the adjacent non-overhang nucleoside by a nuclease resistant linkage such as phosphorothioate, phosphonoacetate, thiophosphonoacetate, methylborane phosphine, amide, carbamate or urea (Sheehan et al., Nucleic Acids Res 31: 4109, 2003; Krishna & Caruthers, J Amer Chem Soc 133: 9844, 2011; Iwase et al., Nucleic Acids Symposium Series 50: 175, 2006; Iwase et al., Nucleosides Nucleotides Nucleic Acids 26: 1451, 2007; Iwase et al., Nucleic Acids Symposium Series 53: 119, 2009; Ueno et al. Biochem Biophys Res Comm 330: 1168, 2005). In addition unmodified nucleosides can be used in overhangs when they are joined together using these linkages but preferably not phosphorothioate with ribonucleosides. These linkages can also be used in 5′-end overhangs but preferably the nucleosides are limited to the following: (1) 2′-0-Methyl; (2) 2′-fluoro; (3) FANA; and (4) RNA (native ribose). In the case of seqMiRs, however, such 5′-end modifications have to be evaluated for their effects on what mRNAs will be targeted for silencing.

Further, 3′-end overhang precursors can be comprised of certain hydrophobic aromatic moieties. For example, those that are comprised of one to three units containing two six membered rings joined by phosphodiester or one of the other linkages just listed where the unit(s) are attached to the oligonucleotide by the same linkage and when multiple units are used they are also joined by the same linkage. Two unit structures are preferred. Suitable ring structures include benzene, pyridine, morpholine and piperazine (U.S. Pat. No. 6,841,675). Structures based on the benzene and pyridine rings have been previously described for 3′-end overhang use in conventional siRNA by Ueno et al., (Bioorg Med Chem Lett 18:194, 2008; Bioorganic & Medicinal Chemistry 17: 1974, 2009). Specifically, these units are 1,3-bis(hydroxymethyl)benzene, 1,3-bis(hydroxymethyl)pyridine and 1,2-bis(hydroxymethyl)benzene. These are also suitable for seqRNAi use as overhang precursors.

In another example of possible non-nucleoside overhang precursors the aromatic moieties can be biaryl units where the linkages holding the units together and to the oligo are covalently attached to benzene rings where the benzene ring is further covalently attached to a non-bridging moiety selected from the group benzene, naphthalene, phenanthrene, and pyrene. Further, one such biaryl group may be attached to the 5′-end of the intended sense strand to substantially reduce the likelihood it will be selected as the antisense strand by RISC once the complementary seqRNAi strands form a duplex in cells. (Ueno et al., Nucleic Acids Symposium Series 53: 27, 2009; Yoshikawa et al., Bioconjugate Chem 22: 42, 2011) When these units are used as overhang precursors one to three units are preferred and two are most preferred.

In addition, the 3′-end overhangs, or lack thereof, can affect the distribution of seqRNAi-based duplexes between the cytoplasm and nucleus. Individual seqRNAi strands released into the cytoplasm and the duplexes formed by a seqRNAi strand pair can diffuse into the nucleus. Once in the nucleus individual seqRNAi strands can form seqRNAi-based duplexes and any duplexes that were formed in the cytoplasm that subsequently diffused into the nucleus can be expelled from the nucleus by Exportin-5 (Exp-5). This activity of Exp-5 can be rate-limiting for silencing activity at low doses of duplexes. Exp-5 binds to the first two nucleosides or their analogs in any 3′-end overhang(s) while possibly binding more weakly to the duplexed portion. Thus, seqRNAi strands designed to have 3′-end overhang precursors comprising nucleosides have a potential advantage over seqRNAi strands that do not have overhang precursors because they can produce a greater duplex presence in the cytoplasm particularly at lower seqRNAi concentrations. Finally, the nature of the 3′-end overhang precursors, if any, affects the overall and regional interstrand affinities of seqRNAi-based duplexes. This topic is discussed in the section dealing with thermodynamics.

### O. Methods of Administration of the Single Strand Oligo Compounds of the Invention

A major advantage of the present invention in effecting RNAi is that many of the modifications described employ chemistries commonly used in conventional antisense oligos where the pharmacology and toxicology of the compounds is already largely understood described in the literature. References that summarize much of pharmacology for a range of different types of oligo therapeutics includes the following: Antisense Drug Technology: Principles, Strategies, and Applications, 2^{nd} ed., Stanley T. Crooke (ed.) CRC Press July 2007; Encyclopedia of Pharmaceutical Technology,—6 Volume Set, J Swarbrick (Editor) 3rd edition, 2006, Informa HealthCare; Pharmaceutical Perspectives of Nucleic Acid-Based Therapy, RI Mahato and SW Kim (Editora) 1 edition, 2002, CRC press; Pharmaceutical Aspects of Oligonucleotides, P Couvreur and C Malvy (Editors) 1st edition, 1999, CRC press; Therapeutic Oligonucleotides (RSC Biomolecular Sciences) (RSC Biomolecular Sciences) (Hardcover) by Jens Kurreck (Editor) Royal Society of Chemistry; 1 edition, 2008, CRC press; Clinical Trials of Genetic Therapy with Antisense DNA and DNA Vectors, E Wickstrom (Editor) 1st edition, 1998, CRC press.

The fact the compounds of the present invention are sequentially delivered does add an additional complication. There must be a long enough period between the administration of the first strand and the second for cells to have taken up most of the first strand. The periods of time involved have been worked out for conventional antisense oligos and can be applied here. For example, when these compounds are infused into the circulation the clearance time half-life from the plasma to the tissues is about 20 minutes. Thus, after one hour most of the compound is in the tissues. The tissue retention time depends on dose but within the dose range commonly used to treat subjects the tissue retention can be measured in days or weeks. The compound in the tissues is distributed between a bioavailable form and a unavailable form, but it is clear the former can exist at effective levels for days or weeks based on the protracted suppression of the target in tissues.

It follows, therefore, that the seqRNAi compounds of the present invention will be given to subjects in the dose range established for conventional antisense oligos and that the spacing between the two strands for i.v. or i.a. administration will range from about one hour to a week, but 4 hours to 24 hours between strand administrations is preferred. For most systemic in vivo purposes administration of a strand over one hour at an infusion rate of up to 6 mg/kg/h is appropriate.

The timing of strand administration i.v. or i.a. can also serve for a number of other administrative routes where the compounds are juxtaposed to the target tissue such as i.p., intrathecal, intraocular and intravesical. The treatment regimens will for the seqRNAi compounds will also mirror those used for conventional antisense oligos. For the ss-RNAi compounds of the present invention the sequential delivery related issues do not apply so they can be fully treated like conventional antisense oligos.

In certain embodiments, (e.g., for the treatment of lung disorders, such as pulmonary fibrosis or asthma or to allow for self administration for local or systemic purposes) it may desirable to deliver the oligos described herein in aerosolized form. A pharmaceutical composition comprising at least one oligo can be administered as an aerosol formulation that contains the oligos in dissolved, suspended or emulsified form in a propellant or a mixture of solvent and propellant. The aerosolized formulation is then administered through the respiratory system or nasal passages.

An aerosol formulation used for nasal administration is generally an aqueous solution designed to be administered to the nasal passages as drops or sprays. Nasal solutions are generally prepared to be similar to nasal secretions and are generally isotonic and slightly buffered to maintain a pH of about 5.5 to about 6.5, although pH values outside of this range can also be used. Antimicrobial agents or preservatives can also be included in the formulation.

An aerosol formulation for use in inhalations and inhalants is designed so that the oligos are carried into the respiratory tree of the patient. See (WO 01/82868; WO 01/82873; WO 01/82980; WO 02/05730; WO 02/05785 Inhalation solutions can be administered, for example, by a nebulizer Inhalations or insufflations, comprising finely powdered or liquid drugs, are delivered to the respiratory system as a pharmaceutical aerosol of a solution or suspension of the drug in a propellant.

An aerosol formulation generally contains a propellant to aid in disbursement of the oligos. Propellants can be liquefied gases, including halocarbons, for example, fluorocarbons such as fluorinated chlorinated hydrocarbons, hydrochlorofluorocarbons, and hydrochlorocarbons as well as hydrocarbons and hydrocarbon ethers (Remington's Pharmaceutical Sciences 18th ed., Gennaro, A. R., ed., Mack Publishing Company, Easton, Pa. (1990)).

Halocarbon propellants useful in the invention include fluorocarbon propellants in which all hydrogens are replaced with fluorine, hydrogen-containing fluorocarbon propellants, and hydrogen-containing chlorofluorocarbon propellants. Halocarbon propellants are described in Johnson, U.S. Pat. No. 5,376,359, and Purewal et al., U.S. Pat. No. 5,776,434.

Hydrocarbon propellants useful in the invention include, for example, propane, isobutane, n-butane, pentane, isopentane and neopentane. A blend of hydrocarbons can also be used as a propellant. Ether propellants include, for example, dimethyl ether as well as numerous other ethers.

The oligos can also be dispensed with a compressed gas. The compressed gas is generally an inert gas such as carbon dioxide, nitrous oxide or nitrogen.

An aerosol formulation of the invention can also contain more than one propellant. For example, the aerosol formulation can contain more than one propellant from the same class such as two or more fluorocarbons. An aerosol formulation can also contain more than one propellant from different classes. An aerosol formulation can contain any combination of two or more propellants from different classes, for example, a fluorohydrocarbon and a hydrocarbon.

Effective aerosol formulations can also include other components, for example, ethanol, isopropanol, propylene glycol, as well as surfactants or other components such as oils and detergents (Remington's Pharmaceutical Sciences, 1990; Purewal et al., U.S. Pat. No. 5,776,434). These aerosol components can serve to stabilize the formulation and lubricate valve components.

The aerosol formulation can be packaged under pressure and can be formulated as an aerosol using solutions, suspensions, emulsions, powders and semisolid preparations. A solution aerosol consists of a solution of an active ingredient such as oligos in pure propellant or as a mixture of propellant and solvent. The solvent is used to dissolve the active ingredient and/or retard the evaporation of the propellant. Solvents useful in the invention include, for example, water, ethanol and glycols. A solution aerosol contains the active ingredient peptide and a propellant and can include any combination of solvents and preservatives or antioxidants.

An aerosol formulation can also be a dispersion or suspension. A suspension aerosol formulation will generally contain a suspension of an effective amount of the oligos and a dispersing agent. Dispersing agents useful in the invention include, for example, sorbitan trioleate, oleyl alcohol, oleic acid, lecithin and corn oil. A suspension aerosol formulation can also include lubricants and other aerosol components.

An aerosol formulation can similarly be formulated as an emulsion. An emulsion can include, for example, an alcohol such as ethanol, a surfactant, water and propellant, as well as the active ingredient, the oligos. The surfactant can be nonionic, anionic or cationic. One example of an emulsion can include, for example, ethanol, surfactant, water and propellant. Another example of an emulsion can include, for example, vegetable oil, glyceryl monostearate and propane.

Oligos may be formulated for oral delivery (Tillman et al., J Pharm Sci 97: 225, 2008; Raoof et al., J Pharm Sci 93: 1431, 2004; Raoof et al., Eur J Pharm Sci 17: 131, 2002; U.S. Pat. No. 6,747,014; US 2003/0040497; US 2003/0083286; US 2003/0124196; US 2003/0176379; US 2004/0229831; US 2005/0196443; US 2007/0004668; US 2007/0249551; WO 02/092616; WO 03/017940; WO 03/018134; WO 99/60012). Such formulations may incorporate one or more permeability enhancers such as sodium caprate that may be incorporated into an enteric-coated dosage form with the oligo.

There are also delivery mechanisms applicable to oligos with or without carriers that can be applied to particular parts of the body such as the CNS. These include the use of convection-enhanced delivery methods such as but not limited to intracerebral clysis (convection-enhanced microinfusion into the brain—Jeffrey et al., Neurosurgery 46: 683, 2000) to help deliver the cell-permeable carrier/NABT complex to the target cells in the CNS as described in WO 2008/033285.

Drug delivery mechanisms based on the exploitation of so-called leverage-mediated uptake mechanisms are also suitable for the practice of this invention (Schmidt and Theopold, Bioessays 26: 1344, 2004). These mechanisms involve targeting by means of soluble adhesion molecules (SAMs) such as tetrameric lectins, cross-linked membrane-anchored molecules (MARMs) around lipoproteins or bulky hinge molecules leveraging MARMs to cause a local inversion of the cell membrane curvature and formation of an internal endosome, lysosome or phagosome. More specifically leverage-mediated uptake involves lateral clustering of MARMs by SAMs thus generating the configurational energy that can drive the reaction towards internalization of the oligo carrying complex by the cell. These compositions, methods, uses and means of production are provided in WO 2005/074966.

As for many drugs, dose schedules for treating patients with oligos can be readily extrapolated from animal studies. The extracellular concentration that must be generally achieved with highly active conventional antisense or complementary sense and antisense oligos for use in the two-step method is in the 1-200 nanomolar (nM) range. Higher extracellular levels, up to 1.5 micromolar, may be more appropriate for some applications as this can result in an increase in the speed and the amount of the oligos driven into the tissues. Such levels can readily be achieved in the plasma.

For in vivo applications, the concentration of the oligos to be used is readily calculated based on the volume of physiologic balanced-salt solution or other medium in which the tissue to be treated is being bathed. With fresh tissue, 1-1000 nM represents the concentration extremes needed for oligos with moderate to excellent activity. Two hundred nanomolar (200 nM) is a generally serviceable level for most applications. With most cell lines a carrier will typically be needed for in vitro administration. Incubation of the tissue with the oligos at 5% rather than atmospheric (ambient) oxygen levels may improve the results significantly.

Pharmacologic/toxicologic studies of phosphorothioate oligos, for example, have shown that they are adequately stable under in vivo conditions, and that they are readily taken up by all the tissues in the body following systemic administration with a few exceptions such as the central nervous system (Iversen, Anticancer Drug Design 6:531, 1991; Iversen, Antisense Res. Develop. 4:43, 1994; Crooke, Ann. Rev. Pharm. Toxicol. 32: 329, 1992; Cornish et al., Pharmacol. Comm. 3: 239, 1993; Agrawal et al., Proc. Natl. Acad. Sci. USA 88: 7595, 1991; Cossum et al., J. Pharm. Exp. Therapeutics 269: 89, 1994). These compounds readily gain access to the tissue in the central nervous system in large amounts following injection into the cerebral spinal fluid (Osen-Sand et al., Nature 364: 445, 1993; Suzuki et al., Amer J. Physiol. 266: R1418, 1994; Draguno et al., Neuroreport 5: 305, 1993; Sommer et al., Neuroreport 5: 277, 1993; Heilig et al., Eur. J. Pharm. 236: 339, 1993; Chiasson et al., Eur J. Pharm. 227: 451, 1992). Phosphorothioates per se have been found to be relatively non-toxic, and the class specific adverse effects that are seen occur at higher doses and at faster infusion rates than is needed to obtain a therapeutic effect with a well-chosen sequence. In addition to providing for nuclease resistance, one potential advantage of phosphorothioate and boranophosphate linkages over the phosphodiester linkage is the promotion of binding to plasma proteins and albumin in particular with the resulting effect of an increased plasma half-life. By retaining the oligo for a longer period of time in plasma the oligo has more time to enter tissues as opposed to being excreted by the kidney. Oligos with primarily or exclusively phosphodiester linkages have a plasma half-life of only a few minutes. Thus, they are of little use for in vivo applications when used without a carrier. In the case of oligos with a preponderance of or exclusively phosphodiester linkages, plasma protein binding can be improved by covalently attaching the oligo a molecule that binds a plasma protein such as serum albumin. Such molecules include, but are not limited to, an arylpropionic acid, for example, ibuprofen, suprofen, ketoprofen, pranoprofen, tiaprofenic acid, naproxen, flurpibrofen and carprofen (U.S. Pat. No. 6,656,730). As for other moieties that might be linked to the oligos suitable for use with the present invention the preferred site is the 3′-end of the oligo. Intravenous administrations of oligos can be continuous for days or be administered over a period of minutes depending on the particular oligos and the medical indication. Phosphorothioate-containing oligos have been tested containing 18 nucleotides (e.g., oblimersen) to 20 nucleotides (e.g., cenersen, alicaforsen, aprinocarsen, ISIS 14803, ISIS 5132 and ISIS 2503) in length. When so administered such oligos show an alpha and a beta phase of elimination from the plasma. The alpha phase oligo half-life is 30 to 60 minutes while the beta phase is longer than two weeks for oligos with both phosphorothioate linkages and 2′-0 substitutions in at least the terminal four nucleosides on each end of the oligo.

The most prominent toxicities associated with intravenous administration of phosphorothioates have been related to the chemical class and generally independent of the mRNA target sequence and, therefore, independent of hybridization. The observed and measured toxicities have been consistent from drug to drug pre-clinically and clinically, with non-human primates being most similar to humans for certain key toxicities.

The class-related toxicities that have been most compelling in choosing dose and schedule for pre-clinical and clinical evaluation occur as a function of binding to specific plasma proteins and include transient inhibition of the clotting cascade and activation of the complement cascade. Both of these toxicities may be related to the polyanionic nature of the molecules.

The effect of phosphorothioates on the clotting cascade results in plasma concentration-related prolongation of the activated partial thromboplastin (aPPT) time. Maximum prolongation of the aPTT correlates closely with the maximum plasma concentration so doses and schedules that avoid high peak concentrations can be selected to avoid significant effects on the aPTT. Because the plasma half-life of these drugs is short (30 to 60 minutes), the effect on clotting is transient. Several of these drugs have been evaluated in the clinic with prolonged intravenous infusions lasting up to 3 weeks. Shorter IV infusions (e.g., 2 hours) have also been studied. For example, aprinocarsen (ISIS 3521) and ISIS 5132 were studied with both 2 hour and 3-week continuous infusion schedules. At a dose of 3 mg/kg/dose over 2 hours, transient prolongation of the aPTT was observed. When 3 mg/kg was given daily by continuous infusion for 21 days, there was no effect on aPTT. The effect of antisense molecules of this chemical class on the clotting cascade is consistent.

Similarly, the activation of complement is a consistent observation; however, the relationship between plasma concentration of oligonucleotides and complement activation is more complex than the effect on clotting. Also, while the effect on clotting is found in rats as well as monkeys, the effect on the complement cascade has only been observed in monkeys and humans.

When these drugs are given to cynomolgus monkeys by 2-hour infusion, increases in complement split products (i.e., C3a, C5a, and Bb) occur only when plasma concentrations exceed a threshold value of 40-50 μg/mL. In monkeys, there is a low incidence of cardiovascular collapse associated with increases in these proteins. For the most part, clinical investigations of phosphorothioates have been designed to avoid these high plasma concentrations.

When ISIS 3521 was given as a weekly 24 hour infusion at doses as high as 24 mg/kg (1 mg/kg/hour×24 hours), the steady state plasma concentrations reached approximately 12 μg/mL at the high dose. On this schedule, however, there were substantial increases in C3a and Bb even though these plasma levels were much lower than those seen with the shorter infusions. Thus, activation of complement may be associated with both dose and schedule where plasma concentrations that are well tolerated over shorter periods of time (e.g. 2 hours), are associated with toxicity when the plasma concentrations are maintained for longer. This likely provides the explanation for the findings with cenersen in rhesus monkeys where complement activation was observed at concentrations of 14-19 μg/mL.

When ISIS 3521 was given at 1.0 and 1.25 mg/kg/hour×2 hours, the mean peak plasma concentrations were 11.1±0.98 and 6.82±1.33 μg/mL, respectively. There was no complement activation at these or other higher doses and no other safety issues. It is expected that the maximum peak plasma concentrations for similarly sized phosphorothioate given at 1.2 mg/kg/hour×1 hour would be similar to that observed with ISIS 3521.

Thus, limiting infusion rates for phosphorothioates to 3.6 mg/kg/h or less is highly preferred. With somewhat higher infusion rates the effects of complement activation can be expected. Decisions made about the sequential shortening of the infusion below one hour using a constant total dose of approximately 22 mg/kg should be readily achieved based on review of the safety information, including evaluation of complement split products.

The following examples are provided to illustrate certain embodiments of the present invention. They are not intended to limit the invention in any way.

### Example I

### Applications for seqsiRNA

The seqsiRNA genes targeted for silencing are shown in Table 6 and in the examples. They are not meant to provide an exhaustive set of illustrations of how the designs presented herein can be applied in general or in particular. One skilled in the art can readily use the design principles and the examples provided herein to arrive at a very limited set of compounds that can be generated in accordance with the present invention using any given gene target in a subject.

TABLE 6
EXAMPLES OF COMMERCIAL APPLICATIONS FOR seqsiRNA INHIBITORS
FOR ILLUSTRATIVE GENE TARGETS

| GENE | MEDICAL CONDITIONS TO BE TREATED OR OTHER |
|---|---|
| TARGET | COMMERCIAL OBJECTIVES FOR seqsiRNA p53 INHIBITORS |
| Apoliprotein B | Atherosclerosis; Congestive heart failure; Familial hypercholesterolemia; Statin |
| (Apo B) | resistant hypercholesterolemia; HDL/LDL cholesterol imbalance; dyslipidemias; |
| | Acquired hyperlipidemia; Coronary artery disease; Thrombosis |
| FAS/APO-1 | Myocardial infarction; Fatty liver disease; Fulminant hepatitis; Cirrhosis of the |
| (CD-95; | liver; Alcoholic hepatitis; Cholestatic liver injury; Acute liver failure; Cystic |
| Tnfrsf6) | fibrosis; Systemic lupus erythematosus; Arthritis; Parkinson's Disease; |
| | Autoimmune diabetes; Central nervous system injuries, Demyelinating diseases; |
| | Stroke; Chemotherapy-induced neuropathy; Neurodegenerative diseases; Spinal |
| | cord injury; Ischemia—reperfusion injury |
| p53 | Sensitize cancers with wild type p53 to cytotoxic therapies; Cancers with mutant |
| | p53; Sensitize cancers with mutant p53 to the induction of apoptosis by |
| | anyapoptosis inducer; Stem cell quiescence including malignant stem cells |
| | (expand normal stem cells and progeny or put malignant stem cells in cycle so |
| | they can be attacked by cell cycle dependent anti-cancer agents; Heart failure; |
| | Medical conditions where apoptosis is promoted; Inhibiting apoptosis in non- |
| | malignant stem cells; Huntington's disease; Diamond-Blackfan syndrome; |
| | Shwachman Diamond Syndrome and other disorders involving defective |
| | ribosomes and/ or imbalances in ribosomal components (ribosomopathies); Fatty |
| | liver disease; Stress induced immunosuppression; Sequellae associated with |
| | subarachnoid hemorrhage; Pathologic hyperpigmentation; Hyperkeratosis; Toxic |
| | effects of cancer chemotherapy and radiation including but not limited to the |
| | following: hair loss, mucositis, myelosupression, hearing loss, peripheral nerve |
| | damage, impaired brain function and kidney damage; Inflammatory bowel |
| | disease; Crohn's disease; ARDS; Multiple organ failure; Sensitize cancers to |
| | cytotoxic treatments dependent on cell proliferation and/or DNA replication; |
| | Amyloid deposition; Neurodegenerative diseases; Ischemia-reperfusion injury; |
| | Avoidance of effects of cytotoxic therapy due to quiescence of malignant stem |
| | cells; Reduced expansion of non-malignant tissue due to stem cell quiescence; |
| | Prevent demyelination; Multiple sclerosis; Alzheimer's Disease; Parkinson's |
| | disease; Prevent cell death associated with diabetic ischemia; Spontaneous |
| | apoptosis, cell cycle arrest, senescence and differentiation in stem cells including |
| | embryonic stem cells and iPS such as reduces the efficiency of preparing such |
| | cells for transplantation organ generation, the generation of animals or for use in |
| | scientific research; Prevent cell death associated with cerebral ischemia; Prevent |
| | cell death associated with myocardial infarction including consequent heart wall |
| | rupture; Schizophrenia; Psoriasis; AIDS; Prevent rupture of atherosclerotic |
| | plaques; Prevent aneurysm rupture; Graft vs host disease; Systemic lupus |
| | erythematosus; Promote healing of hard to heal wounds; Capillary leak |
| | syndrome; Emphysema; Reduce enodosomal, lysosomal or phagosomal |
| | sequestration of oligo therapeutics with the effect of increasing their biologic |
| | activity; Promote proliferation of stem cells such as hematopoietic or neural; |
| | Diabetes mellitus including insulin resistant diabetes; 5q- syndrome; |
| | Porokeratosis; Ferritin induced cell death such as occurs in iron overload; |
| | Anemia; Dyskeratosis congentia including that form with telomerase |
| | insufficiency; Prevent emphysema; Prevent COPD; Insulin resistance in heart |
| | failure |
| PCSK9 | Atherosclerosis; Hypercholesterolemia; Statin resistant hypercholesterolemia; |
| (NARC-1) | HDL/LDL cholesterol imbalance; dyslipidemias; Acquired hyperlipidemia; |
| | Coronary artery disease |
| PTEN | Cancers with mutated p53; Activate cell proliferation including hematopoietic |
| (MMAC1; | stem and progenitor cells; Increase efficiency of gene transfer including into |
| TEP1) | hematopoietic stem and progenitor cells; Nerve cell regeneration |
| PTP-1B | Insulin resistance; Type II Diabetes |
| Stat3 | Cancer, Autoimmune disease |

### a. Compounds for Down-Regulating p53 Expression

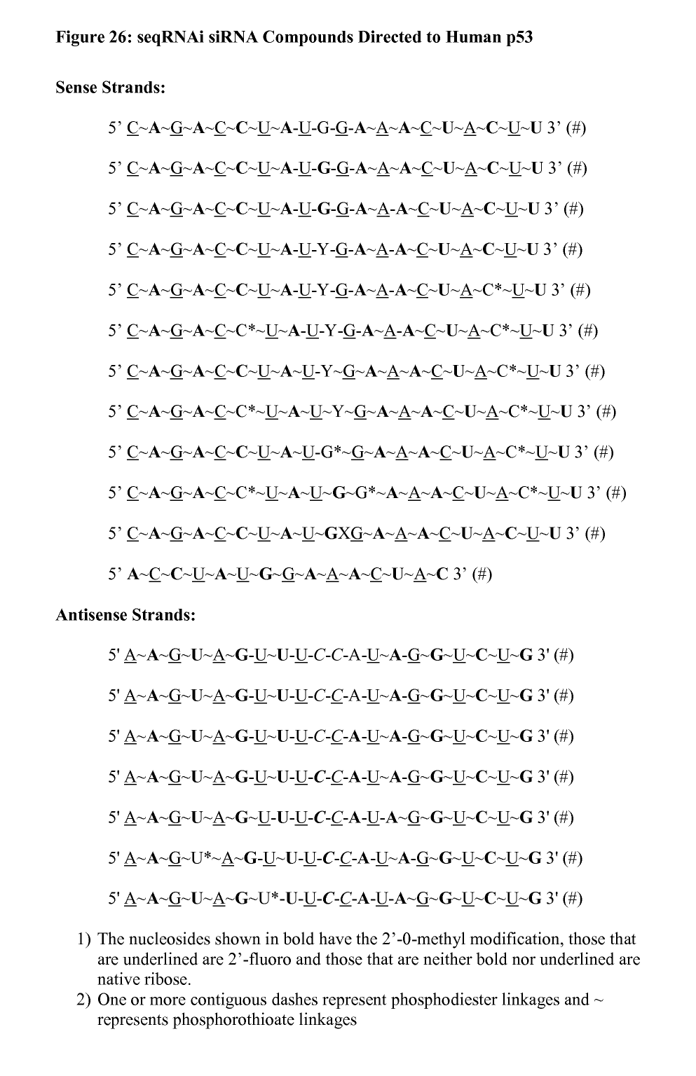

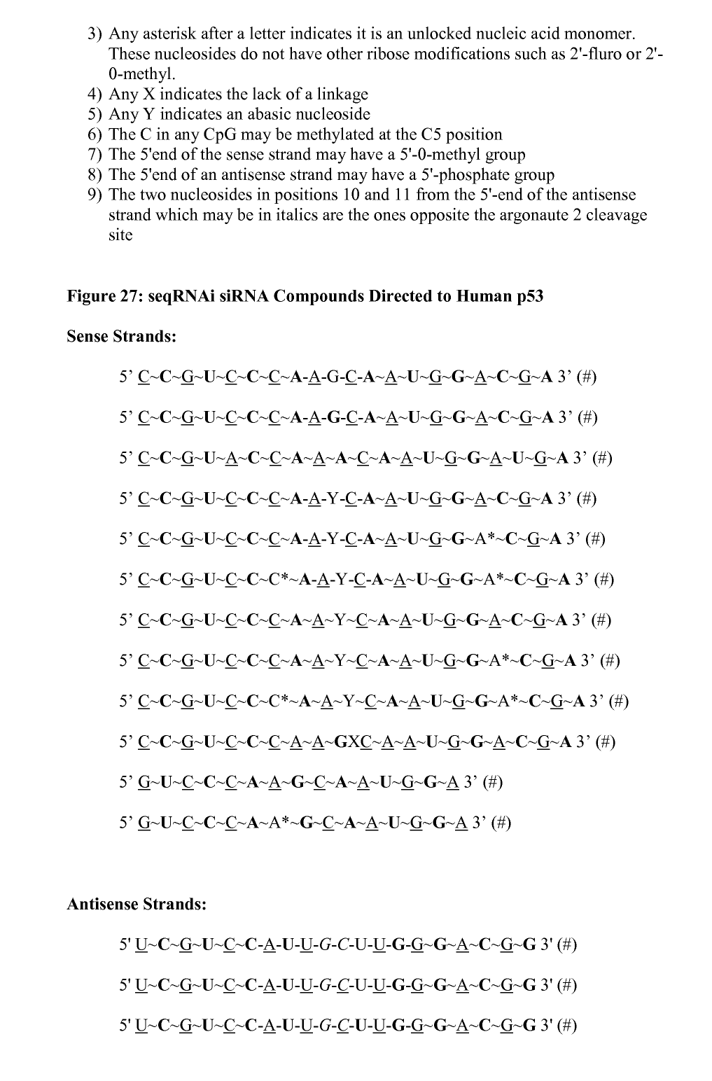

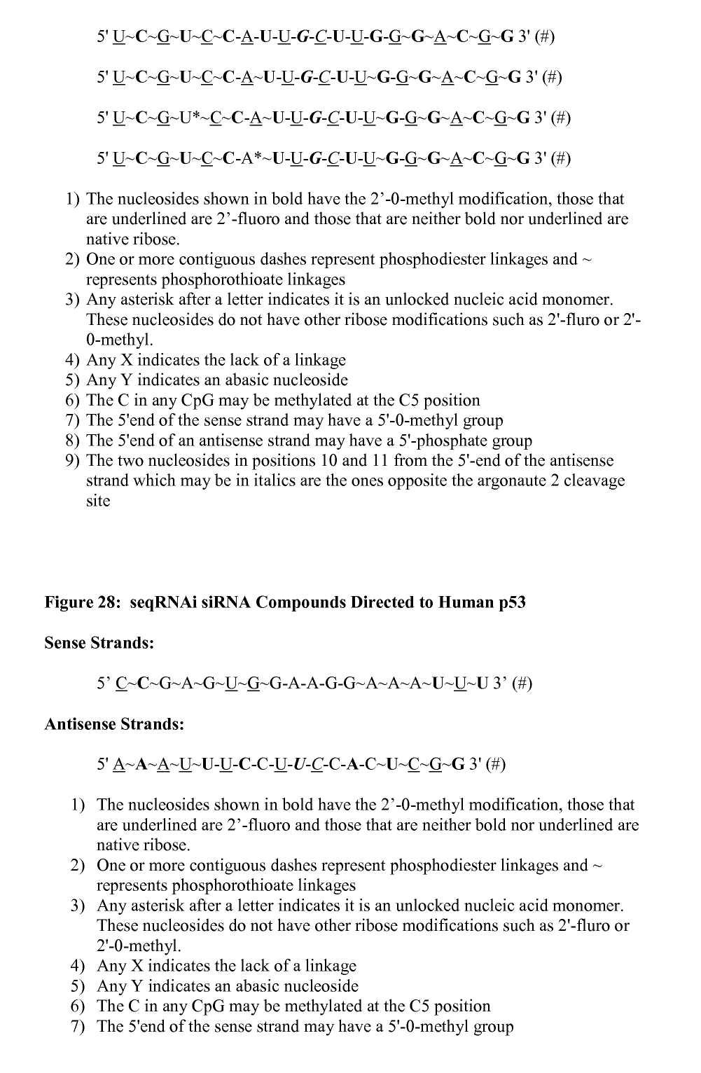

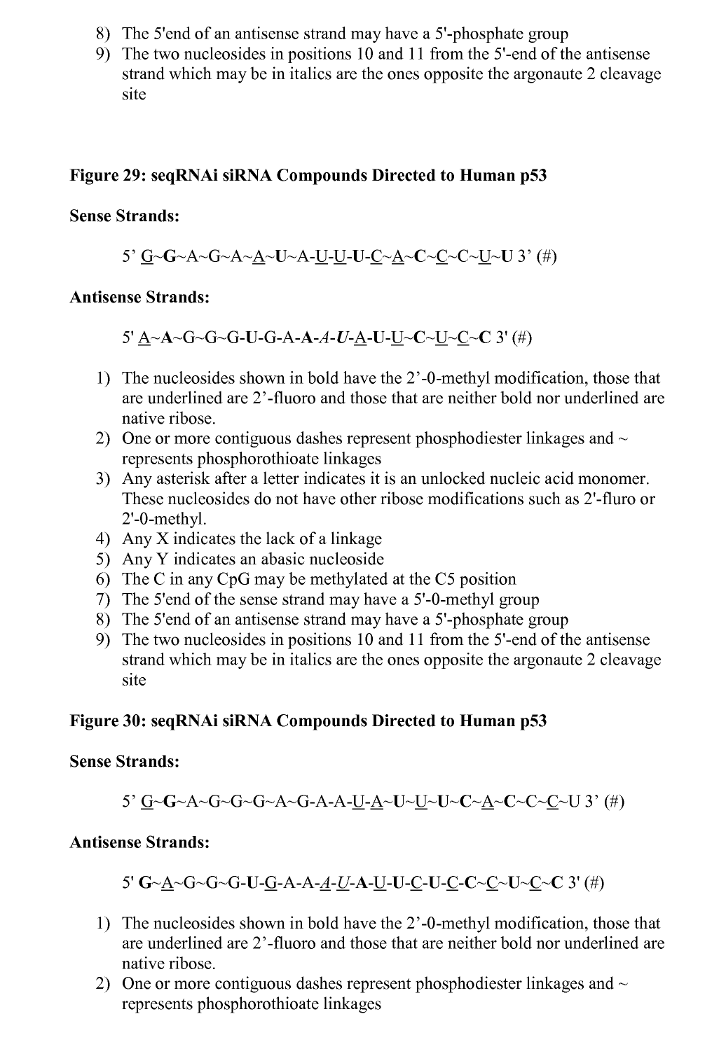

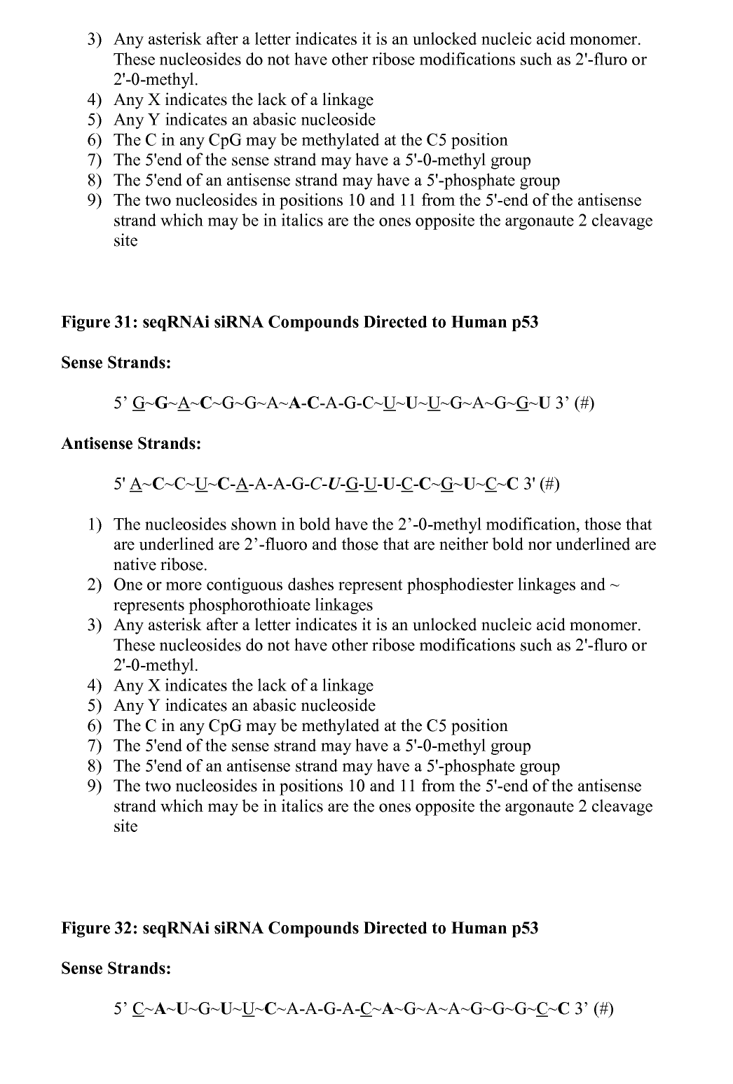

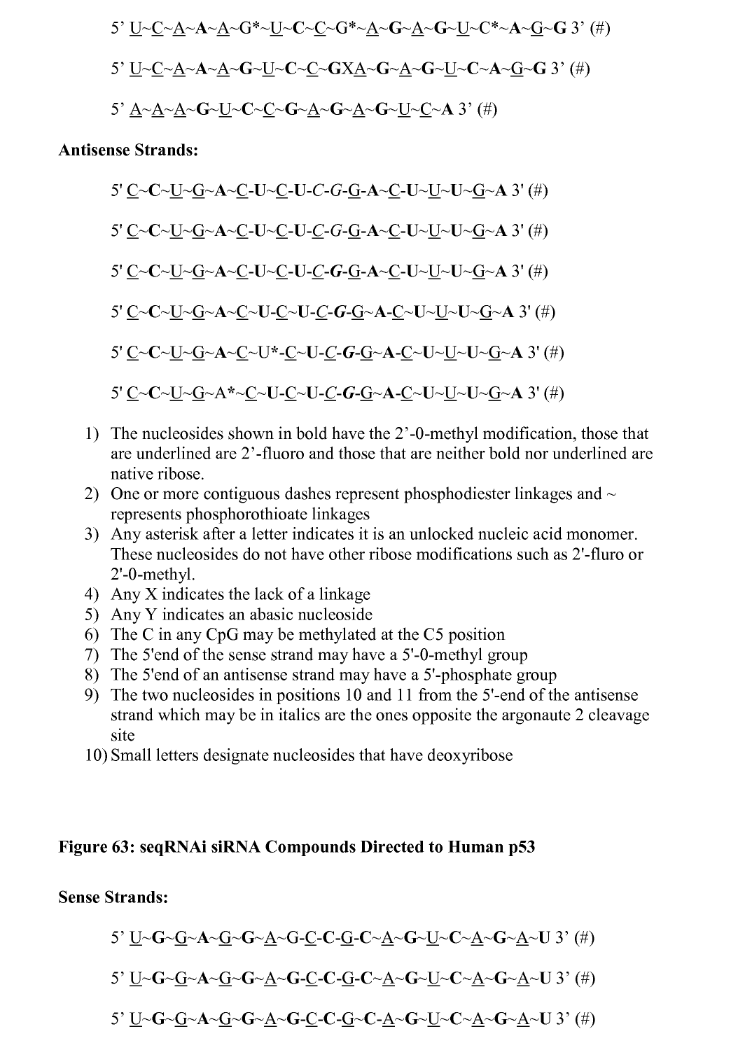

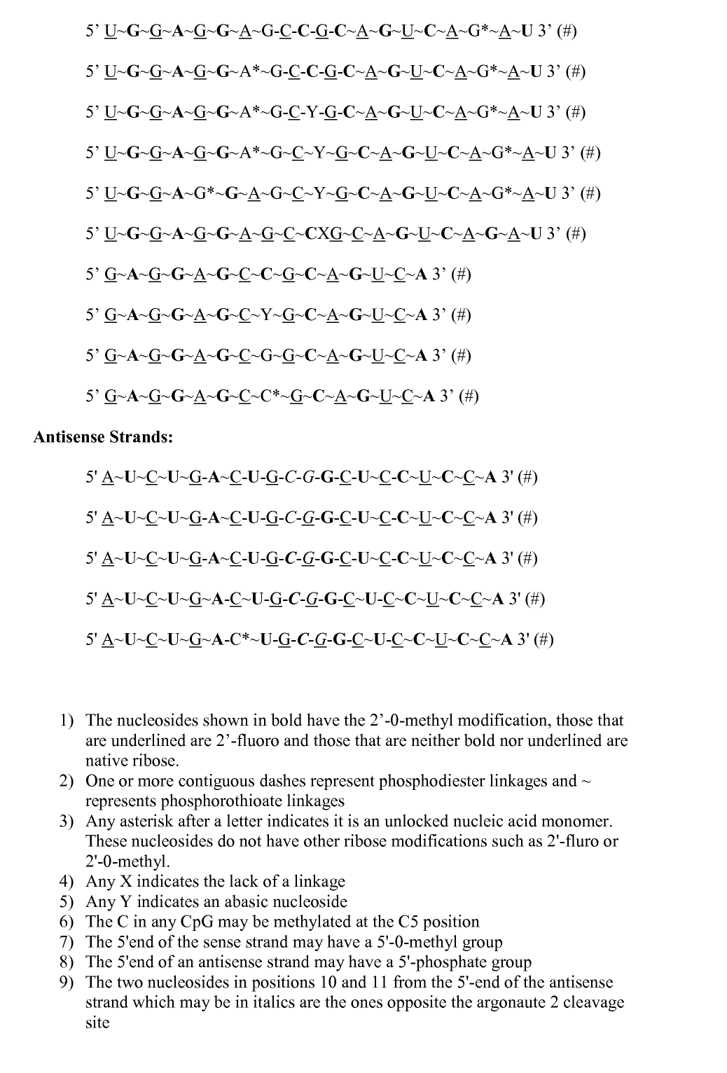

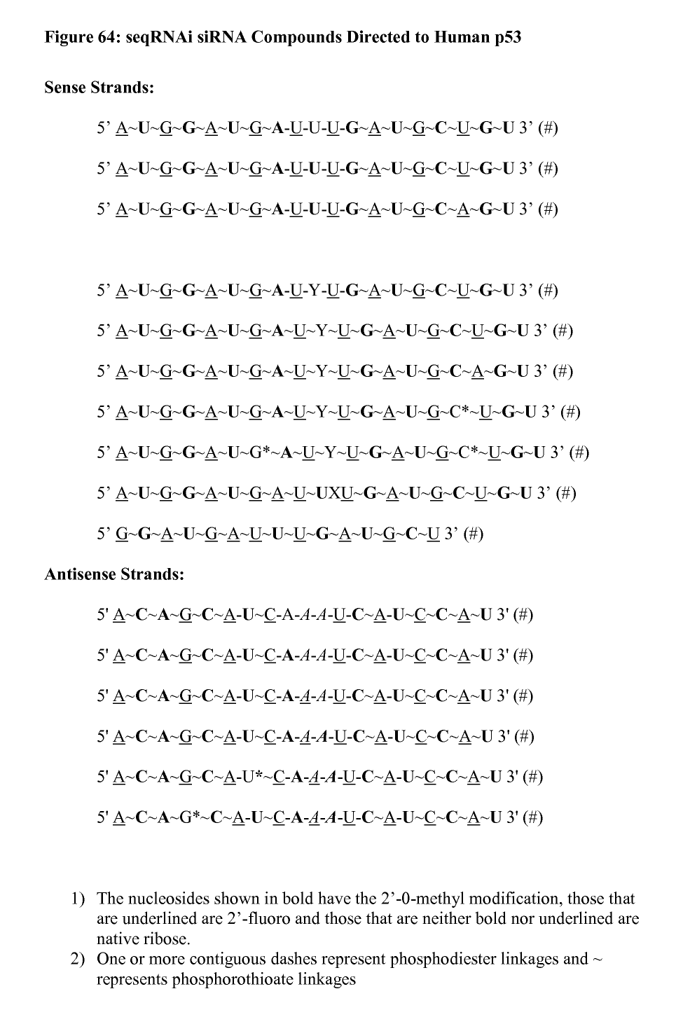

p53 is involved in the regulation of a variety of cellular programs including those involving stem cell self-renewal, cellular proliferation and viability such as proliferation, differentiation, apoptosis, senescence, mitotic catastrophe and autophagy. FIGS. 26-32, 63 and 64 provide compounds suitable for use in accordance with the present invention.

The pathological expression or failure of expression of such programs, and the death programs in particular, underlie many of the morbidities associated with a wide variety of medical conditions where blocking p53 function can prevent much if not all of such morbidity.

In cancer, for example, both wild type and mutant p53 play key roles in tumor maintenance that include increasing the threshold for the induction of programs that can lead to the death of the cancer cells. Typically the use of a p53 inhibitor, such as a siRNA directed to the p53 gene target, in combination with an inducer of a cell death program, such as a DNA damaging agent, can be used to promote the death of cancer cells. At the same time inhibition of p53 protects many normal tissues from the toxic effects of many such second agents including chemotherapy and radiation.

Further, the present inventor has found that Boron Neutron Capture Therapy (BNCT) can be used in combination with ss-siRNA, double stranded siRNA or conventional antisense oligos that inhibit p53 (such as but not limited to those described in PCT/US09/02365) as a method for treating cancer (Brownell et al., “Boron Neutron Capture Therapy” In; “Therapy of Nuclear Medicine,” RP Spencer (ed), Grune and Stratton, NY, 1978; Barth et al. Cancer Res 50: 1061, 1990; Summers and Shaw, Curr Med Chem 8: 1147, 2001). Specifically, the ¹⁰B atom undergoes fission to generate ⁷Li and energetic alpha (helium) particles following capturing a thermal neutron. Within their 10-14 mm path, such particles cause DNA and other types of damage that enhance apoptosis and other inactivating effects on cancer cells when wild type or mutated p53 is inhibited.

The use of conventional antisense oligos which function using an RNAse H mechanism of action and directed to the p53 gene target have been studied in vitro and in patients. These oligos have been shown to promote the anti-cancer effects of certain conventional treatments and to protect normal tissues from genome damaging agents. Few cell types, with the exception of stem cells, possess sufficient levels of RNase H to support conventional antisense oligos dependent on this enzyme for their activity. Consequently, RNAi directed to the p53 gene target which are not dependent on RNAse H activity for function offer the potential advantage of being active in vivo in a broader range of cell types while still being catalytic. As for RNAi, generally this potential is severely limited by the well known problems associated with the poor uptake of conventional siRNA uptake in vivo and the lack of carriers that can broadly address this problem.

Molitoris et al. (J Am Soc Nephrol 20: 1754, 2009) presents data showing that conventional siRNA directed to the p53 gene target can attenuate cisplatin induced kidney damage in rats. The siRNA described was a blunt ended 19-mer with alternating 2′-0-methy/native ribose nucleosides. A carrier was not needed because the proximal tubule cells in the kidney are both a major site of kidney injury associated with ischemia or nephrotoxicity such as that caused by cisplatin and is the site of oligo reabsorption by the kidney. Thus, this carrier free approach with conventional siRNA is of very limited use for preventing the pathologic effects of p53-dependent programs that kill cells or otherwise incapacitate them, but it does illustrate the potential usefulness of inhibiting p53 for this medical indication.

Zhao et al. (Cell Stem Cell 3: 475, 2008) demonstrated that inhibiting p53 expression with siRNA can be used to enhance the production of iPSC. Human fibroblasts, for example, were converted to iPSC by using expression vectors for several genes to gain their expression in the cells. The efficiency of iPSC production was very low but was increased approximately two logs when shRNA directed to the p53 gene target was installed in the cells using a lentiviral vector. The approach described herein provides the means to transiently suppress p53 compared to the long term suppression provided by shRNA. This is important when the iPSC are to be induced to differentiate into particular cell type such as would be needed in tissue repair applications. As described herein the two-step administration approach combined with the linkage of a short cell penetrating peptide (CPP) to each strand provides an efficient way to obtain RNAi activity in stem cells in vitro with minimal carrier related toxicity.

RNAi compounds directed to the human p53 gene target that can be reconfigured for use in the two-step method provided by the present invention are found in WO 2006/035434, US 2009/0105173 and US 2004/0014956.

Table 6 lists a variety of disorders that would benefit with treatment of the p53 directed compounds described herein. For example, heart failure is a serious condition that results from various cardiovascular diseases. p53 plays a significant role in the development of heart failure. Cardiac angiogenesis directly related to the maintenance of cardiac function as well as the development of cardiac hypertrophy induced by pressure-overload. Upregulated p53 induced the transition from cardiac hypertrophy to heart failure through the suppression of hypoxia inducible factor-1(HIF-1), which regulates angiogenesis in the hypertrophied heart. In addition, p53 is known to promote apoptosis, and apoptosis is thought to be involved in heart failure. Thus, p53 is a key molecule that triggers the development of heart failure via multiple mechanisms.

Accordingly, the p53 directed compounds of the invention can be employed to diminish or alleviate the pathological symptoms associated with cardiac cell death due to apoptosis of heart cells. Initially the compound(s) will be incubated with a cardiac cell and the ability of the oligo to modulate p53 gene function (e.g., reduction in production p53, apoptosis, improved cardiac cell signaling, Ca++ transport, or morphology etc.) can be assessed. For example, the H9C2 cardiac muscle cell line can be obtained from American Type Culture Collection (Manassas, Va., USA) at passage 14 and cultured in DMEM complete culture medium (DMEM/F12 supplemented with 10% fetal calf serum (FCS), 2 mM α-glutamine, 0.5 mg/1 Fungizone and 50 mg/1 gentamicin). This cell line is suitable for characterizing the inhibitory functions of the p53 directed compounds of the invention and for characterization of modified versions thereof. HL-1 cells, described by Clayton et al. (1998) PNAS 95:2979-2984, can be repeatedly passaged and yet maintain a cardiac-specific phenotype. These cells can also be used to further characterize the effects of the oligos described herein.

It appears that expression of the apoptosis regulator p53 is governed, in part, by a molecule that in mice is termed murine double minute 2 (MDM2), or in man, human double minute 2 (HDM2), an E3 enzyme that targets p53 for ubiquitination and proteasomal processing, and by the deubiquitinating enzyme, herpesvirus-associated ubiquitin-specific protease (HAUSP), which rescues p53 by removing ubiquitin chains from it. Birks et al. (Cardiovasc Res. 2008 Aug. 1; 79 (3):472-80) examined whether elevated expression of p53 was associated with dysregulation of ubiquitin-proteasome system (UPS) components and activation of downstream effectors of apoptosis in human dilated cardiomyopathy (DCM). In these studies, left ventricular myocardial samples were obtained from patients with DCM (n=12) or from non-failing (donor) hearts (n=17). Western blotting and immunohistochemistry revealed that DCM tissues contained elevated levels of p53 and its regulators HDM2, MDM2 or the homologs thereof found in other species, and HAUSP (all P<0.01) compared with non-failing hearts. DCM tissues also contained elevated levels of polyubiquitinated proteins and possessed enhanced 20S-proteasome chymotrypsin-like activities (P<0.04) as measured in vitro using a fluorogenic substrate. DCM tissues contained activated caspases 9 and 3 (P<0.001) and reduced expression of the caspase substrate PARP-1 (P<0.05). Western blotting and immunohistochemistry revealed that DCM tissues contained elevated expression levels of caspase-3-activated DNAse (CAD; P<0.001), which is a key effector of DNA fragmentation in apoptosis and also contained elevated expression of a potent inhibitor of CAD (ICAD-S; P<0.01). These investigators concluded that expression of p53 in human DCM is associated with dysregulation of UPS components, which are known to regulate p53 stability. Elevated p53 expression and caspase activation in DCM was not associated with activation of both CAD and its inhibitor, ICAD-S. These findings are consistent with the concept that apoptosis may be interrupted and therefore potentially reversible in human HF.

In view of the foregoing, it is clear that the p53 directed compounds provided herein should exhibit efficacy for the treatment of heart failure. Accordingly, in one embodiment of the invention, p53 directed compounds are administered to patients to inhibit cardiac cell apoptosis, thereby reducing the incidence of heart failure.

Cellular transformation during the development of cancer involves multiple alterations in the normal pattern of cell growth regulation and dysregulated transcriptional control. Primary events in the process of carcinogenesis can involve the activation of oncogene function by some means (e.g., amplification, mutation, chromosomal rearrangement) or altered or aberrant expression of transcriptional regulator functions, and in many cases the removal of anti-oncogene function. One reason for the enhanced growth and invasive properties of some tumors may be the acquisition of increasing numbers of mutations in oncogenes and anti-oncogenes, with cumulative effect (Bear et al., Proc. Natl. Acad. Sci. USA 86:7495-7499, 1989). Alternatively, insofar as oncogenes function through the normal cellular signaling pathways required for organismal growth and cellular function (reviewed in McCormick, Nature 363:15-16, 1993), additional events corresponding to mutations or deregulation in the oncogenic signaling pathways may also contribute to tumor malignancy (Gilks et al., Mol. Cell Biol. 13:1759-1768, 1993), even though mutations in the signaling pathways alone may not cause cancer.

p53 provides a powerful target for efficacious anti-cancer agents. Combination of the p53 directed compounds with one or more therapeutic agents that promote apoptosis effectively induces cell death in cancer cells. Such agents include but are not limited to conventional chemotherapy, radiation and biologic agent such as monoclonal antibodies and agents that manipulate hormone pathways.

p53 protein is an important transcription factor which plays a central role in cell cycle regulation mechanisms and cell proliferation control. Baran et al. performed studies to identify the expression and localization of p53 protein in lesional and non-lesional skin samples taken from psoriatic patients in comparison with healthy controls (Acta Dermatovenerol Alp Panonica Adriat. (2005) 14:79-83). Sections of psoriatic lesional and non-lesional skin (n=18) were examined. A control group (n=10) of healthy volunteers with no personal and family history of psoriasis was also examined. The expression of p53 was demonstrated using the avidin-biotin complex immunoperoxidase method and the monoclonal antibody D07. The count and localization of cells with stained nuclei was evaluated using a light microscope in 10 fields for every skin biopsy. In lesional psoriatic skin, the count of p53 positive cells was significantly higher than in the skin samples taken from healthy individuals (p<0.01) and non-lesional skin taken from psoriatic patients (p=0.02). No significant difference between non-lesional psoriatic skin and normal skin was observed (p=0.1). A strong positive correlation between mean count and mean per cent of p53 positive cells was found (p<0.0001). p53 positive cells were located most commonly in the basal layer of the epidermis of both healthy skin and non-lesional psoriatic skin. In lesional psoriatic skin p53 positive cells were present in all layers of the epidermis. In view of these data, it is clear that p53 protein appears to be an important factor in the pathogenesis of psoriasis. Accordingly, compounds which effectively down regulate p53 expression in the skin used alone or in combination with other agents used to treat psoriasis should alleviate the symptoms of this painful and unsightly disorder.

### B. Compounds for Down-Regulating Fas (Apo-1 or CD95) Expression

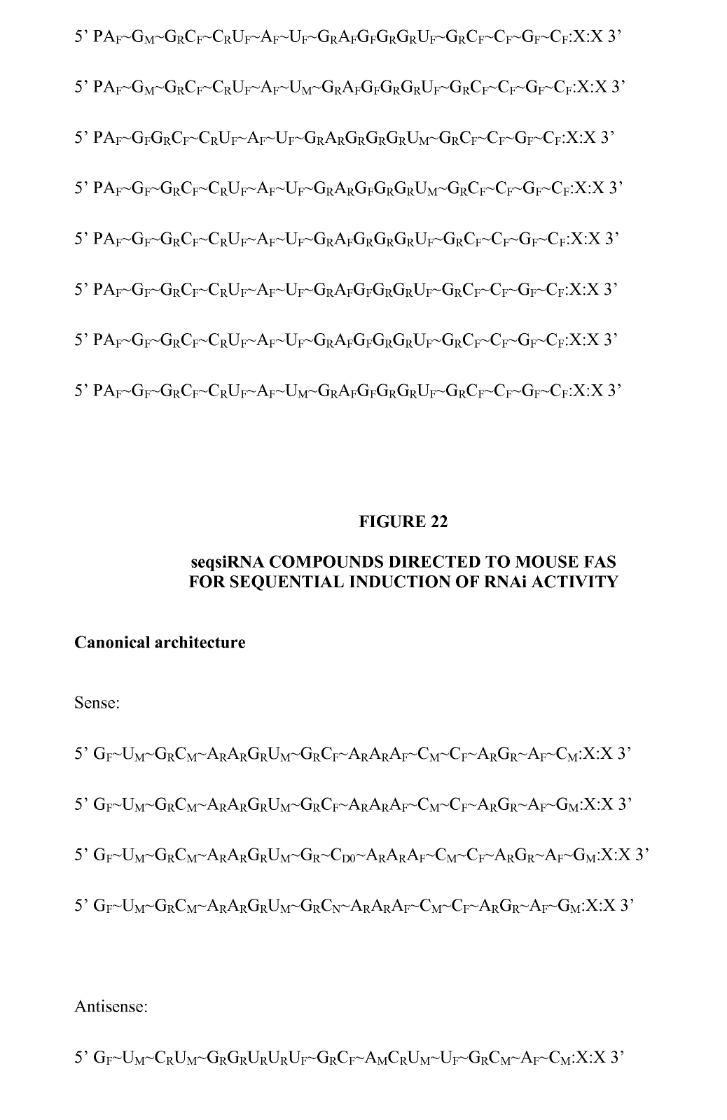

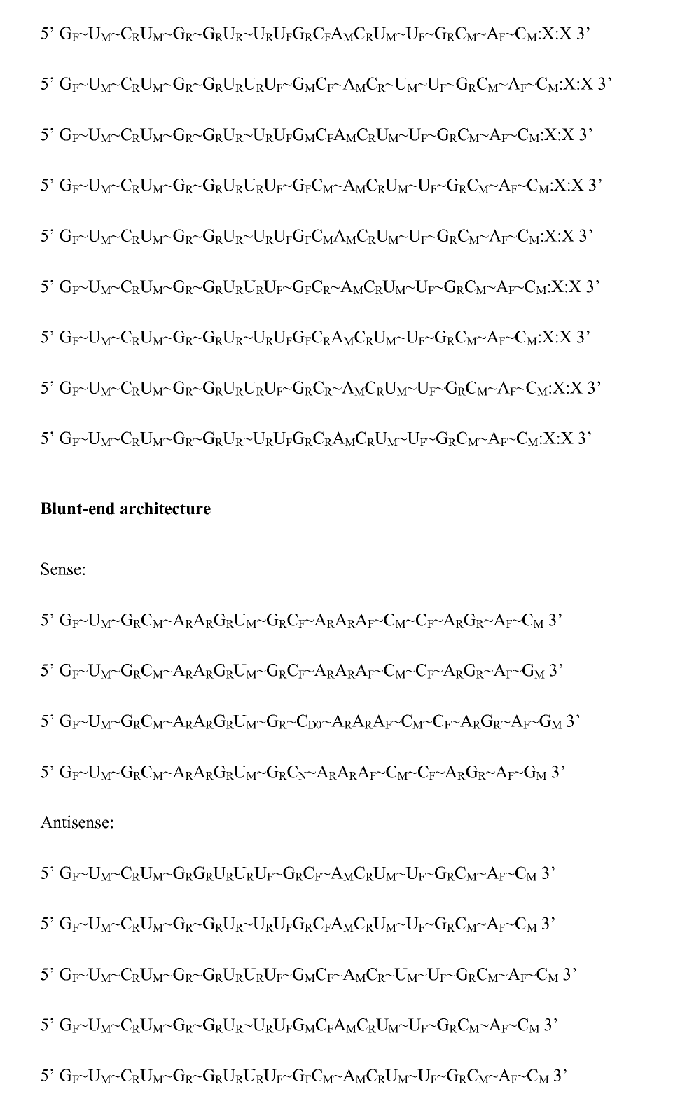

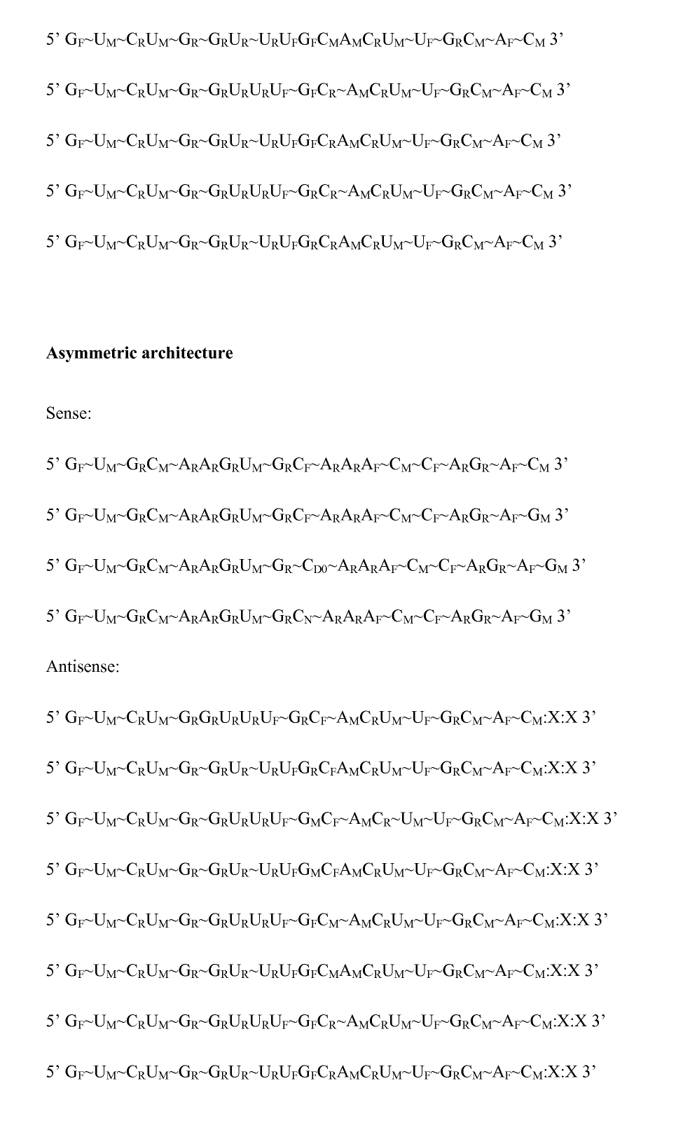

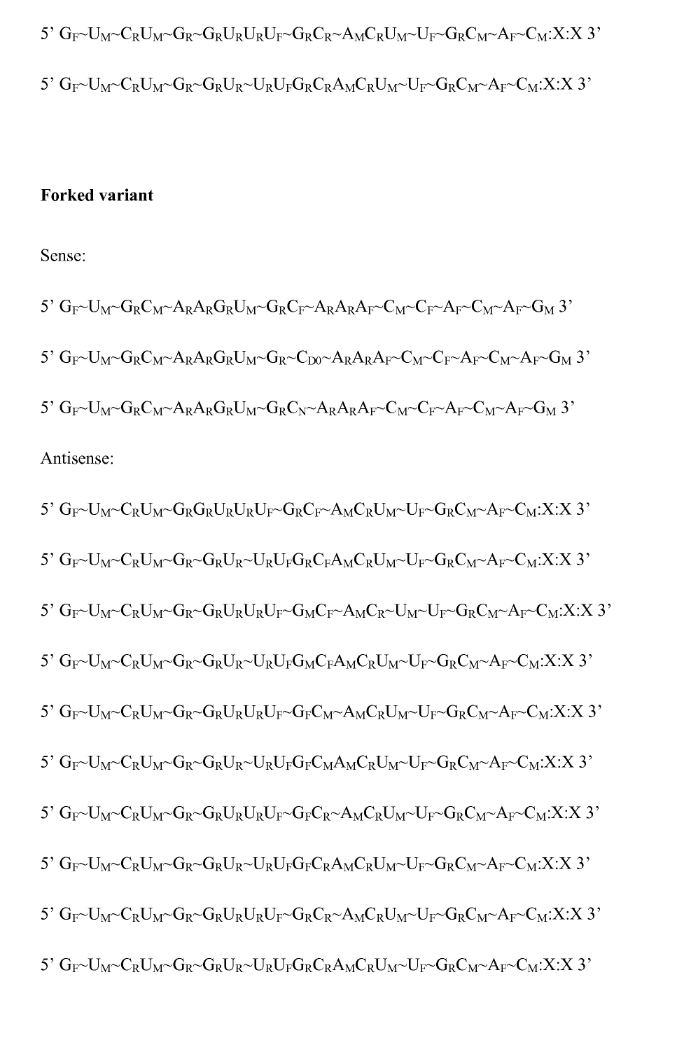

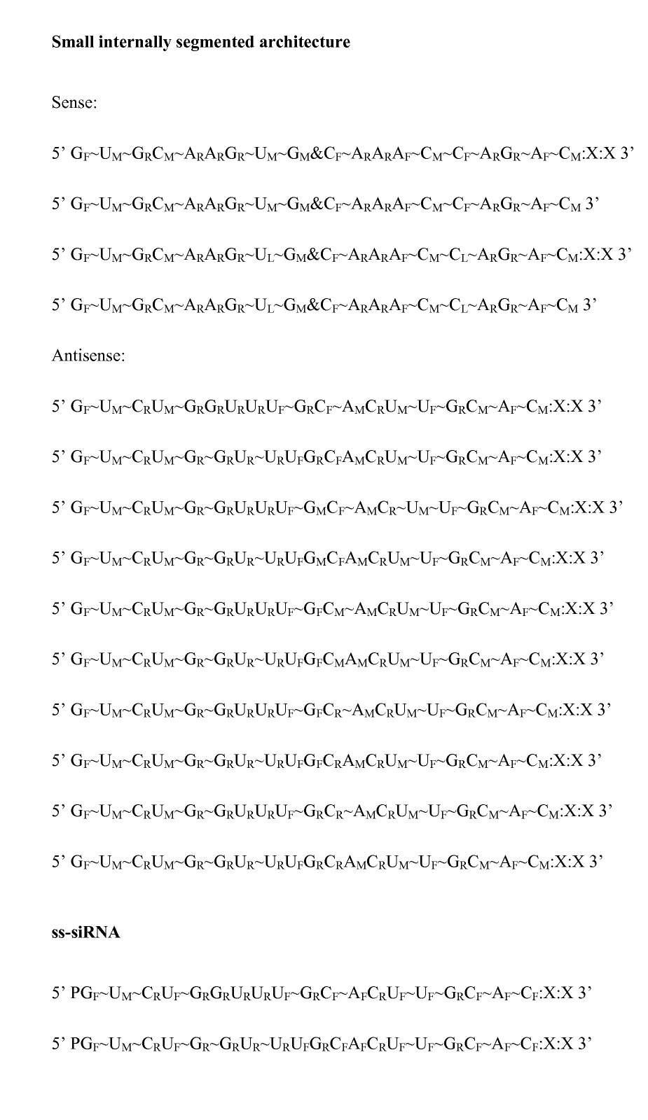

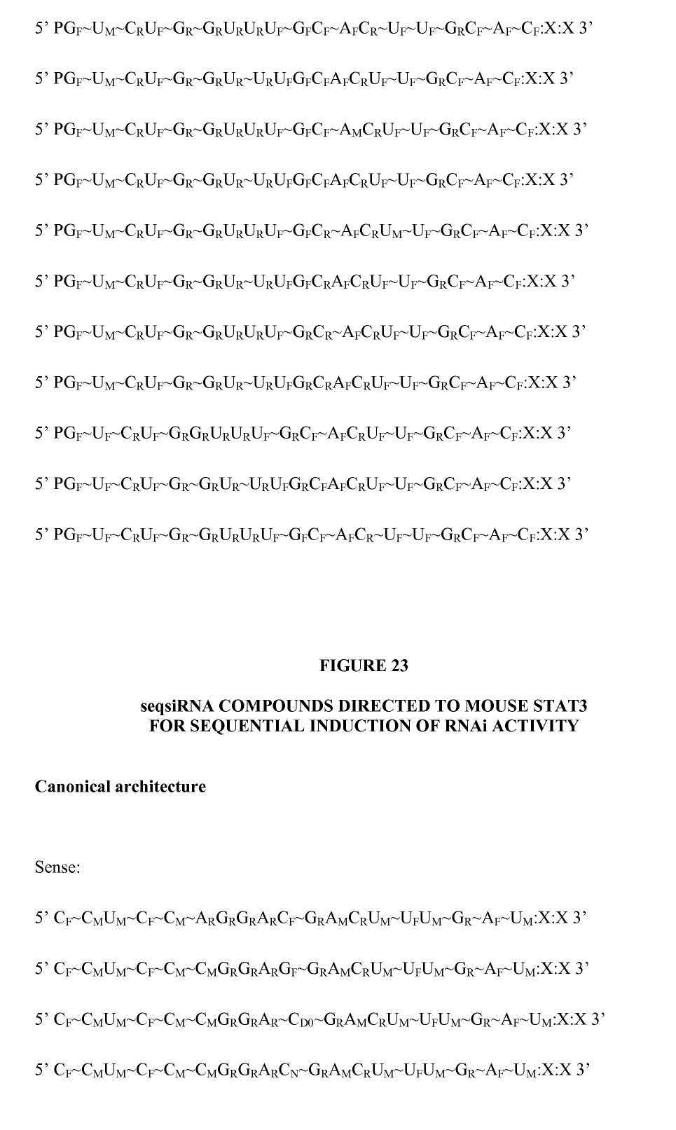

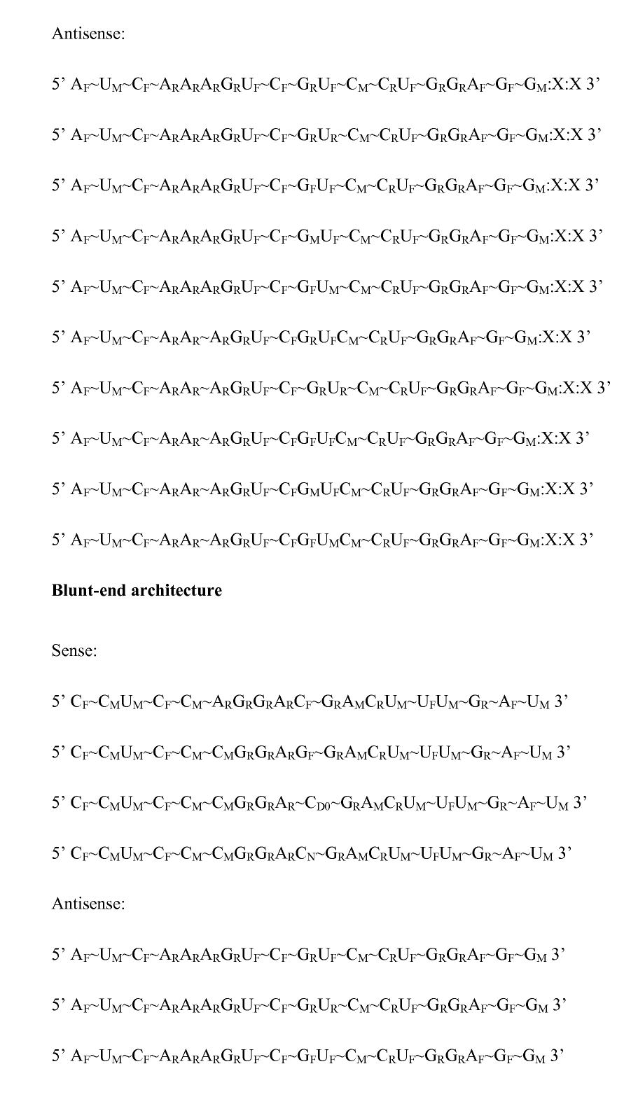

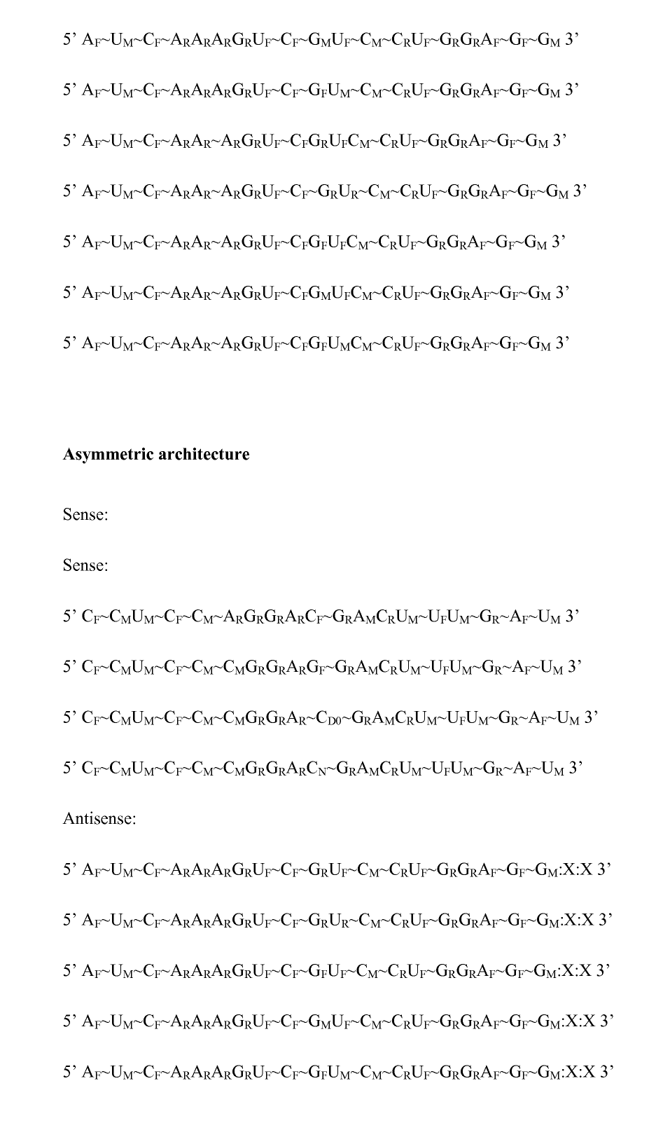

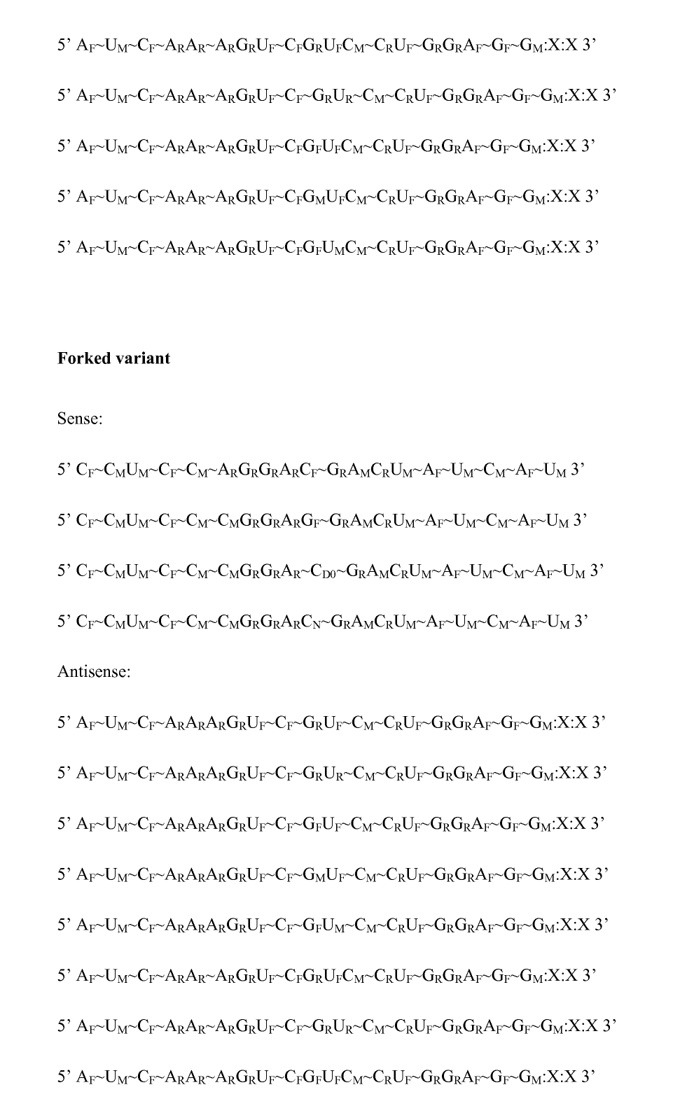

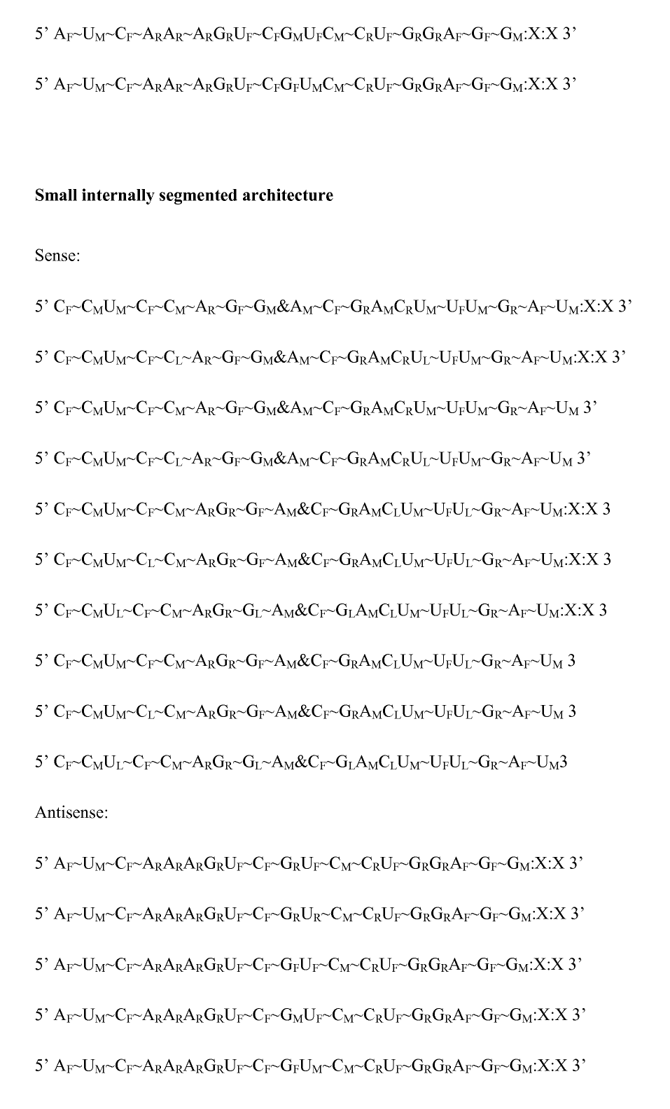

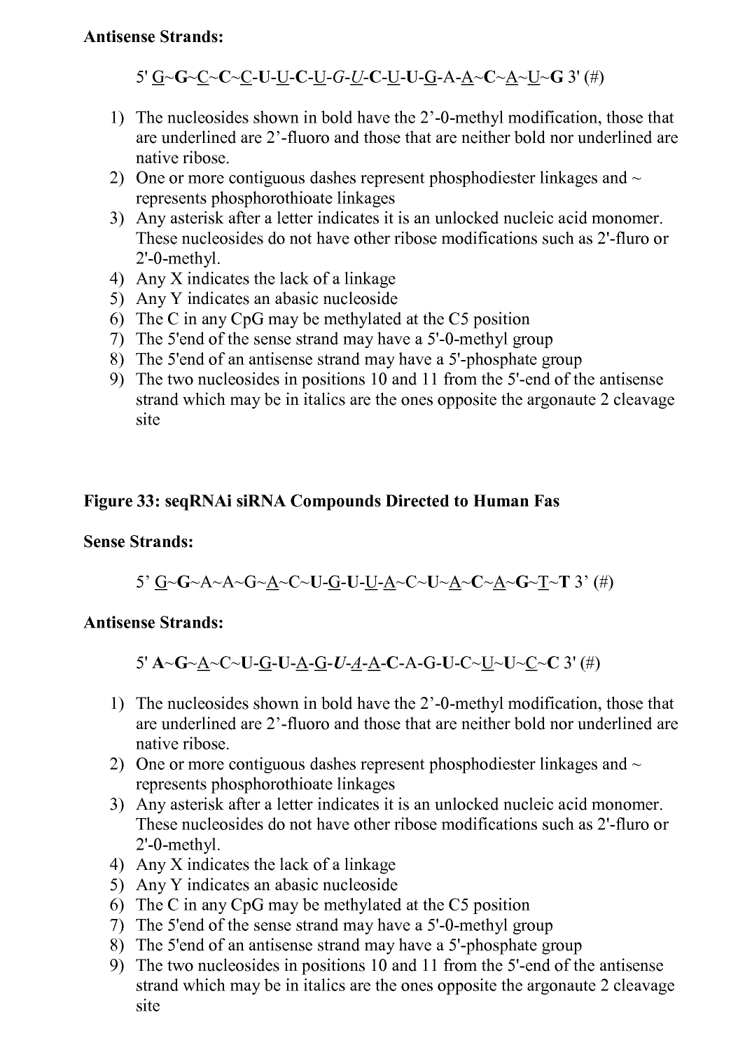

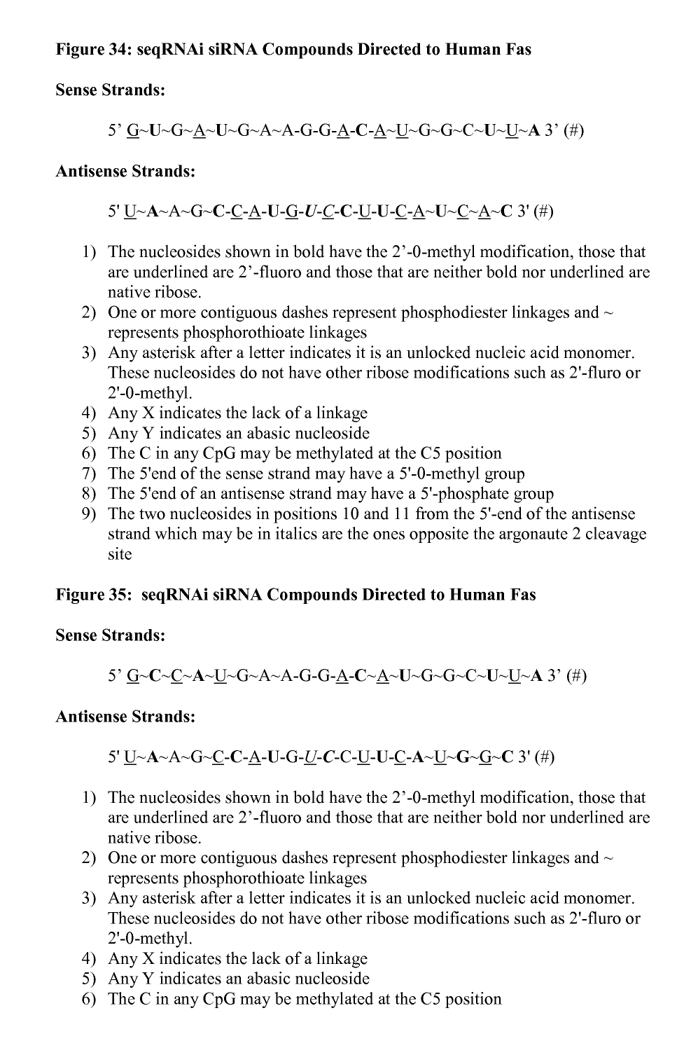

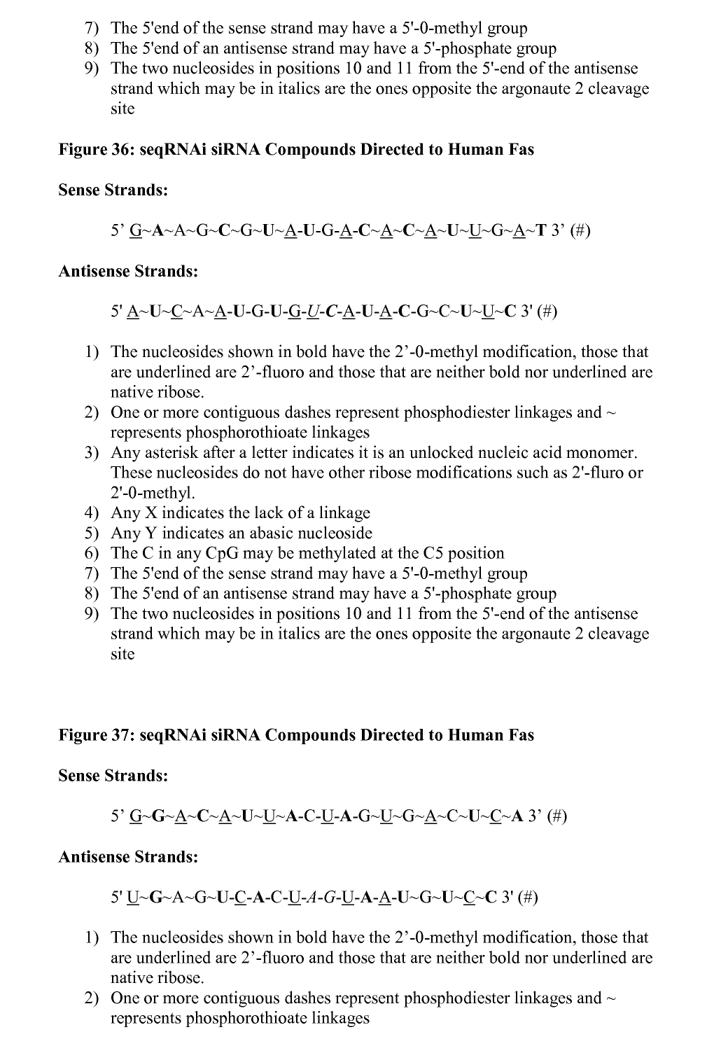

Fas (APO-1 or CD95) is a cell surface receptor that controls a pathway leading to cell death via apoptosis. This pathway is involved in a number of medical conditions where blocking fas function can provide a clinical benefit. See Table 6. Fas-mediated apoptosis, for example, is a key contributor to the pathology seen in a broad spectrum of liver diseases where inhibiting hepatocyte death can be life saving. FIGS. 22 and 33-37 provide novel compositions of matter that include many of the features heretofore described for increasing cellular uptake and/or stability for down modulating fas expression in target cells.

Lieberman and her associates have studied the effects of siRNA directed to the murine fas receptor gene target in murine models of fulminant hepatitis and renal ischemia-reperfusion injury (Song et al., Nature Med 9: 347, 2003; Hamar et al., Proc Natl Acad Sci USA 101: 14883, 2004). siRNA delivered by a hydrodynamic transfection method showed that such siRNA protects mice from concanavalin A generated hepatocyte apoptosis as evidenced by a reduction in liver fibrosis or from death associated with injections of a more hepatotoxic fas specific antibody. In the second study, siRNA was shown to protect mice from acute renal failure after clamping of the renal artery.

RNAi compounds directed to the human fas (apo-1 or CD95) receptor or ligand gene target are provided in WO 2009/0354343, US 2005/0119212, WO 2005/042719 and US 2008/0227733.

Recently, Feng et al. reported that during myocardial ischemia, cardiomyocytes can undergo apoptosis or compensatory hypertrophy (Coron Artery Dis. 2008 November; 19(7):527-34). Fas expression is upregulated in the myocardial ischemia and is coupled to both apoptosis and hypertrophy of cardiomyocytes. Some reports suggested that Fas might induce myocardial hypertrophy. Apoptosis of ischemic cardiomyocytes and Fas expression in the nonischemic cardiomyocytes occurs during the early stage of ischemic heart failure. Hypertrophic cardiomyocytes easily undergo apoptosis in response to ischemia, after which apoptotic cardiomyocytes are replaced by fibrous tissue. In the late stage of ischemic heart failure, hypertrophy, apoptosis, and fibrosis are thought to accelerate each other and might thus form a vicious circle that eventually results in heart failure. Based on these observations, it is clear that Fas directed compounds provide useful therapeutic agents for ameliorating the pathological effects associated with myocardial ischemia and hypertrophy. Accordingly, fas directed oligos will beadministered cardiac cells and their effects on apoptosis assessed. As discussed above, certain modifications of the fas directed compounds will also be assessed. These include conjugation to heart homing peptides, inclusion of CPPs, as well as encapsulation in liposomes or nanoparticles as appropriate.

In their article entitled, “Fas Pulls the Trigger on Psoriasis”, Gilhar et al. describe an animal model of psoriasis and the role played by Fas mediated signal transduction (2006) Am. J. Pathology 168:170-175). Fas/FasL signaling is best known for induction of apoptosis. However, there is an alternate pathway of Fas signaling that induces inflammatory cytokines, particularly tumor necrosis factor alpha (TNF-α) and interleukin-8 (IL-8). This pathway is prominent in cells that express high levels of anti-apoptotic molecules such as Bcl-xL. Because TNF-α is central to the pathogenesis of psoriasis and psoriatic epidermis has a low apoptotic index with high expression of Bcl-xL, these authors hypothesized that inflammatory Fas signaling mediates induction of psoriasis by activated lymphocytes. Noninvolved skin from psoriasis patients was grafted to beige-severe combined immunodeficiency mice, and psoriasis was induced by injection of FasL-positive autologous natural killer cells that were activated by IL-2. Induction of psoriasis was inhibited by injection of a blocking anti-Fas (ZB4) or anti-FasL (4A5) antibody on days 3 and 10 after natural killer cell injection. Anti-Fas monoclonal antibody significantly reduced cell proliferation (Ki-67) and epidermal thickness, with inhibition of epidermal expression of TNF-α, IL-15, HLA-DR, and ICAM-1. Fas/FasL signaling is an essential early event in the induction of psoriasis by activated lymphocytes and is necessary for induction of key inflammatory cytokines including TNF-α and IL-15.

Such data provide the rationale for therapeutic regimens entailing topical administration of Fas directed compounds and/or BCL-xL directed compounds for the treatment and alleviation of symptoms associated with psoriasis.

### C. Compounds for Down-Regulating Apo-B Expression

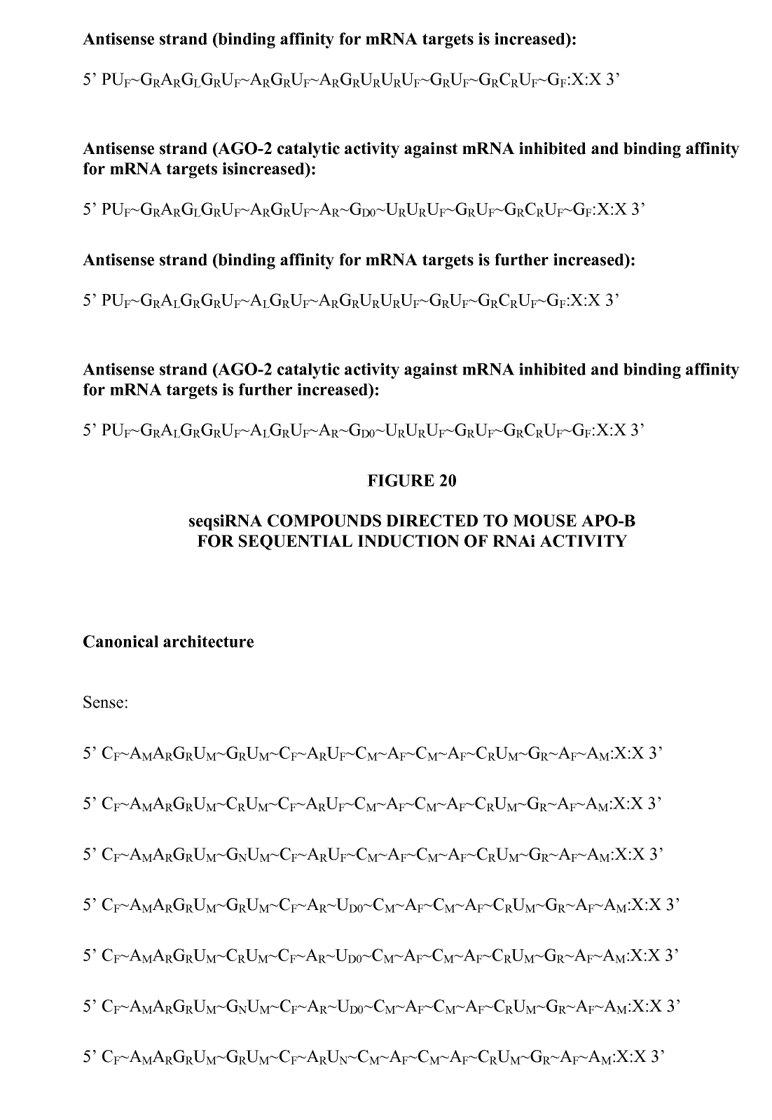

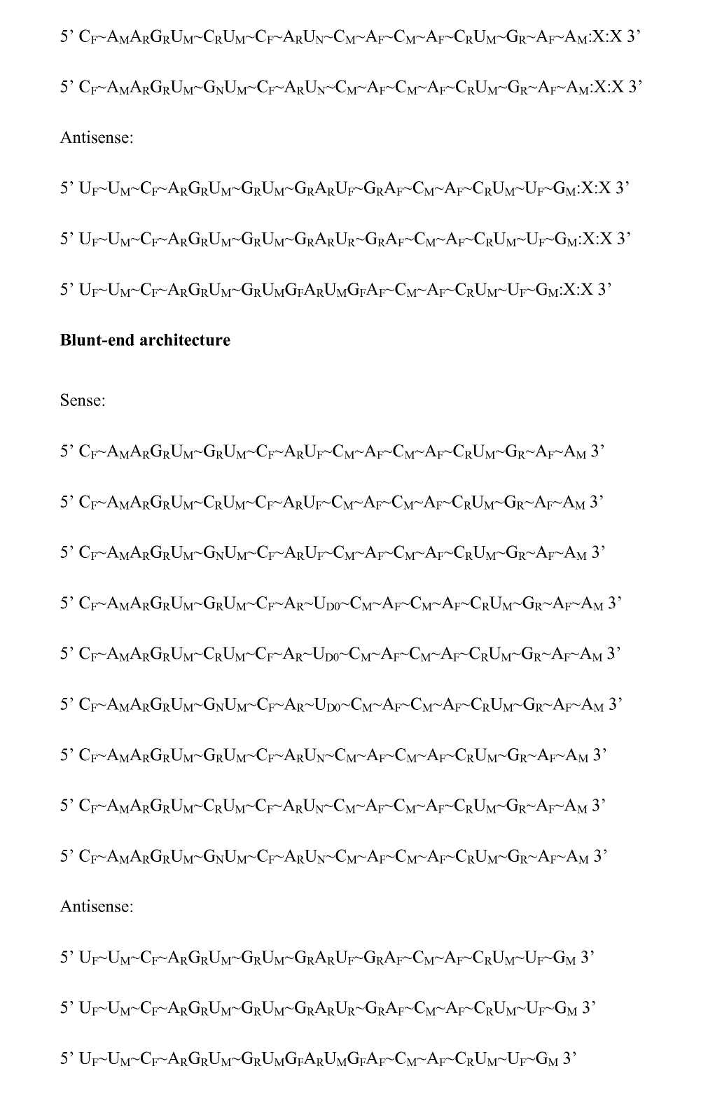

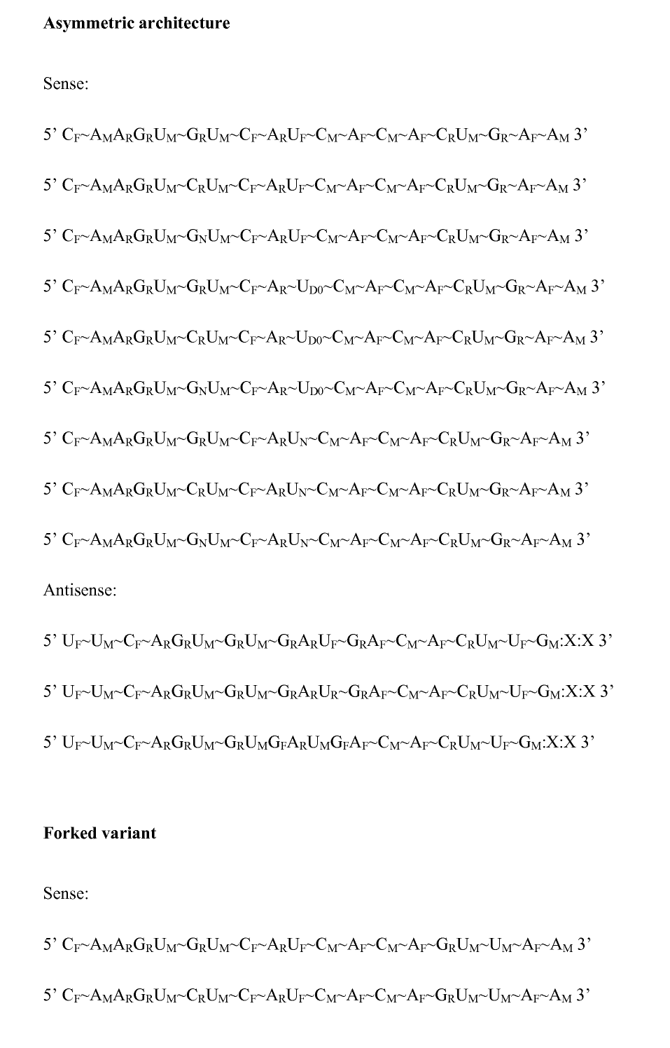

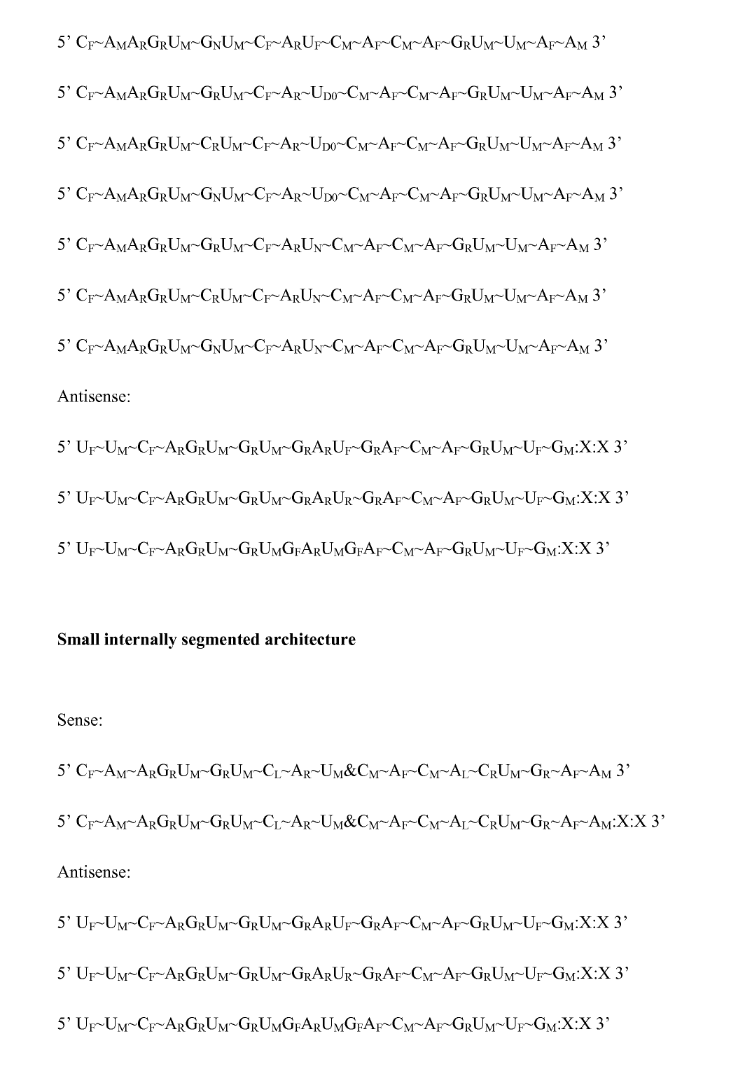

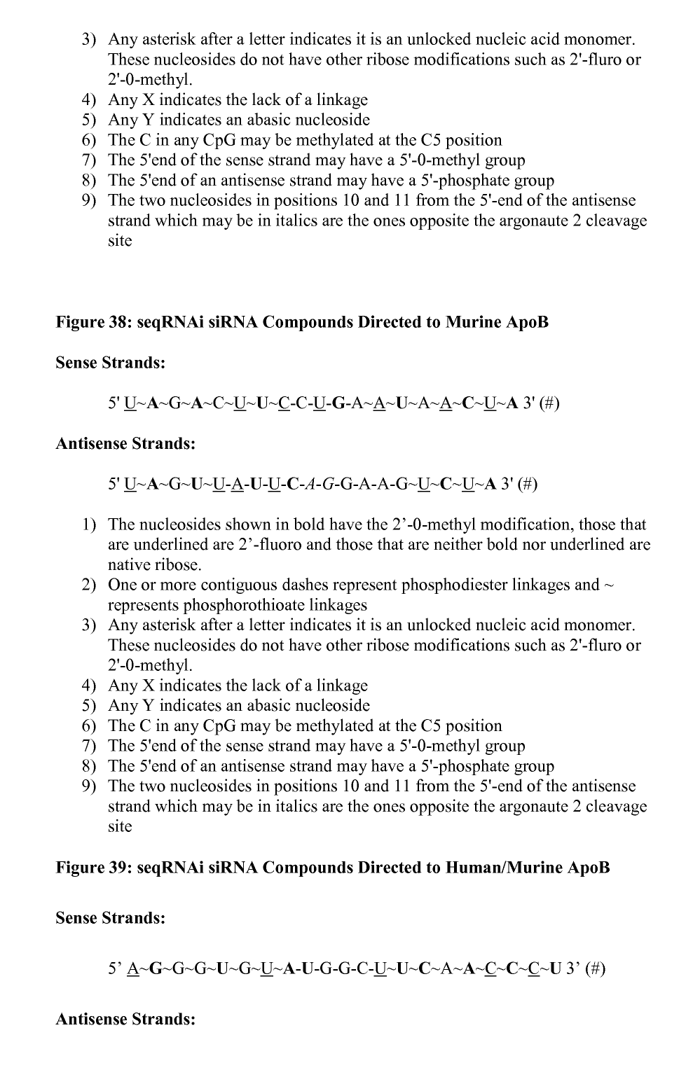

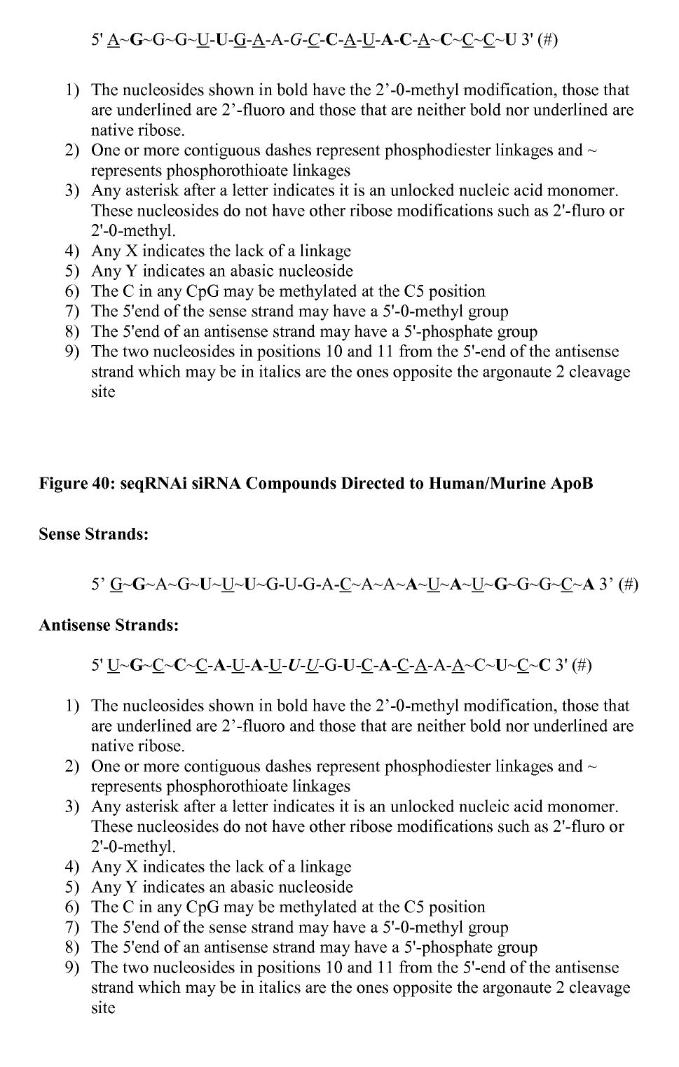

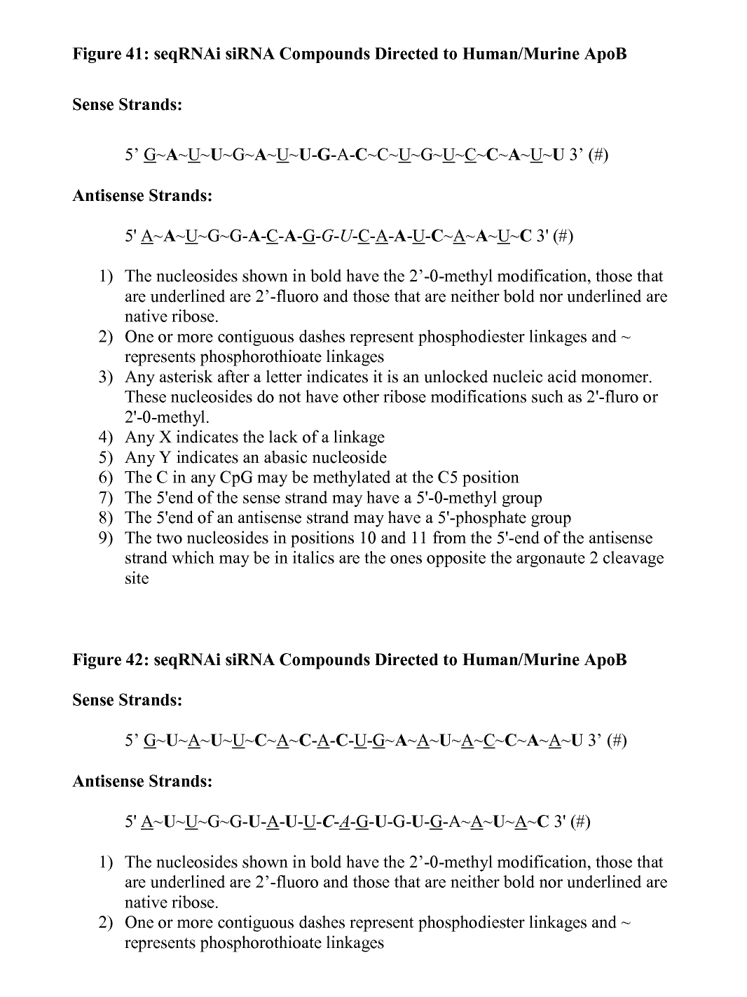

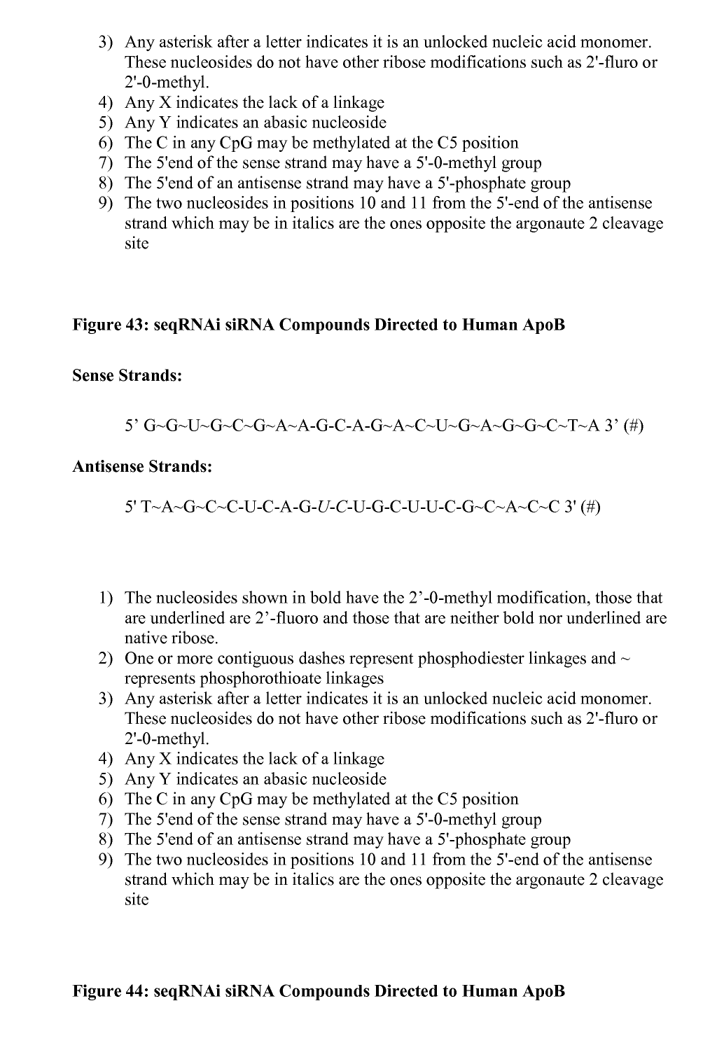

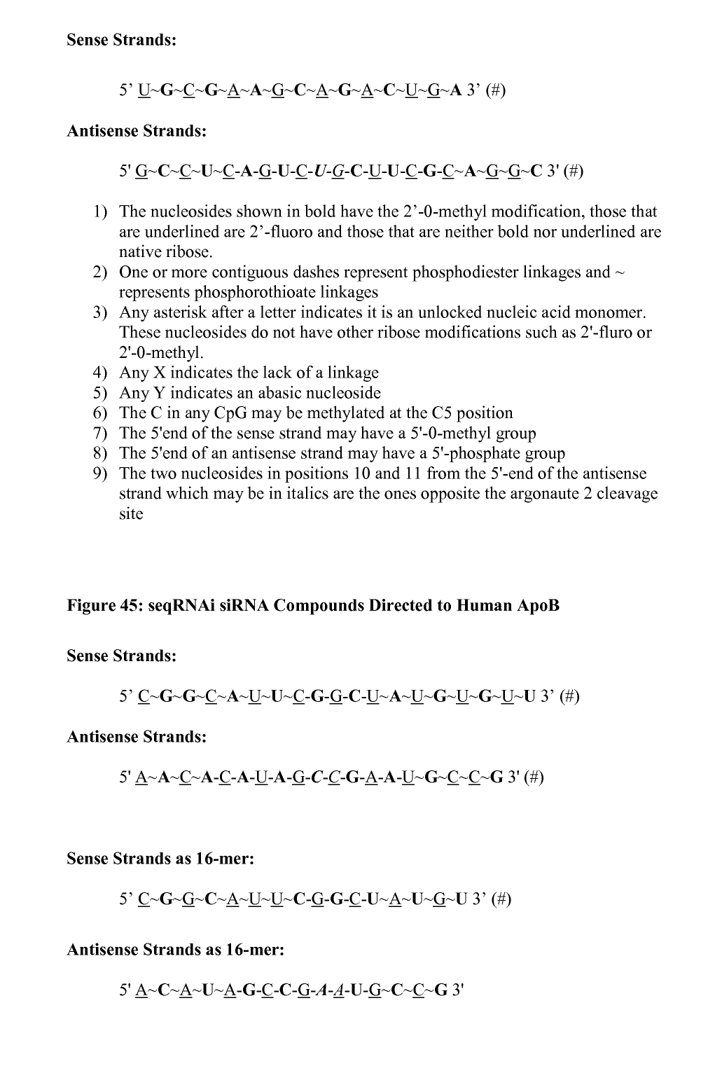

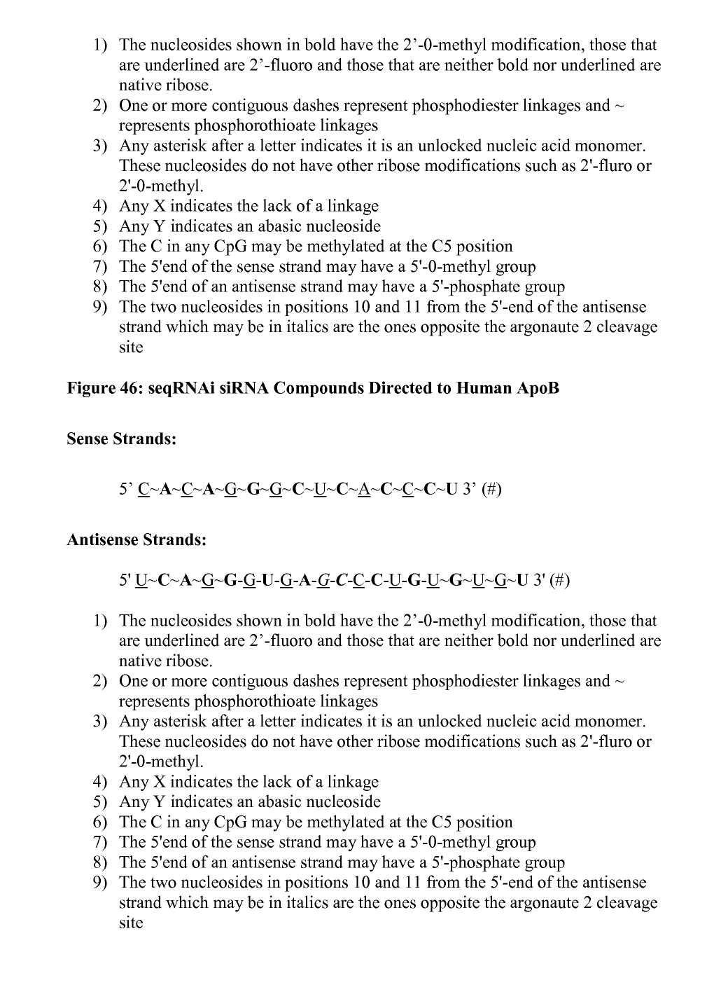

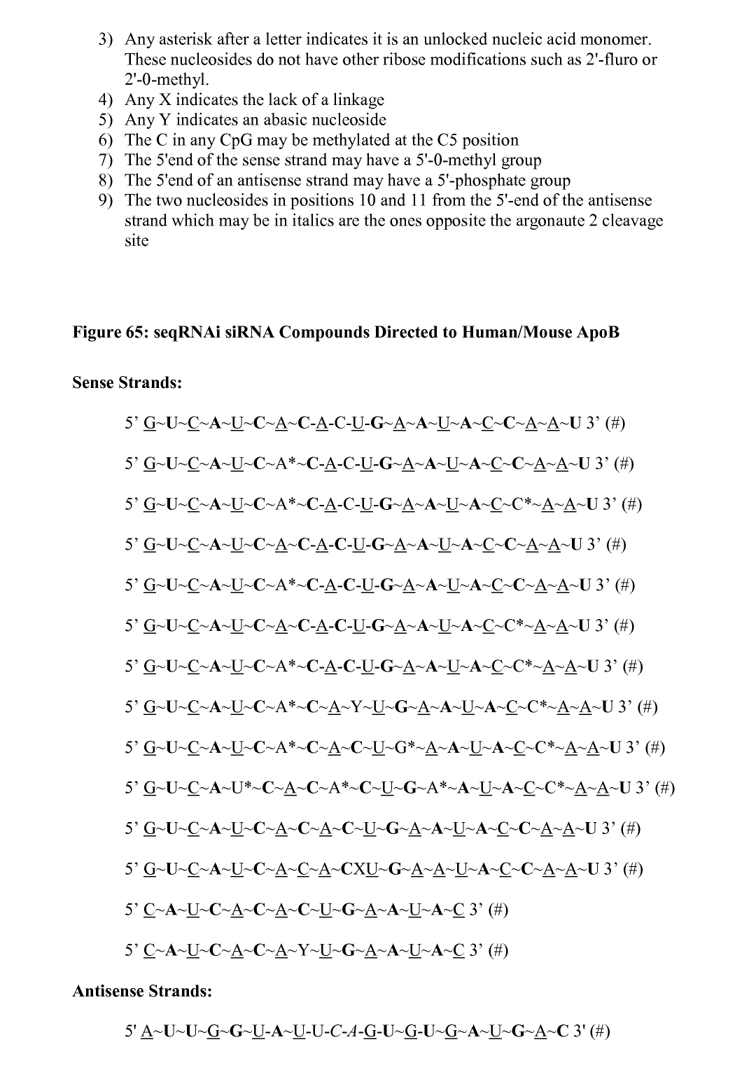

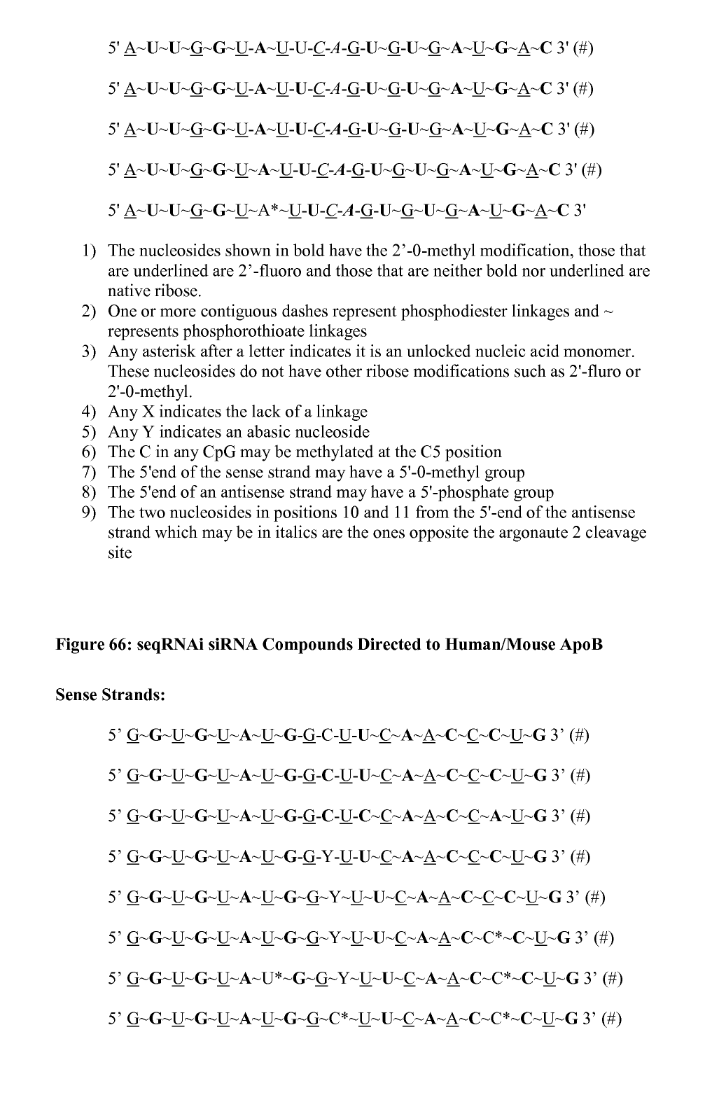

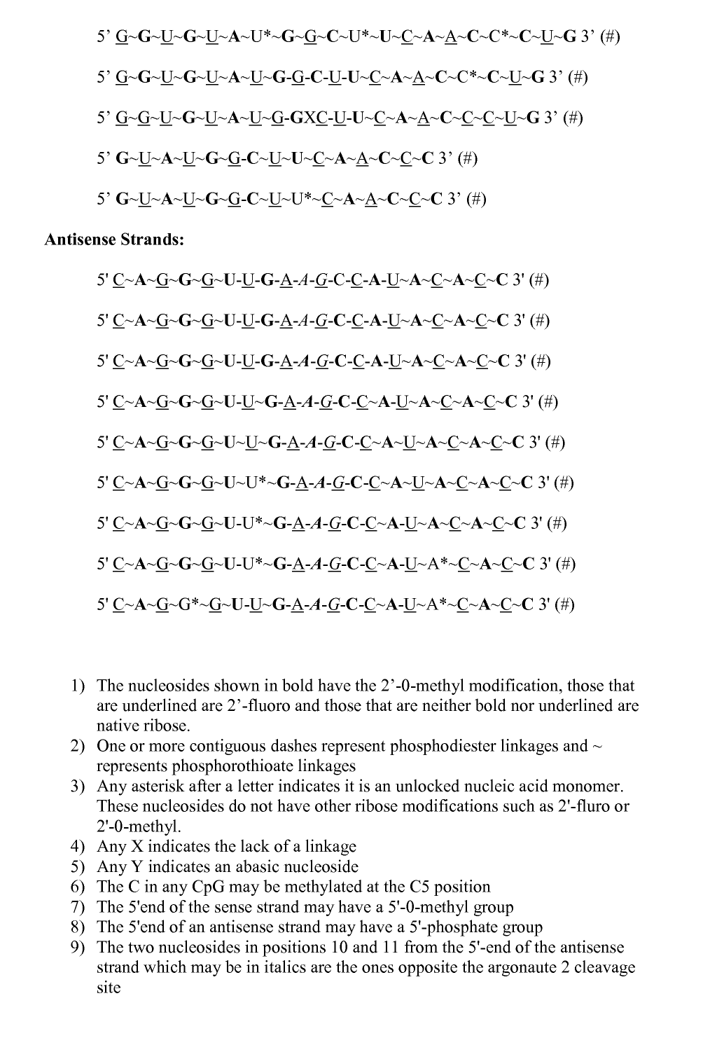

Apolipoprotein B (apoB) is an essential protein for the formation of low-density lipoproteins (LDL) and is the ligand for LDL receptor. LDL is responsible for carrying cholesterol to tissues. High levels of apoB can lead to plaques that cause atherosclerosis. Accordingly, blocking apo B expression is a useful treatment modality for a variety of medical disorders including those listed in Table 6. FIGS. 20, 38-46, 65 and 66 provide compounds suitable for use in accordance with the present invention to silence apoB expression.

Soutschek et al. (Nature 432: 173, 2004) have described two siRNA compounds simultaneously directed to both the murine and human apoB gene targets suitable for use in the present invention. These compounds have 21-mer passenger and 23-mer guide strands with cholesterol conjugated to the 3′-ends of the passenger strand. The cholesterol promoted both nuclease resistance and cellular uptake into the target tissues. The reductions in apoB expression in liver and jejunum were associated with reductions in plasma levels of apoB-100 protein and LDL. The authors indicated that the unconjugated compounds (lacking cholesterol) were inactive and concluded that the conjugated compounds need further optimization to achieve improved in vivo potency at doses and dose regimens that are clinically acceptable.

The same group of investigators filed US20060105976, WO06036916 and U.S. Pat. No. 7,528,118 that also provide siRNA compounds suitable for down modulating both human and mouse apoB gene expression. Eighty-one distinct RNAi compounds with demonstrated activity in the human HepG2 and/or the murine liver cell line NmuLi that expresses apoB were described. Twenty-seven of these double stranded siRNA compounds were found to reduce apoB protein expression in HepG2 cells to less than 35% of control. One of these siRNA was tested in human apoB-100 transgenic mice where following three daily tail vein injections, the siRNA reduced mouse apoB mRNA levels 43+/−10% in liver and 58+/−12% in jejunum and also reduced human apoB mRNA in livers to 40+/−10%. Other siRNA compounds directed to apoB suitable for use in the present invention have been disclosed in US 2006/0134189. These have been described for use in combination with the SNALP (stable nucleic acid lipid particles) delivery technology.

Conventional antisense oligos directed to gene targets such as the apoB can be converted to RNAi compounds in accordance with the present invention and can be used as described herein. A series of conventional antisense oligos directed to apoB and suitable for use with the present invention have been described in Merki et al., Circulation 118: 743, 2008; Crooke et al., J Lipid Res 46: 872, 2005; Kastelein et al., Circulation 114: 1729, 2006; U.S. Pat. No. 7,407,943, US 2006/0035858 and WO 2007/143315.

The conventional antisense oligos described in filing WO 2007/143315 are 8-16-mers. It is known that guide strands shorter than 15-mers are not active. Further 16-mer guide strands are the shortest suggested for use with the present invention. Thus, the compounds listed in this filing that are suitable for use in the present example are limited to 16-mers or to 15-12-mers that are extended to 16-mers using the human ApoB sequence. Such 16-mers can be further lengthened by the use of overhangs which as described herein do not necessarily need to base pair with the gene target.

A number of treatment regimens suitable for use with such conventional antisense oligos or for use with the two-step administration described by the present invention are provided in WO 2008/118883. The sequence used for human ApoB is provided in GenBank, Accession No. X04714.1.

Atherosclerosis is a condition in which vascular smooth muscle cells are pathologically reprogrammed. Fatty material collects in the walls of arteries and there is typically chronic inflammation. This leads to a situation where the vascular wall thickens, hardens, forms plaques, which may eventually block the arteries or promote the blockage of arteries by promoting clotting. The latter becomes much more prevalent when there is a plaque rupture.

If the coronary arteries become narrow due to the effects of the plaque formation, blood flow to the heart can slow down or stop, causing chest pain (stable angina), shortness of breath, heart attack, and other symptoms. Pieces of plaque can break apart and move through the bloodstream. This is a common cause of heart attack and stroke. If the clot moves into the heart, lungs, or brain, it can cause a stroke, heart attack, or pulmonary embolism.

Risk factors for atherosclerosis include: diabetes, high blood pressure, high cholesterol, high-fat diet, obesity, personal or family history of heart disease and smoking. The following conditions have also been linked to atherosclerosis: cerebrovascular disease, kidney disease involving dialysis and peripheral vascular disease. Down modulation of apoB s can have a beneficial therapeutic effect for the treatment of atherosclerosis and associated pathologies. WO/2007/030556 provides an animal model for assessing the effects of apoB directed compounds on the formation of atherosclerotic lesions.

### D. Compounds for Down-Regulating PCSK9 Expression

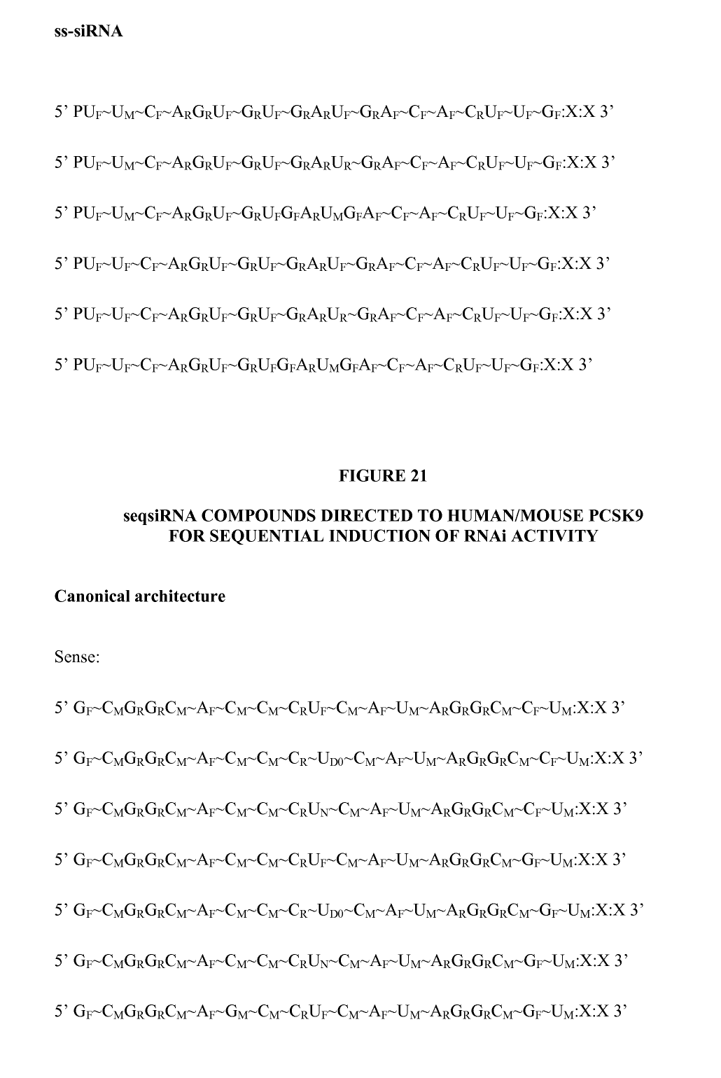

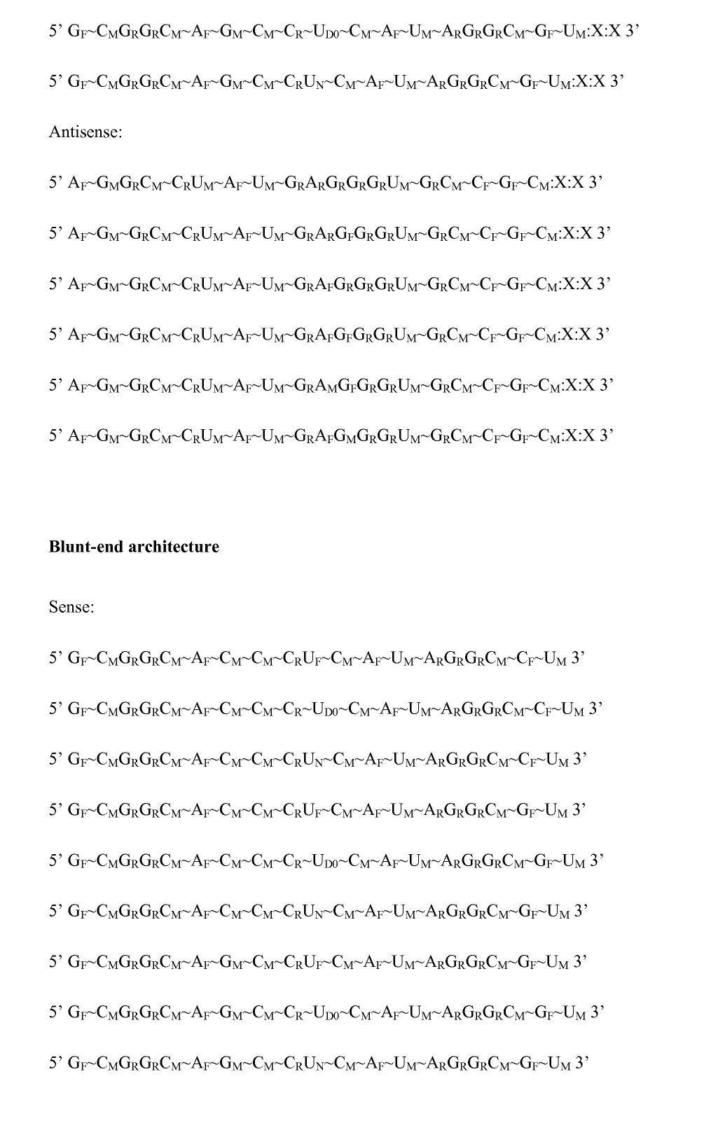

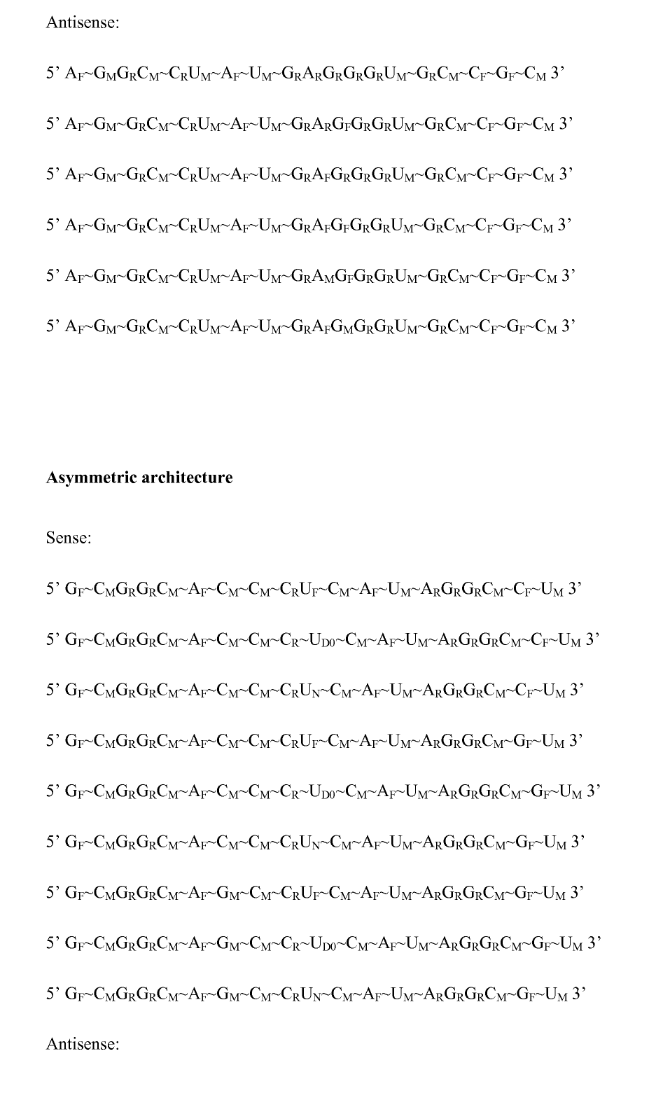

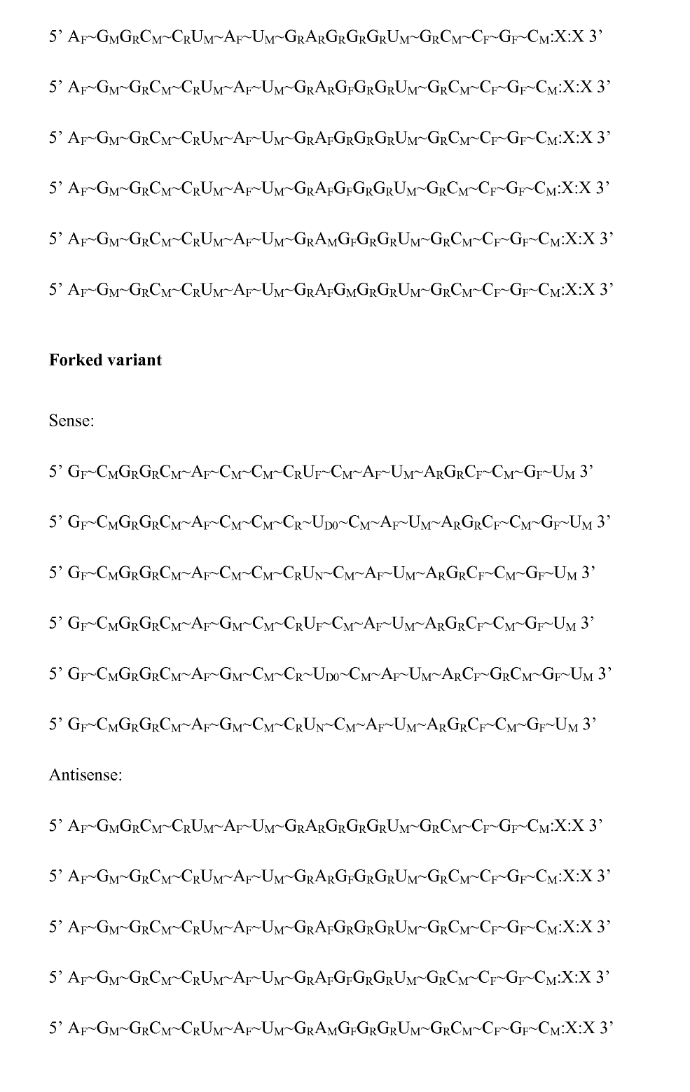

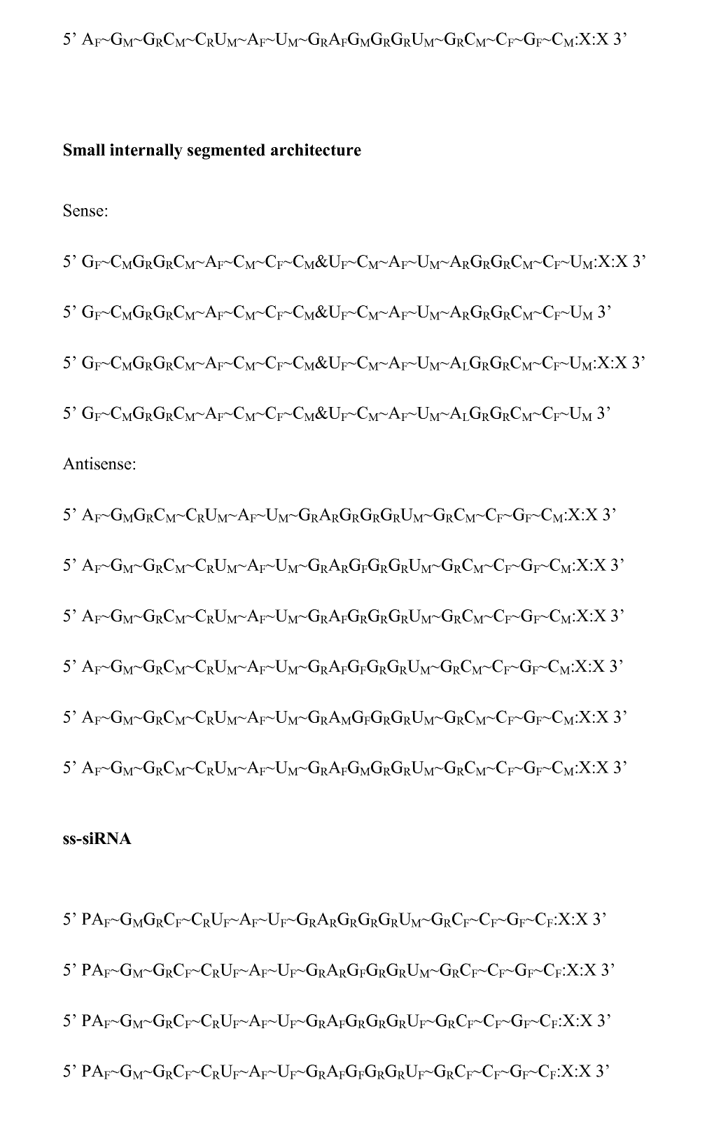

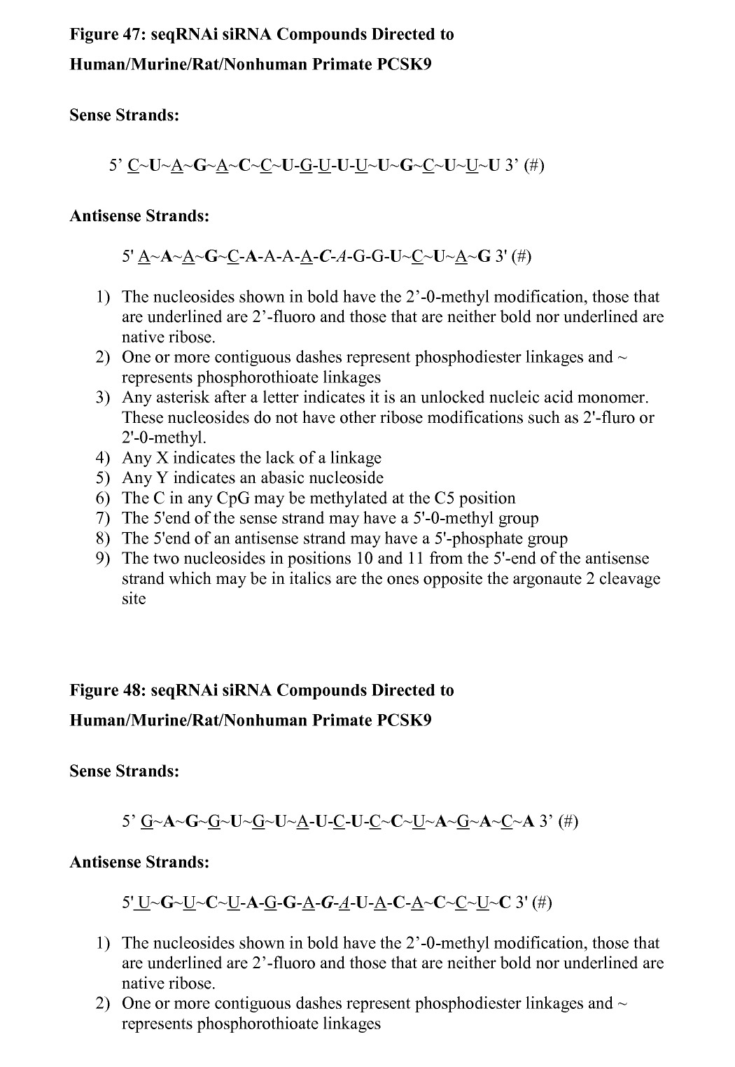

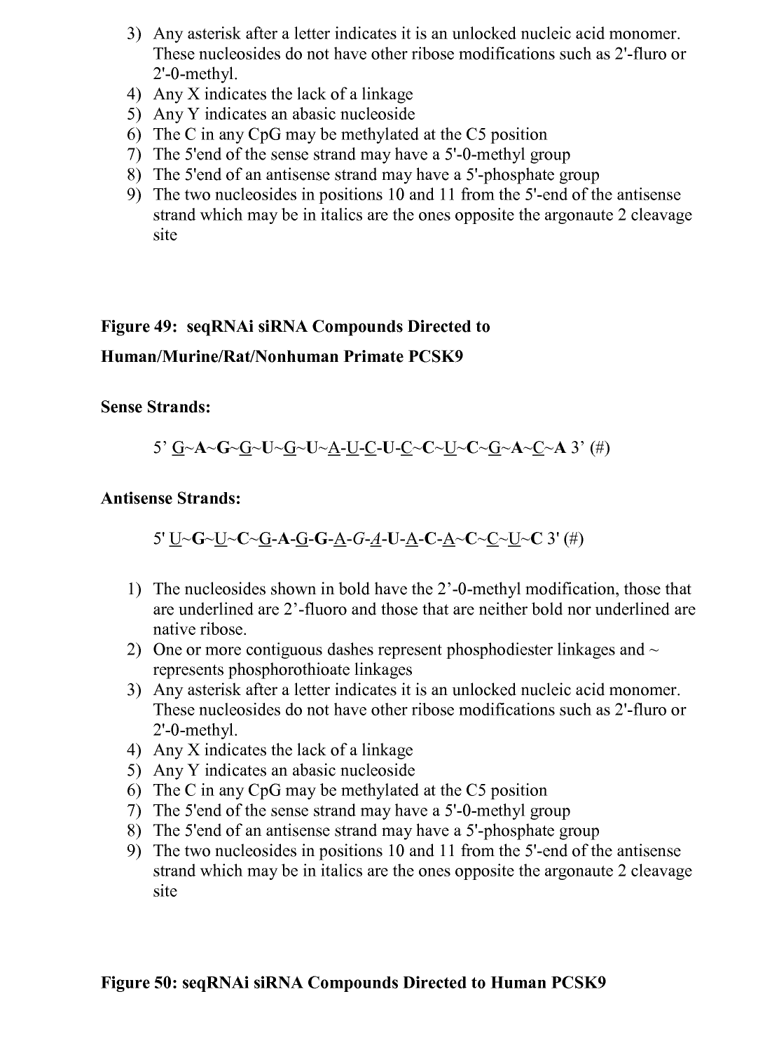

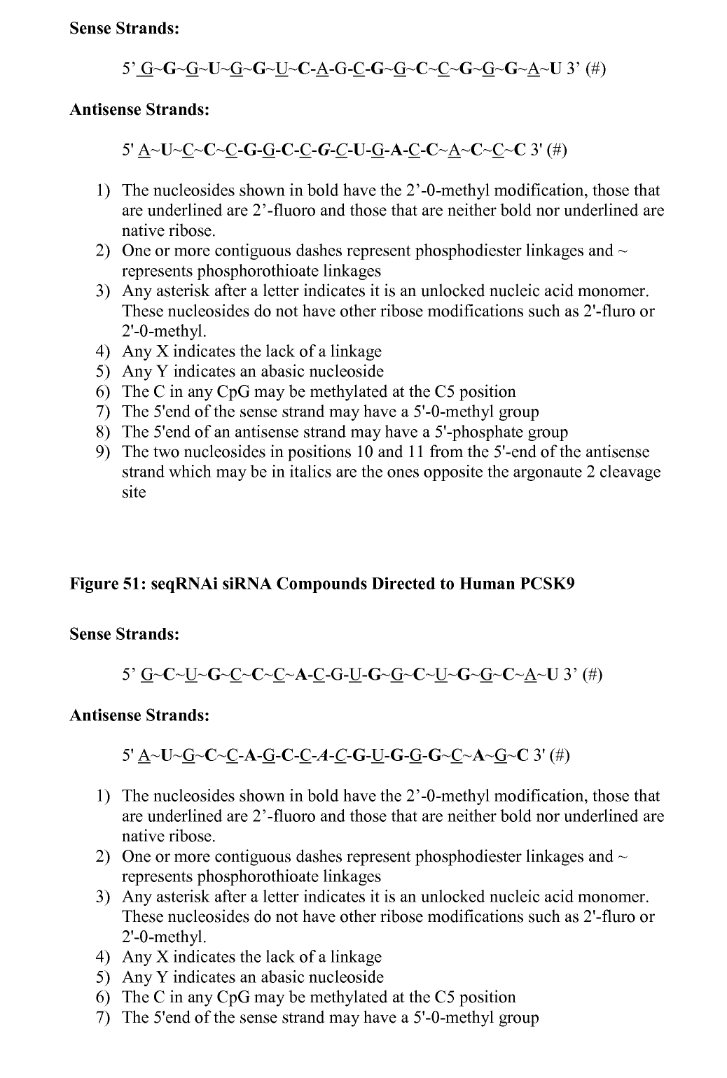

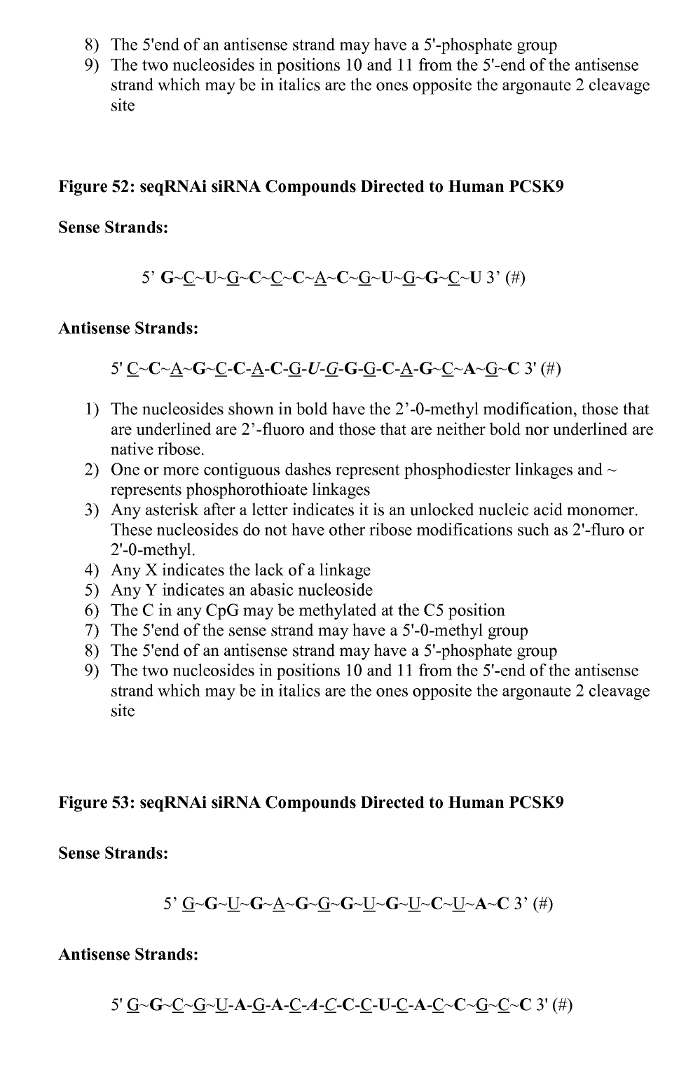

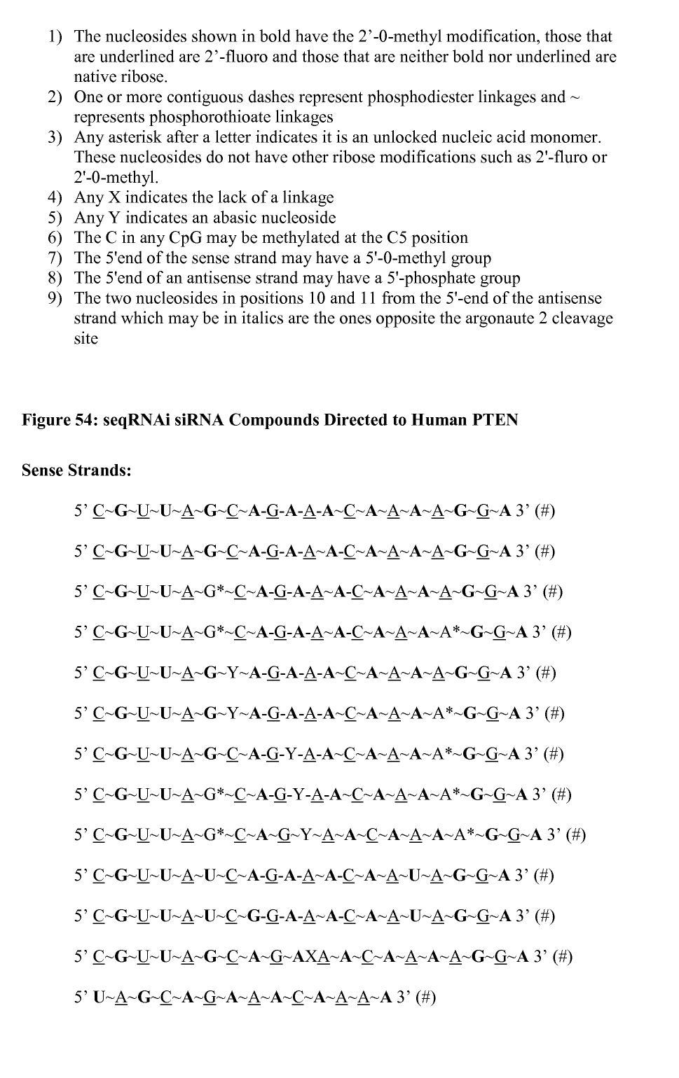

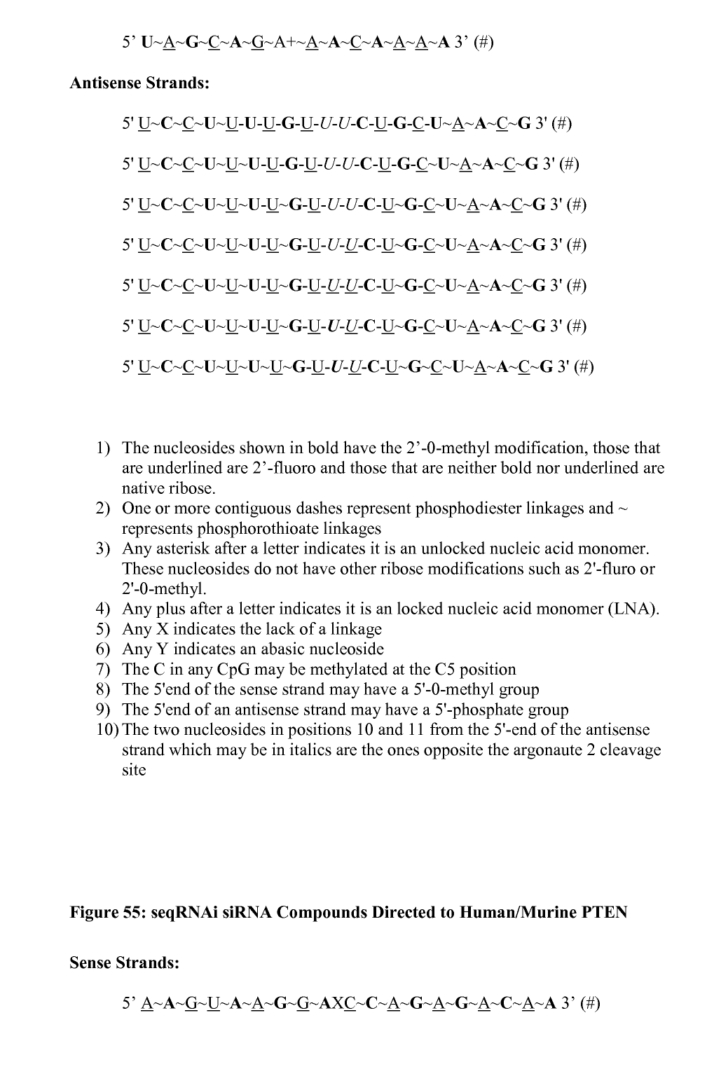

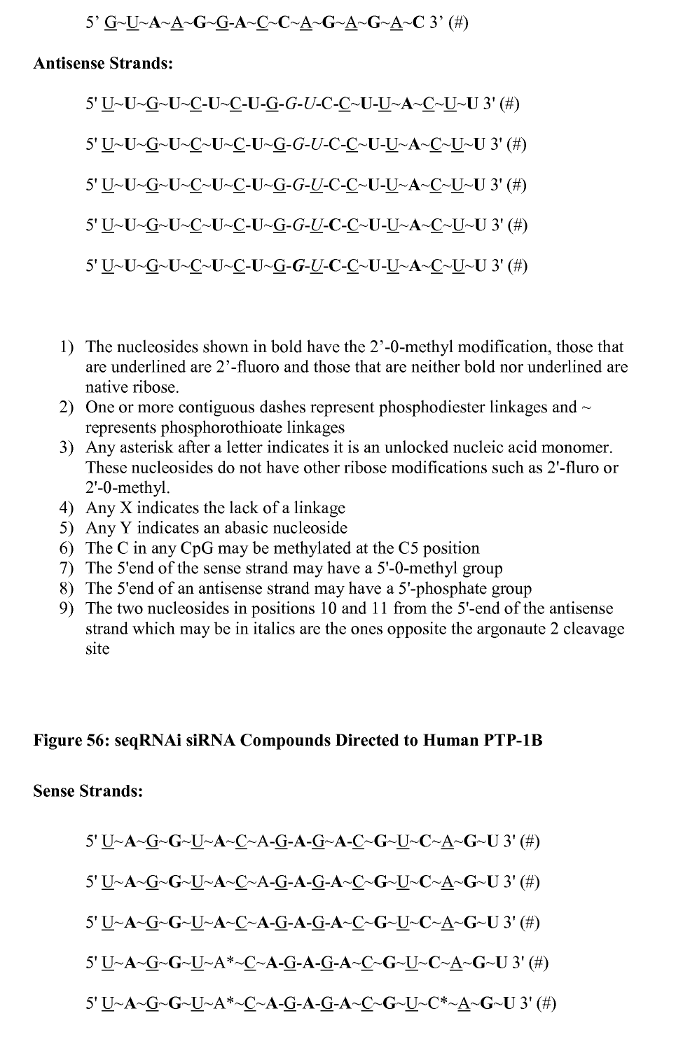

Protein convertase subtilisin-like kexin type 9 (PCSK9) is a serine protease that destroys LDL receptors in liver and consequently the level of LDL in plasma. PCSK9 mutants can have gain-of-function attributes that promote certain medical disorders associated with alterations in the proportions of plasma lipids. Agents that inhibit PCSK9 function have a role to play in the treatment of such medical disorders including those listed in Table 6. FIGS. 21 and 47-53 provide compounds suitable for use in accordance with the present invention to silence PCSK9 expression.

Frank-Kamenetsky et al. (Proc Natl Acad Sci USA 105: 11915, 2008) have described four siRNA compounds suitable for use in the present invention with three different sequences directed to the PCSK9 gene targets of human, mouse, rats, and nonhuman primates (and have characterized their activity in model systems. These siRNA were selected from a group of 150 by screening for activity using HepG2 cells. These compounds were formulated in lipidoid nanoparticles for in vivo testing. These compounds reduced PCSK9 expression in the livers of rats and mice by 50-70% and this was associated with up to a 60% reduction in plasma cholesterol levels. In transgenic mice carrying the human PCSK9 gene siRNA compounds were shown to reduce the levels of the transcripts of this gene in livers by >70%. In nonhuman primates after a single bolus injection of PCSK9 siRNA the negative effect on PCSK9 expression lasted 3 weeks. During this time apoB and LDL cholesterol (LDLc) levels were reduced. There were no detectable effects on HDL cholesterol or triglycerides. US2008/0113930 and WO 2007/134161 disclose additional PCSK9 RNAi compounds which can be modified as disclosed herein.

Conventional antisense oligos directed to the PCSK9 gene target provide another example showing how conventional antisense oligos can be reconfigured to provide novel compositions of matter suitable for use in the present invention. Such a reconfiguration is useful in situations where siRNA has advantages over conventional antisense oligos as described herein. A series of conventional antisense oligos directed to human PCSK9 and suitable for use with the present invention have been described in WO 2007/143315. These sequences were among the most active of those that were screened for PCSK9 inhibiting activity in vitro using the Hep3B cell line. The conventional antisense oligos described in this filing are 8-16-mers. It is known that guide strands shorter than 15-mers are not active. Further 16-mer guide strands are the shortest suggested for use with the present invention. Such 16-mers can be further lengthened by the use of overhangs which as described herein do not necessarily need to base pair with the gene target in the case of the guide strand.

A number of treatment regimens suitable for use with such conventional antisense oligos or for use with the two-step administration of strands capable of forming siRNA in cells and where the guide strand is directed to PCSK9 are described in WO 2008/118883. The conventional antisense oligos in this filing are targeted to apoB but the tissues involved and the therapeutic purposes involving PCSK9 are the same and thus essentially the same treatment regimens can be used.

This protein plays a major regulatory role in cholesterol homeostasis. PCSK9 binds to the epidermal growth factor-like repeat A (EGF-A) domain of the low-density lipoprotein receptor (LDLR), inducing LDLR degradation. Reduced LDLR levels result in decreased metabolism of low-density lipoproteins, which could lead to hypercholesterolemia Inhibition of PSCK9 function provides a means of lowering cholesterol levels. PCSK9 may also have a role in the differentiation of cortical neurons.

Further, the usefulness of conventional antisense oligos directed to the murine PCSK9 gene target for the treatment of hypercholesterolemia has been demonstrated by Graham et al. (J lipid Res 48: 763, 2007). A series of antisense oligos were screened for activity and the most active (ISIS 394814) selected for in vivo studies. Administration of ISIS 394814 to high fat fed mice for 6 weeks resulted in a 53% reduction in total plasma cholesterol and a 38% reduction in plasma LDL. This was accompanied by a 92% reduction in liver PCSK9 expression.

### E. Compounds for Down-Regulating Phosphatase and Tensin Homolog (PTEN) Expression

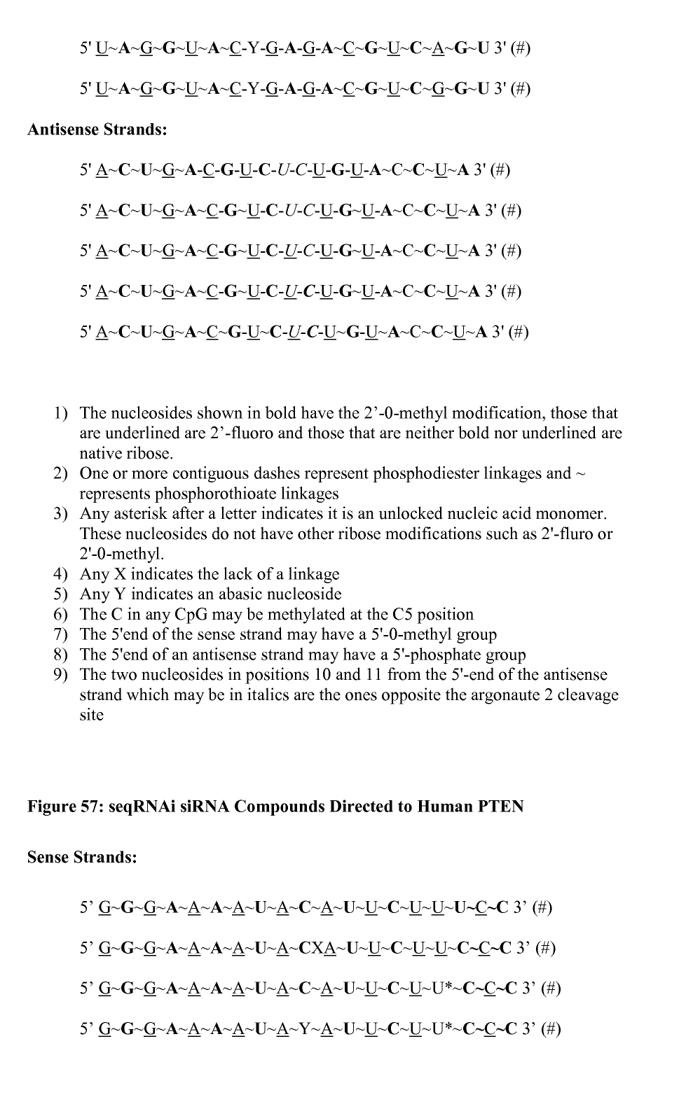

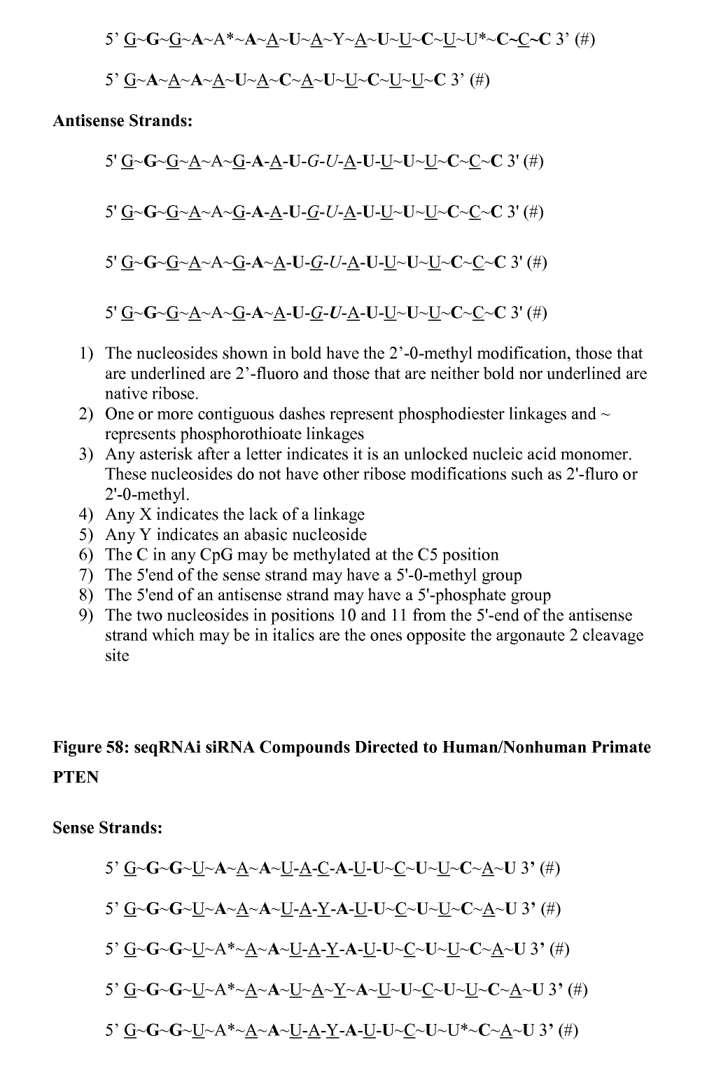

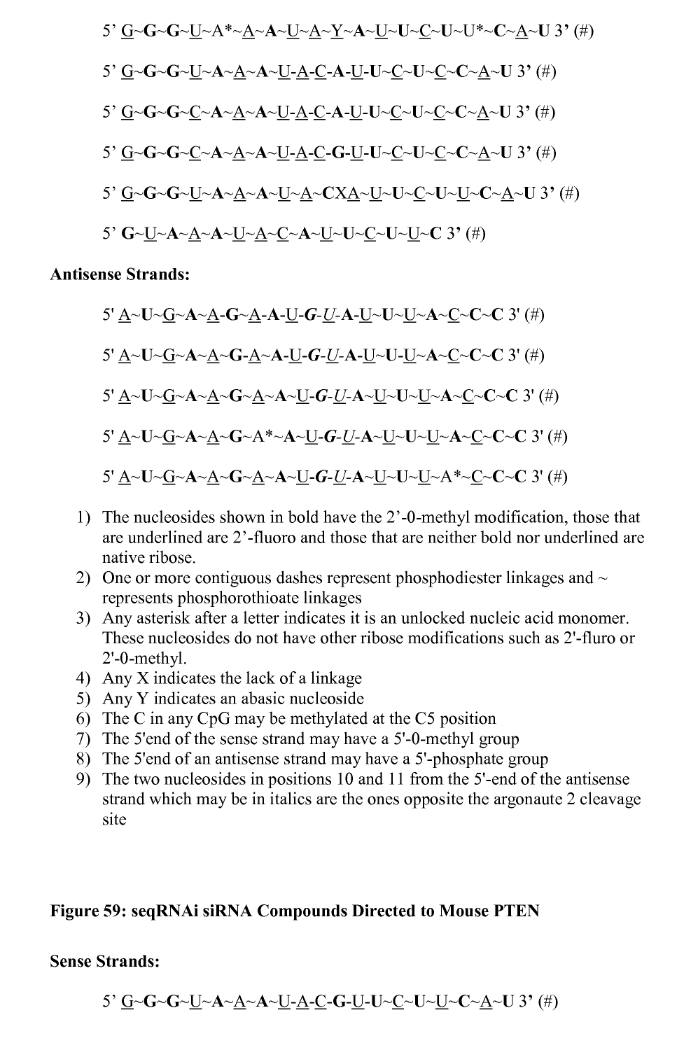

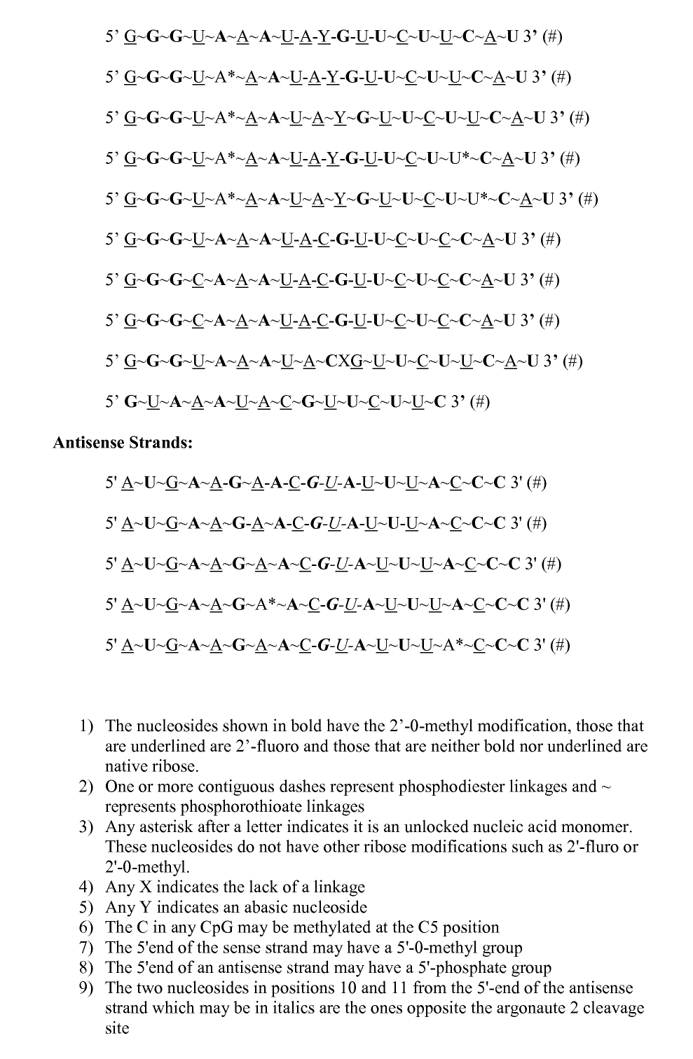

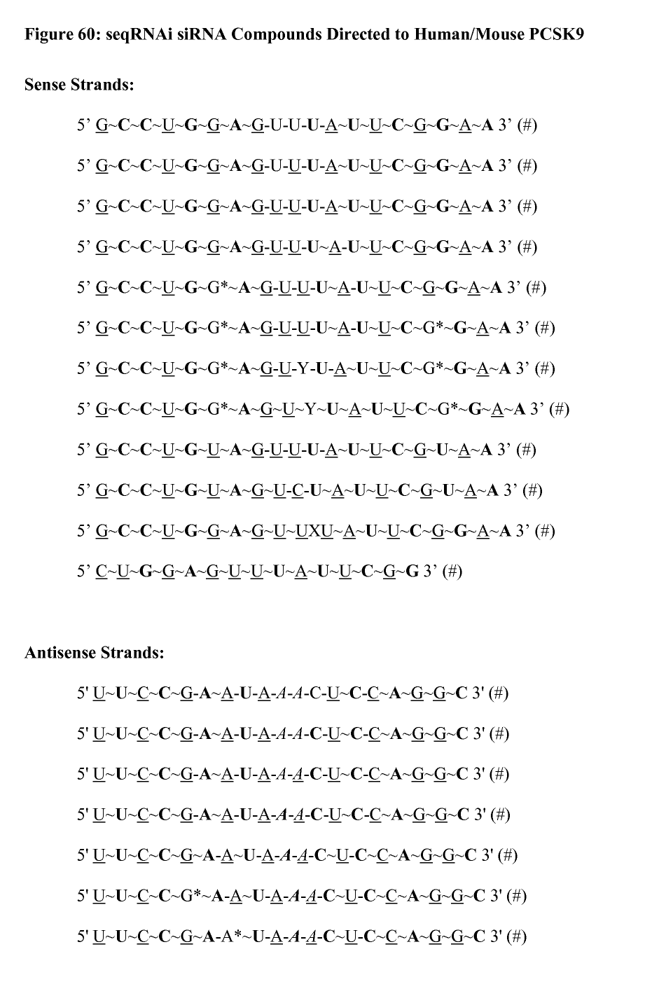

PTEN is a phosphatase (phosphatidylinositol-3,4,5-trisphosphate 3-phosphatase) that is frequently mutated in cancers with wild type p53 where the effect or the mutation is to inhibit its enzymatic activity. In this context, PTEN is thought to function as a tumor suppressor. In cancers with mutated p53, however, PTEN supports the viability and growth of the tumor in part by increasing the levels of gain-of-function p53 mutants (Li et al., Cancer Res 68: 1723, 2008). PTEN also modulates cell cycle regulatory proteins with the effect of inhibiting cell proliferation. Thus, PTEN inhibitors have a role in the treatment of some cancers and in promoting cell proliferation such as expanding cell populations for purposes such as transplantation. FIGS. 8, 10, 12, 14, 16, 18, 54, 55 and 57-59 provide compounds suitable for use in accordance with the present invention to silence PTEN expression.

In vivo regeneration of peripheral neurons is constrained and rarely complete, and unfortunately patients with major nerve trunk transections experience only limited recovery. Intracellular inhibition of neuronal growth signals may be among these constraints. Christie et al. investigated the role of PTEN (phosphatase and tensin homolog deleted on chromosome 10) during regeneration of peripheral neurons in adult Sprague Dawley rats (J. Neuroscience 30:9306-9315 (2010). PTEN inhibits phosphoinositide 3-kinase (PI3-K)/Akt signaling, a common and central outgrowth and survival pathway downstream of neuronal growth factors. While PI3-K and Akt outgrowth signals were expressed and activated within adult peripheral neurons during regeneration, PTEN was similarly expressed and poised to inhibit their support. PTEN was expressed in neuron perikaryal cytoplasm, nuclei, regenerating axons, and Schwann cells. Adult sensory neurons in vitro responded to both graded pharmacological inhibition of PTEN and its mRNA knockdown using siRNA. Both approaches were associated with robust rises in the plasticity of neurite outgrowth that were independent of the mTOR (mammalian target of rapamycin) pathway. Importantly, this accelerated outgrowth was in addition to the increased outgrowth generated in neurons that had undergone a preconditioning lesion. Moreover, following severe nerve transection injuries, local pharmacological inhibition of PTEN or siRNA knockdown of PTEN at the injury site accelerated axon outgrowth in vivo. The findings indicated a remarkable impact on peripheral neuron plasticity through PTEN inhibition, even within a complex regenerative milieu. Overall, these findings identify a novel route to propagate intrinsic regeneration pathways within axons to benefit nerve repair. In view of these findings, it is clear that the PTEN directed compounds of the invention can be useful for the treatment of nerve injury and damage. In a preferred embodiment, such agents would be administered intrathecally as described for insulin in Toth et al., Neuroscience(2006) 139:429-49. Czauderna et al. (Nuc Acids Res 31: 2705, 2003) have described an active siRNA compound that is directed to the human PTEN gene target which is suitable for use in accordance with the present invention as described herein. Allerson et al. (J Med Chem 48: 901, 2005) have described two siRNA compounds suitable for use in the present invention that are targeted to human PTEN.

### F. Compounds for Down-Regulating PTP1B Expression

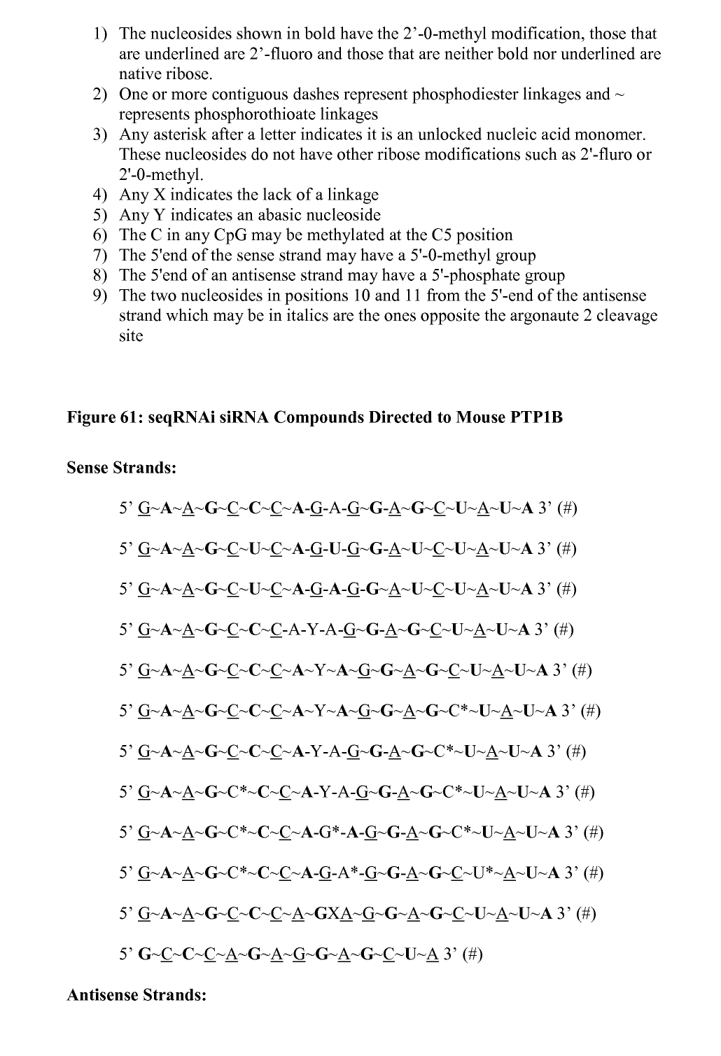

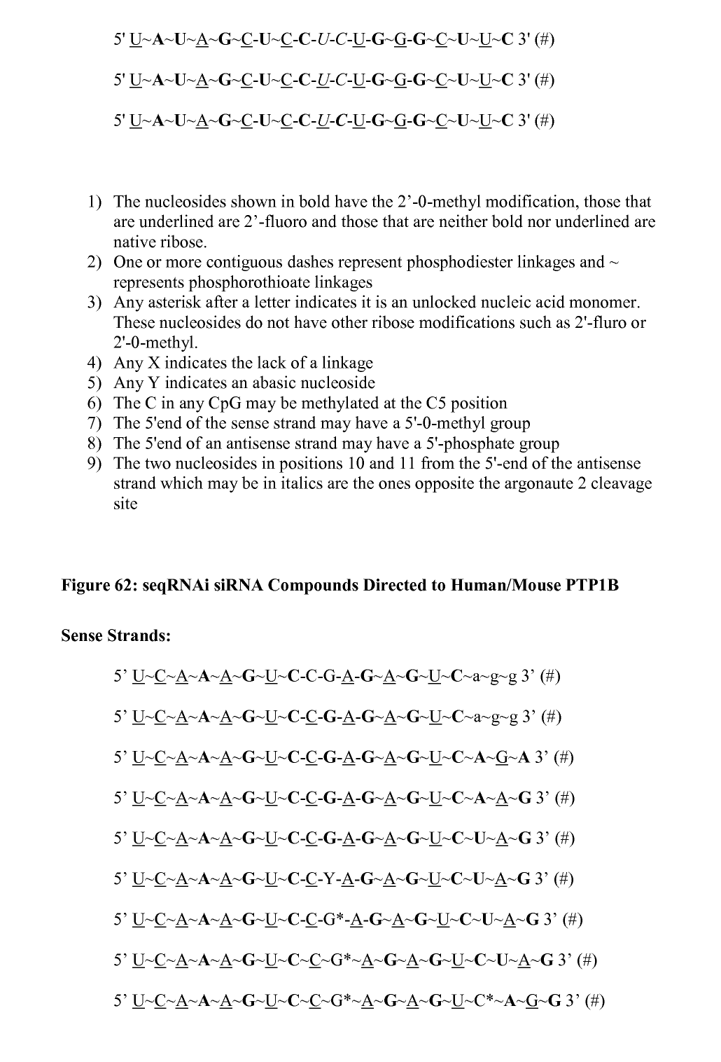

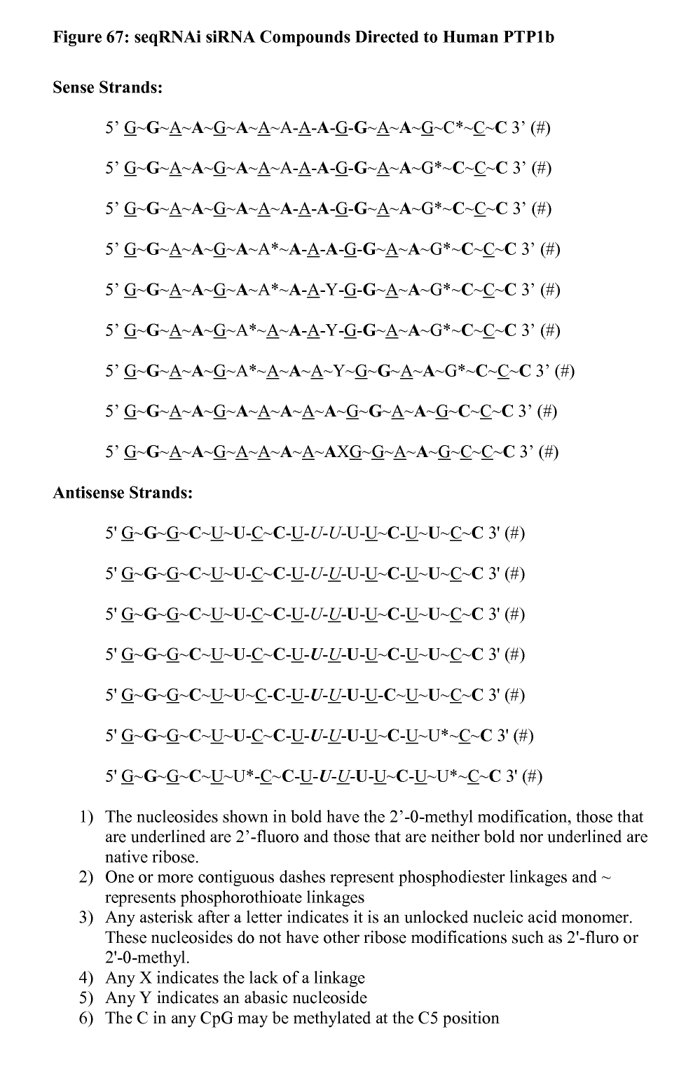

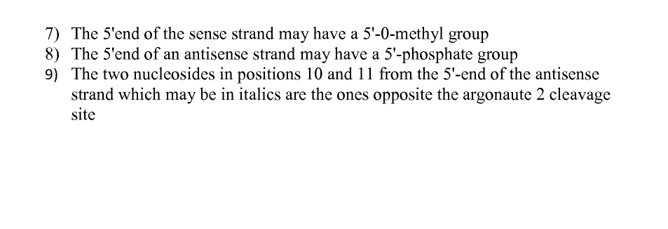

PTP1B, a non-transmembrane protein tyrosine phosphatase that has long been studied as a negative regulator of insulin and leptin signaling, has received renewed attention as an unexpected positive factor in tumorigenesis. These dual characteristics make PTP1B a particularly attractive therapeutic target for diabetes, obesity, and perhaps breast cancer. FIGS. 56, 61, 62 and 67 provide compounds suitable for use in accordance with the present invention to silence PTP expression.

In the case of insulin signaling, PTP1B dephosphorylates the insulin receptor (IR) as well as its primary substrates, the IRS proteins; by contrast, in leptin signaling a downstream element, the tyrosine kinase JAK2(Janus kinase 2), is the primary target for dephosphorylation. However, hints that PTP1B might also play a positive signaling role in cell proliferation began to emerge a few years ago, with the finding by a number of groups that PTP1B dephosphorylates the inhibitory Y529 site in Src, thereby activating this kinase. Other PTP1B substrates might also contribute to pro-growth effects. Indeed, the idea that PTP 1B can serve as a signaling stimulant in some cases received key confirmation in previous work that showed PTP1B plays a positive role in a mouse model of ErbB2-induced breast cancer. See Yip et al. Trends in Biochemical Sciences 35:442-449 (2010). For these reasons, PTP1B has attracted particular attention as a potential therapeutic target in obesity, diabetes, and now, cancer. Accordingly, the compounds directed at PTP1B can be used to advantage for the treatment of such disorders.

### Example II

### Applications for seqIMiRs

MiRNAs have been shown to have wide ranging effects on gene expression. In certain instances, these effects are detrimental and related to certain pathologies. Accordingly, specific miRNA inhibitors which target such miRNAs for degradation are highly desirable. The present inventor has devised strategies for the synthesis of miRNA inhibitors suitable for in vivo delivery which exhibit enhanced stability, the ability to form active duplexes in cells, which act in turn to inhibit the activity of endogenous miRNAs associated with disease. These design paradigms and the resulting miRNA inhibitors are described herein below.

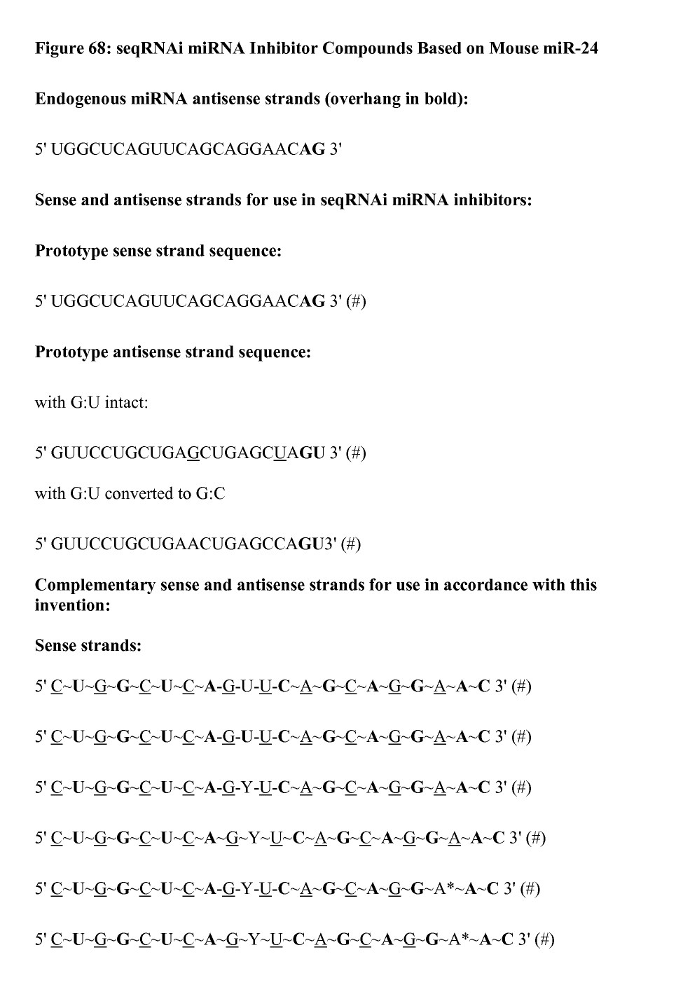

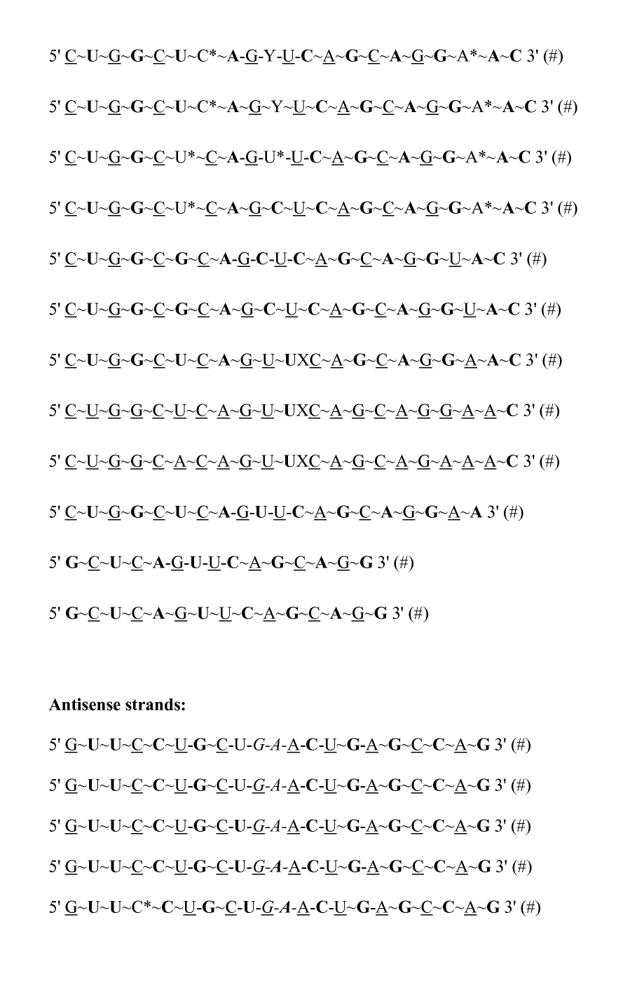

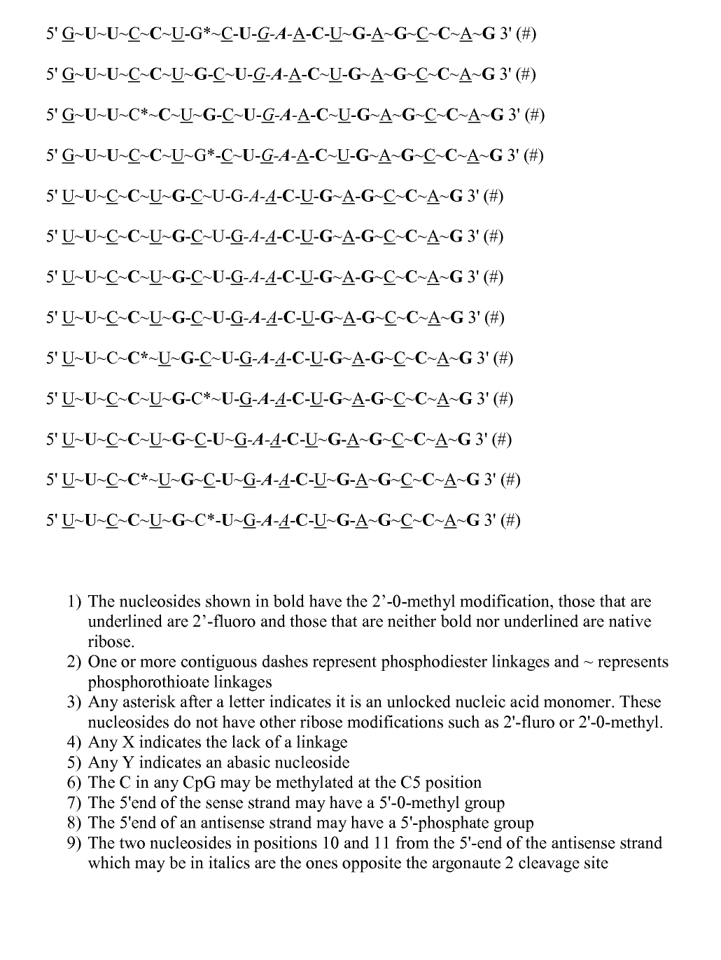

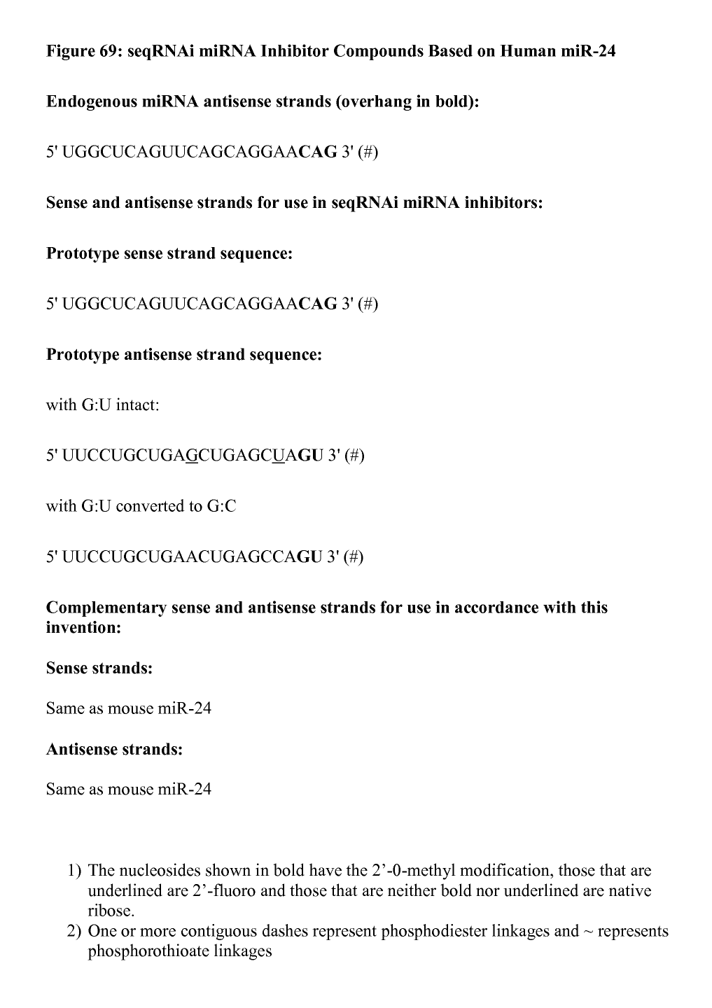

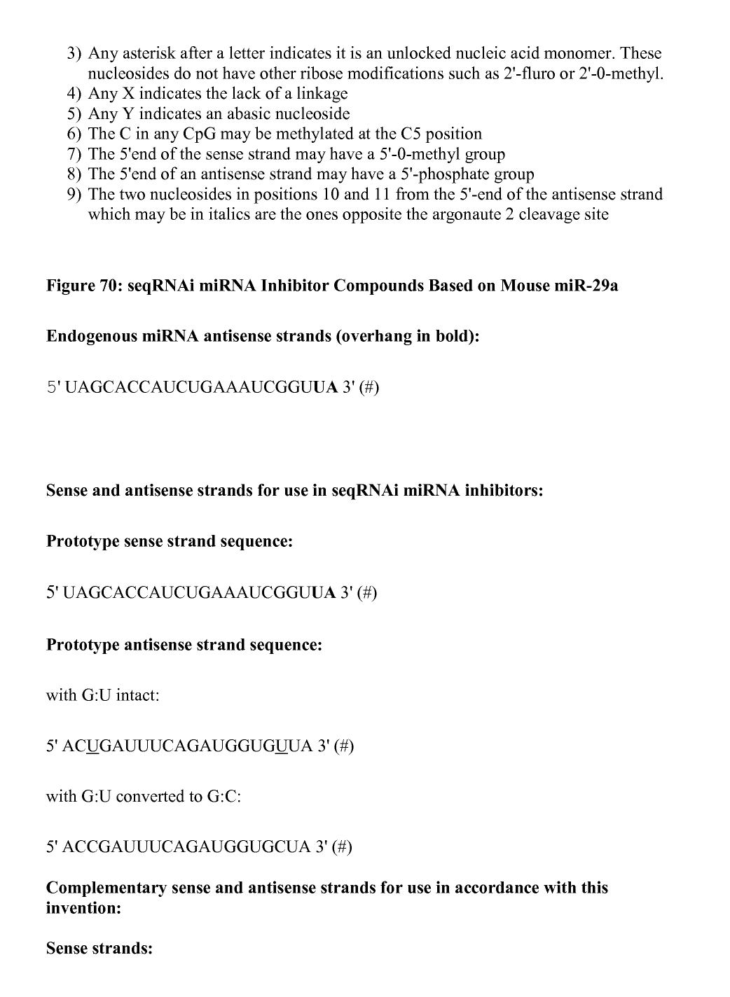

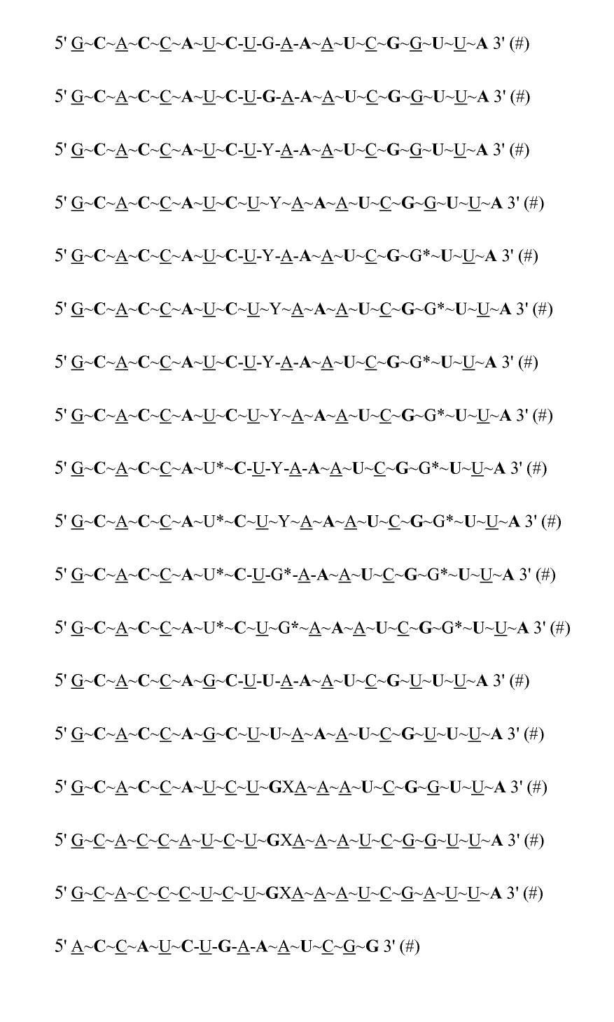

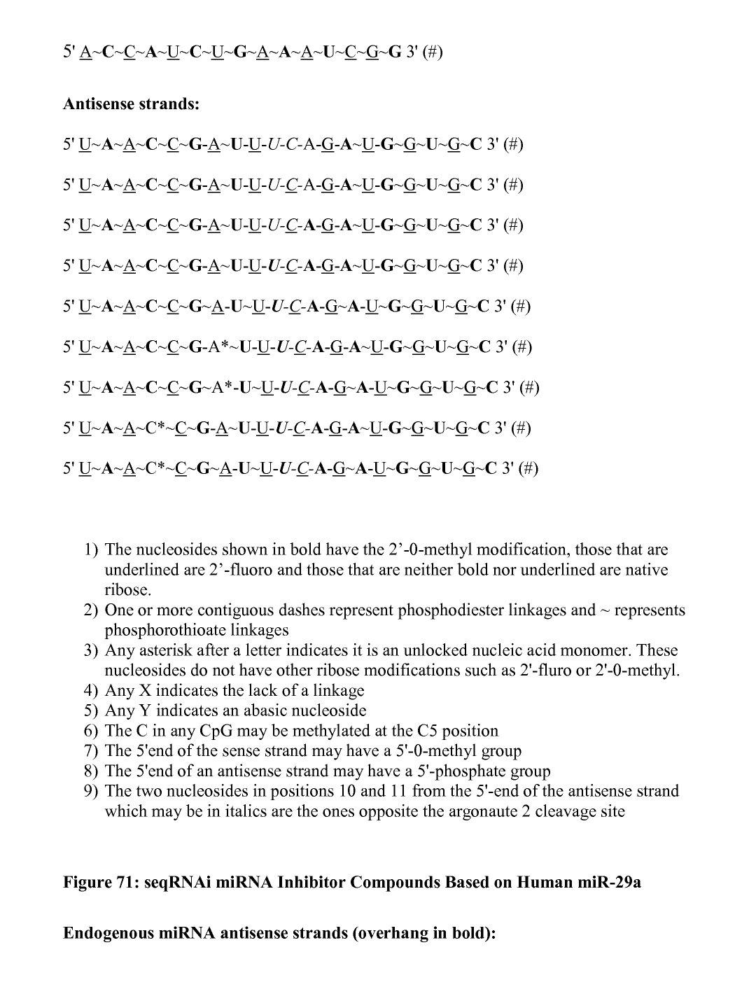

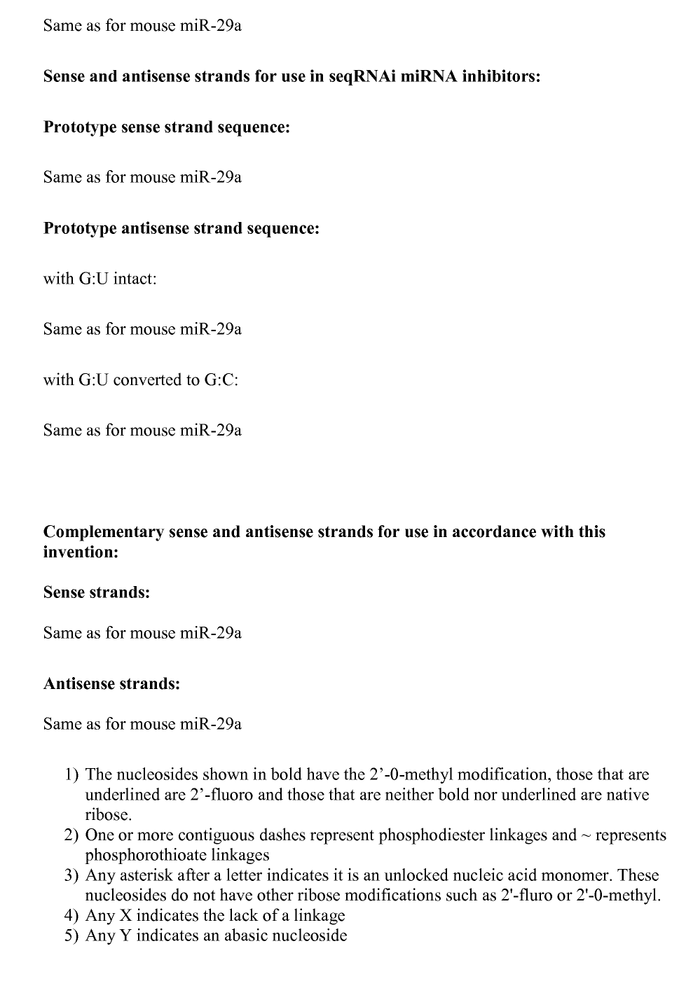

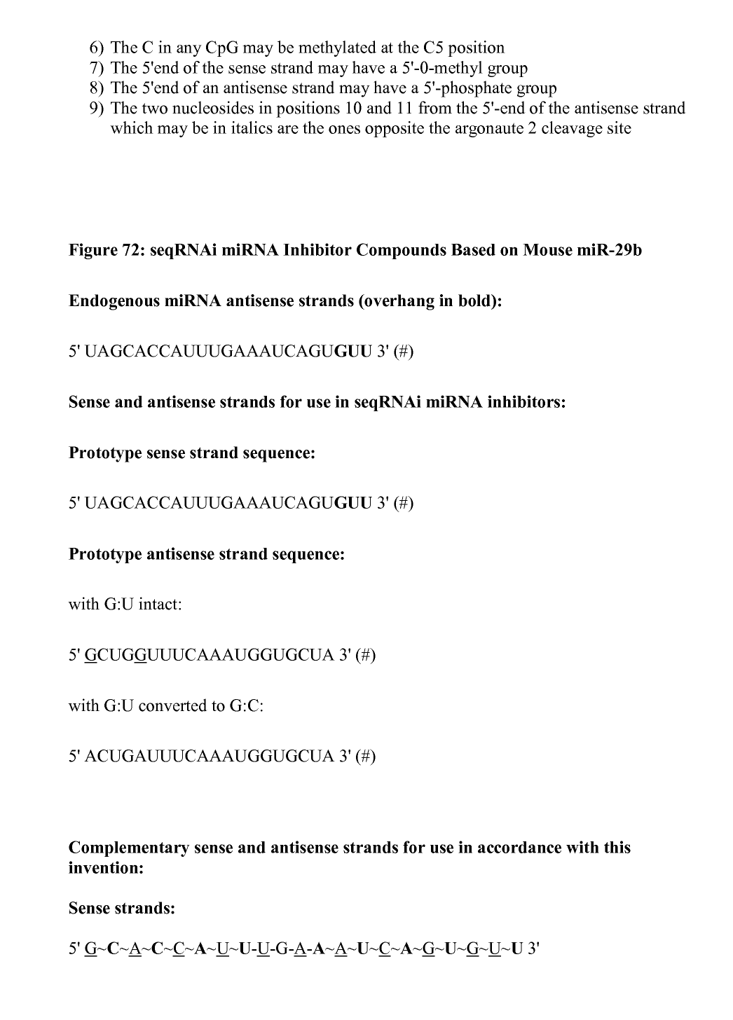

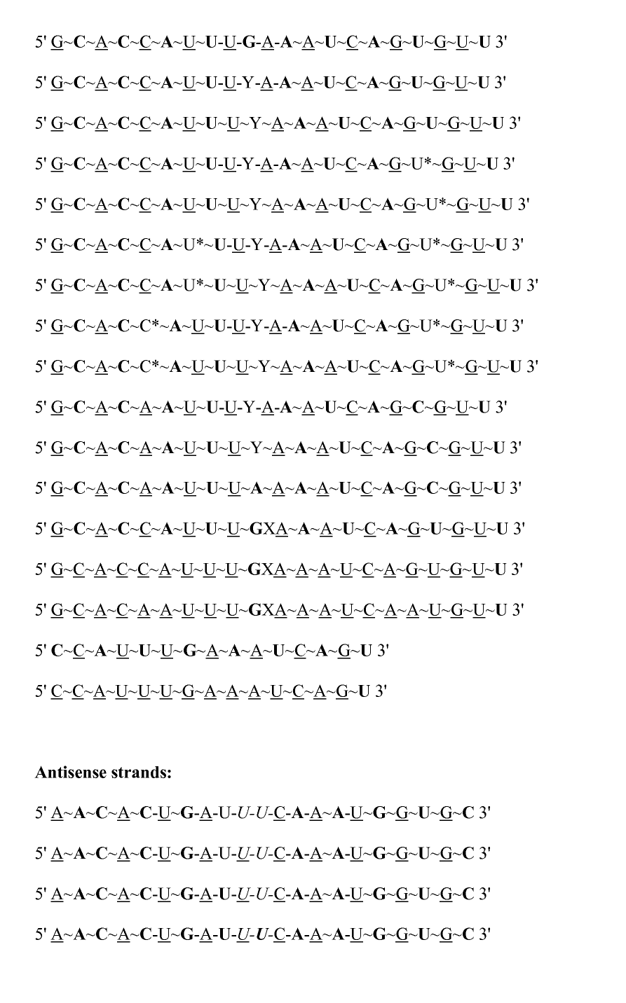

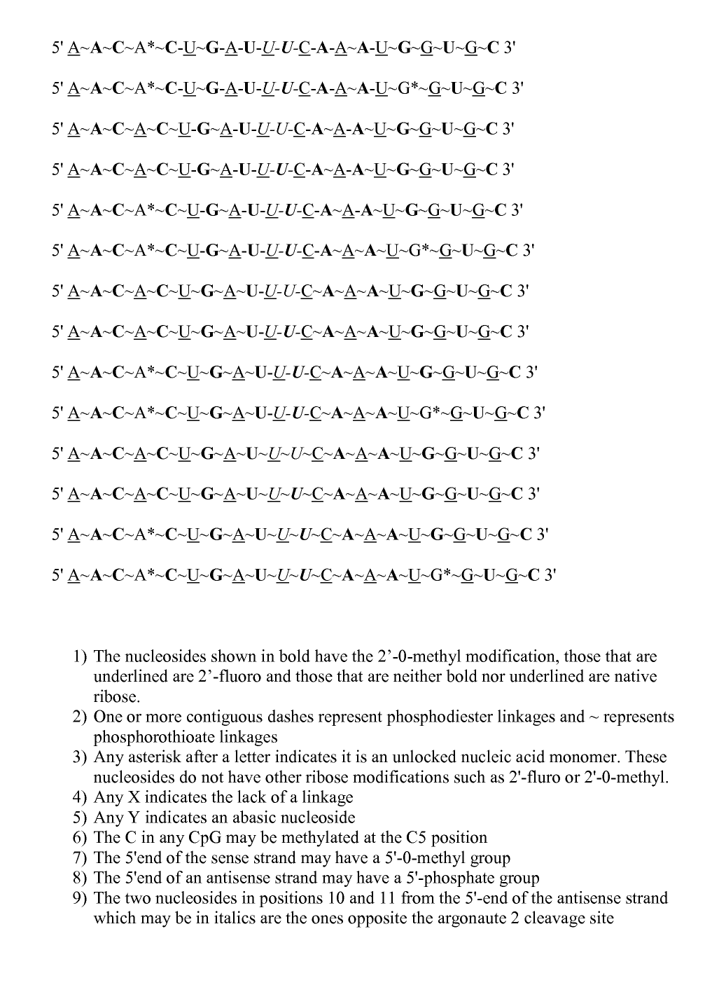

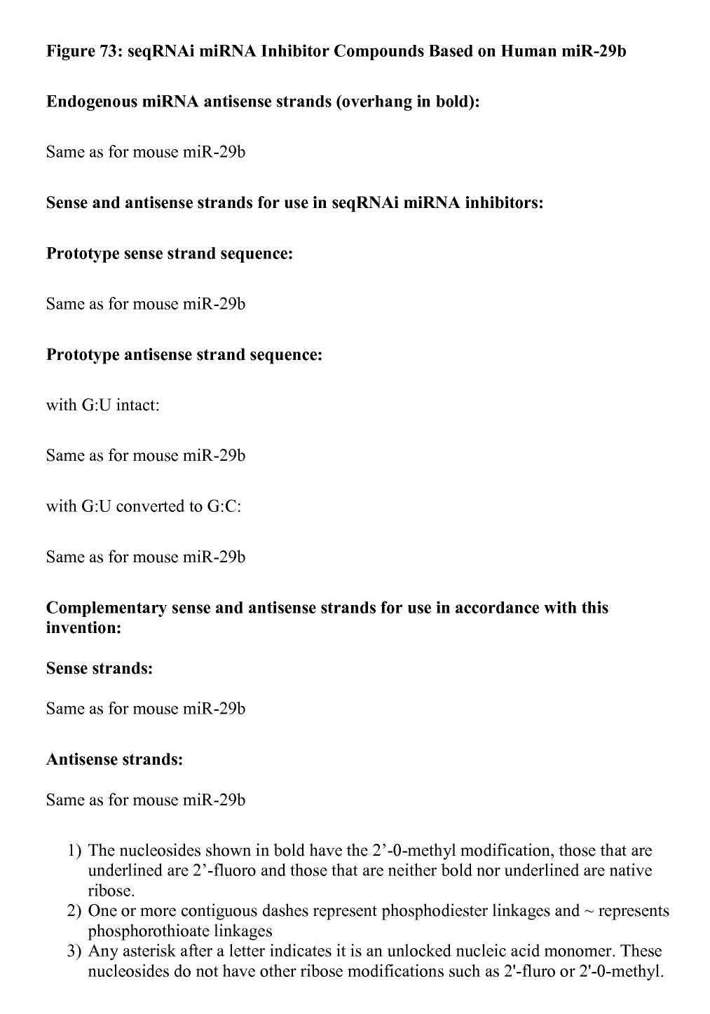

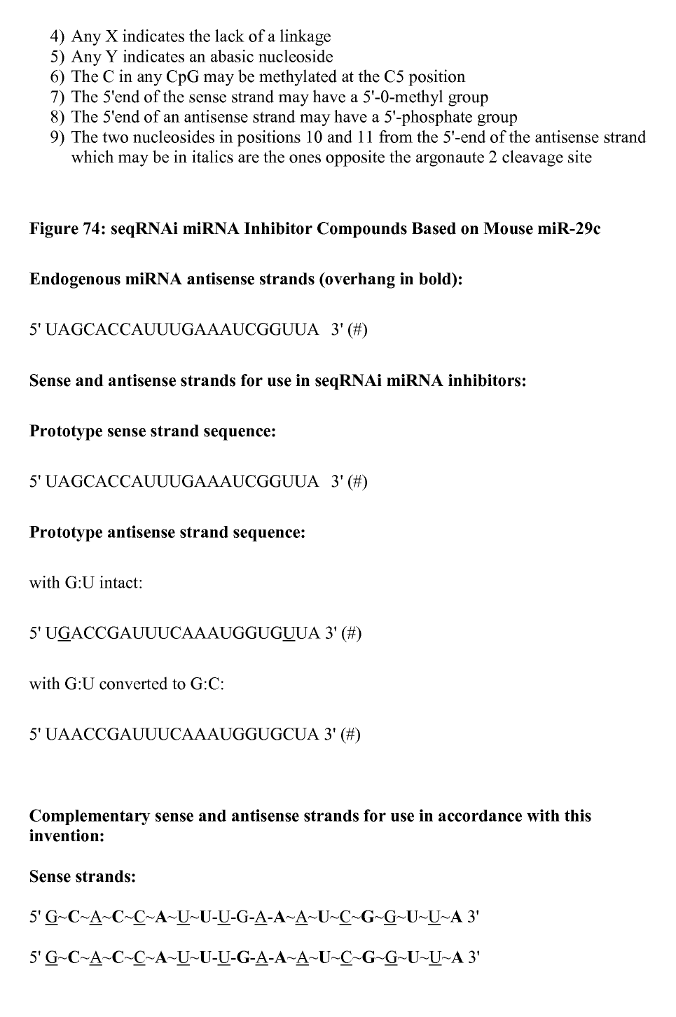

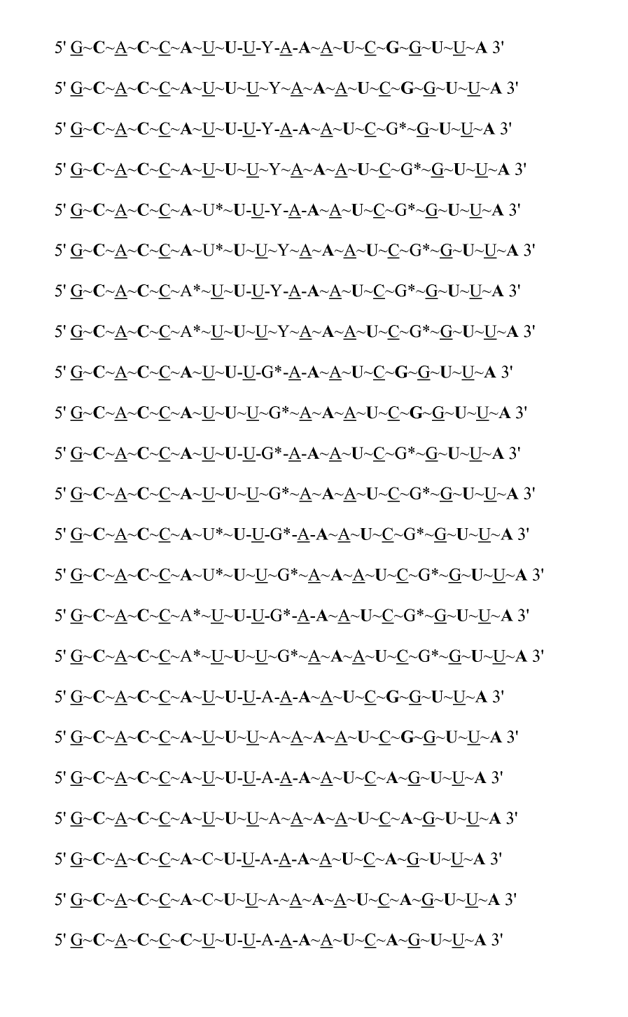

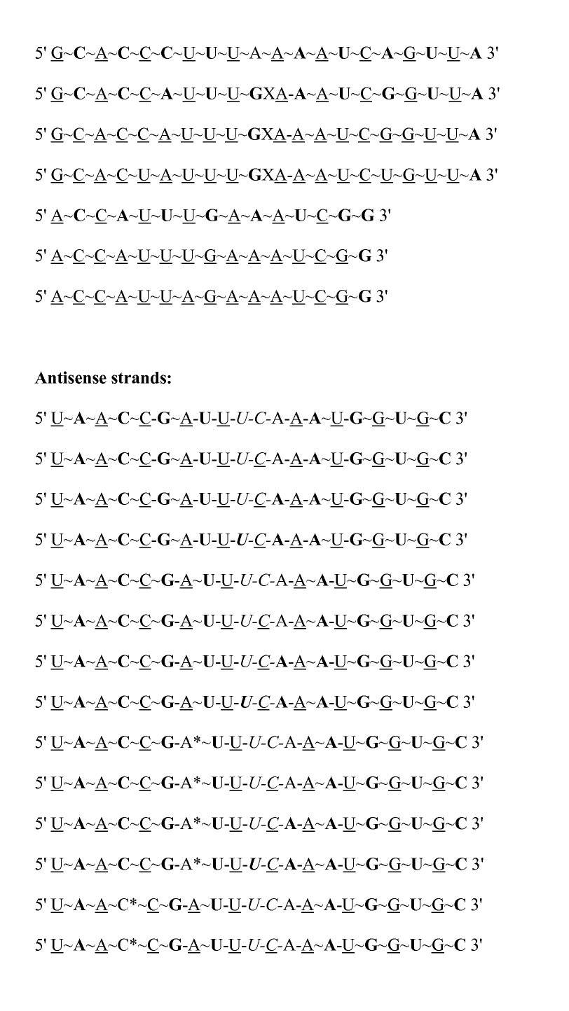

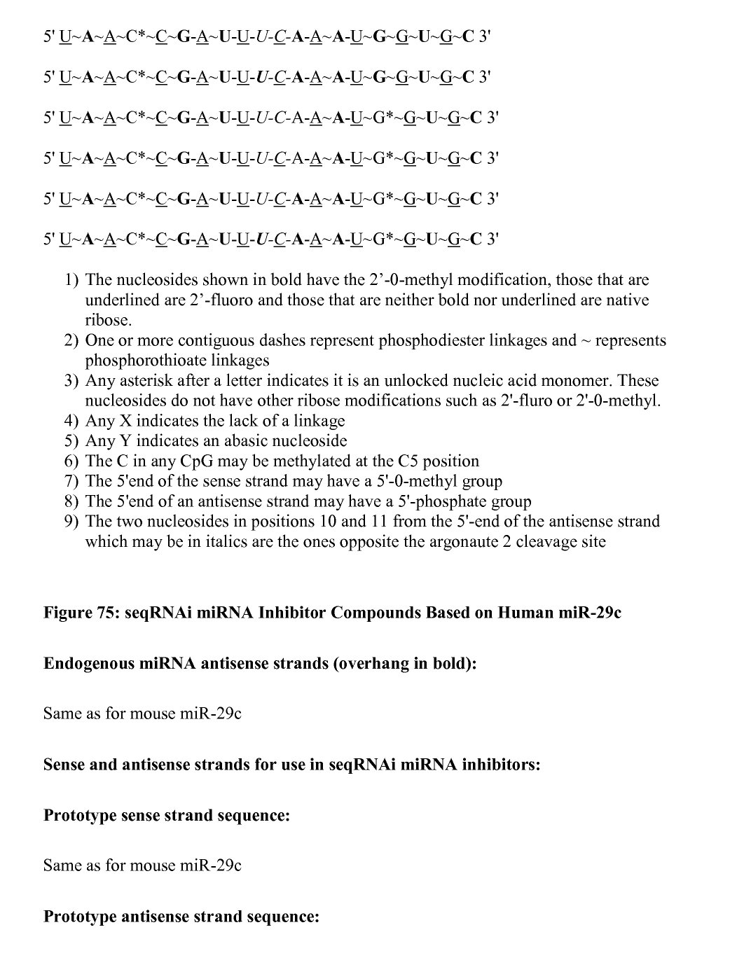

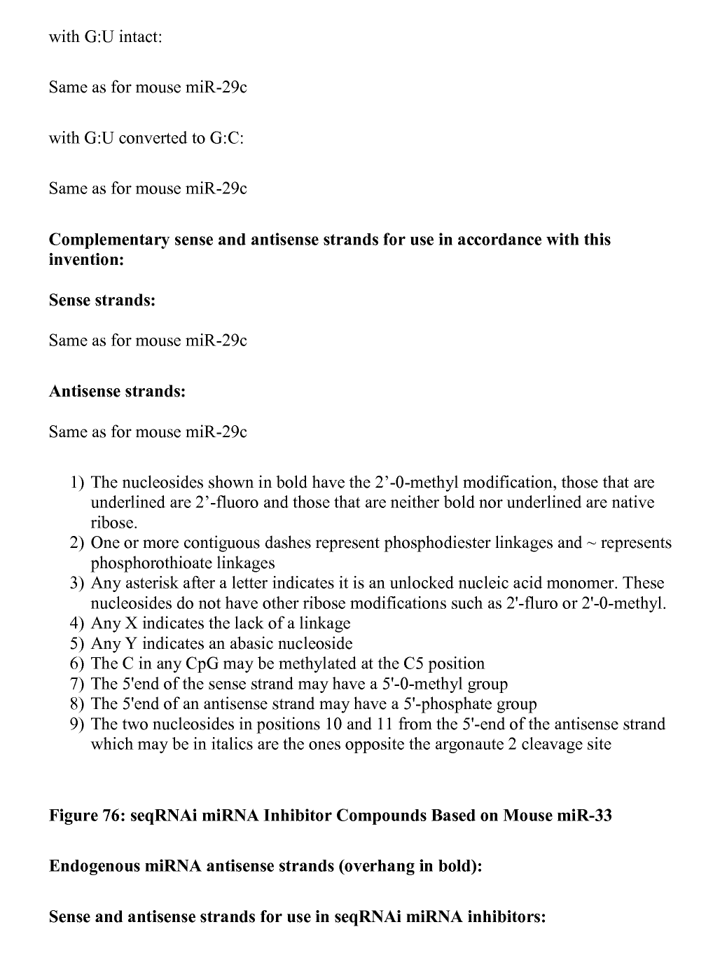

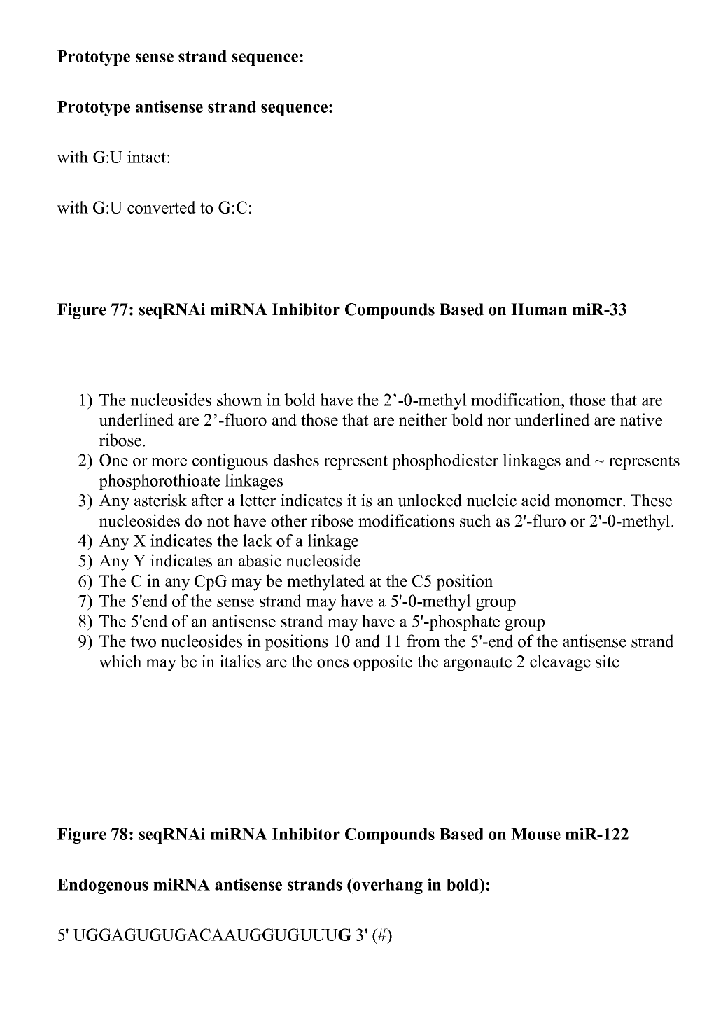

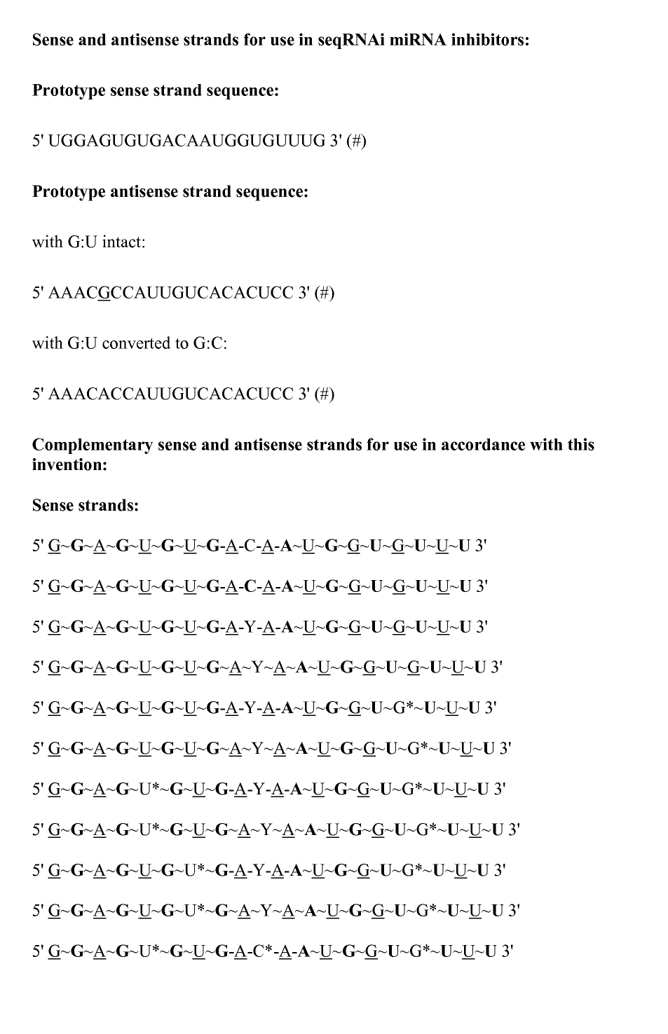

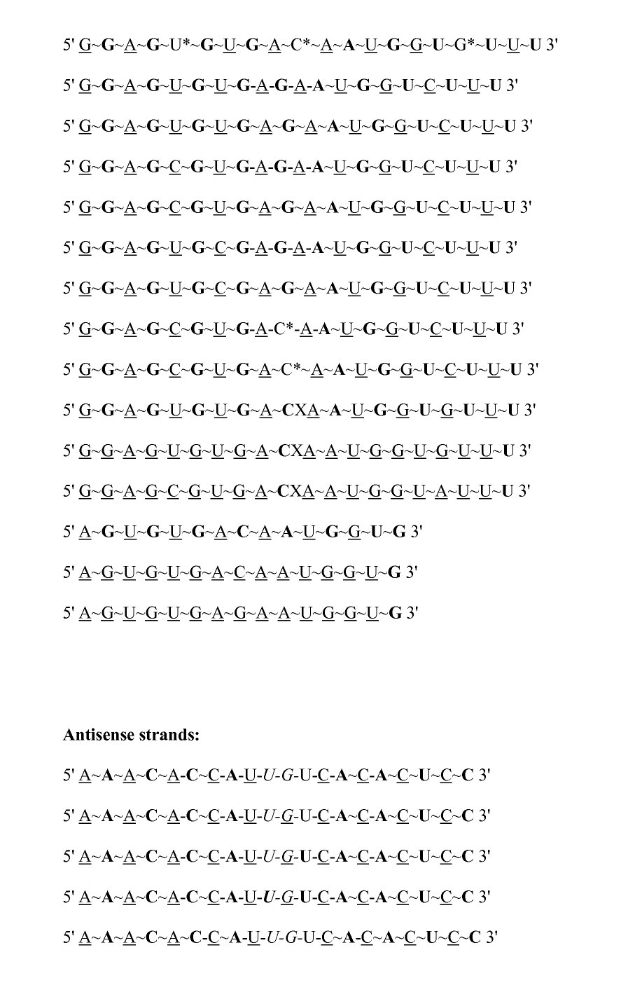

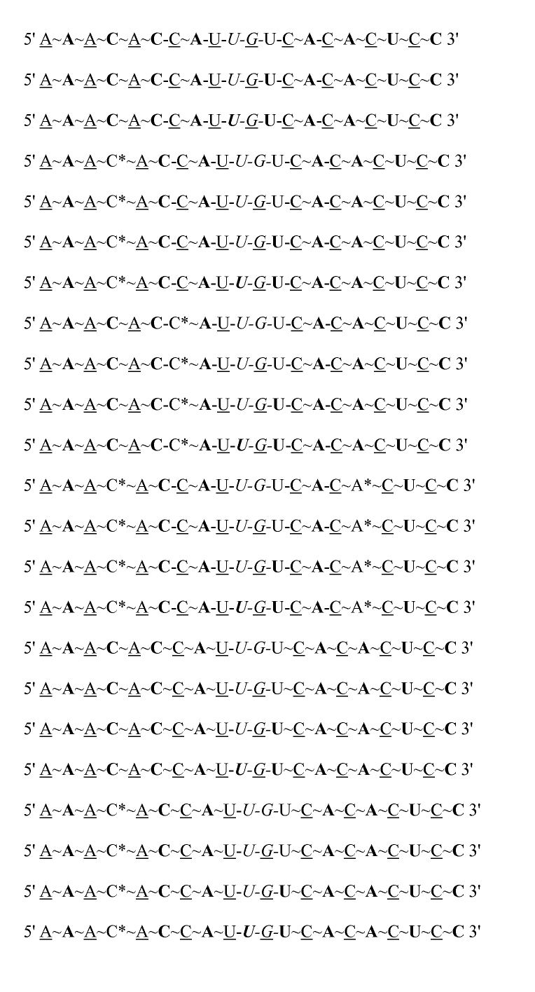

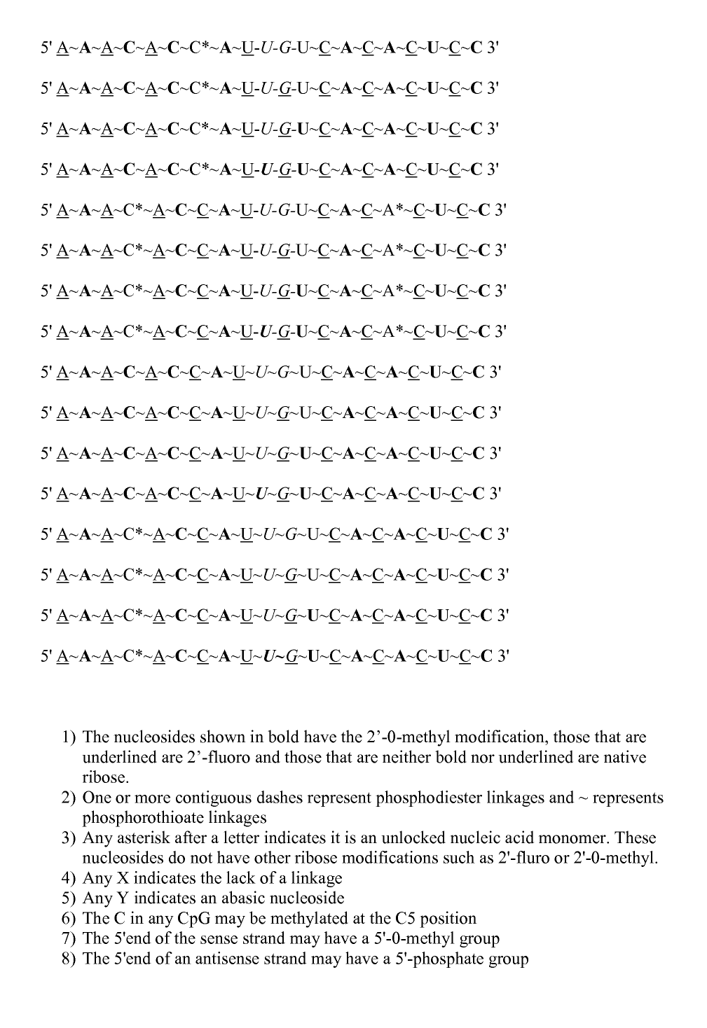

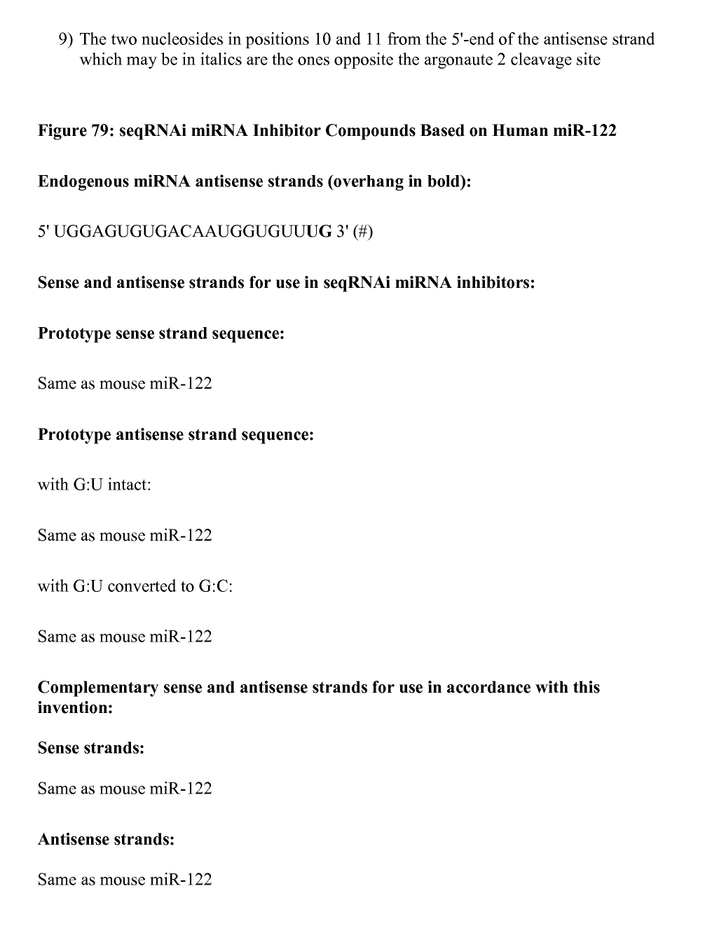

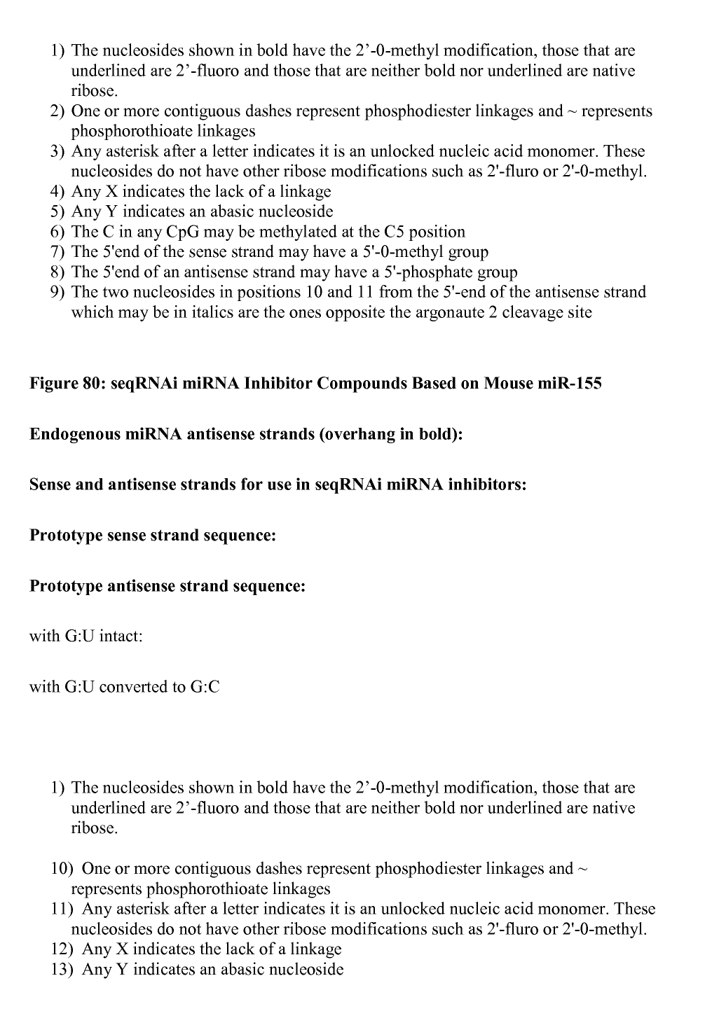

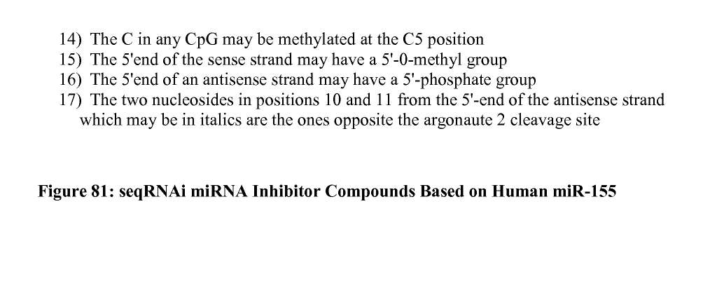

Table 7 provides a listing of some of the medical uses of the seqIMiRs directed to the indicated miRNAs. FIGS. 68-81 provide pairs of seqIMiR strands that are effective to inhibit the actions of these miRNA targets. The methods of the present invention, however, can be used to generate seqIMiRs against any miRNA. Methods for administration of the oligos of the invention are provided in detail above.

TABLE 7
MICRORNA TARGETS FOR INHIBITION BY seqIMiRs
AND COMMERCIAL APPLICATIONS

| MicroRNA | Medical Conditions to be Treated using the |
|---|---|
| Targets | seqIMiR Compounds of the Invention |
| miR-24 | Treat cancer including hormone resistant prostate |
| miR-29a | Inhibit pathologic apoptosis including that due to ischemia |
| | reperfusion injury such as occurs after the removal of a clot |
| miR-29b | Inhibit pathologic apoptosis |
| miR-29c | Inhibit pathologic apoptosis including that due to ischemia |
| | reperfusion injury such as occurs after the removal of a clot |
| miR-33 | Raise good cholesterol (HDL) levels |
| miR-122 | Hepatitis C |
| miR-155 | Arthritis; Autoimmune inflammation including that |
| | associated with cystic fibrosis; Atopic dermatitis |
| miR-208a | Chronic heart failure |

Conventional antisense oligos of different types are under development for potential use as competitive inhibitors of particular endogenous miRNAs for research, development and therapeutic purposes. Such oligos are designed to bind particularly tightly one strand of the miRNA whose actions are to be inhibited. These oligos work by a steric hindrance mechanism.

Elevated levels of miR-21, for example, occur in numerous cancers where it promotes oncogenesis at least in part by preventing the translation and accumulation of PDCD4. Another example is miR-122 a liver specific miRNA that promotes replication of the hepatitis C virus. Conventional antisense oligos that inhibit these miRNAs are in development as potential therapeutic agents.

Compared to antisense oligos that engender catalytic activity against their targets, such as those that are RNase H dependent, the antisense oligos that function as competitive inhibitors must be used at substantially higher concentrations. In vivo various tissues take up oligos in widely ranging amounts. For example, liver and kidney take up relatively large amounts while resting lymphocytes, testis, skeletal muscle the CNS and other tissues take up much smaller amounts. Further, antisense oligos that have a competitive inhibitor function have been shown to perform poorly in tissues that do not avidly take up oligos. Therefore, it would be highly desirable to have oligonucleotide based miRNA inhibitors that have a catalytic activity against them so that a wider range of tissues types can be subject to efficient miRNA inhibition. The present invention provides a solution to this pressing need.

### Example III

### Examples of Applications for seqMiRs

Table 8 below provides a listing of miRNAs for which examples of specific seqMiR compounds have been provided herein. The methods of the present invention can be used to mimic any endogenous miRNA, to improve on the mRNA type silencing pattern of an endogenous miRNA for commercial purposes and can be used to generate designer novel miRNA-like compounds.

TABLE 8
MICRORNAS MIMICKED BY seqMiRs AND COMMERCIAL APPLICATIONS

| MicroRNA | |
|---|---|
| Mimicked by | Medical Conditions to be Treated using the seqMiR Compounds |
| seqMiR | of the Invention |
| Let-7i and Let-7 | Cancer |
| family generally | |
| miR-24-1 | Ischemia reperfusion injury including that associated with myocardial |
| | infarction; Diabetes |
| miR-24-2 | Ischemia reperfusion injury including that associated with myocardial |
| | infarction; Diabetes |
| miR-26a-1 | Cancer including liver, head and neck, breast |
| miR-26a-2 | Cancer including liver, head and neck, breast |
| miR-29a | Fibrosis including liver, lung, kidney and heart; Systemic sclerosis; Cancers |
| | including lung, liver, chronic lymphocytic leukemia; Osteoporosis; Systemic |
| | sclerosis; |
| miR-29b-1 | Fibrosis including liver, lung, kidney and heart; Systemic sclerosis; Cancers |
| | including lung, liver, colon breast, chronic lymphocytic leukemia, acute |
| | myeloid leukemia |
| miR-29b-2 | Fibrosis including liver, lung, kidney and heart; Systemic sclerosis; Cancers |
| | including lung, liver, colon, breast, rhabdomyosarcoma, chronic lymphocytic |
| | leukemia, acute myeloid leukemia; |
| miR-29c | Fibrosis including liver, lung, kidney and heart; Systemic sclerosis; Cancers |
| | including lung, liver, rhabdomyosarcoma, chronic lymphocytic leukemia; |
| miR-34a | Cancer including prostate, ovarian, non-small cell lung cancer, pancreatic |
| | cancer, stomach cancer, retinoblastoma and chronic lymphocytic leukemia; |
| miR-34b | Cancer including prostate, ovarian, non-small cell lung cancer, pancreatic |
| | cancer, stomach cancer, retinoblastoma and chronic lymphocytic leukemia; |
| miR-34c | Cancer including prostate, ovarian, non-small cell lung cancer, pancreatic |
| | cancer, stomach cancer, retinoblastoma and chronic lymphocytic leukemia; |
| miR-122 | Cancer including liver, lung and cervical; |
| miR-146a | Atherosclerosis |
| miR-203 | Sensitize cancers with mutant p53 including colon cancer to chemotherapy |
| | including taxanes |
| miR-214 | Nerve regeneration; Diabetes including type 2; |
| miR-499 | Myocardial infarction including the ischemia-reperfusion injury related to |
| | reversing vessel occlusion; |

It is now well established that post-transcriptional gene silencing (PTGS) by miRNA and other RNAi-associated pathways represents an essential layer of complexity to gene regulation. Gene silencing using RNAi additionally demonstrates huge potential as a therapeutic strategy for eliminating gene expression associated with the pathology underlying a number of different disorders.

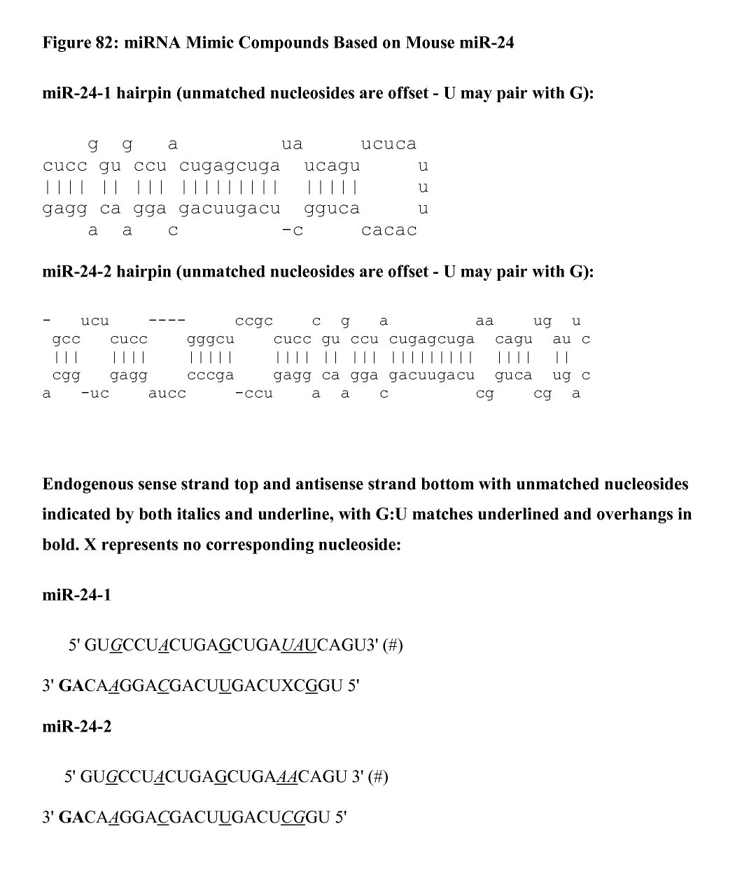

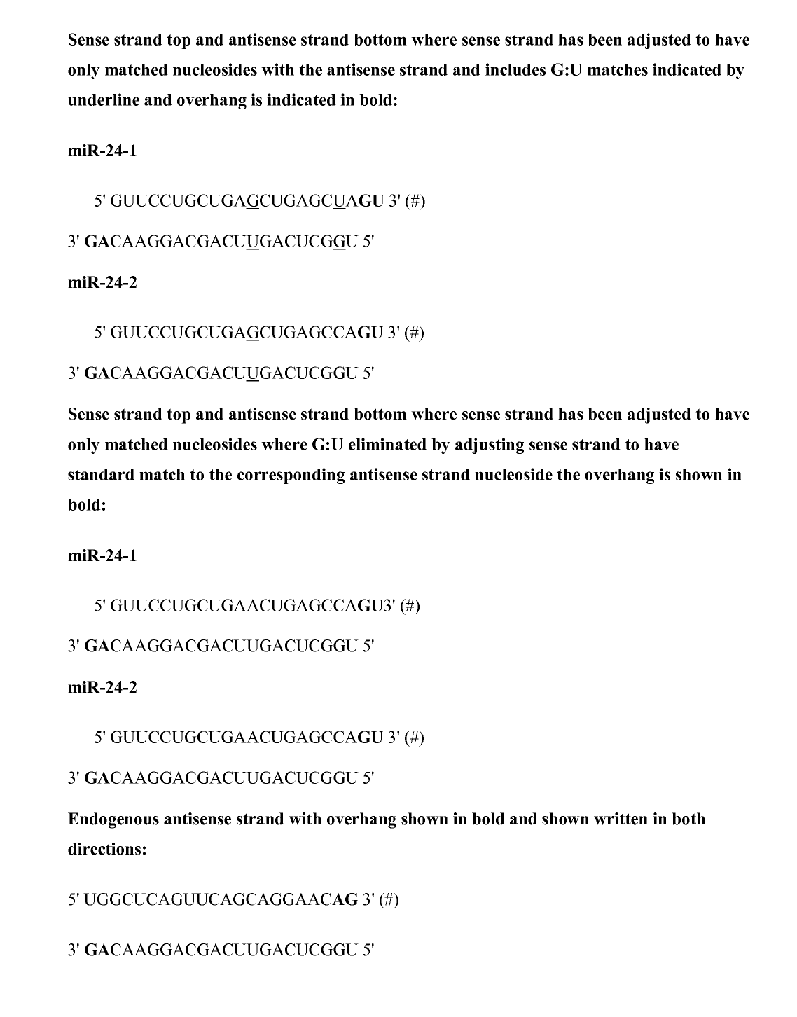

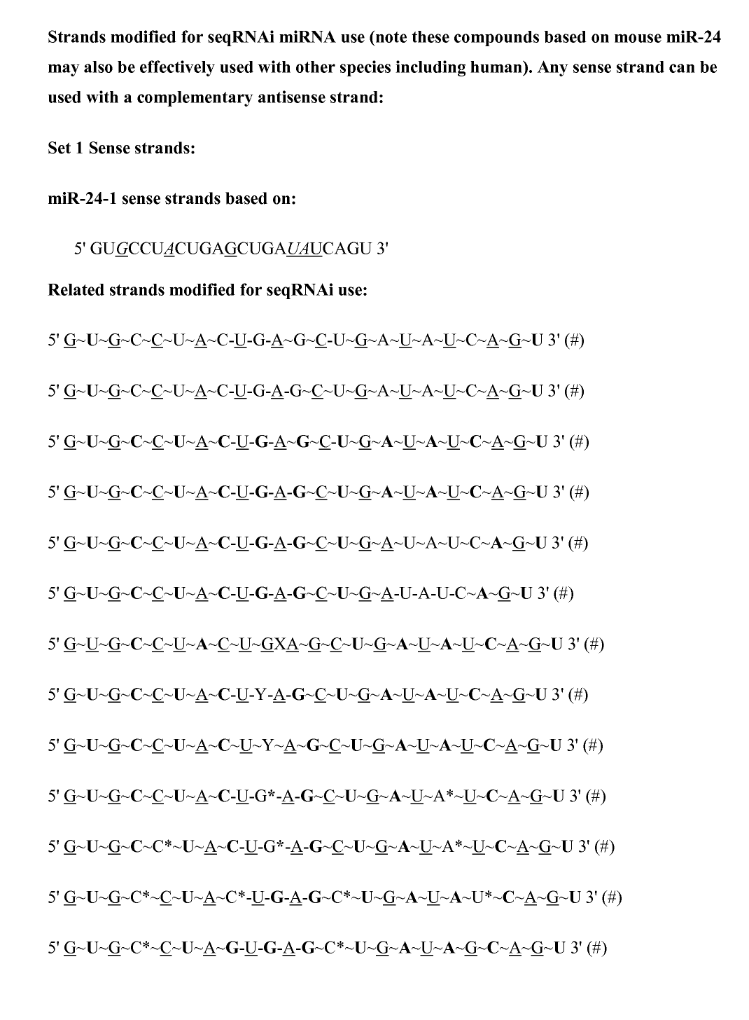

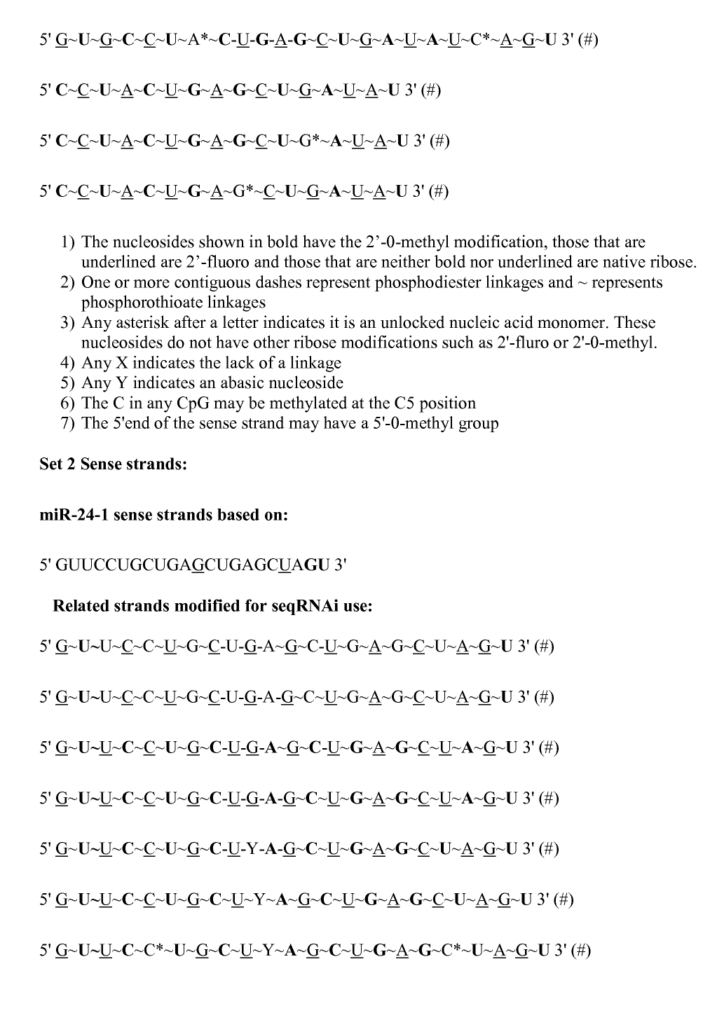

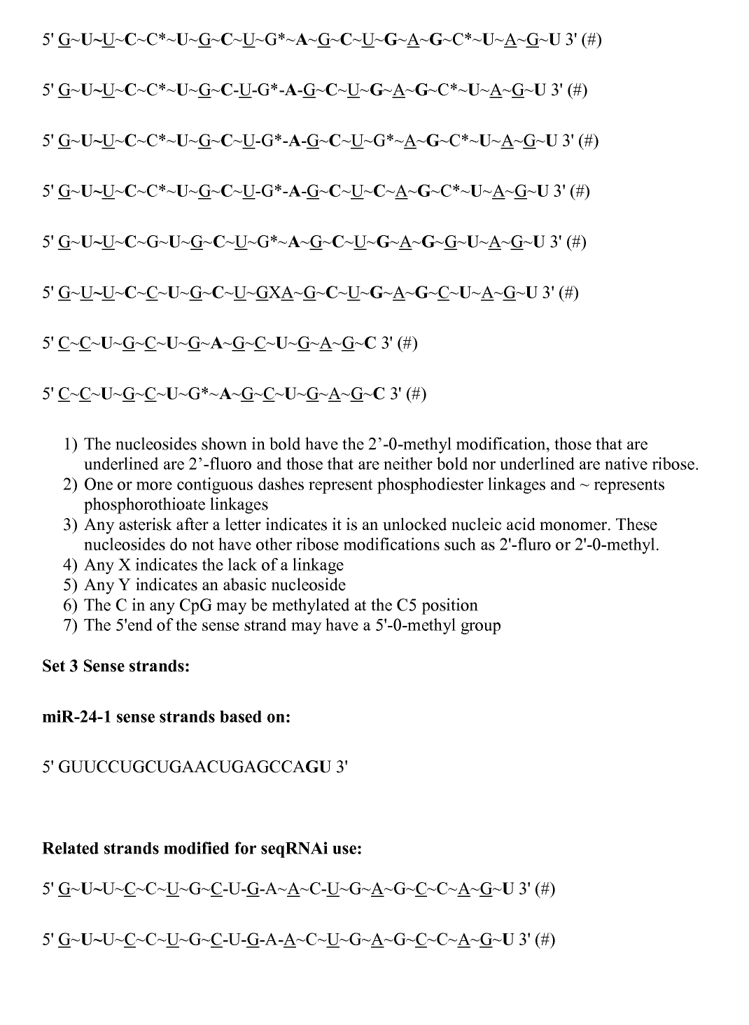

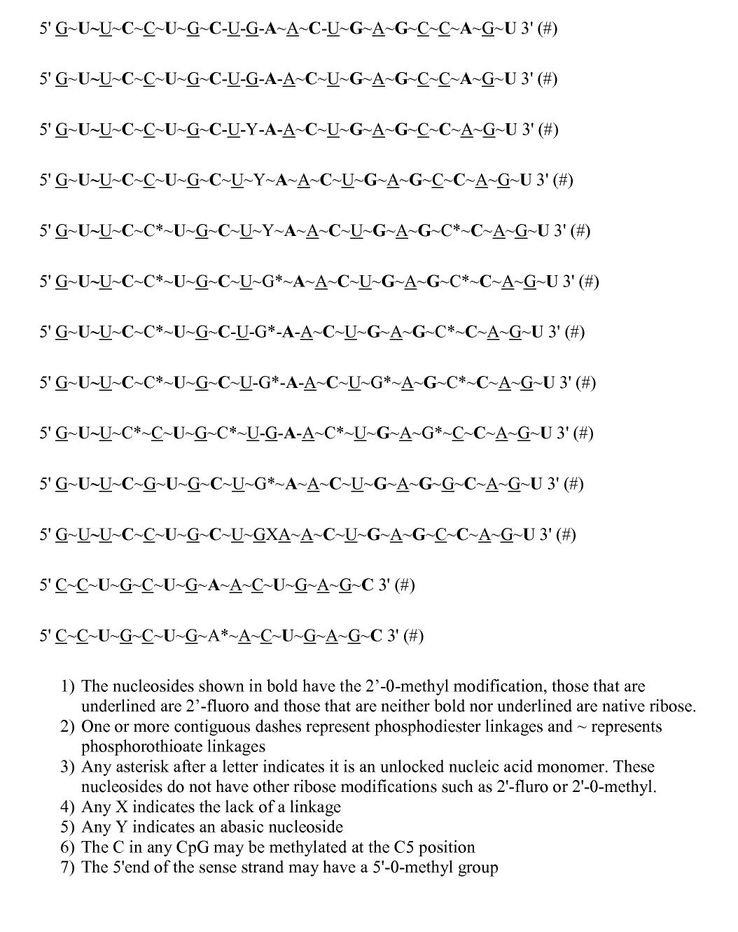

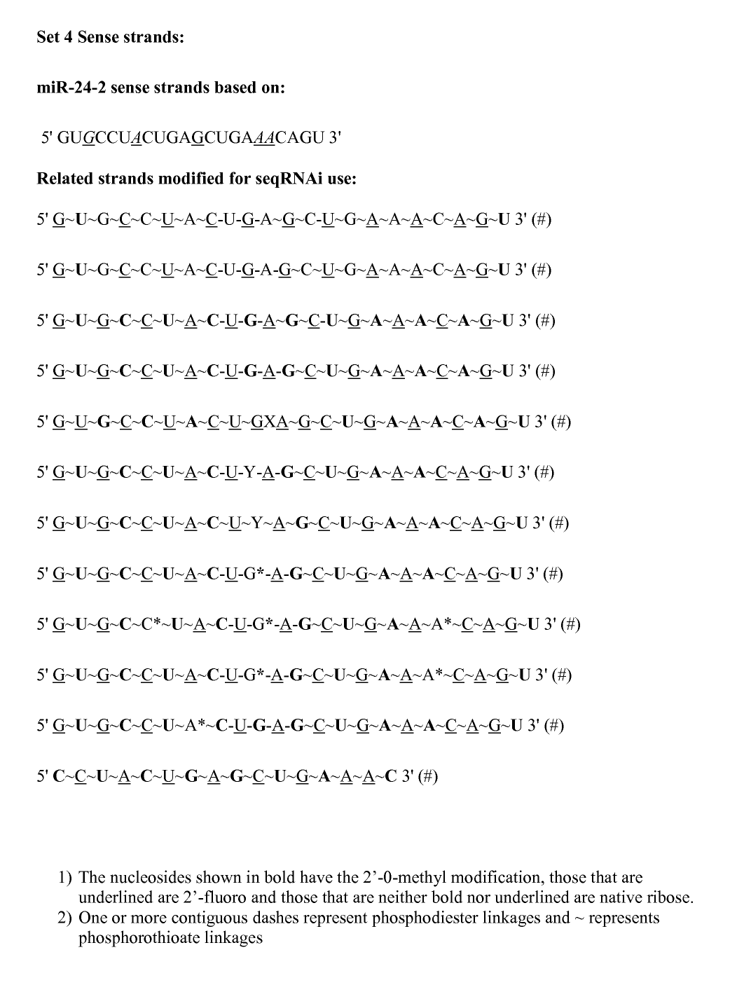

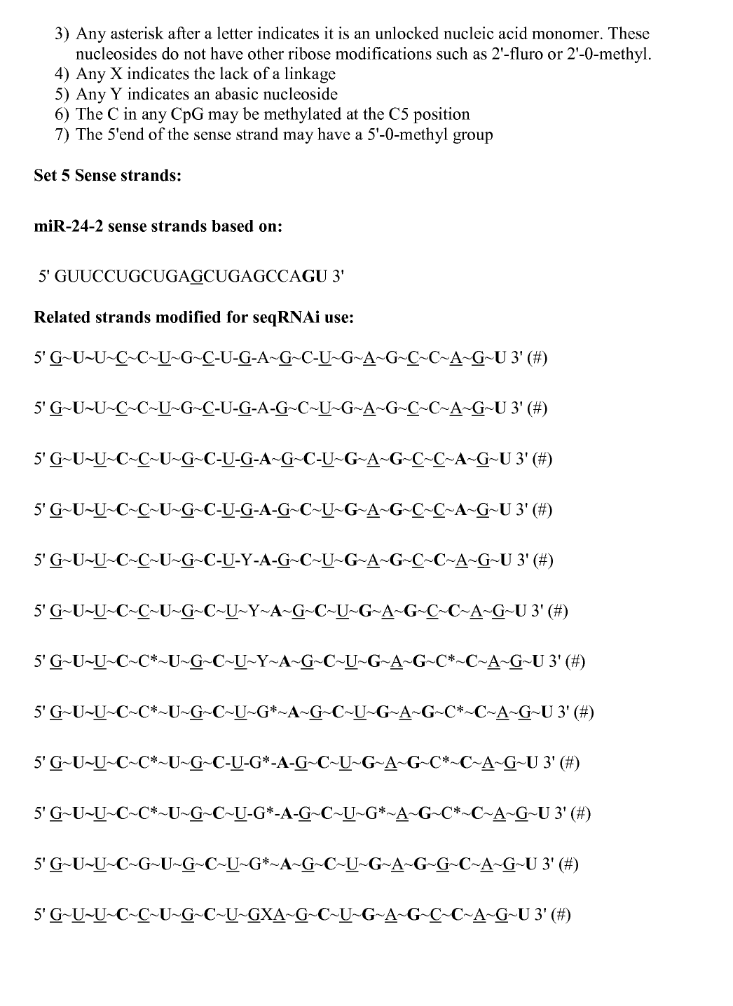

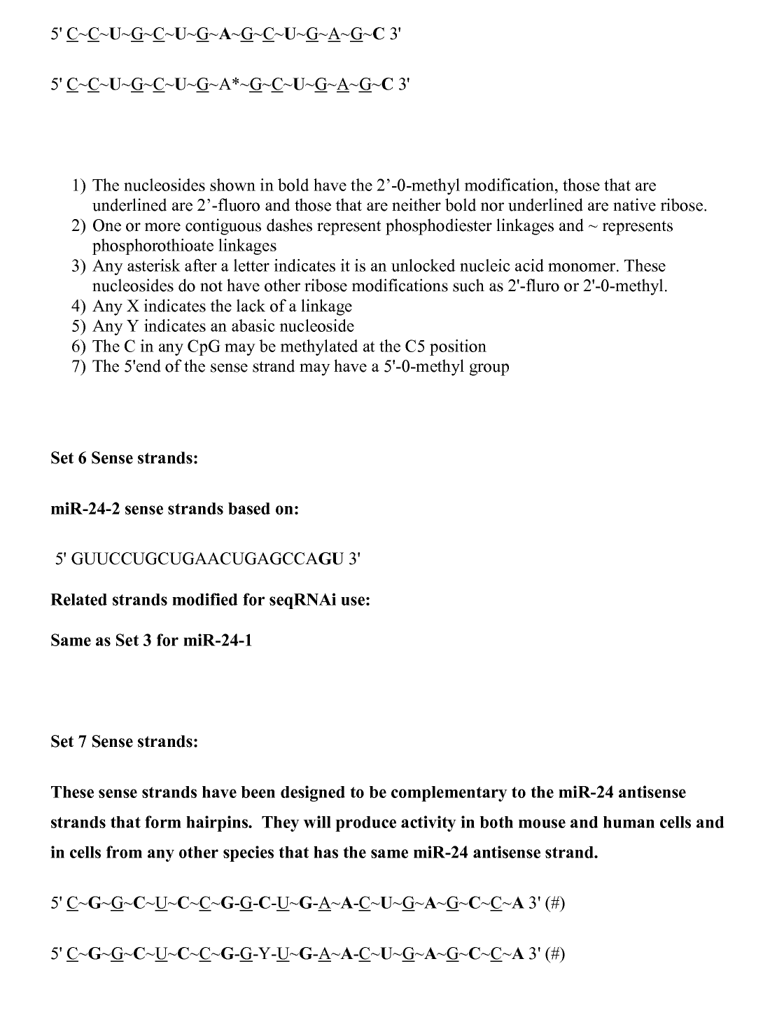

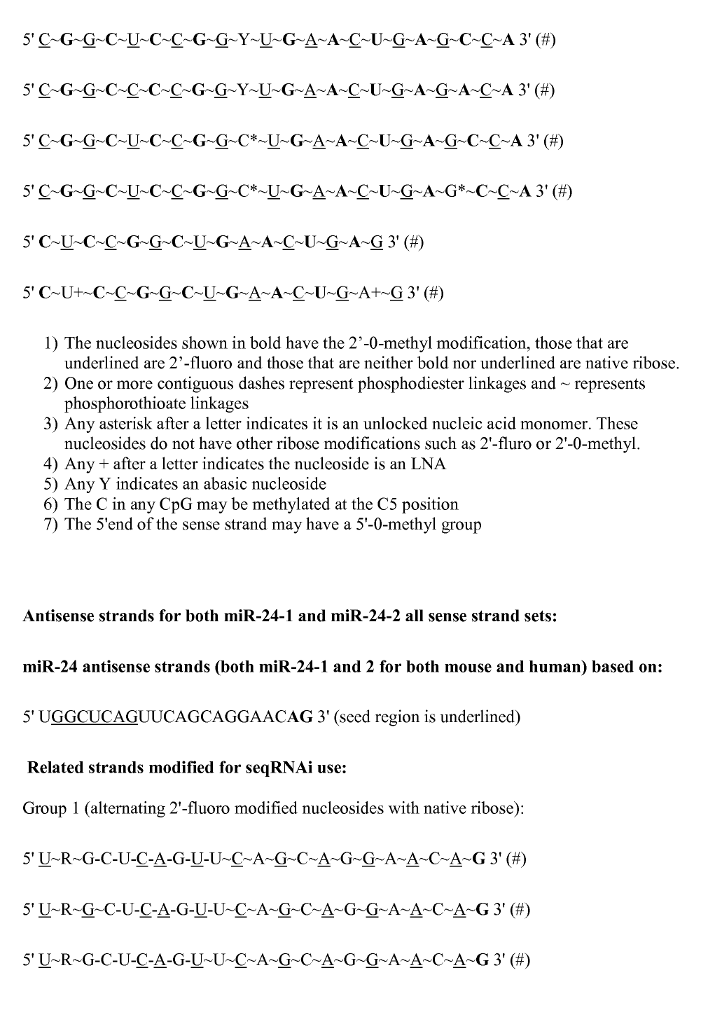

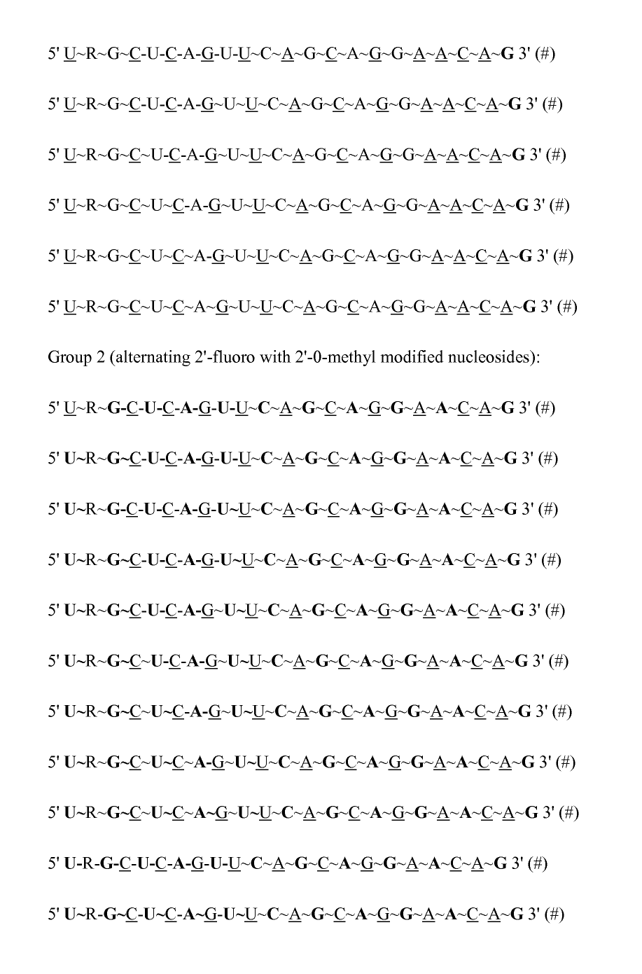

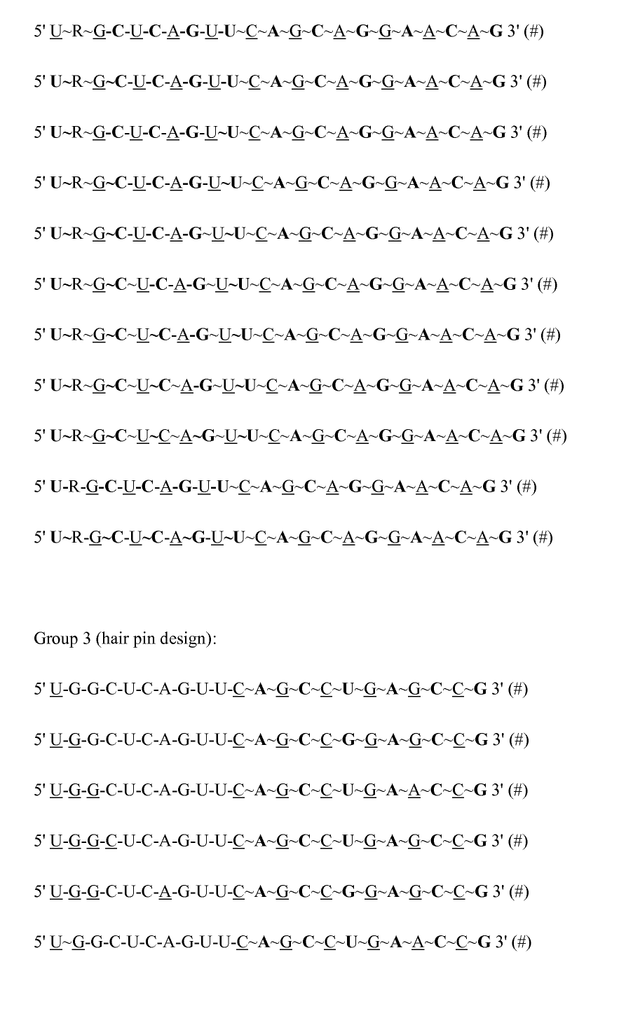

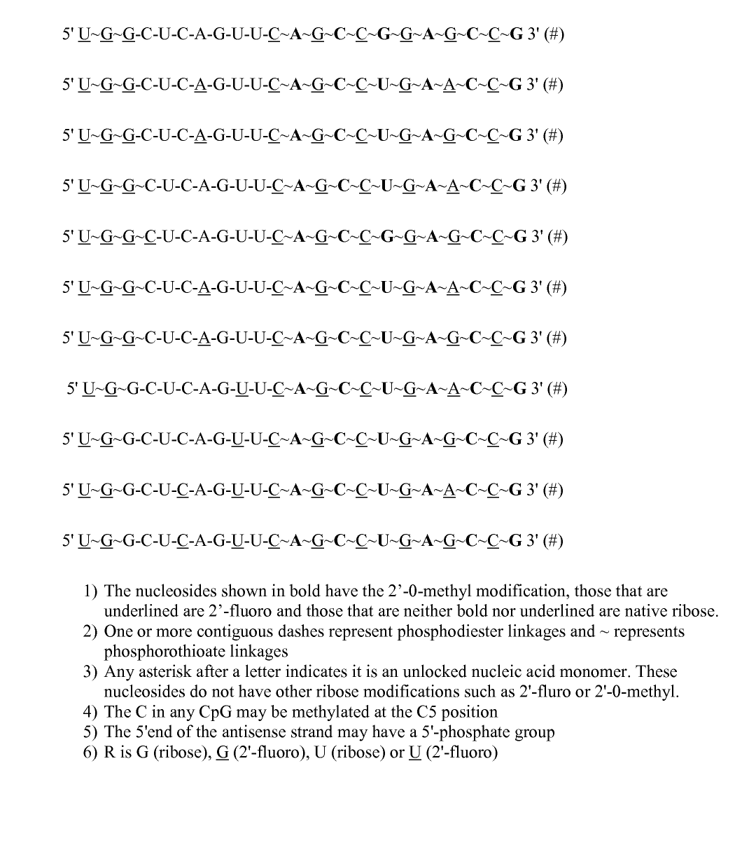

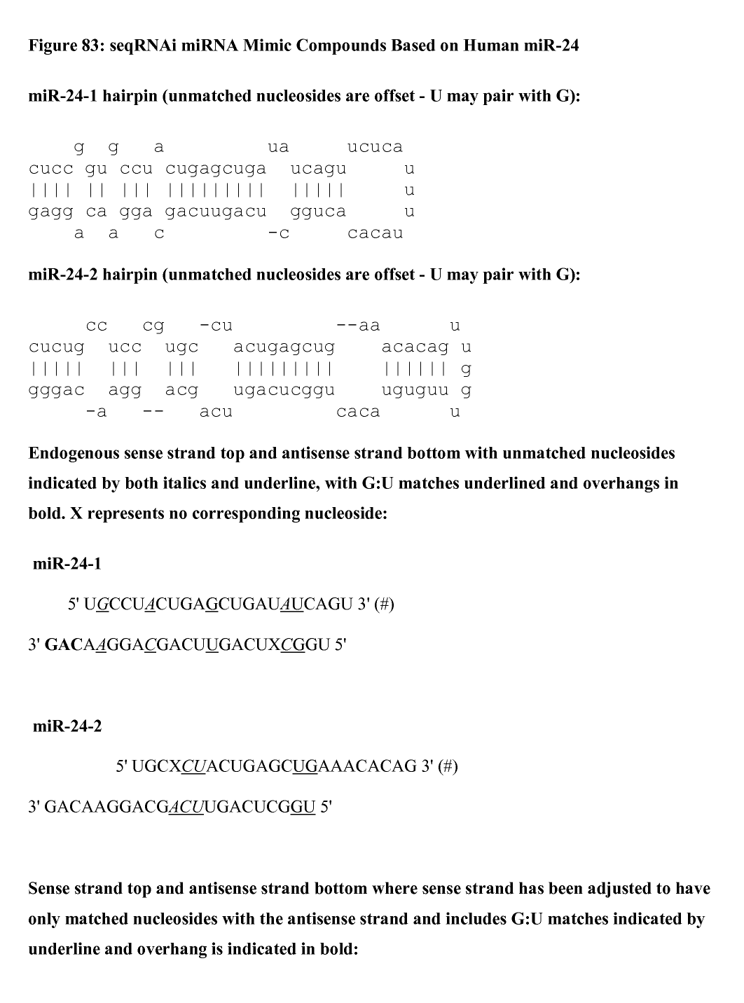

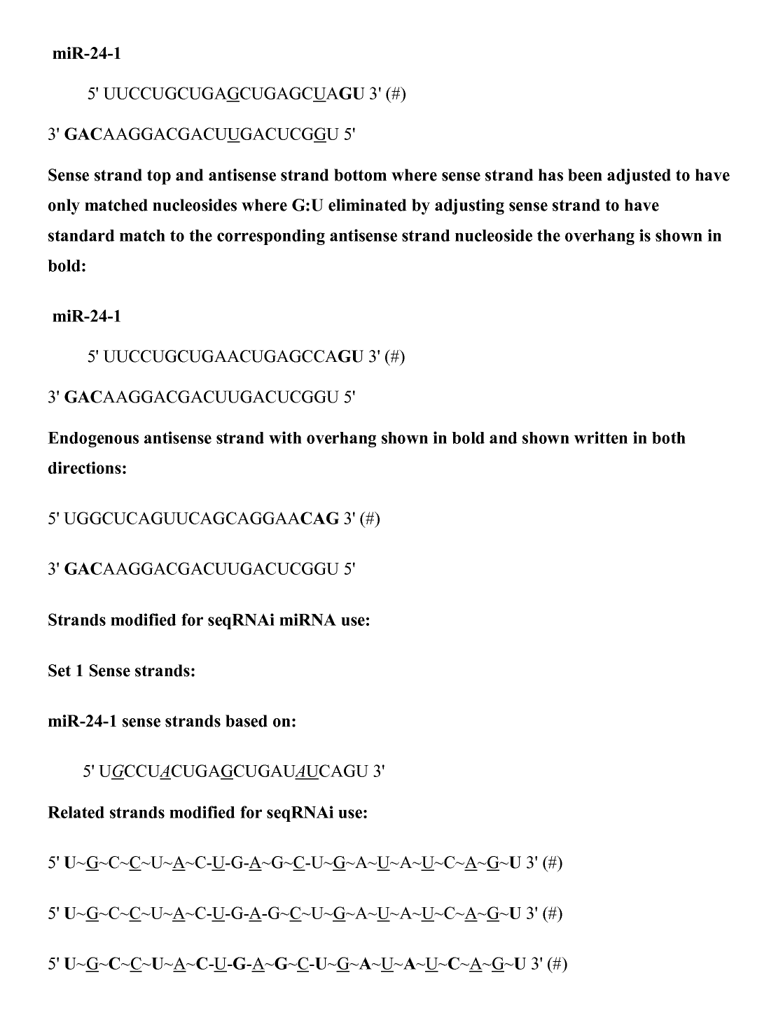

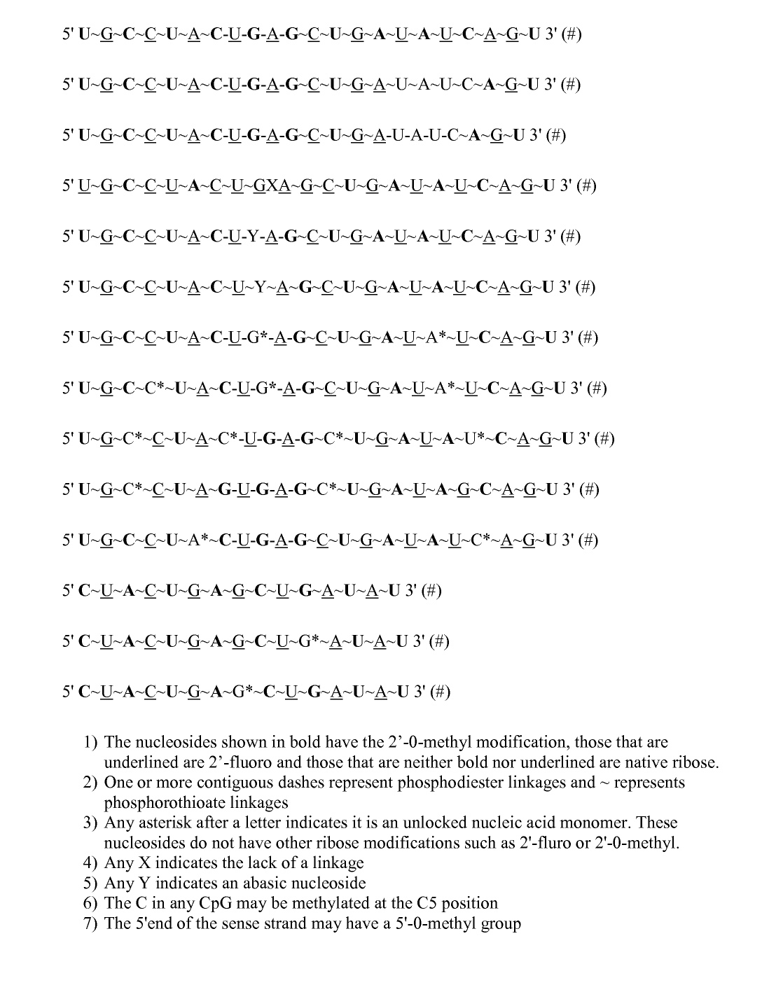

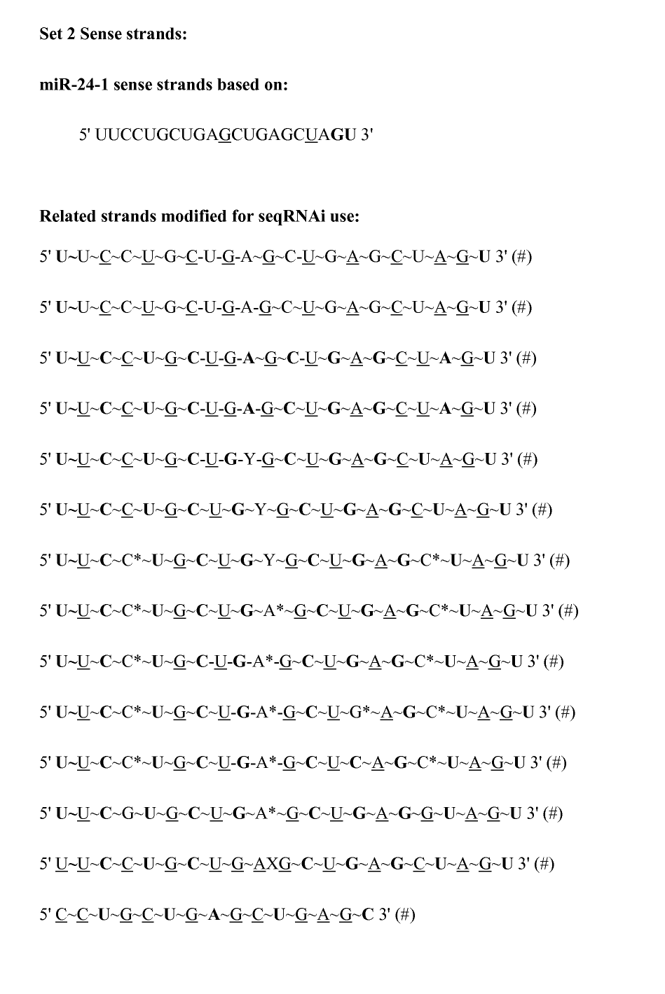

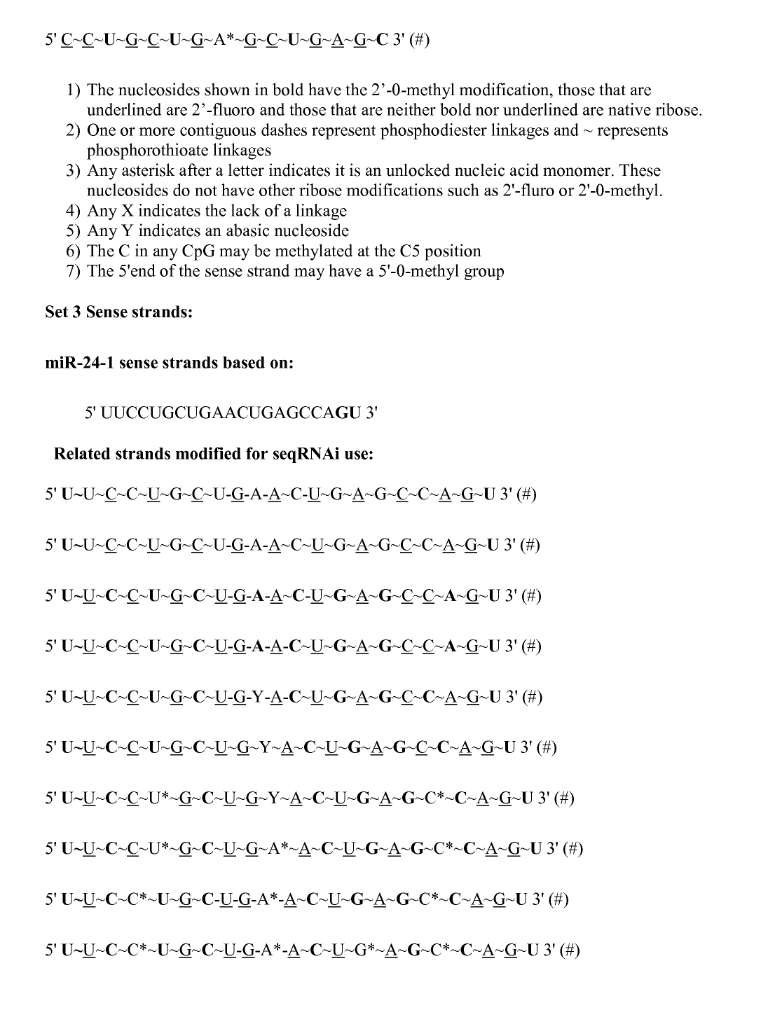

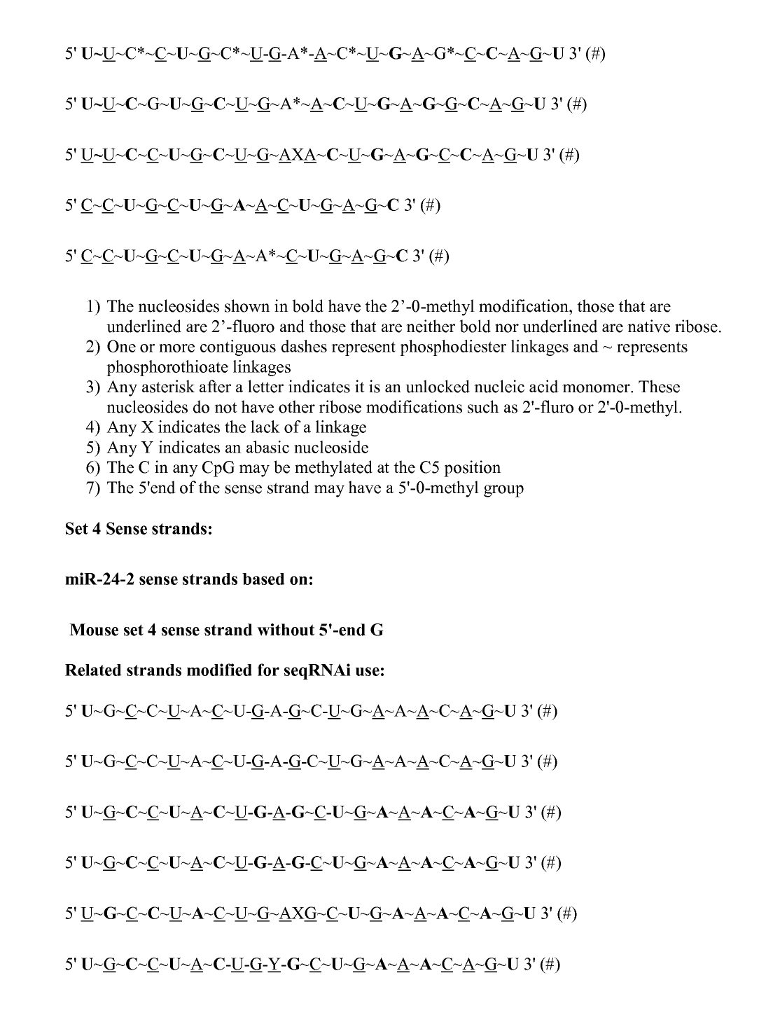

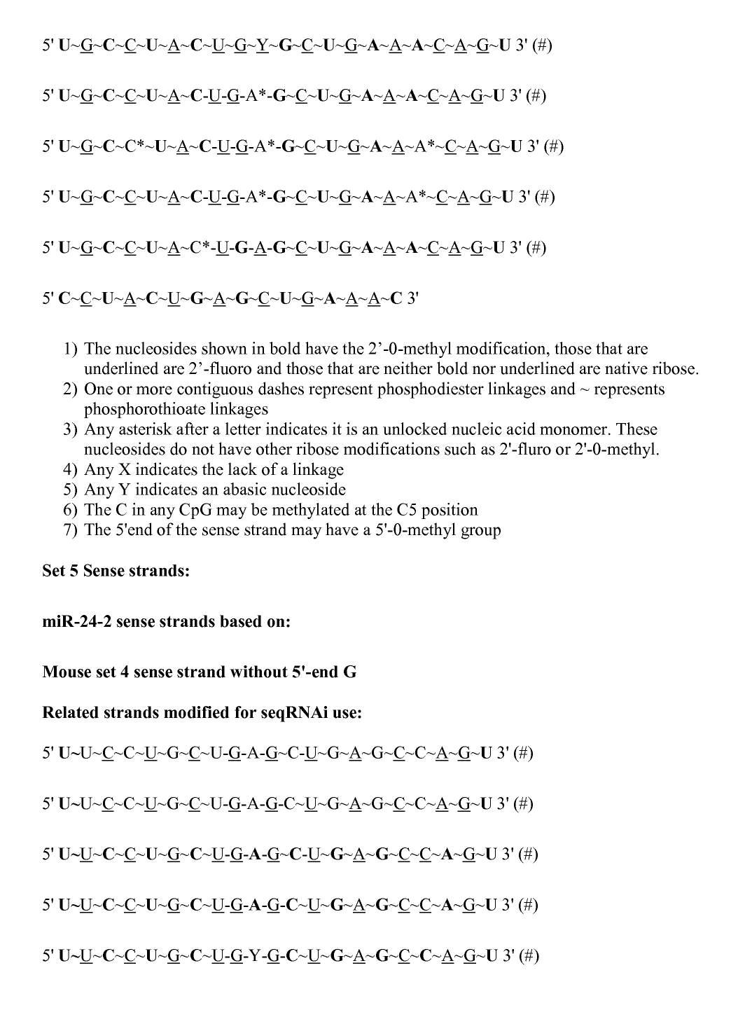

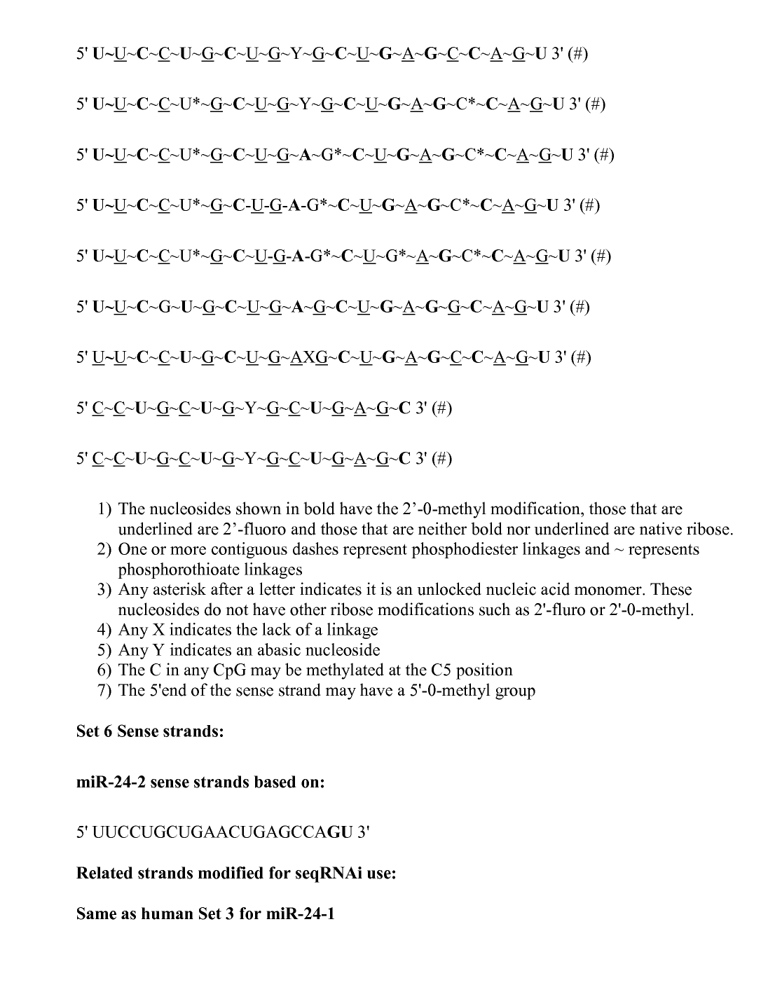

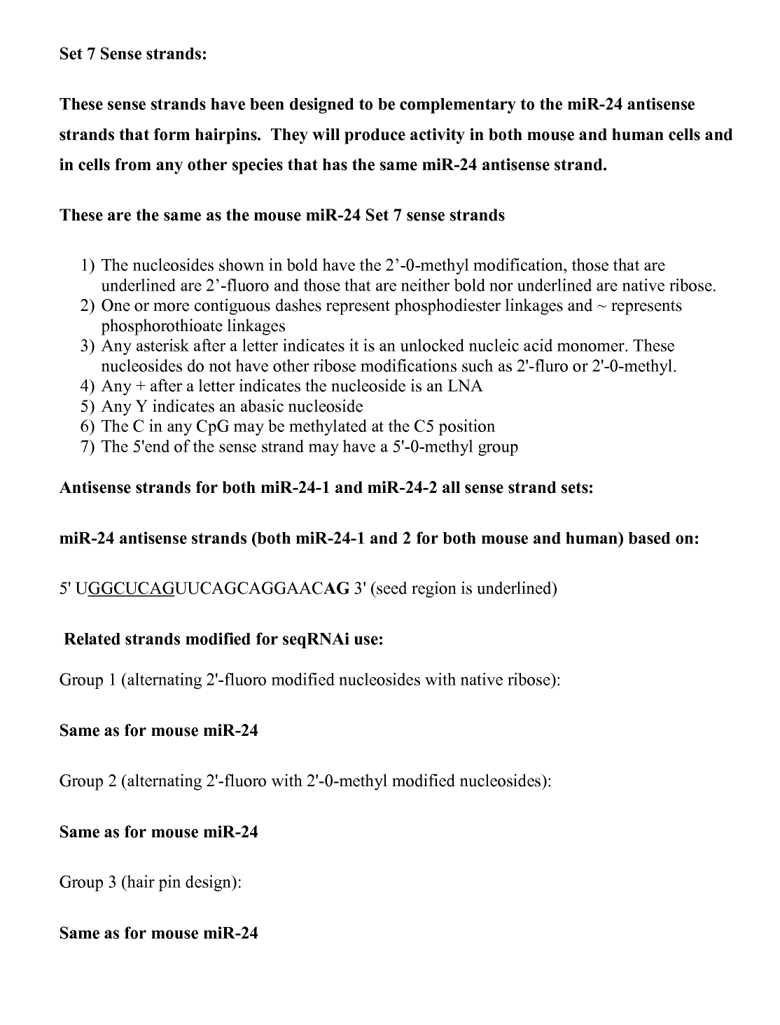

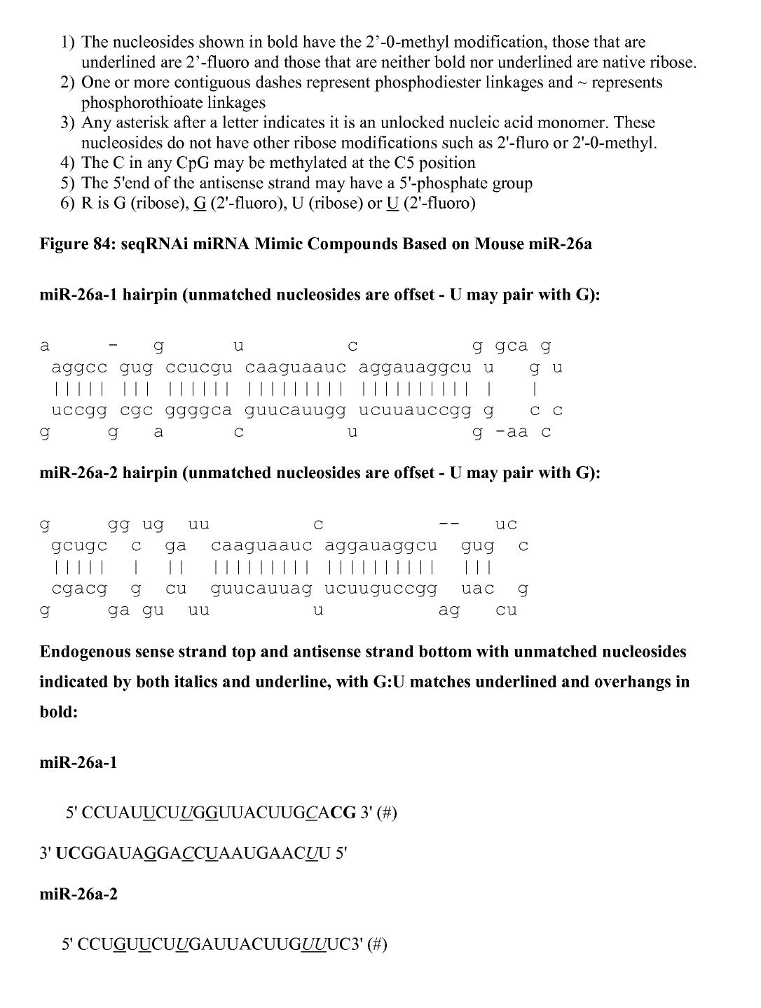

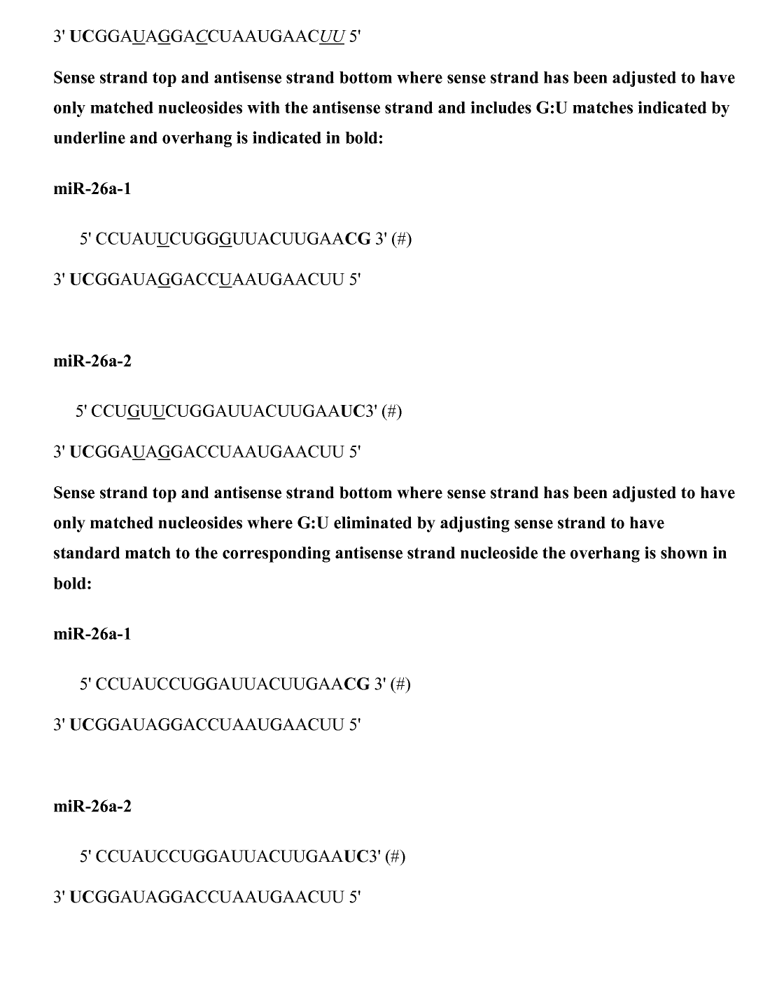

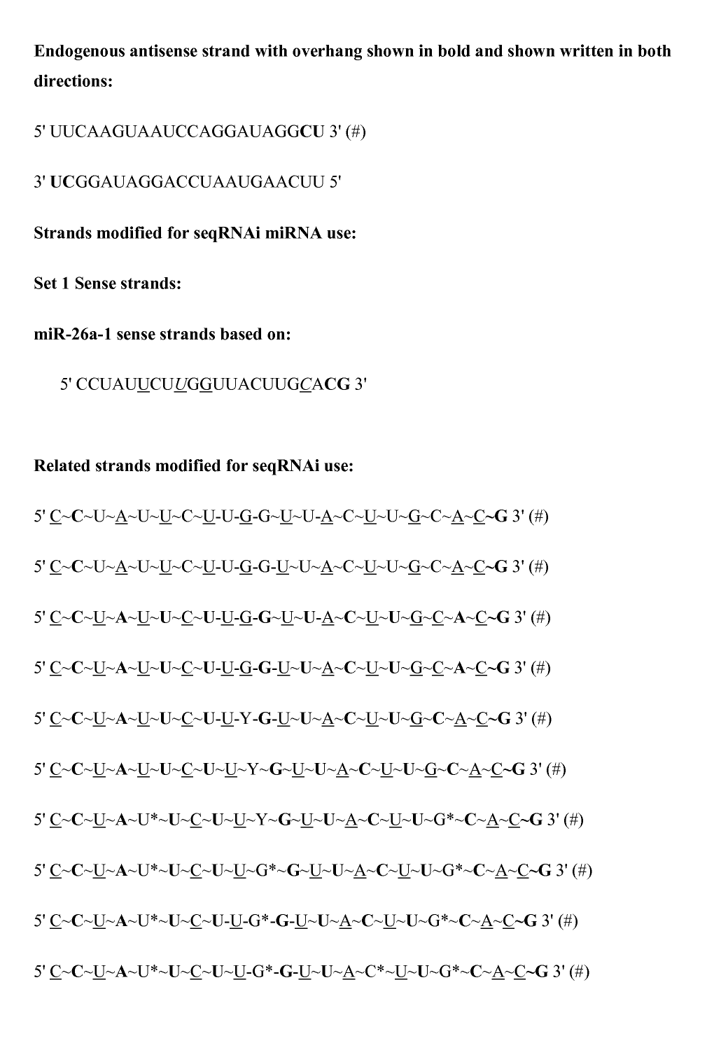

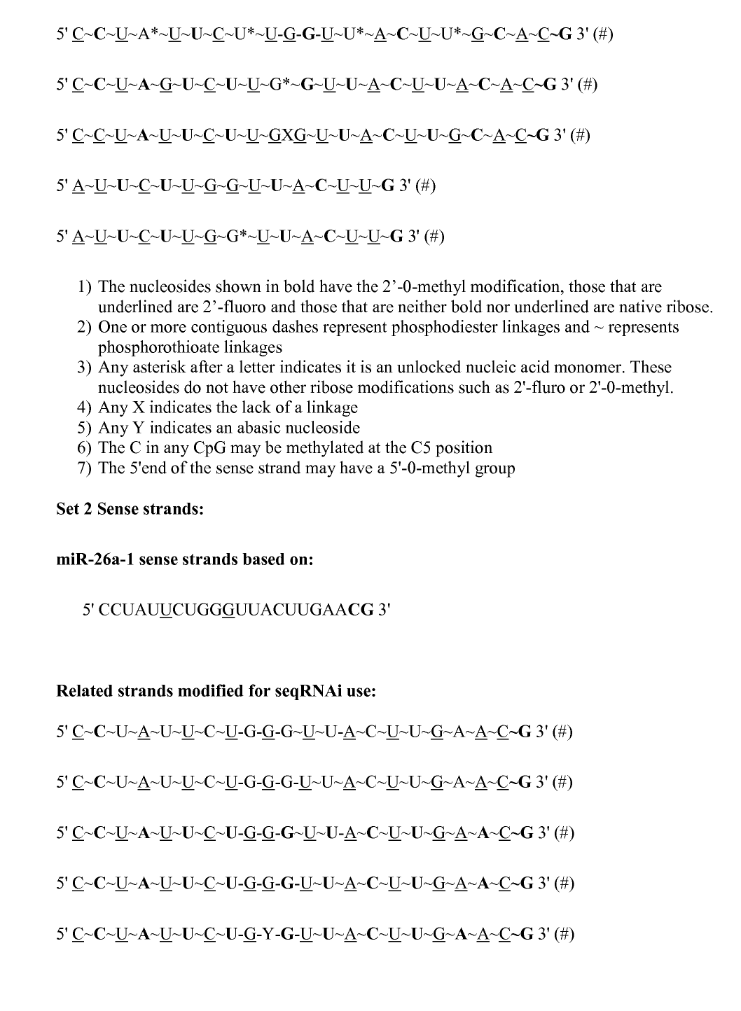

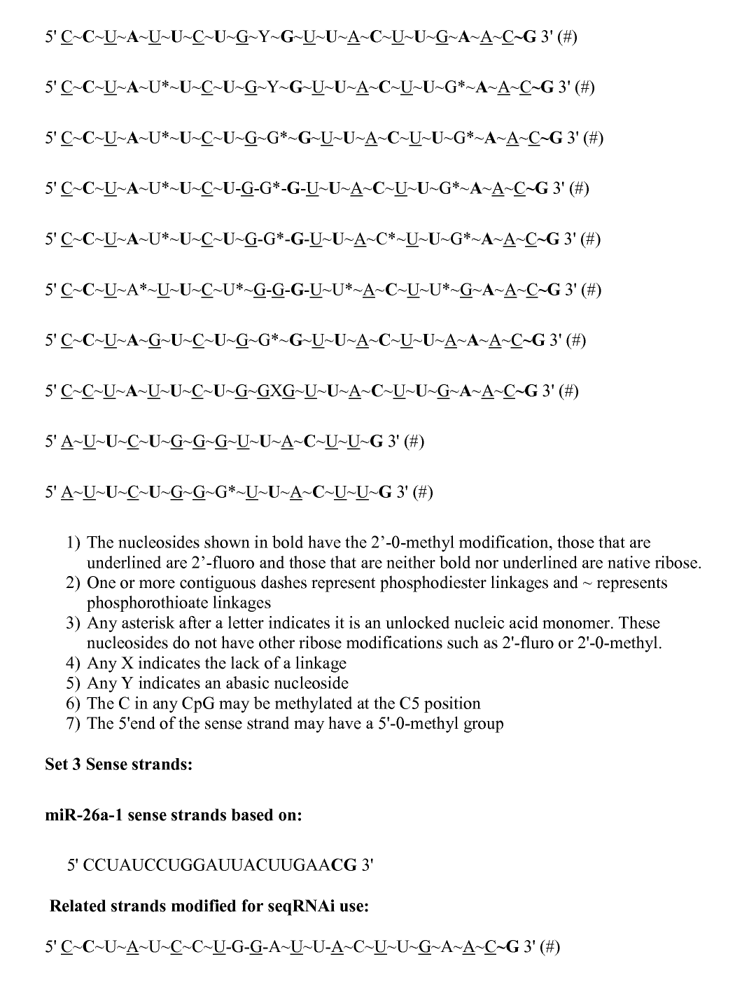

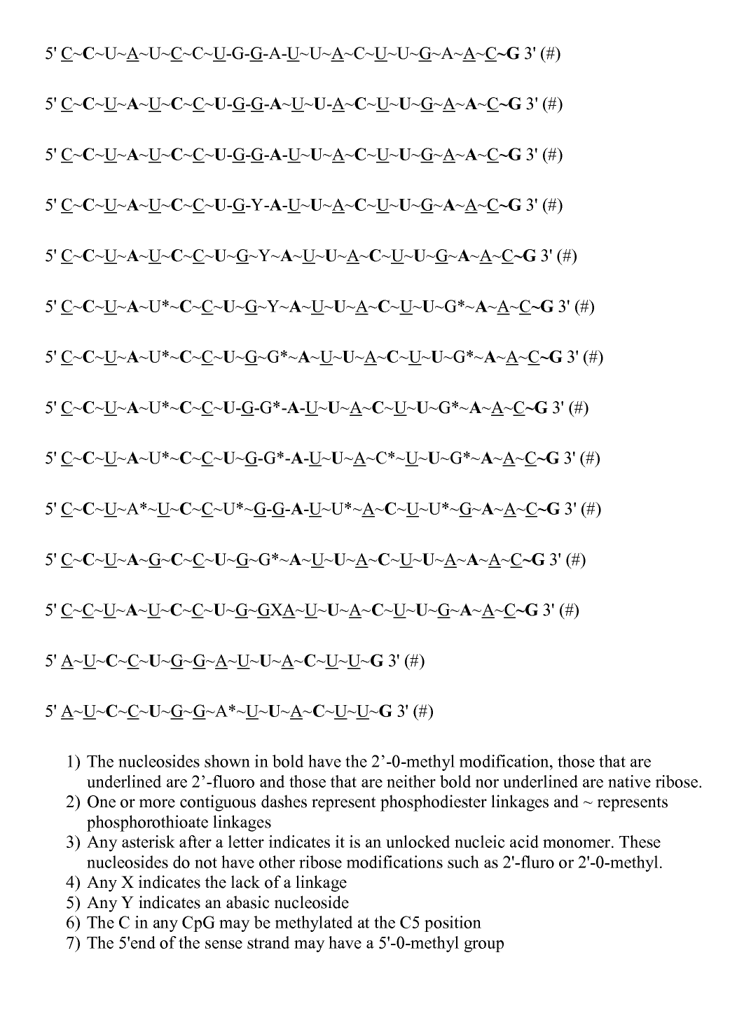

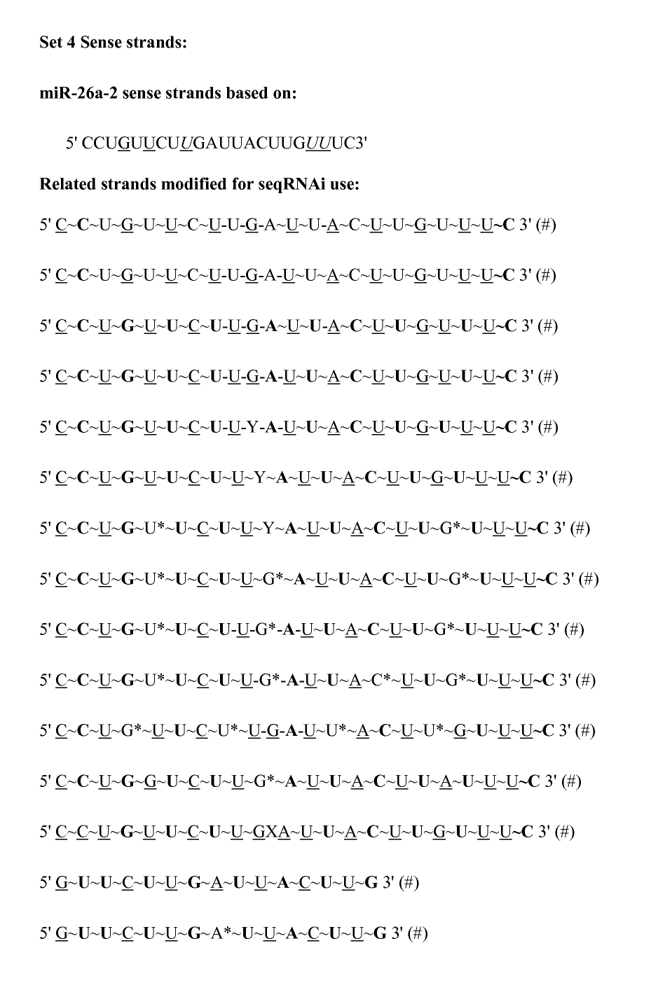

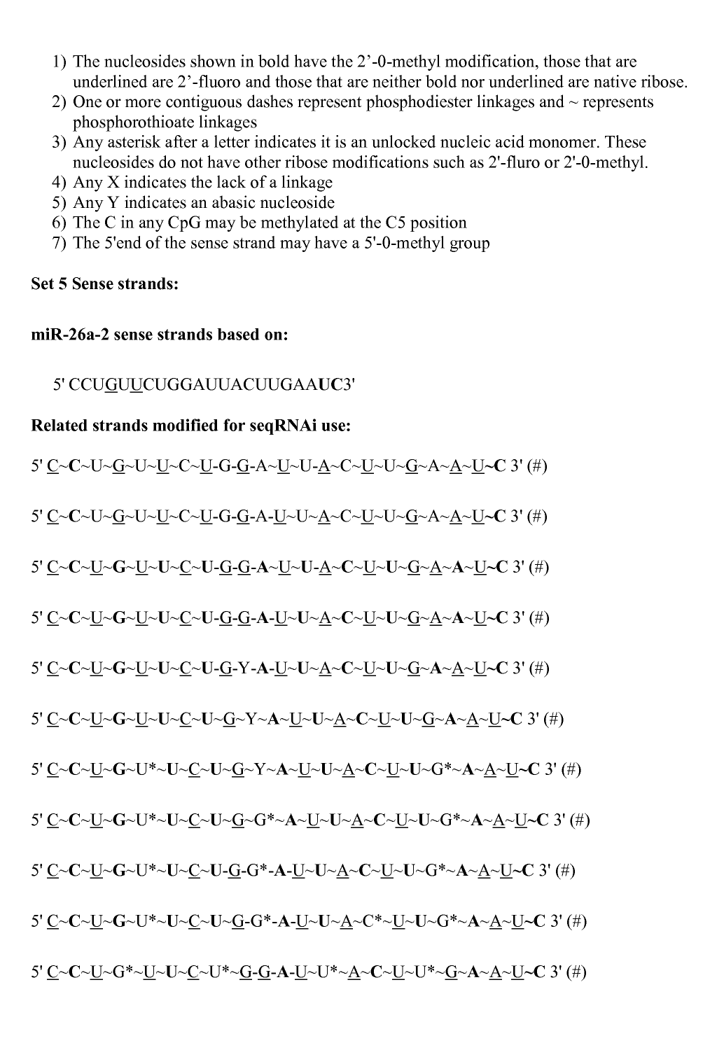

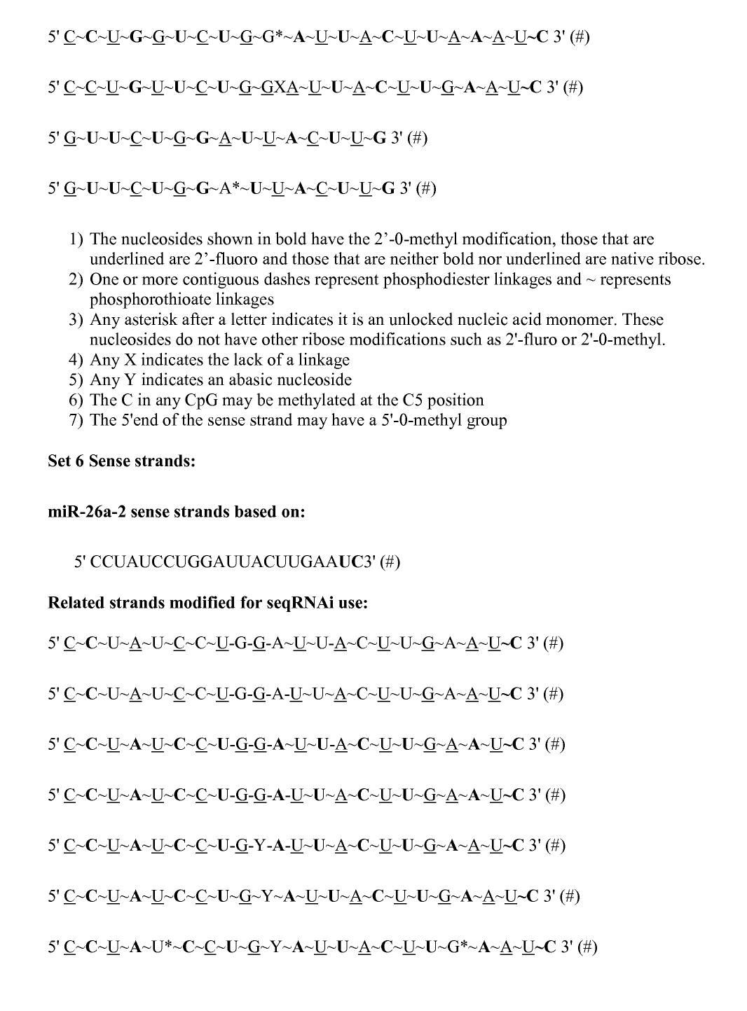

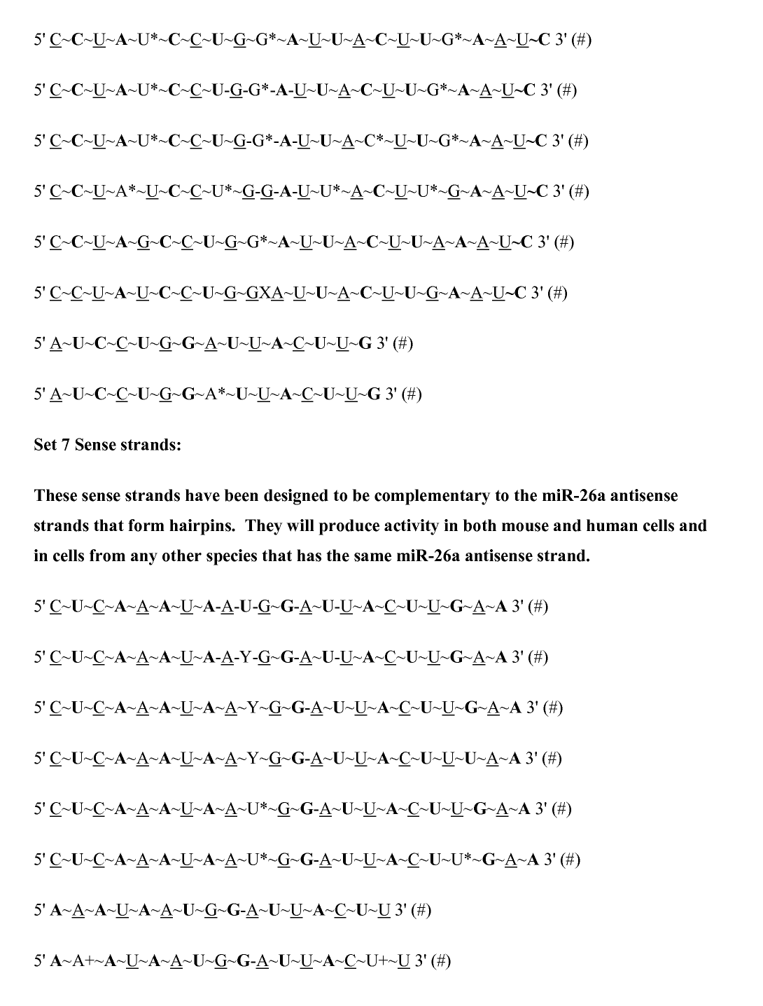

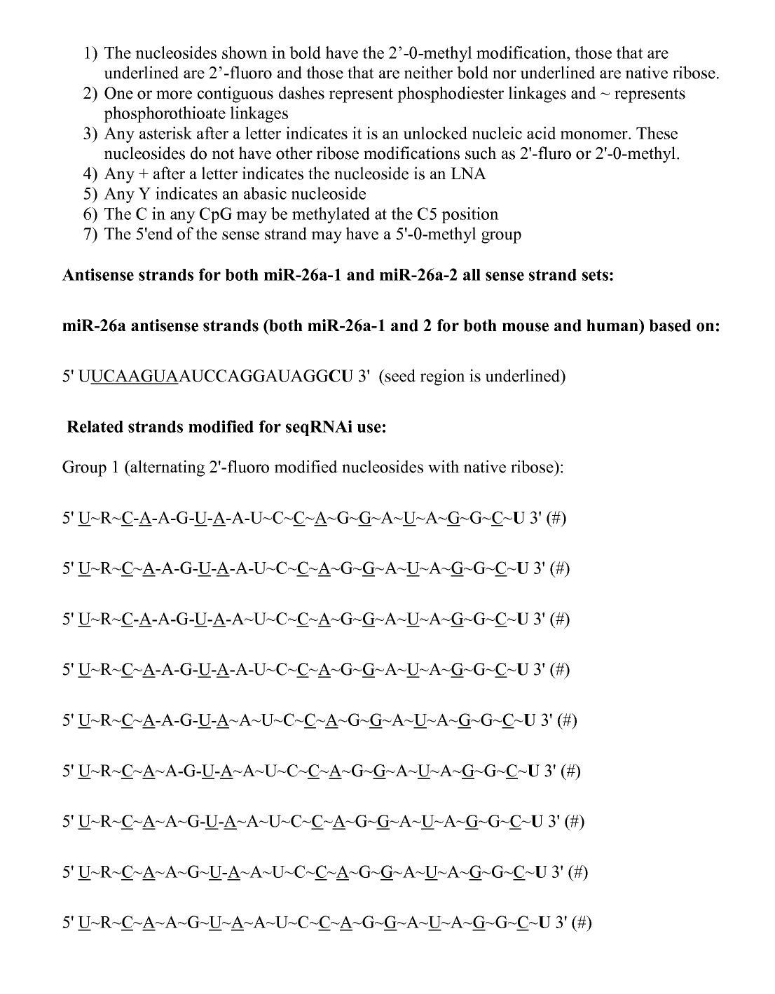

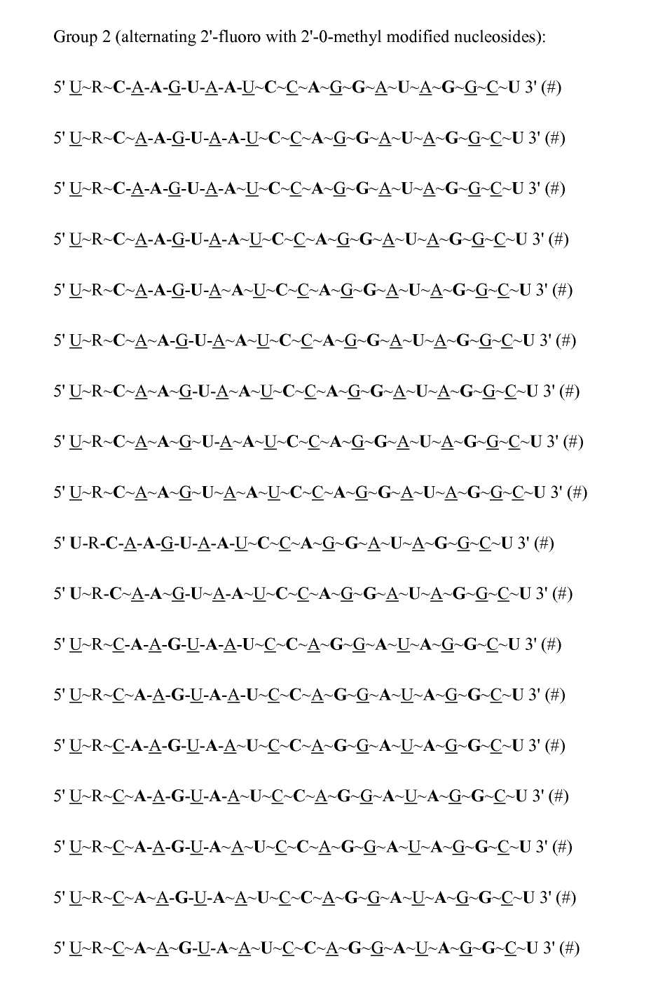

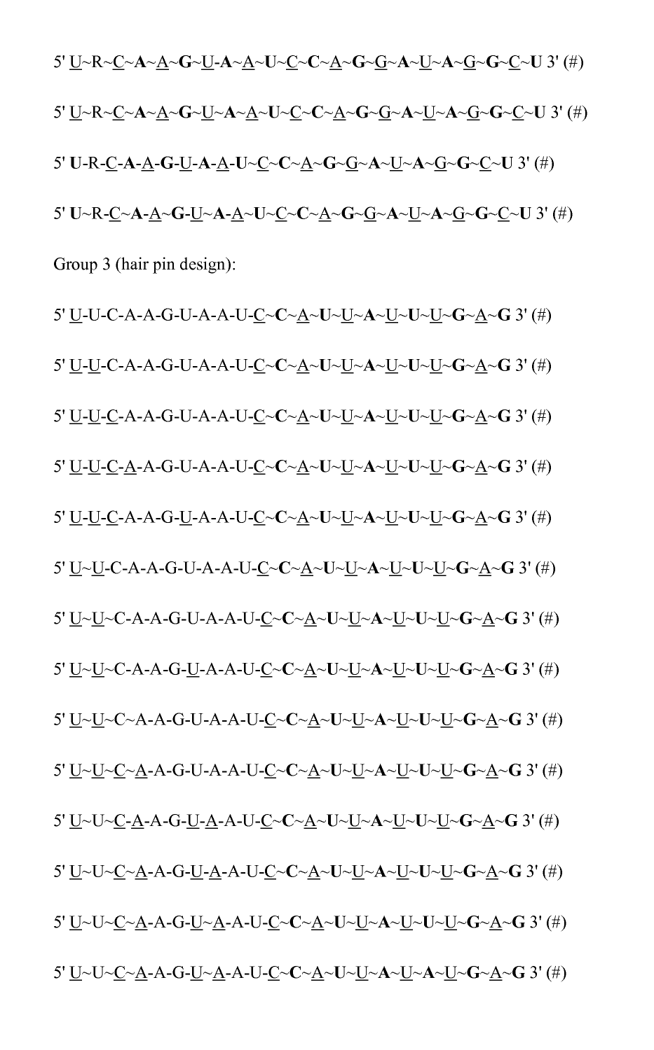

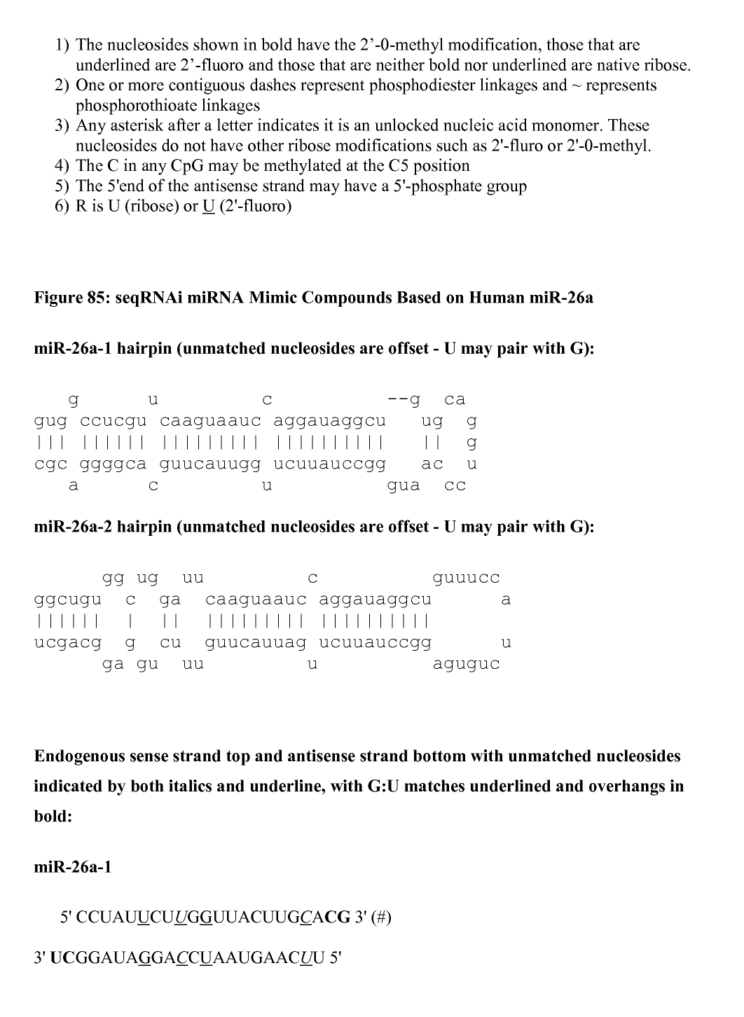

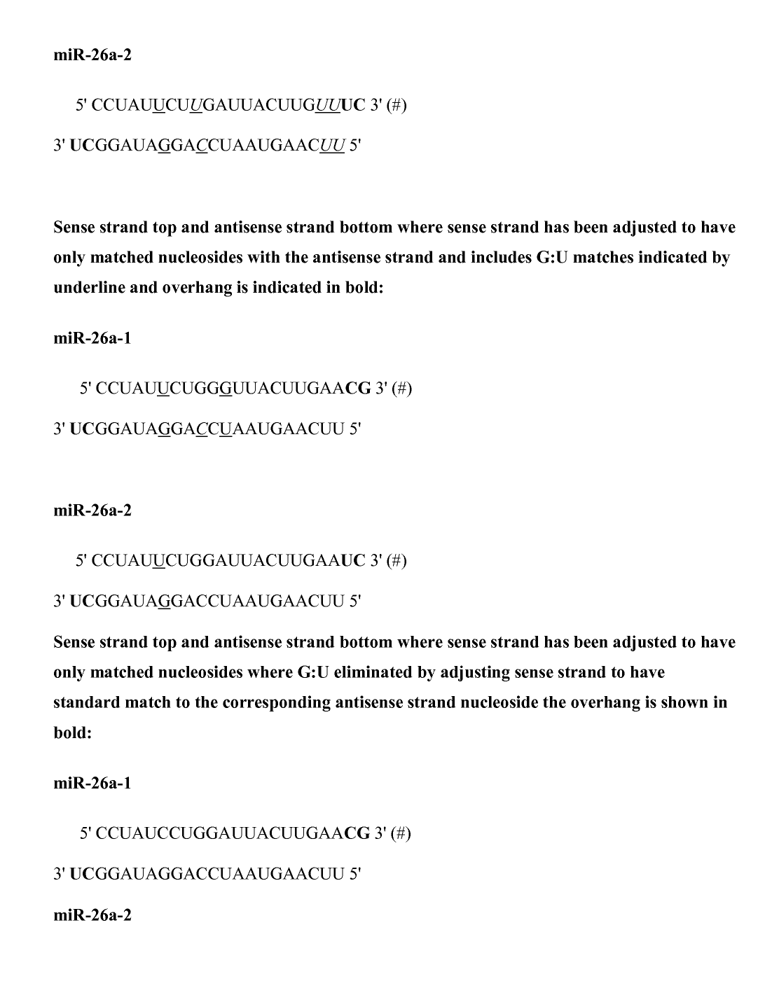

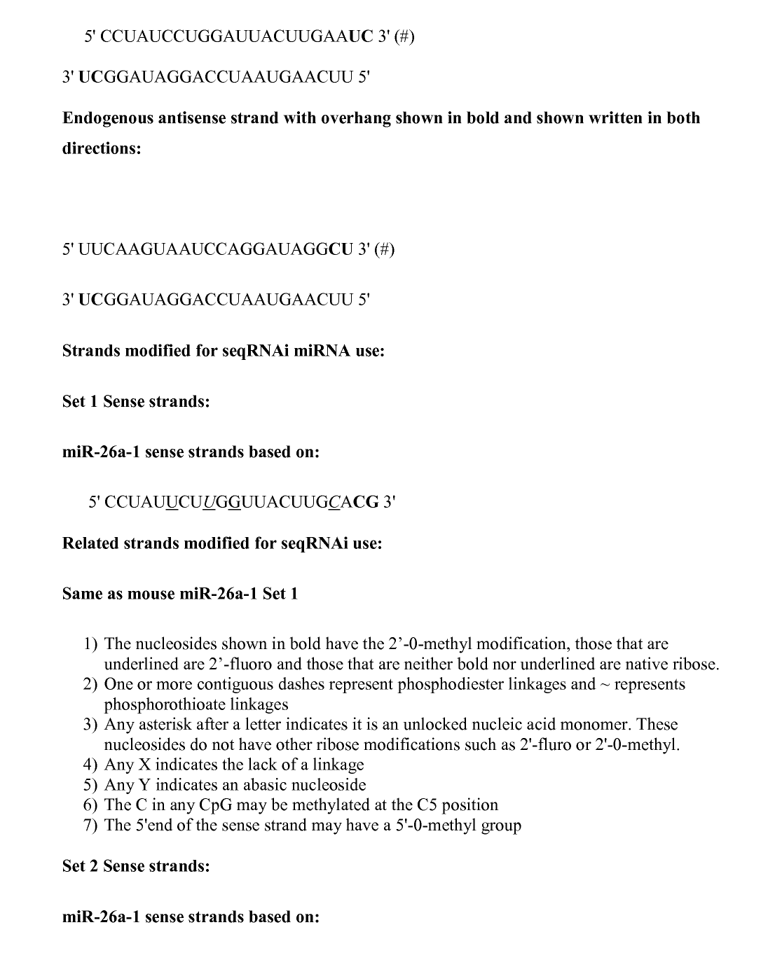

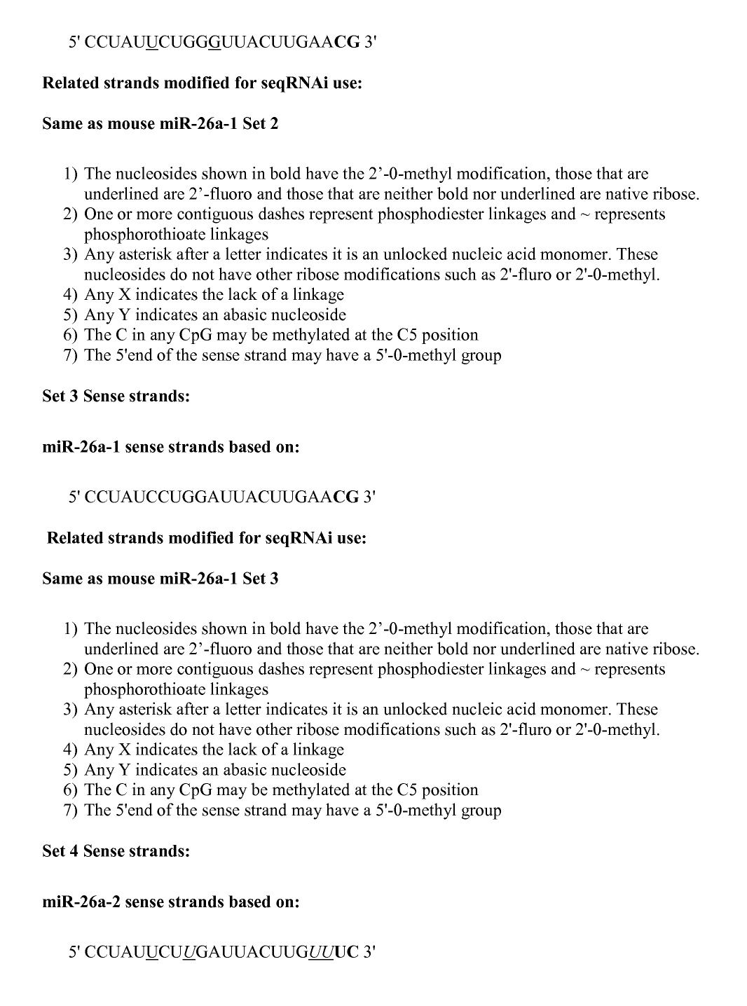

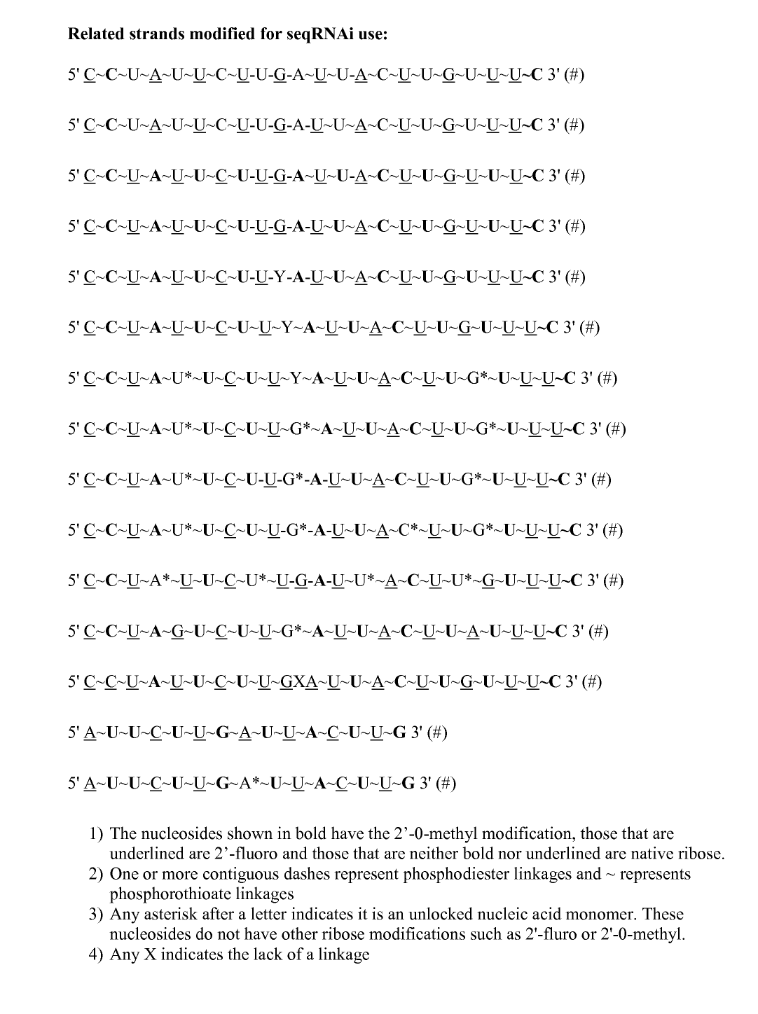

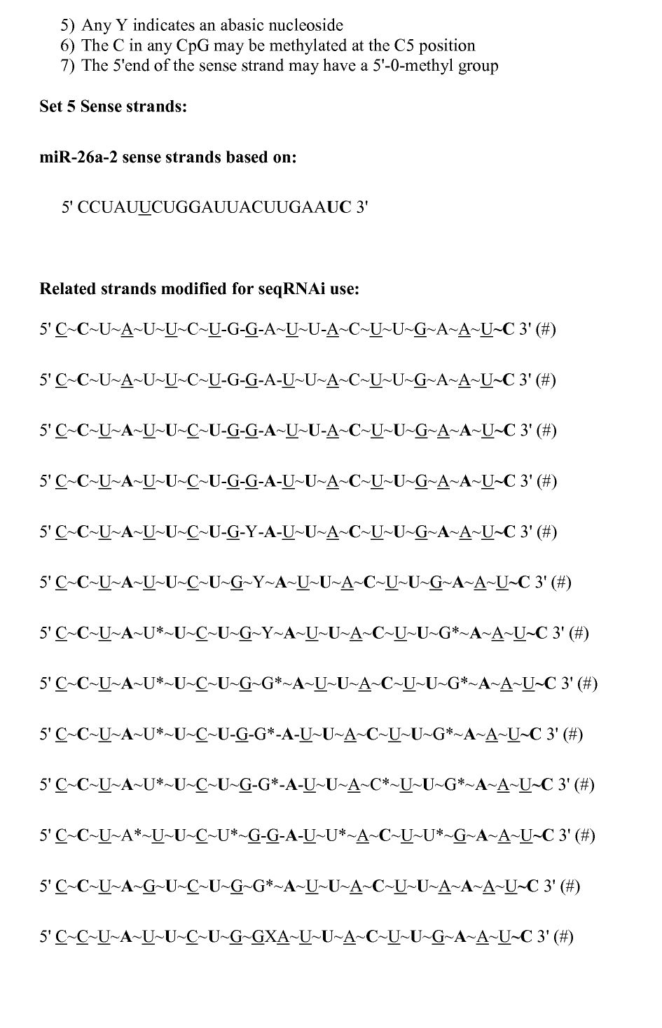

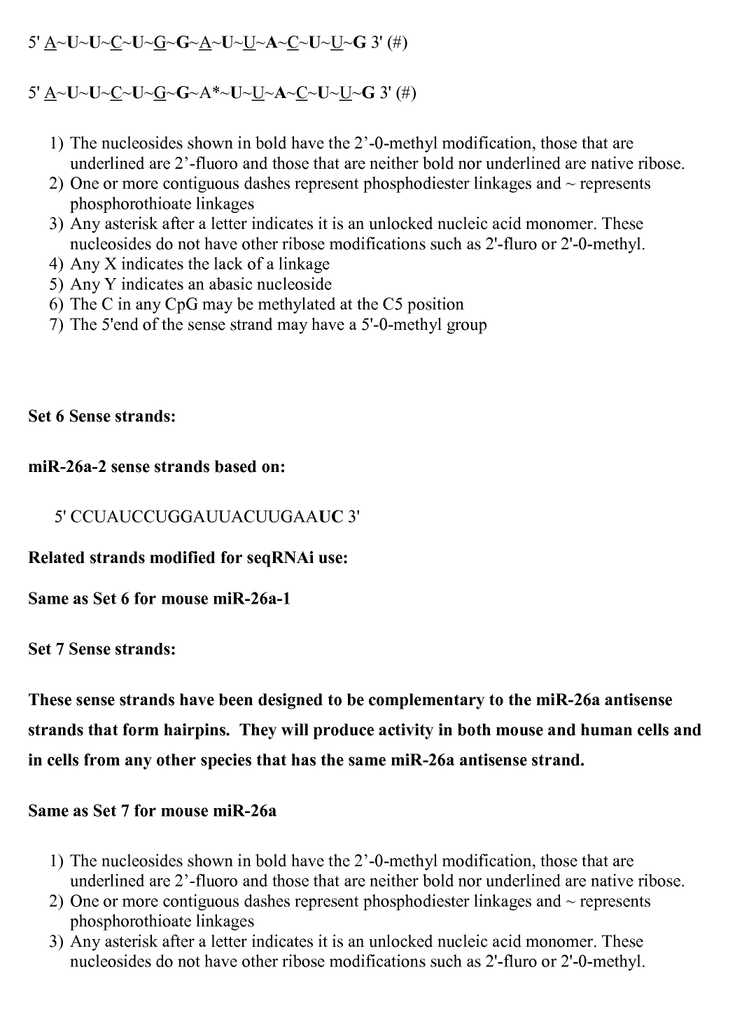

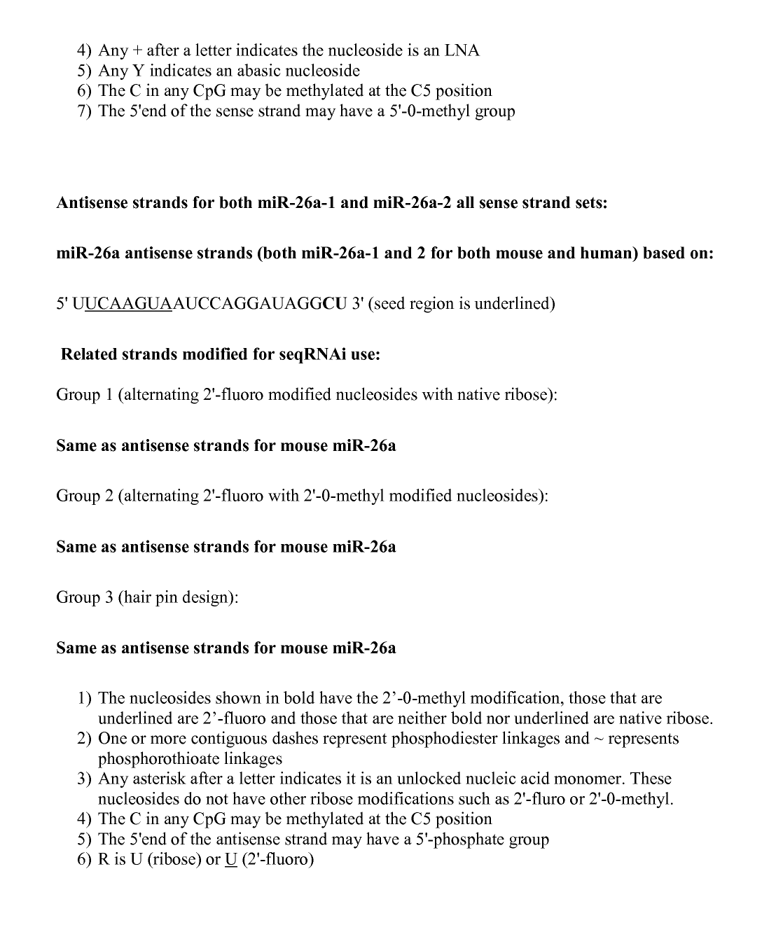

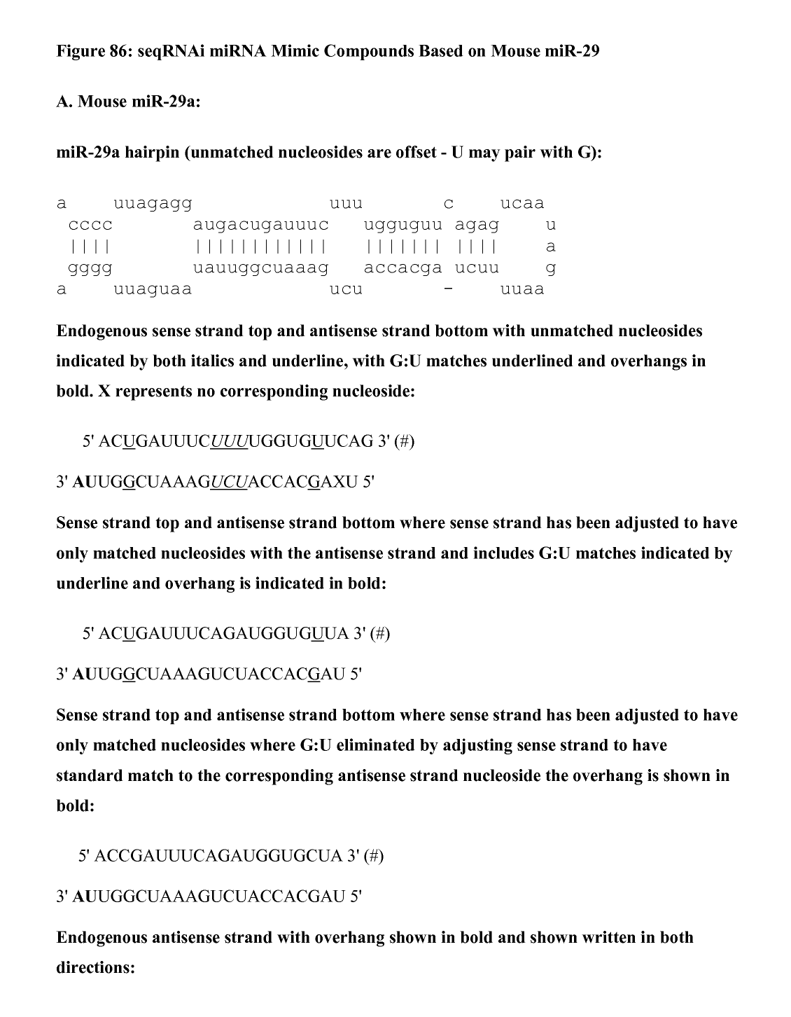

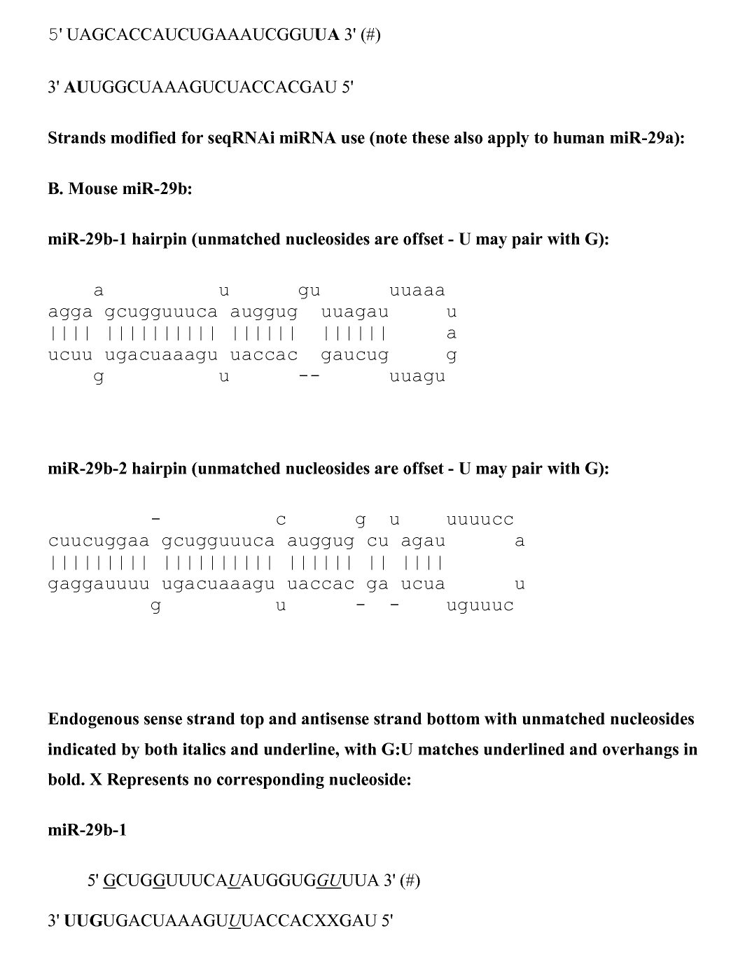

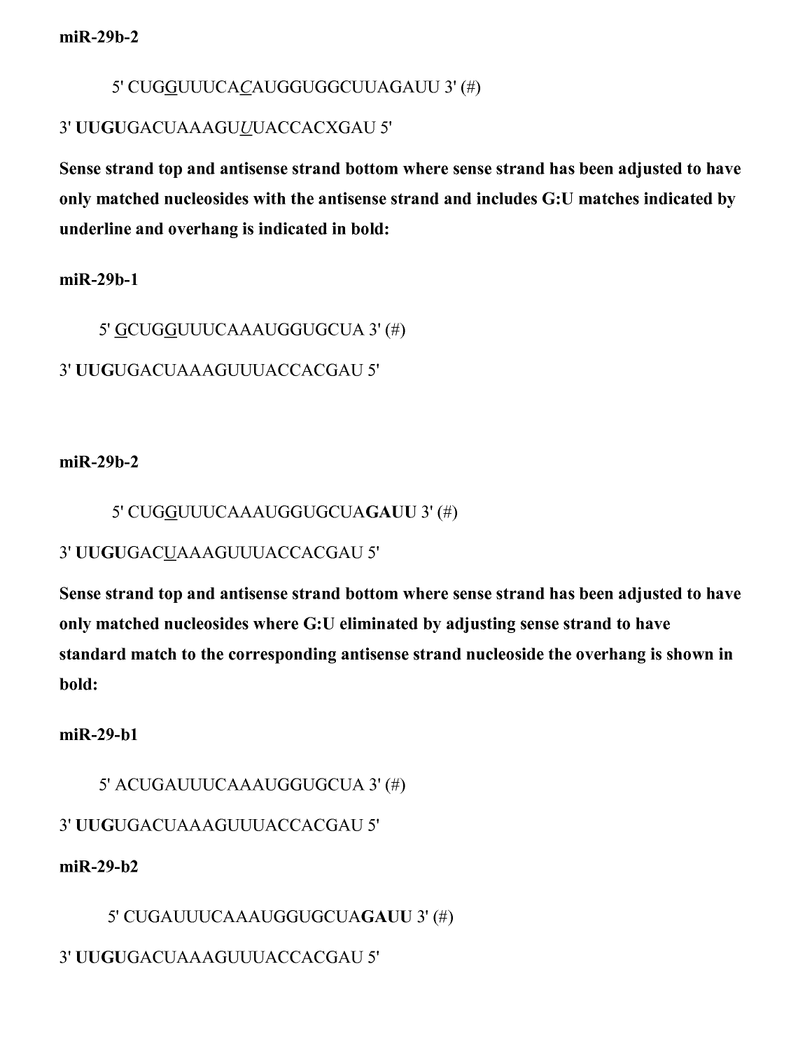

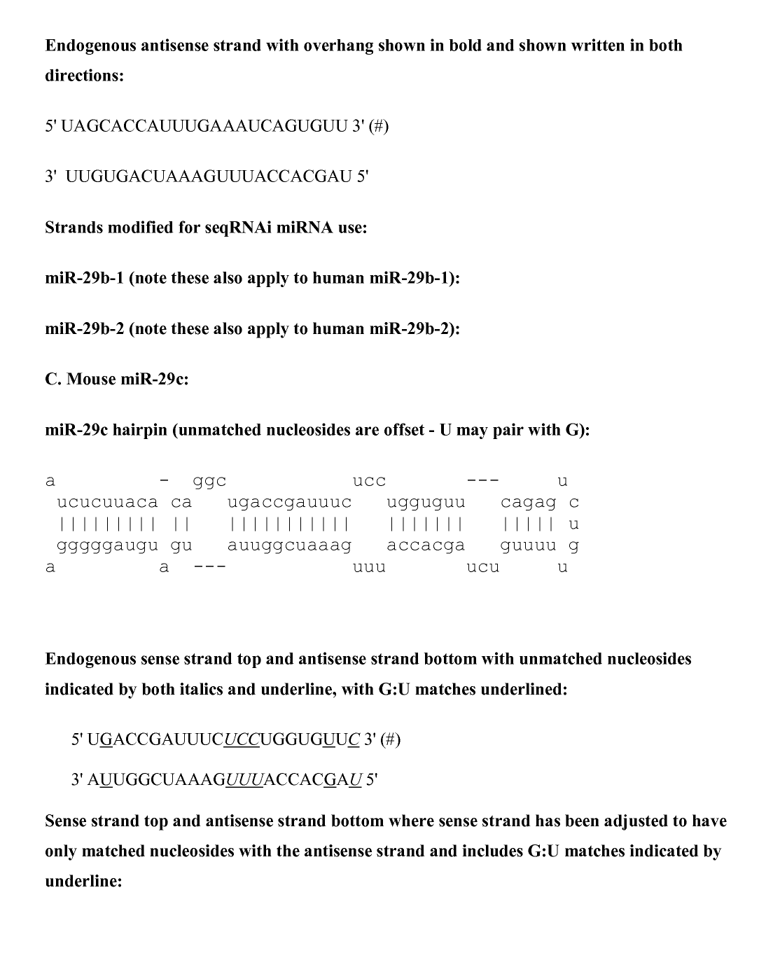

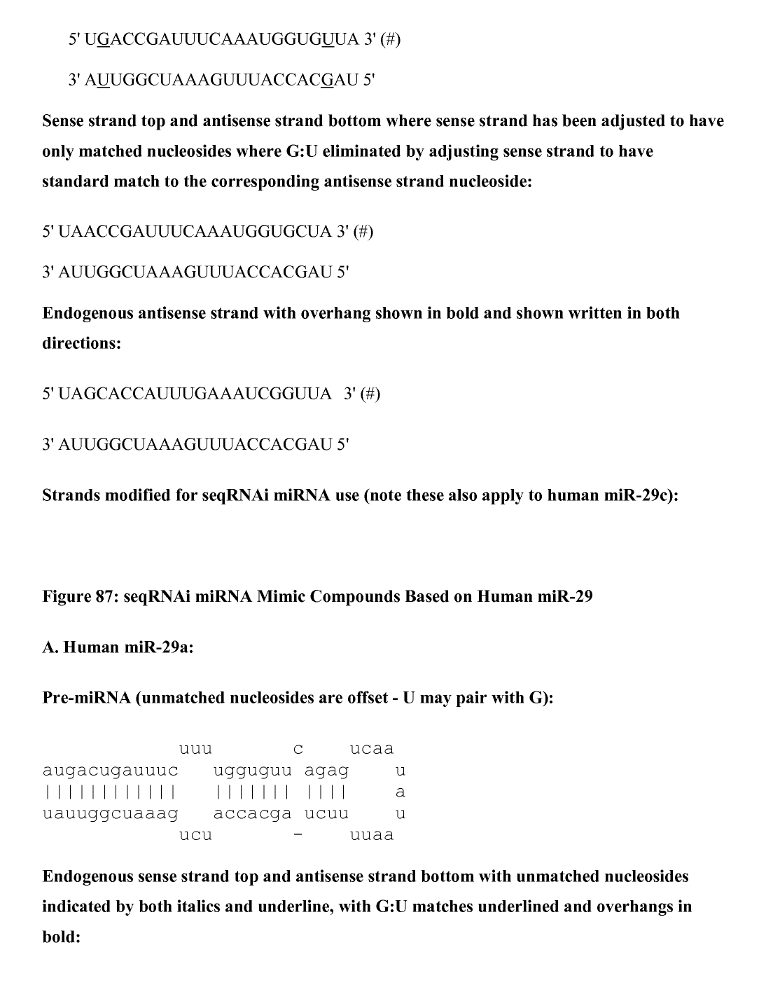

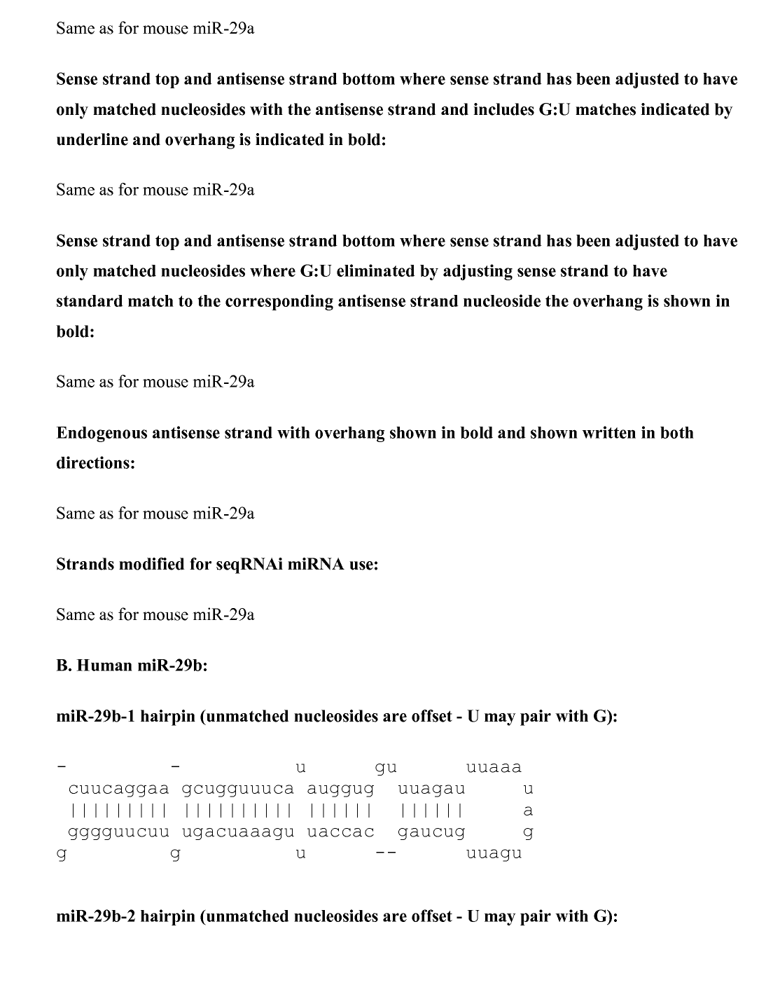

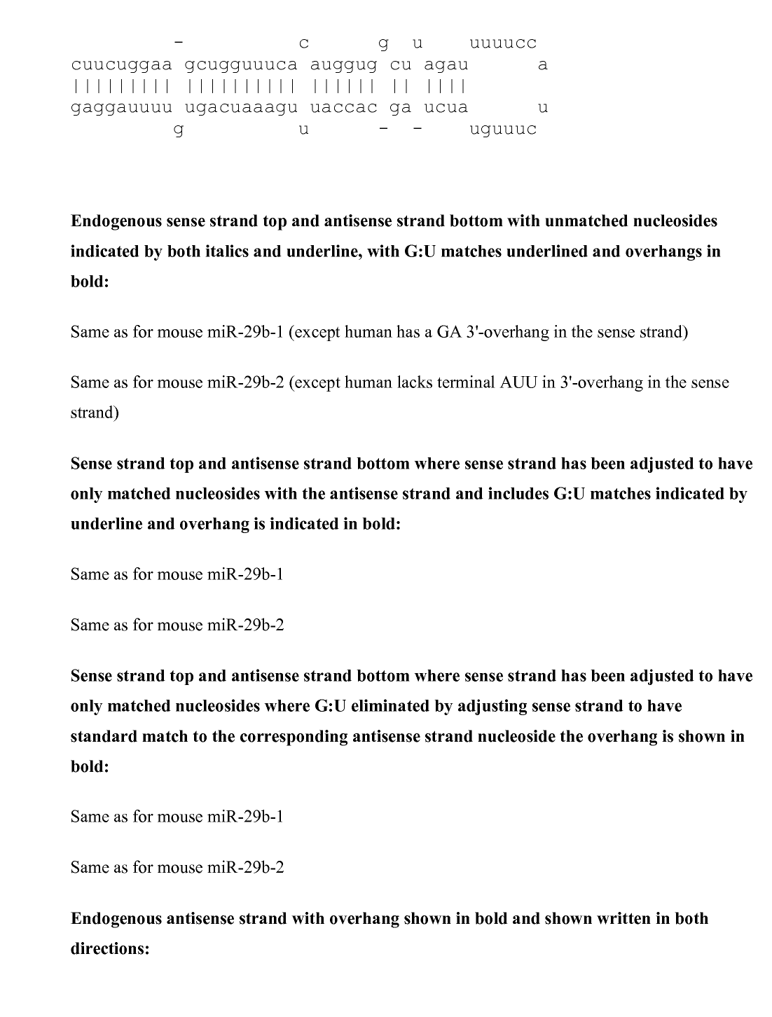

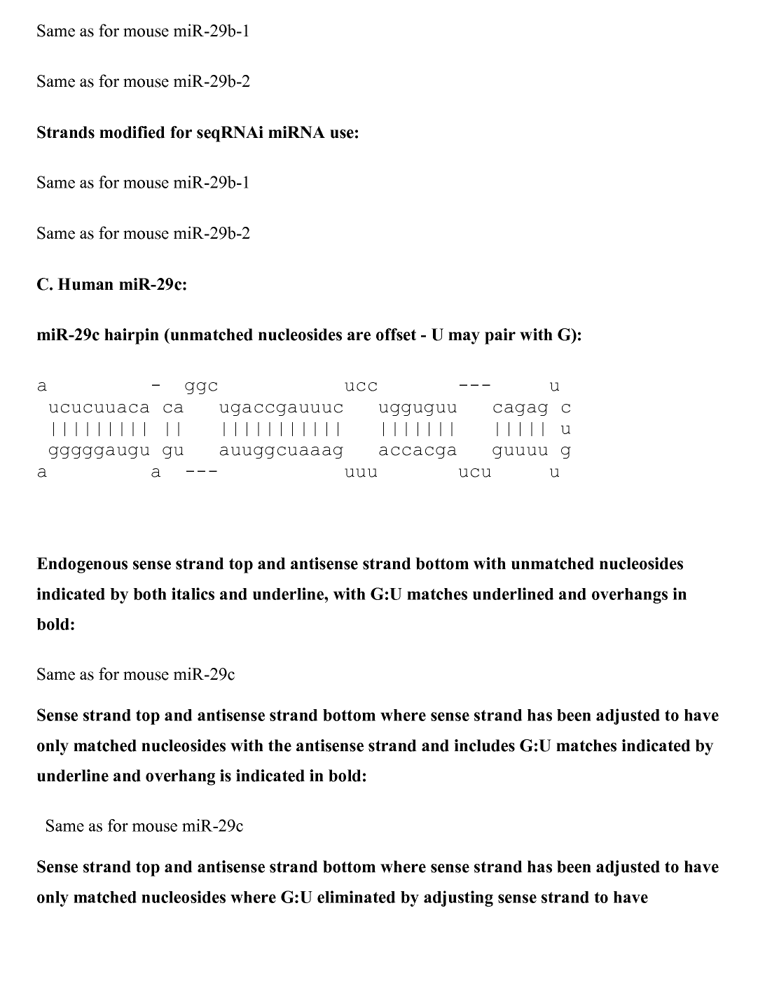

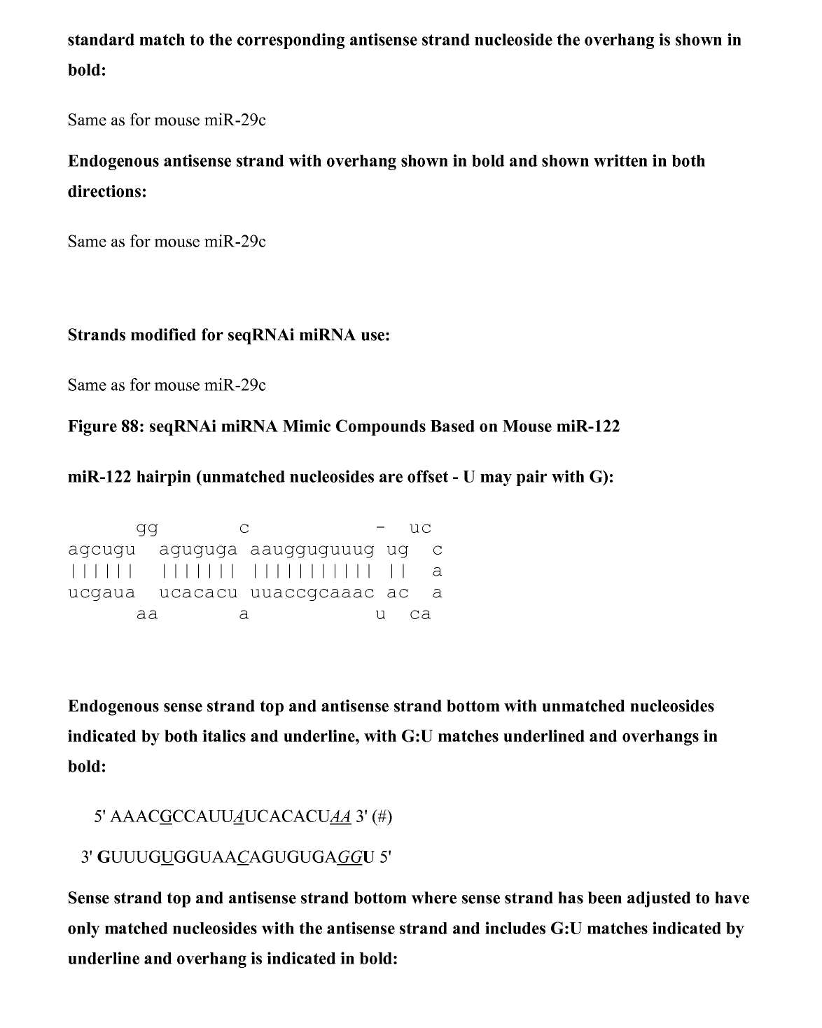

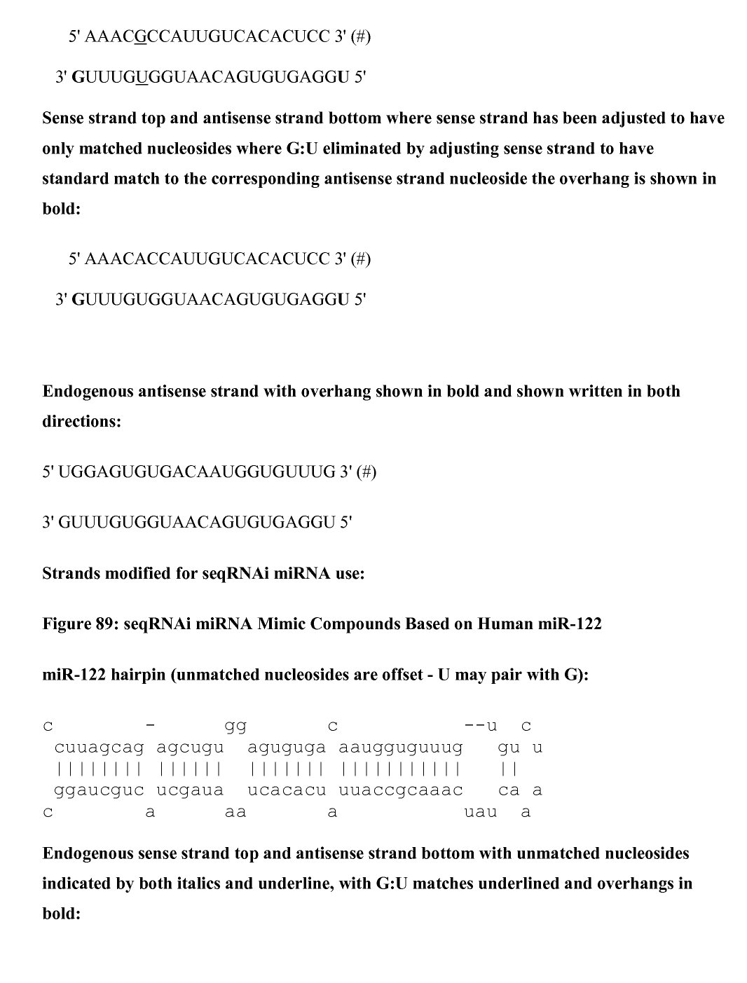

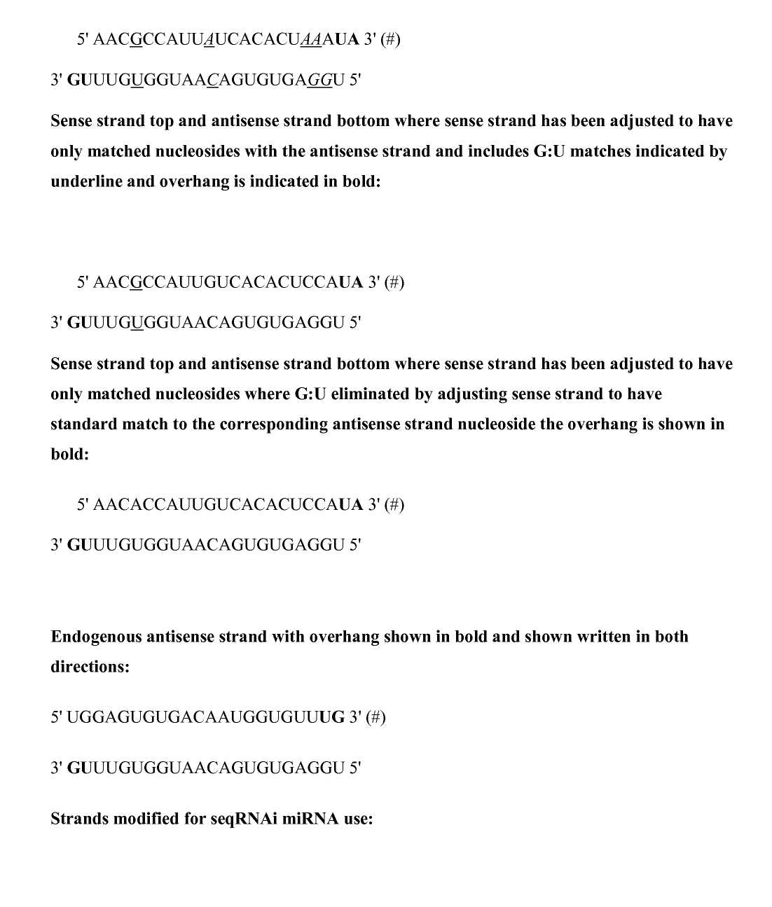

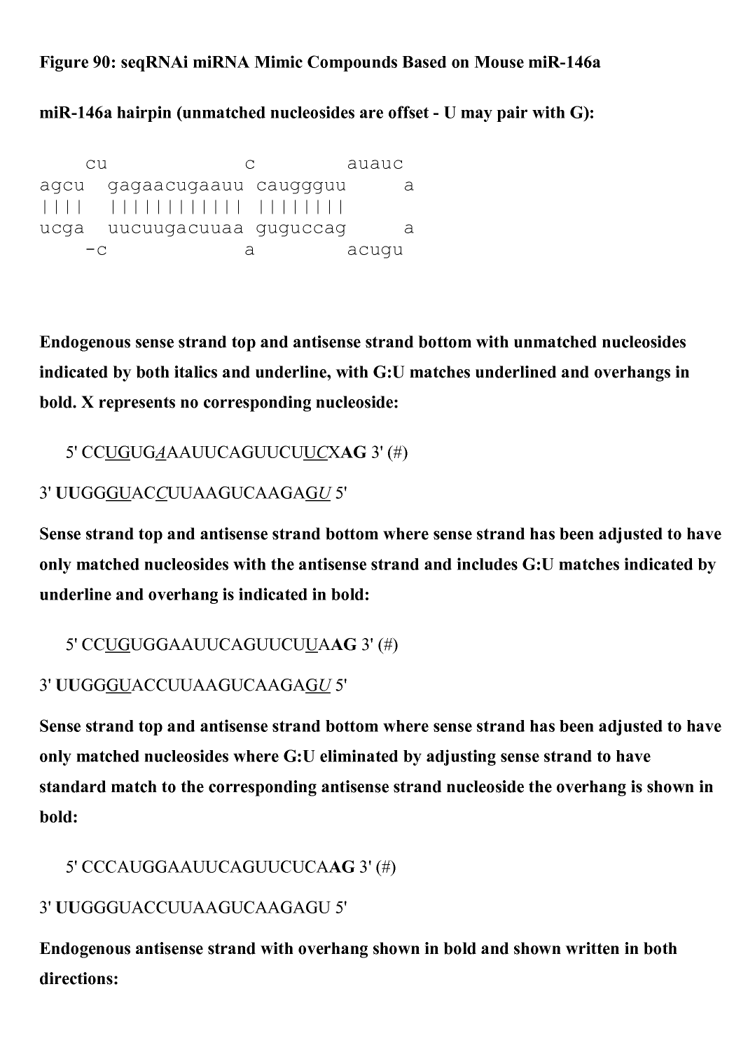

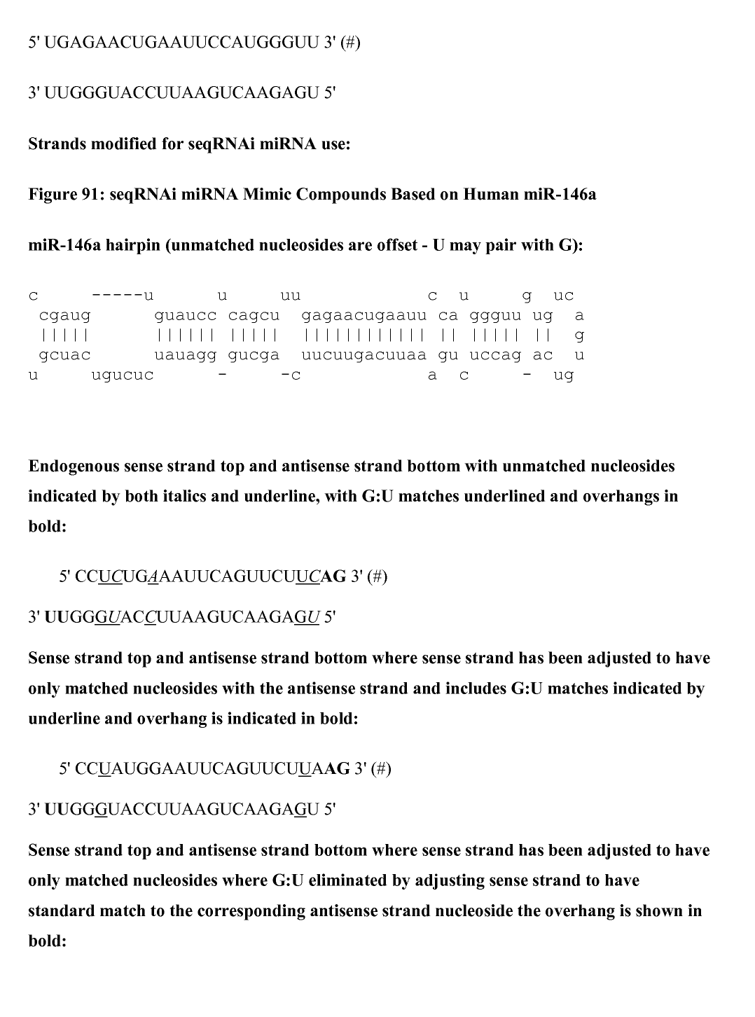

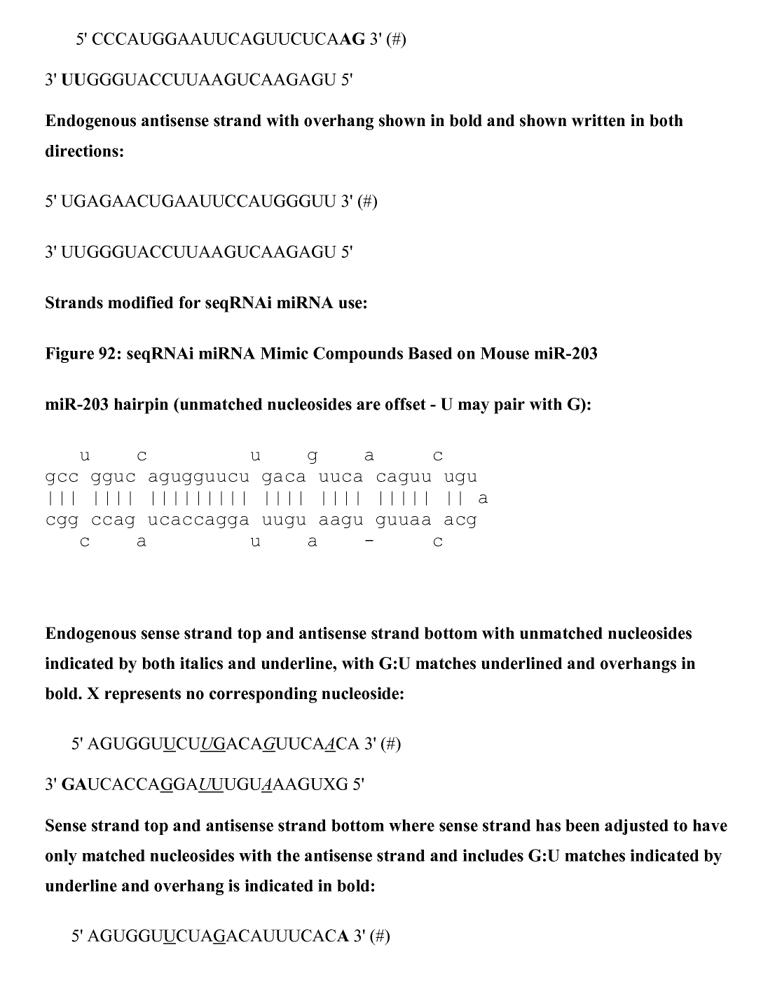

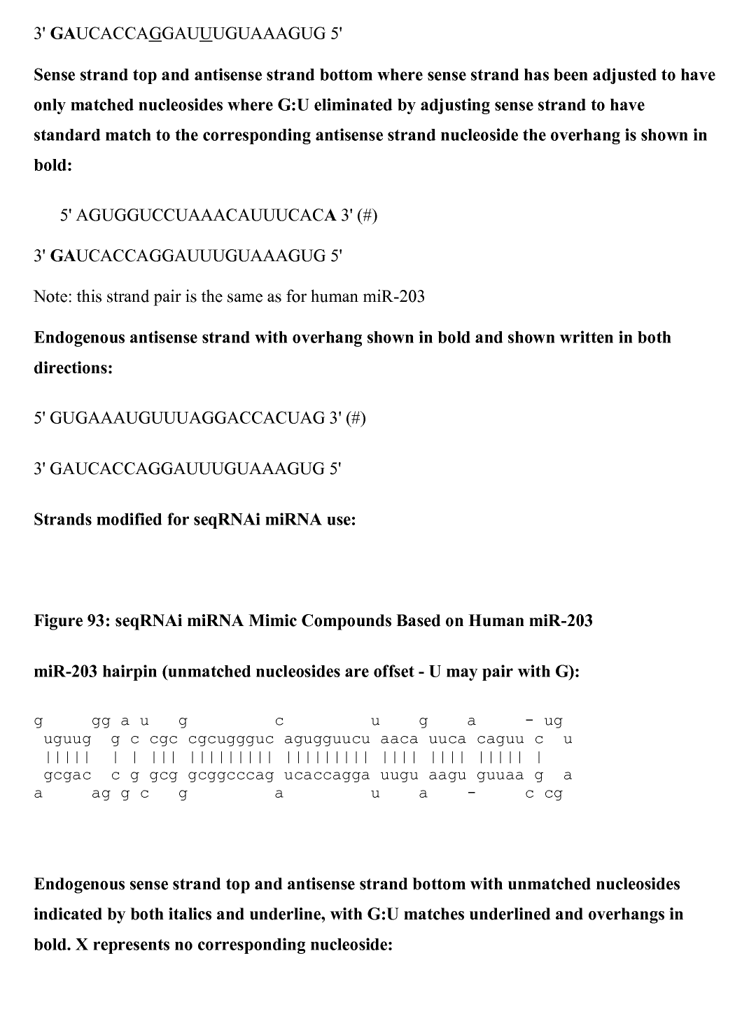

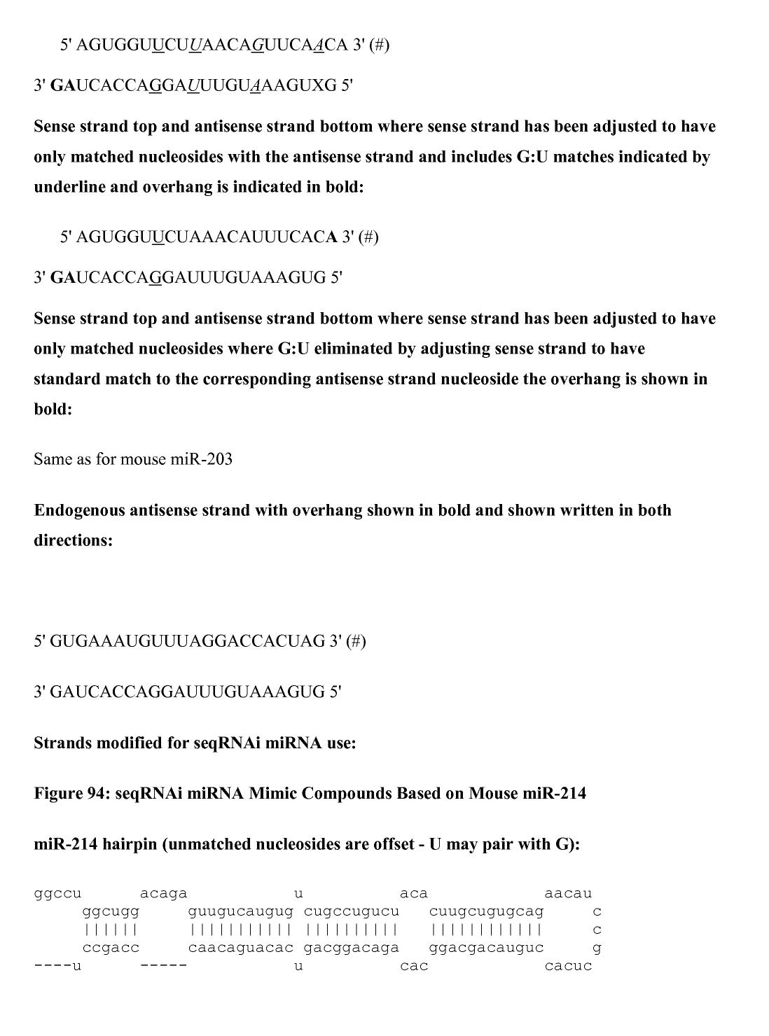

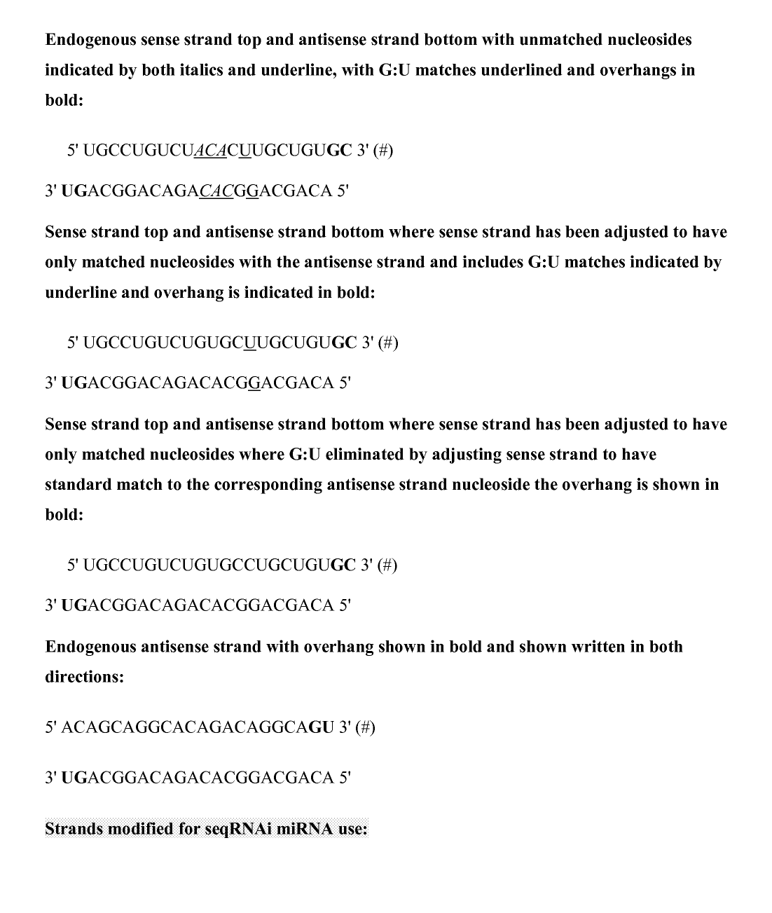

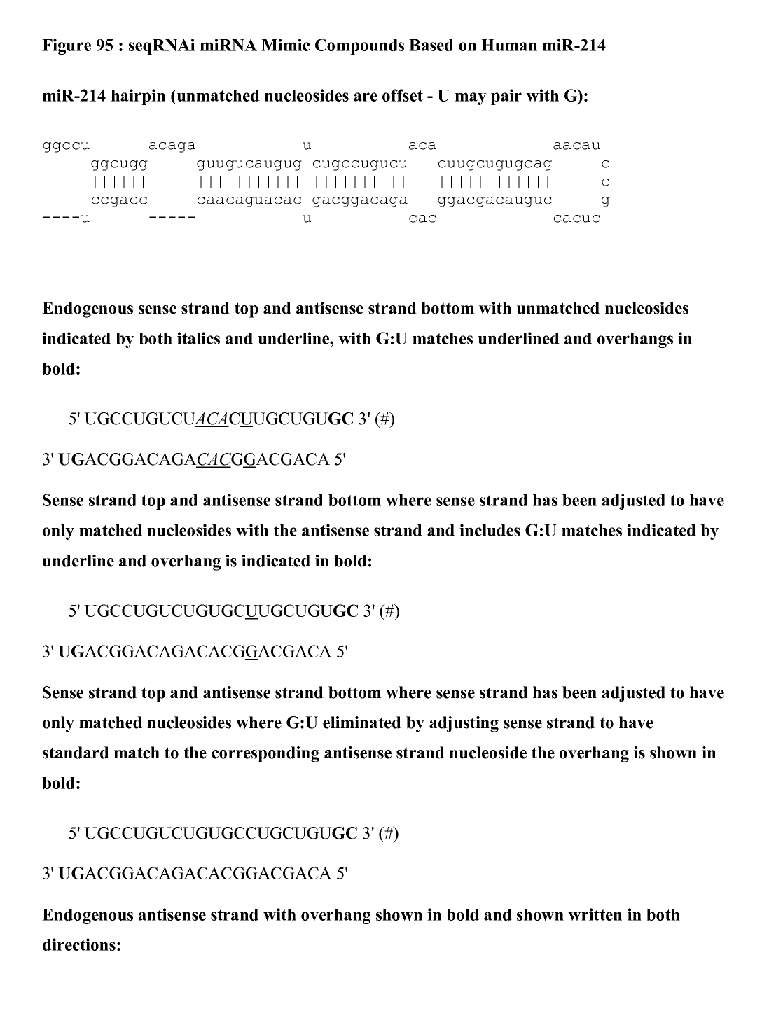

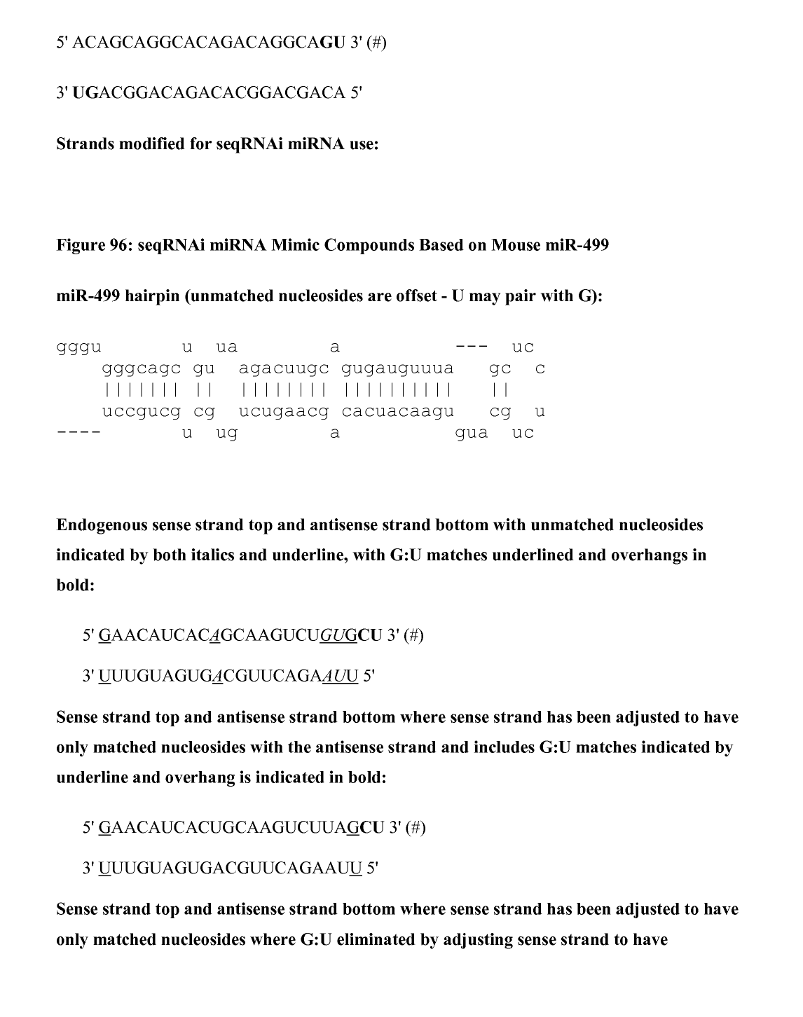

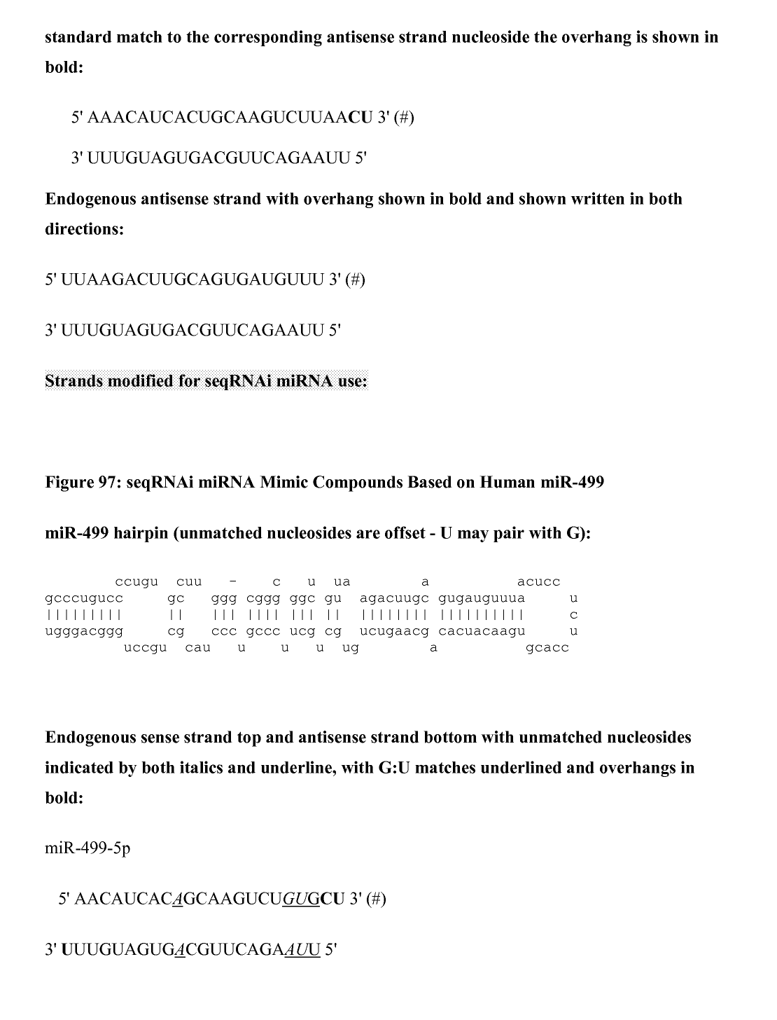

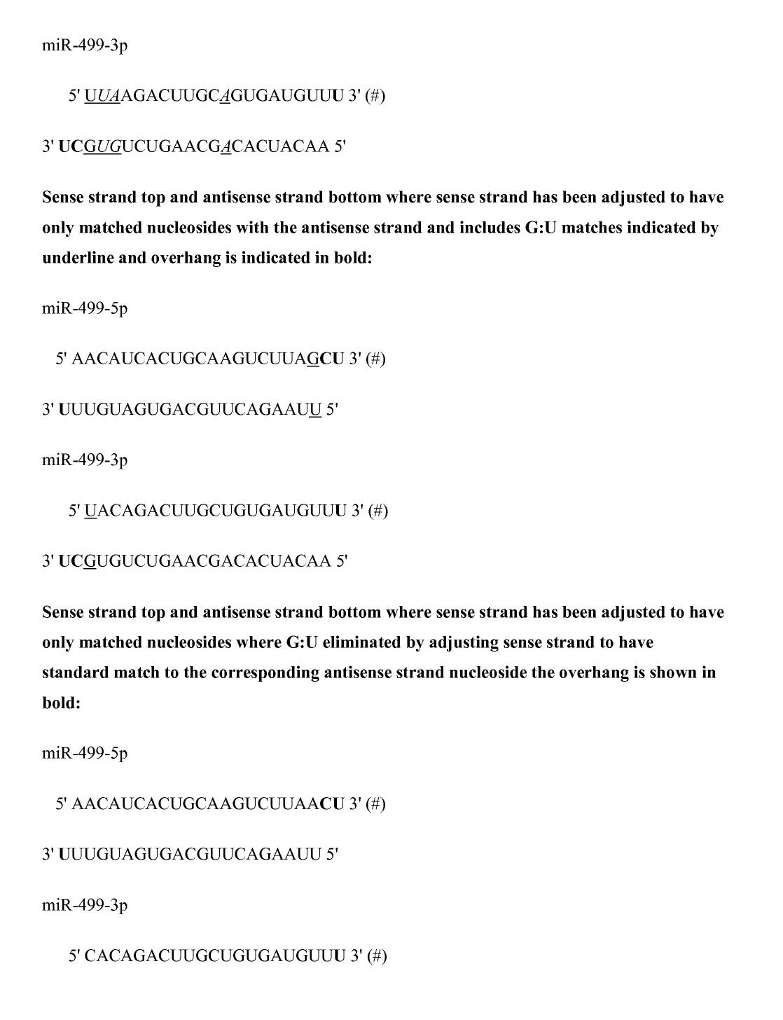

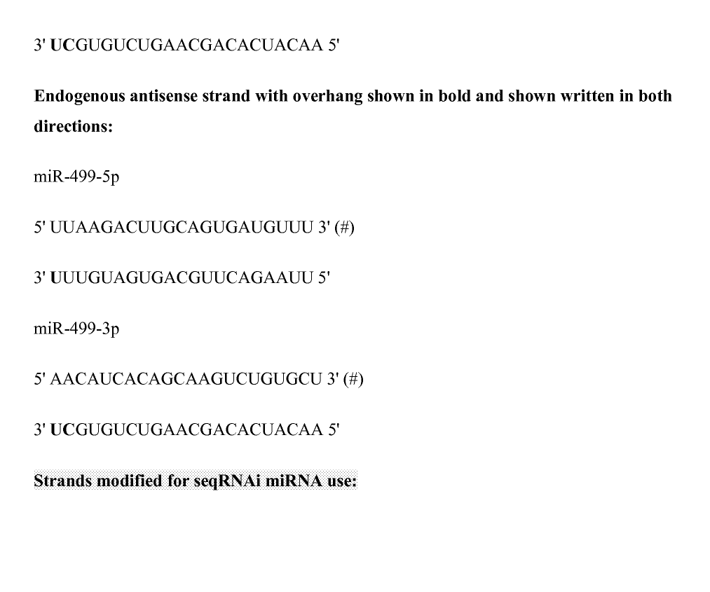

A number of conventional miRNA compounds closely based on their endogenous miRNA counterparts are in development as possible therapeutic agents. Cancer is one area of focus since it has been found that several different miRNAs are expressed at very low levels in cancer cells compared to their normal counterparts. Further, it has been shown that replacing these miRNAs can have profound anticancer effects. Several specific examples are provided in the Table. FIGS. 2, 9, 11, 13, 15, 17, 19 and 82-97 provide a variety of different seqMiR compounds, including potential anticancer agents that are based on the endogenous miRNAs shown in Table 8 that should be useful for the treatment of the indicated conditions.

The miRNA mimics provided should also be effective in cell culture in vitro. In this approach, the first strand can be transfected into the target cells following by subsequent transfection of the second strand after a certain time frame has elapsed. This method should facilitate drug discovery efforts, target validation and also provide the means to reduce or eliminate any undesirable off target effects.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A nucleic acid based compound which inhibits expression of a target sequence in a cell within a mammal, said nucleic acid based compound comprising at least one single stranded oligonucleotide modified to
i) increase nuclease resistance;
ii) bioavailability; and
iii) inhibitory activity within said cell;
said compound being deliverable in vivo in single stranded form in a vehicle, wherein said compound is selected from the group consisting of
a) compounds shown in the Figures or
b) duplexes of the compounds of a),
said duplexes being formed intracellularly following sequential administration of single strand oligonucleotides in vivo, said duplexes being effective cause RNAi dependent silencing of expression of said gene product upon intracellular duplex formation;
said compounds exhibiting enhanced silencing activity in said target cell in vivo relative to nucleic acid compounds which lack said modifications.

2. The compound of claim 1, wherein said target sequence is selected from the group consisting of a mRNA, a plurality of mRNA molecules or a miRNA molecule

3. The compound of claim 1, wherein said compound triggers AGO-2 cleavage of a target mRNA sequence.

4. The compound of claim 1, wherein said compound triggers degradation of at least one therapeutically relevant miRNA.

5. The compound of claim 1, which mimics the action of an endogenous therapeutically relevant miRNA.

6. The compound of claim 1, which forms an intracellular duplex of sense and antisense oligonucleotide strands after sequential administration of single strands, wherein said sense strand is 10 to 25 nucleosides in length and said antisense strand is 16 to 25 nucleosides in length.

7. The compound of claim 1, wherein said oligonucleotide comprises at least one modified sugar selected from the group consisting of 2′ fluoro, 2′ fluoro substituted ribose, 2′-fluoro-D-arabinonucleic acid (FANA), 2′-O-methoxyethyl ribose, 2′-O-methoxyethyl deoxyribose, 2′-O-methyl substituted ribose, a morpholino, a piperazine, and a locked nucleic acid (LNA).

8. The compound of claim 1, wherein said oligonucleotide comprises at least one modified backbone linkage selected from the group consisting of phosphorothioate linkages, methylphosphonate linkages, ethylphosphonate linkages, boranophosphate linkages, sulfonamide, carbonylamide, phosphorodiamidate, phosphorodiamidate linkages comprising a positively charged side group, phosphorodithioates, aminoethylglycine, phosphotriesters, aminoalkylphosphotriesters; 3′-alkylene phosphonates; 5′-alkylene phosphonates, chiral phosphonates, phosphinates, 3′-amino phosphoramidate, aminoalkylphosphoramidates, thionophosphoramidates; thionoalkyl-phosphonates, thionoalkylphosphotriesters, selenophosphates, 2′-5′ linked boranophosphonate analogs, linkages having inverted polarity, abasic linkages, short chain alkyl linkages, cycloalkyl internucleoside linkages, mixed heteroatom and alkyl or cycloalkyl internucleoside linkages, short chain heteroatomic or heterocyclic internucleoside linkages with siloxane backbones, sulfide, sulfoxide, sulfone, formacetyl linkages, thioformacetyl linkages, methylene formacetyl linkages, thioformacetyl linkages, riboacetyl linkages, alkene linkages, sulfamate backbones, methyleneimino linkages, methylenehydrazino linkages, sulfonate linkages, and amide linkages, said linkage optionally being present in an overhang precursor.

9. The compound of claim 1, wherein said oligo comprises at least one boranophosphate linkage.

10. The compound of claim 1 wherein said oligo comprises at least one 2′-fluoro or 2′-O-methyl substituted ribose.

11. The compound of claim 1, which having an architectural configuration selected from the group consisting of canonical, blunt-ended, asymmetric, forked variant, or small internally segmented configurations.

12. The compound of claim 11 which has a canonical configuration.

13. The compound of claim 11 which has a blunt ended configuration.

14. The compound of claim 11 which has an asymmetric configuration.

15. The compound of claim 11 which as small internally segmented configuration.

16. The compound of claim 6 wherein the sense and antisense strands comprise at least one overhang precursor consisting of 1 to 4 units at one or both ends and at least one modified linkage in said precursor.

17. The compound as claimed in claim 16 wherein said antisense strand has a 3′-end overhang precursor consisting of 1 to 4 units and at least one modified linkage in said precursor.

18. The compound of claim 1, comprising a targeting code of nucleosides in positions 10 and 11 from the 5′-end of the antisense strand and adjacent 3 nucleosides on either side of these, further comprising intervening linkages and at least one nuclease resistance modification set forth in Table 2.

19. The compound as claimed in claim 18, wherein the targeting code forms a hairpin in the strand that includes the targeting code in the hairpin duplex, thereby protecting the targeting code from degradation.

20. The compound as claimed in claim 18 which targets a messenger RNA for cleavage at the exact site to which the targeting code forms a complementary duplex.

21. The compound as claimed in claim 6, wherein said compound has a targeting code based on the nucleosides in positions 2 through 8 from the 5′-end of the antisense strand and which inhibits the expression of a group of messenger RNAs possessing sufficient complementarity at the 3′ end with that of said compound.

22. The compound as claimed in claim 21, wherein the targeting code is further protected by the formation of a hairpin in the strand that includes the targeting code in the hairpin duplex.

23. The compound as claimed in claim 21 wherein said targeting code within said compound comprises modifications selected from the group consisting of at least one modification listed in Table 2, the presence of which increasing the affinity of the targeting code sequence for its target sequence in the 3′-end untranslated region of said at least one target mRNA, wherein the corresponding sequence in the sense strand comprises mismatches with the targeting code of the antisense strand sequence, and further wherein there are no more than two affinity altering modifications in one strand relative to the other.

24. The compound as claimed in claim 21 where said targeting code and the corresponding sense strand sequence are inserted into the corresponding regions of a duplex vehicle where said duplex vehicle is selected from the group consisting of a particular endogenous miRNA duplex after dicer processing, an active siRNA duplex, a miRNA/siRNA negative control duplex capable of loading RISC.

25. The compound of claim 1, which targets p53.

26. A compound as claimed in claim 1, comprising a first and a second oligonucleotide for use in inhibiting expression of a target gene in a method of medical treatment, said method comprising the sequential administration of the first and second oligonucleotides such that the first oligonucleotide is taken up by a cell expressing the target gene before the second oligonucleotide, such that the first and the second oligonucleotides form a duplex intracellularly thereby triggering inhibition of gene target expression; wherein said first and/or said second oligonucleotides have been adapted to increase nuclease resistance.

27. A first and a second oligonucleotide according to claim 26 wherein said first oligonucleotide is truncated with respect to the second oligonucleotide.

28. A first and a second oligonucleotide according to claim 26 wherein said first oligonucleotide has a lower Tm between its 5′-end and its 3′-end.

29. A first and a second oligonucleotide according to claim 26 wherein said first oligonucleotide comprises an Argonaute 2 cleavage site.

30. A first and a second oligonucleotide according to claim 26 which form an overhang at the 3′ and/or 5′ end when the duplex assembles intracellularly.

31. A first and a second oligonucleotide according to claim 26 wherein two or more first oligonucleotides are provided as a contiguous sequence.

32. A first and a second oligonucleotide according to claim 26 wherein at least one of said first and second oligonucleotide is capable of forming a hairpin.

33. A first and a second oligonucleotide according to claim 26 wherein said first oligonucleotide is a sense strand and the second oligonucleotide is an antisense sense strand.

34. A first and a second oligonucleotide according to claim 26 wherein said first oligonucleotide is an antisense strand and the second oligonucleotide is a sense strand.

35. A formulation comprising the compound of claims 1 to 34, wherein the formulation is selected from the group consisting of oral, intrabuccal, intrapulmonary, intrathecal, rectal, intrauterine, intratumor, intracranial, nasal, intramuscular, subcutaneous, intravascular, intrathecal, inhalable, transdermal, intradermal, intracavitary, implantable, iontophoretic, ocular, vaginal, intraarticular, otical, intravenous, intramuscular, intraglandular, intraorgan, intralymphatic, implantable, slow release, and enteric coating formulations.

36. A method for down modulating expression of a nucleic acid sequence target in a cell or tissue of interest within a mammal, said method comprising
a) administering an effective amount of a compound of claims 1 to 34, and
b) optionally administering an augmentation agent selected from the group consisting of antioxidants, polyunsaturated fatty acids, chemotherapeutic agents, genome damaging agents, and ionizing radiation,
said compound being effective to
i) induce degradation of a target mRNA nucleic acid sequence; or
ii) inhibit translation of mRNA encoding a protein produced by said nucleic acid sequence; or
iii) induce degradation of a target miRNA nucleic acid sequence; or
iii) mimic the action of a miRNA target nucleic acid sequence;
in an RNAi dependent manner within said cell or tissue in vivo.

37. The method of claim 36, wherein said compound comprises two complementary strands, said method comprising
a) administering a first strand to said cell within a mammal and providing a suitable time period for cellular uptake of said first strand to occur,
b) administering a second strand to the cell of a), said first strand and said second strand forming an intracellular duplex which is effective to down modulate expression of said targeted nucleic acid sequence.

38. The method of claim 37, wherein step b) is carried out between about 4 and about 24 hours after step a).

39. The method of claim 37 for the treatment of disease, wherein said disease is selected from Cancer, AIDS, Alzheimer's disease, Amyotrophic lateral sclerosis, Atherosclerosis, Autoimmune Diseases, Cerebellar degeneration, Cancer, Diabetes Mellitus, Glomerulonephritis, Heart Failure, Macular Degeneration, Multiple sclerosis, Myelodysplastic syndromes, Parkinson's disease, Prostatic hyperplasia, Psoriasis, Asthma, Retinal Degeneration, Retinitis pigmentosa, Rheumatoid arthritis, Rupture of atherosclerotic plaques, Systemic lupus erythematosis, Ulcerative colitis, viral infection, ischemia reperfusion injury, spinal cord injury, nerve damage, cardiohypertrophy, and Diamond Black Fan anemia.

40. The method of claim 39, wherein said disease is cancer.

41. The method of claim 39, wherein said disease is AIDS.

42. An in vitro method of improving the an RNAi effect in vivo against a target gene, said method comprising;
(i) obtaining a first and a second oligonucleotide sequence capable of forming a duplex intracellularly;
(ii) modifying said first and/or said second oligonucleotide sequence to increase its nuclease resistance;
(iii) contacting said first oligonucleotide sequence with a cell expressing the target gene;
(iv) following step (iii) contacting said second oligonucleotide sequence with a said cell and;
(v) determine the expression of the target gene as compare to the expression of the target gene without step (ii).

43. An in vitro method according to claim 42 wherein said first oligonucleotide is truncated with respect to the second oligonucleotide.

44. An in vitro method according to claim 42 wherein said first oligonucleotide has a lower Tm between its 5′-end and its 3′-end.

45. An in vitro method according to claim 42 wherein said first oligonucleotide comprises an Argonaute 2 cleavage site.

46. An in vitro method according to claim 42 which are designed to create an overhang at the 3′ and/or 5′ end when the duplex is formed intracellularly.

47. An in vitro method according to claim 42 wherein two or more first oligonucleotides are provided as a contiguous sequence.

48. An in vitro method according to claim 42 wherein said first oligonucleotide is a sense strand and the second oligonucleotide is an antisense sense strand.

49. An in vitro method according to claim 42 wherein said oligonucleotides are selected from the group of oligonucleotides shown in the Figures.

50. A method for the treatment of a disorder listed in Table 6, 7 or 8 comprising administration of at least one oligonucleotide selected from the group consisting of oligonucleotides shown in the Figures.

51. A method for the treatment of a disorder mediated by a gene target listed in Table 6, 7 or 8 comprising sequential administration of at least two oligos selected from the group consisting of oligonucleotides shown in the Figures said oligos forming a duplex within a target cell, said duplex being effective to down modulate expression of said target gene, thereby ameliorating symptoms or treating said disorder.
